(19) United States
(12) Patent Application Publication
(10) Pub. No.: US 2023/0278984 A1
(43) Pub. Date: Sep. 7, 2023

(54) UREA DERIVATIVES AS PYRUVATE KINASE ACTIVATORS

(71) Applicant: Global Blood Therapeutics, Inc., South San Francisco, CA (US)

(72) Inventors: Lina Q. Setti, Fremont, CA (US); Brian Cathers, South San Fracisco, CA (US); Zhe Li, San Dirgo, CA (US); Shahul Nilar, Foster city, CA (US); James Partridge, South San Fracisco, CA (US); Chul Yu, Foster City, CA (US); Ming Yu, Foster City, CA (US); Manuel Zancanella, San Mateo, CA (US)

(21) Appl. No.: 18/019,379

(22) PCT Filed: Aug. 3, 2021
(86) PCT No.: PCT/US2021/044388

§ 371 Date: Feb. 2, 2023

Related U.S. Application Data
(60) Provisional application No. 63/060,345, filed on Aug. 3, 2020.

(51) Int. Cl. C07D 401/14; C07D 257/04; C07D 403/14; C07D 405/14; C07D 417/14; C07D 249/08

(52) U.S. Cl. CPC C07D 401/14; C07D 257/04; C07D 403/14; C07D 405/14; C07D 417/14; C07D 249/08

(57) ABSTRACT

The subject matter described herein is directed to pyruvate kinase activating compounds of Formula I and pharmaceutical salts thereof, methods of preparing the compounds, pharmaceutical compositions comprising the compounds and methods of administering the compounds for the treatment of diseases associated with PKR and/or PKM2, such as pyruvate kinase deficiency, sickle cell disease, and beta-thalassemia.

### CROSS REFERENCE TO RELATED APPLICATIONS

This application claims the benefit of and priority to U.S. Provisional Application No. 63/060,345, filed on Aug. 3, 2020, which is hereby incorporated by reference in its entirety.

### FIELD

The subject matter described herein is directed to pyruvate kinase activating compounds, methods of making the compounds, pharmaceutical compositions, and their use in the treatment of diseases associated with PKR and/or PKM2.

### BACKGROUND

Pyruvate kinase (PK) is an essential component of cellular metabolism, converting ADP and phosphoenolpyruvate (PEP) to pyruvate in the final step of glycolysis. There are four unique isoforms of pyruvate kinase that vary in concentration by different tissue types (Dayton et al., EMBO Rep. 2016 17(12):1721-1730); Israelsen and Vander Heiden, Semin. Cell Dev. Biol. 2015 July; 43:43-51; Mazurek, Int. J. Biochem. Cell Biol. 2011 July; 43(7):969-80). Each isomer is responsible for catalyzing the production of pyruvate and ATP, while being regulated in a manner respective to each tissue type.

Pyruvate kinase in the liver (PKL) and pyruvate kinase from erythrocytes/red blood cells (PKR) are tetrameric enzymes that depend on an endogenous activator called fructose-1,6-bisphosphate (FBP) for activation (Koler and Vanbellinghen, Adv. Enzyme Regul. 1968 6:127-42; Taylor and Bailey, Biochem J. 1967 967 February; 102(2):32C-33C). The PKM1 isoform is found in the brain, heart, and skeletal muscle where it functions as a stable and constitutively active tetrameric protein. PKM1 therefore does not require FBP for activation. The PKM2 isomer is expressed in most tissue types, including cancers, developing embryos, and all proliferative tissues. Similar to PKR and PKL, PKM2 requires FBP for allosteric activation via stabilization of the enzyme in a tetrameric and most active form (Cardenas and Dyson, J. Exp. Zool. 1978 June; 204(3):361-7; Imamura and Tanaka, J. Biochem. 1972 June; 71(6):1043-51; Strandholm et al., Arch. Biochem. Biophys. 1976 March; 173(1):125-31).

Mature red blood cells (RBCs) rely on glycolysis for energy production. All tumor cells exclusively express the PKM2 isoform, suggesting that PKM2 would be a good target for cancer therapy. PKM2 is also expressed in adipose tissue and activated T-cells. Thus, controlling the regulation of PKM2 activity may be effective for treatment of obesity and diabetes in addition to cancer.

Genetic mutations in all glycolytic enzymes result in hemolytic anemia (Van Wijk and Van Solinge, Blood 2005 106 (13): 4034-4042.). Mutations in PKL and PKR that result in a loss of function are known to cause PK deficiency (PKD) and the clinical manifestation of these mutations appear confined to RBCs.

There are greater than 200 known and reported mutations associated with PKD reported worldwide (Zanella et al., J. Haematol. 2005 July; 130(1):11-25). Some mutations directly disrupt catalytic activity of the PK enzyme while other mutations disrupt the interactions between monomers that stabilize the active tetrameric enzyme. The mutation of Arginine residue 510 to Glutamine is one of the most common mutations found in North American and European patients, ˜40% of patients, and is known to disrupt stability of the PKR tetramer (Kedar et al., Clin. Genet. 2009 February; 75(2):157-62; Wang et al., Blood 2001 Nov. 15; 98(10):3113-20).

Patients with PKD suffer from chronic hemolytic anemia in addition to multiple co-morbidities. Blood transfusions and splenectomy are common treatments and it has been suggested that gene therapies could be used for treatment of PKD in the near future (Garcia-Gomez et al., Molecular Therapy 2016 Aug. 1; 24(7); Grace et al., Am. J. Hematol. 2015 September; 90(9):825-30).

The number of PKD patients worldwide is unknown; however, the prevalence in the general Caucasian population is estimated to be around 1:20,000 people with 51 cases per million people in North America (Beutler and Gelbart, Blood 2000 Jun. 1; 95(11):3585-8).

There are no approved drugs for the treatment of PKD. Clinically, hereditary PKR deficiency disorder manifests as a non-spherocytic hemolytic anemia. The clinical severity of this disorder ranges from no observable symptoms in fully-compensated hemolysis to potentially fatal severe anemia requiring chronic transfusions and/or splenectomy at early development or during physiological stress. For some of the most severe cases, while extremely rare population-wise with estimated frequency of 1 in 20,000 patients, there is no disease modifying treatment besides transfusions. These hereditary non-spherocytic hemolytic anemia patients present a clear unmet medical need. RBCs from patients with either sickle cell anemia or with beta-thalassemia suffer from increased ATP demand to maintain overall RBC health. The activation of PKR in both sickle cell disease patients and beta-thalassemia patients could lead to improved cell fitness and survival.

What is therefore needed and not effectively addressed by the art are compounds that act as pyruvate kinase activators that have desired efficacy and therapeutic potential. This problem as well as others stemming from pyruvate kinase deficiency are addressed by the subject matter described herein.

### BRIEF SUMMARY

In certain embodiments, the subject matter described herein is directed to a compound of Formula I or a pharmaceutically acceptable salt thereof.

In certain embodiments, the subject matter described herein is directed to a pharmaceutical composition comprising a compound of Formula I or a pharmaceutically acceptable salt thereof.

In certain embodiments, the subject matter described herein is directed to a method of treating a disease or disorder associated with modulation of a pyruvate kinase in a subject, comprising administering to the subject an effective amount of a compound of Formula I, a pharmaceutically acceptable salt thereof, or a pharmaceutical composition comprising a compound of Formula I.

In certain embodiments, the subject matter described herein is directed to a method of activating PKR and/or PKM2 in a subject, comprising administering to the subject an effective amount of a compound of Formula I, a pharmaceutically acceptable salt thereof, or a pharmaceutical composition comprising a compound of Formula I.

In certain embodiments, the subject matter described herein is directed to a method of treating a subject afflicted with a disease associated with decreased activity of PKR and/or PKM2, comprising administering to the subject an effective amount of a compound of Formula I, a pharmaceutically acceptable salt thereof, or a pharmaceutical composition comprising a compound of Formula I.

In certain embodiments, the subject matter described herein is directed to a method for regulating 2,3-diphosphoglycerate levels in blood comprising contacting the blood with an effective amount of a compound of Formula I, a pharmaceutically acceptable salt thereof, or a pharmaceutical composition comprising a compound of Formula I.

In certain embodiments, the subject matter described herein is directed to a method for activating mutant pyruvate kinase R (PKR) in red blood cells in a subject in need thereof comprising administering to the subject an effective amount of a compound of Formula I, a pharmaceutically acceptable salt thereof, or a pharmaceutical composition comprising a compound of Formula I.

In certain embodiments, the subject matter described herein is directed to a method for activating wild-type pyruvate kinase R (PKR) in red blood cells in a subject in need thereof comprising administering to the subject an effective amount of a compound of Formula I, a pharmaceutically acceptable salt thereof, or a pharmaceutical composition comprising a compound of Formula I.

Other embodiments are also described.

### DETAILED DESCRIPTION

Described herein are pyruvate kinase activators of Formula I, methods of making the compounds, pharmaceutical compositions comprising the compounds, and their use in the treatment of diseases associated with decreased pyruvate kinase activity.

PKR activating compounds could be used to treat patients with beta-thalassemia and sickle cell anemia (Alli et al., Hematology 2008 December; 13(6):369-72; Kung et al., Blood 2017 Sep. 14; 130(11):1347-135). As shown in a mouse model of beta-thalassemia, the PKR activator in clinical trials, AG-348, increased PK activity and ATP levels, as well as improved RBC parameters. Similar results were obtained from treating human beta-thalassemia RBCs ex vivo (Kuo et al., Mitapivat (AG-348), an oral PK-R activator, in adults with non-transfusion-dependent thalassemia: A phase 2, open-label, multicenter study in progress; 61^{st} Am. Soc. Hematol. Annual Meeting, December 2019).

The compounds of Formula I described herein are useful in the treatment of diseases or disorders associated with pyruvate kinase function. As demonstrated by the biochemical assays described herein, the compounds of Formula I activate PKR and/or PKM2. In certain embodiments, the compounds described herein are more effective at activating PKR and/or PKM2 than AG-348. The compounds of Formula I are useful in the treatment of diseases including, but not limited to, pyruvate kinase deficiency and sickle cell disease, such as sickle cell anemia, and beta-thalassemia. Also, the compounds are methods described herein are useful in treating cancer.

Pyruvate kinase activators are needed that also possess additional beneficial properties such as improved solubility, stability, and/or potency. An advantage of the pyruvate kinase activator compounds of Formula I described herein is their preparation in sufficient yields by the synthetic routes disclosed herein.

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. Unless otherwise defined, all technical and scientific terms used herein have the same meaning as commonly understood by one of ordinary skill in this field. All publications, patent applications, patents, and other references mentioned herein are incorporated by reference in their entirety. In the event that one or more of the incorporated literature, patents, and similar materials differs from or contradicts this application, including but not limited to defined terms, term usage, described techniques, or the like, this application controls.

### I. Definitions

As used in the present specification, the following words, phrases and symbols are generally intended to have the meanings as set forth below, except to the extent that the context in which they are used indicates otherwise.

A dash (“-”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line or a dashed line drawn through or perpendicular across the end of a line in a structure indicates a specified point of attachment of a group. Unless chemically or structurally required, no directionality or stereochemistry is indicated or implied by the order in which a chemical group is written or named.

The prefix “Cᵤ-Cᵥ” indicates that the following group has from u to v carbon atoms. For example, “C₁-C₆ alkyl” indicates that the alkyl group has from 1 to 6 carbon atoms.

Reference to “about” a value or parameter herein includes (and describes) embodiments that are directed to that value or parameter per se. In certain embodiments, the term “about” includes the indicated amount ±50%. In certain other embodiments, the term “about” includes the indicated amount ±20%. In certain other embodiments, the term “about” includes the indicated amount ±10%. In other embodiments, the term “about” includes the indicated amount ±5%. In certain other embodiments, the term “about” includes the indicated amount ±1%. In certain other embodiments, the term “about” includes the indicated amount ±0.5% and in certain other embodiments, 0.1%. Such variations are appropriate to perform the disclosed methods or employ the disclosed compositions. Also, to the term “about x” includes description of “x”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

“Alkyl” refers to an unbranched or branched saturated hydrocarbon chain. As used herein, alkyl has 1 to 20 carbon atoms (i.e., C₁-C₂₀ alkyl), 1 to 12 carbon atoms (i.e., C₁-C₁₂ alkyl), 1 to 8 carbon atoms (i.e., C₁-C₈ alkyl), 1 to 6 carbon atoms (i.e., C₁-C₆ alkyl), 1 to 4 carbon atoms (i.e., C₁-C₄ alkyl), or 1 to 3 carbon atoms (i.e., C₁-C₃ alkyl). Examples of alkyl groups include, e.g., methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl and 3-methylpentyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), sec-butyl (i.e., —CH(CH₃)CH₂CH₃), isobutyl (i.e., —CH₂CH(CH₃)₂) and tert-butyl (i.e., —C(CH₃)₃); and “propyl” includes n-propyl (i.e., —(CH₂)₂CH₃) and isopropyl (i.e., —CH(CH₃)₂).

Certain commonly used alternative chemical names may be used. For example, a divalent group such as a divalent “alkyl” group, a divalent “aryl” group, etc., may also be referred to as an “alkylene” group or an “alkylenyl” group, an “arylene” group or an “arylenyl” group, respectively. Also, unless indicated explicitly otherwise, where combinations of groups are referred to herein as one moiety, e.g., arylalkyl or aralkyl, the last mentioned group contains the atom by which the moiety is attached to the rest of the molecule.

“Alkoxy” refers to the group “alkyl-O” (e.g., C₁-C₃ alkoxy or C₁-C₆ alkoxy). Examples of alkoxy groups include, e.g., methoxy, ethoxy, n-propoxy, iso-propoxy, n-butoxy, tert-butoxy, sec-butoxy, n-pentoxy, n-hexoxy and 1,2-dimethylbutoxy.

“Alkoxyalkyl” refers to the group “alkoxy-alkyl-” (e.g., C₁-C₆ alkoxy-C₁-C₆ alkyl). “C₁-C₆ alkoxy-C₁-C₆ alkyl” refers to an alkyl chain containing 1 to 6 carbon atoms where one of the hydrogens on a carbon atom is replaced with an alkoxy group having one to six carbon atoms.

“Alkylthio” refers to the group “alkyl-S—”.

“Amino” refers to the group —NR^{y}R^{z} wherein R^{y} and R^{z} are independently hydrogen, alkyl, cycloalkyl, heterocyclyl, aryl, or heteroaryl; each of which may be optionally substituted, as defined herein.

“Aryl” refers to an aromatic carbocyclic group having a single ring (e.g., monocyclic) or multiple rings (e.g., bicyclic or tricyclic) including fused systems. As used herein, aryl has 6 to 20 ring carbon atoms (i.e., C₆-C₂₀ aryl), 6 to 12 carbon ring atoms (i.e., C₆-C₁₂ aryl), or 6 to 10 carbon ring atoms (i.e., C₆-C₁₀ aryl). Examples of aryl groups include, e.g., phenyl, naphthyl, fluorenyl and anthryl. Aryl, however, does not encompass or overlap in any way with heteroaryl defined below. If one or more aryl groups are fused with a heteroaryl, the resulting ring system is heteroaryl. If one or more aryl groups are fused with a heterocyclyl, the resulting ring system is heterocyclyl.

“Arylalkyl” or “Aralkyl” refers to the group “aryl-alkyl-”, such as (C₆-C₁₀ aryl)-C₁-C₃ alkyl. A non-limiting example of arylalkyl is benzyl.

“Carboxyl ester” or “ester” refer to both —OC(O)R^{x} and —C(O)OR^{x}, wherein R^{x} is alkyl, cycloalkyl, heterocyclyl, aryl, or heteroaryl; each of which may be optionally substituted, as defined herein.

“Cycloalkyl” refers to a saturated or partially unsaturated cyclic alkyl group having a single ring or multiple rings including fused, bridged and spiro ring systems. The term “cycloalkyl” includes cycloalkenyl groups (i.e., the cyclic group having at least one double bond) and carbocyclic fused ring systems having at least one spa carbon atom (i.e., at least one non-aromatic ring). As used herein, cycloalkyl has from 3 to 20 ring carbon atoms (i.e., C₃-C₂₀ cycloalkyl), 3 to 12 ring carbon atoms (i.e., C₃-C₁₂ cycloalkyl), 3 to 10 ring carbon atoms (i.e., C₃-C₁₀ cycloalkyl), 3 to 8 ring carbon atoms (i.e., C₃-C₈ cycloalkyl), 3 to 7 ring carbon atoms (i.e., C₃-C₇ cycloalkyl), or 3 to 6 ring carbon atoms (i.e., C₃-C₆ cycloalkyl). Monocyclic groups include, for example, cyclopropyl, cyclobutyl, cyclopentyl, cyclohexyl, cycloheptyl and cyclooctyl. Polycyclic groups include, for example, bicyclo[2.2.1]heptanyl, bicyclo[2.2.2]octanyl, adamantyl, norbornyl, decalinyl, 7,7-dimethyl-bicyclo[2.2.1]heptanyl and the like. Further, the term cycloalkyl is intended to encompass any moiety comprising a non-aromatic alkyl ring which may be fused to an aryl ring, regardless of the attachment to the remainder of the molecule. Still further, cycloalkyl also includes “spirocycloalkyl” when there are two positions for substitution on the same carbon atom, for example spiro[2.5]octanyl, spiro[4.5]decanyl, or spiro[5.5]undecanyl.

“Cycloalkylalkyl” refers to the group “cycloalkyl-alkyl-”, such as (C₃-C₆ cycloalkyl)-C₁-C₃ alkyl.

“Halogen” or “halo” refers to atoms occupying group VIIA of the periodic table, such as fluoro, chloro, bromo or iodo.

“Haloalkyl” refers to an unbranched or branched alkyl group as defined above, wherein one or more (e.g., 1 to 6, or 1 to 3) hydrogen atoms are replaced by a halogen. For example, halo-C₁-C₃ alkyl or C₁-C₃ haloalkyl refers to an alkyl group of 1 to 3 carbons wherein at least one hydrogen atom is replaced by a halogen. Halo-C₁-C₆ alkyl or C₁-C₆ haloalkyl refers to an alkyl group of 1 to 6 carbons wherein at least one hydrogen atom is replaced by a halogen. Where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl and the like.

“Haloalkoxy” refers to an alkoxy group as defined above, wherein one or more (e.g., 1 to 6, or 1 to 3) hydrogen atoms are replaced by a halogen. For example, halo-C₁-C₃ alkoxy or C₁-C₃ haloalkoxy refers to an alkoxy group of 1 to 3 carbons wherein at least one hydrogen atom is replaced by a halogen. Halo-C₁-C₆ alkoxy or C₁-C₆ haloalkoxy refers to an alkoxy group of 1 to 6 carbons wherein at least one hydrogen atom is replaced by a halogen.

“Hydroxyalkyl” refers to an alkyl group as defined above, wherein one or more (e.g., 1 to 6, or 1 to 3) hydrogen atoms are replaced by a hydroxy group (e.g., hydroxy-C₁-C₃-alkyl, hydroxy-C₁-C₆-alkyl). The term “hydroxy-C₁-C₃ alkyl” refers to a one to three carbon alkyl chain where one or more hydrogens on any carbon is replaced by a hydroxy group, in particular, one hydrogen on one carbon of the chain is replaced by a hydroxy group. The term “hydroxy-C₁-C₆ alkyl” refers to a one to six carbon alkyl chain where one or more hydrogens on any carbon is replaced by a hydroxy group, in particular, one hydrogen on one carbon of the chain is replaced by a hydroxy group. Non-limiting examples of hydroxyalkyl include —CH₂OH, —CH₂CH₂OH, and —C(CH₃)₂CH₂OH.

“Heteroaryl” refers to an aromatic group having a single ring, multiple rings or multiple fused rings, with one or more ring heteroatoms independently selected from nitrogen, oxygen, and sulfur. As used herein, heteroaryl includes 1 to 20 ring carbon atoms (i.e., C₁-C₂₀ heteroaryl), 3 to 12 ring carbon atoms (i.e., C₃-C₁₂ heteroaryl), or 3 to 8 carbon ring atoms (i.e., C₃-C₈ heteroaryl), and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen and sulfur. In certain instances, heteroaryl includes 9-10 membered ring systems, 6-10 membered ring systems, 5-10 membered ring systems, 5-7 membered ring systems, or 5-6 membered ring systems, each independently having 1 to 4 ring heteroatoms, 1 to 3 ring heteroatoms, 1 to 2 ring heteroatoms, or 1 ring heteroatom independently selected from nitrogen, oxygen and sulfur. Examples of heteroaryl groups include, e.g., acridinyl, benzimidazolyl, benzothiazolyl, benzindolyl, benzofuranyl, benzothiazolyl, benzothiadiazolyl, benzonaphthofuranyl, benzoxazolyl, benzothienyl (benzothiophenyl), benzotriazolyl, benzo[4,6]imidazo[1,2-a]pyridyl, carbazolyl, cinnolinyl, dibenzofuranyl, dibenzothiophenyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, triazolyl, tetrazolyl and triazinyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl and imidazo[1,5-a]pyridinyl, where the heteroaryl can be bound via either ring of the fused system. Any aromatic group, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heterocyclyl” refers to a saturated or partially unsaturated cyclic alkyl group, with one or more ring heteroatoms independently selected from nitrogen, oxygen and sulfur. The term “heterocyclyl” includes heterocycloalkenyl groups (i.e., the heterocyclyl group having at least one double bond), bridged-heterocyclyl groups, fused-heterocyclyl groups and spiro-heterocyclyl groups. A heterocyclyl may be a single ring or multiple rings wherein the multiple rings may be fused, bridged or spiro. Any non-aromatic ring containing at least one heteroatom is considered a heterocyclyl, regardless of the attachment (i.e., can be bound through a carbon atom or a heteroatom). Further, the term heterocyclyl is intended to encompass a moiety comprising any non-aromatic ring containing at least one heteroatom, which ring may be fused to an aryl or heteroaryl ring, regardless of the attachment to the remainder of the molecule. The term heterocyclyl is also intended to encompass a moiety comprising a cycloalkyl ring which is fused to a heteroaryl ring, regardless of the attachment to the remainder of the molecule. Additionally, the term heterocyclyl is intended to encompass a moiety comprising a cycloalkyl ring which is fused to a heterocyclyl ring, regardless of the attachment to the remainder of the molecule. As used herein, heterocyclyl has 2 to 20 ring carbon atoms (i.e., C₂-C₂₀ heterocyclyl), 2 to 12 ring carbon atoms (i.e., C₂-C₁₂ heterocyclyl), 2 to 10 ring carbon atoms (i.e., C₂-C₁₀ heterocyclyl), 2 to 8 ring carbon atoms (i.e., C₂-C₈ heterocyclyl), 3 to 12 ring carbon atoms (i.e., C₃-C₁₂ heterocyclyl), 3 to 8 ring carbon atoms (i.e., C₃-C₈ heterocyclyl), or 3 to 6 ring carbon atoms (i.e., C₃-C₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur or oxygen. When the heterocyclyl ring contains 4- or 6-ring atoms, it is also referred to herein as a 4- or 6-membered heterocyclyl. When the heterocyclyl ring contains 5- to 7-ring atoms, it is also referred to herein as a 5- to 7-membered heterocyclyl. When the heterocyclyl ring contains 5- to 10-ring atoms, it is also referred to herein as a 5- to 10-membered heterocyclyl. Examples of heterocyclyl groups include, e.g., azetidinyl, azepinyl, benzodioxolyl, benzo[b][1,4]dioxepinyl, 1,4-benzodioxanyl, benzopyranyl, benzodioxinyl, benzopyranonyl, benzofuranonyl, dioxolanyl, dihydropyranyl, hydropyranyl, thienyl[1,3]dithianyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, oxetanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, thiophenyl (i.e., thienyl), tetrahydropyranyl, thiomorpholinyl, thiamorpholinyl, 1-oxo-thiomorpholinyl and 1,1-dioxo-thiomorpholinyl. A nonlimiting example of a partially unsaturated heterocyclyl is 4,5-dihydro-1H-1,2,4-triazolyl, which can be further substituted, in certain embodiments, one or more times, with, for example, an oxo group and/or an alkyl group. The term “heterocyclyl” also includes “spiroheterocyclyl” when there are two positions for substitution on the same carbon atom, wherein at least one ring of the spiro system comprises at least one heteroatom. Examples of the spiro-heterocyclyl rings include, e.g., bicyclic and tricyclic ring systems, such as 2-oxa-7-azaspiro[3.5]nonanyl, 2-oxa-6-azaspiro[3.4]octanyl and 6-oxa-1-azaspiro[3.3]heptanyl.

Further nonlimiting examples of spirocyclic heterocyclyls are 4,6-diazaspiro[2.4]hept-4-enyl and 5,7-diazaspiro[3.4]oct-5-enyl, which can be further substituted, in certain embodiments, for example, with an oxo group.

Examples of the fused-heterocyclyl rings include, but are not limited to, 1,2,3,4-tetrahydroisoquinolinyl, 4,5,6,7-tetrahydrothieno[2,3-c]pyridinyl, indolinyl and isoindolinyl, where the heterocyclyl can be bound via either ring of the fused system.

As used herein, “oxo” refers to the group “=0.”

Non-limiting examples of an oxo group formed by an oxygen double bonded to a ring carbon are shown below:

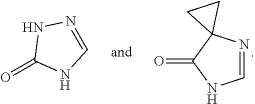 [SMILES: O=c1[nH]cn[nH]1 ; O=C1NC=NC12CC2]

As used herein, “N-oxide” refers to the group “═O”.

The terms “optional” or “optionally” means that the subsequently described event or circumstance may or may not occur and that the description includes instances where said event or circumstance occurs and instances in which it does not. Also, the term “optionally substituted” refers to any one or more (e.g., 1 to 5, 1 to 4, or 1 to 3) hydrogen atoms on the designated atom or group may or may not be replaced by a moiety other than hydrogen.

The term “substituted” used herein means any of the above groups (i.e., alkyl, alkylene, alkoxy, haloalkyl, haloalkoxy, cycloalkyl, aryl, heterocyclyl, and/or heteroaryl) wherein at least one (e.g., 1 to 5, 1 to 4, or 1 to 3) hydrogen atom is replaced by a bond to a non-hydrogen atom such as, but not limited to alkyl, alkoxy, amino, aryl, aralkyl, carboxyl, carboxyl ester, cyano, cycloalkyl, halo, haloalkyl, haloalkoxy, hydroxyalkyl, heteroaryl, heterocyclyl, —NHNH₂, hydroxy, oxo, nitro, —S(O)OH, —S(O)₂OH, N-oxide or —Si(R^{y})₃, wherein each R^{y} is independently hydrogen, alkyl, alkenyl, alkynyl, heteroalkyl, cycloalkyl, aryl, heteroaryl or heterocyclyl.

In certain embodiments, “substituted” includes any of the above alkyl, alkenyl, alkynyl, cycloalkyl, heterocyclyl, aryl or heteroaryl groups in which one or more (e.g., 1 to 5, 1 to 4, or 1 to 3) hydrogen atoms are independently replaced with deuterium, halo, cyano, nitro, oxo, alkyl, haloalkyl, cycloalkyl, heterocyclyl, aryl, heteroaryl, —NR^{g}R^{h}, —NR^{g}C(═O)R^{h}, —NR^{g}C(═O)NR^{g}R^{h}, —NR^{g}C(═O)OR^{h}, —NR^{g}S(═O)₁₋₂ R^{h}, —C(═O)R^{g}, —C(═O)OR^{g}, —OC(═O)OR^{g}, —OC(═O)R^{g}, —C(═O)NR^{g}R^{h}, —OC(═O)NR^{g}R^{h}, —OR^{g}, —SR^{g}, —S(═O)R^{g}, —S(═O)₂R^{g}, —OS(═O)₁₋₂R^{g}, —S(═O)₁₋₂OR^{g}, —NR^{g}S(═O)₁₋₂NR^{g}R^{h}, ═NSO₂R^{g}, ═NOR^{g}, —S(═O)₁₋₂NR^{g}R^{h}, —SF₅, —SCF₃ or —OCF₃. In certain embodiments, “substituted” also means any of the above groups in which one or more (e.g., 1 to 5, 1 to 4, or 1 to 3) hydrogen atoms are replaced with —C(═O)R^{g}, —C(═O)OR^{g}, —C(═O)NR^{g}R^{h}, —CH₂SO₂R^{g}, or —CH₂SO₂NR^{g}R^{h}. In the foregoing, R^{g} and R^{h} are the same or different and independently hydrogen, alkyl, alkoxy, aryl, cycloalkyl, haloalkyl, heterocyclyl, and/or heteroaryl. In certain embodiments, “substituted” also means any of the above groups in which one or more (e.g., 1 to 5, 1 to 4, or 1 to 3) hydrogen atoms are replaced by a bond to an amino, cyano, hydroxyl, nitro, oxo, halo, alkyl, alkoxy, alkylamino, aryl, cycloalkyl, haloalkyl, heterocyclyl, N-heterocyclyl, heteroaryl, or two of R^{g} and R^{h} and R′ are taken together with the atoms to which they are attached to form a heterocyclyl ring optionally substituted with oxo, halo or alkyl optionally substituted with oxo, halo, amino, hydroxyl, or alkoxy.

Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl)substituted aryl) substituted aryl. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan. When used to modify a chemical group, the term “substituted” may describe other chemical groups defined herein.

In certain embodiments, as used herein, the phrase “one or more” refers to one to five. In certain embodiments, as used herein, the phrase “one or more” refers to one to four. In certain embodiments, as used herein, the phrase “one or more” refers to one to three.

Any compound or structure given herein, is intended to represent unlabeled forms as well as isotopically labeled forms (isotopologues) of the compounds. These forms of compounds may also be referred to as and include “isotopically enriched analogs.” Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine and iodine such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Various isotopically labeled compounds of the present disclosure, for example those into which radioactive isotopes such as ³H, ¹³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The term “isotopically enriched analogs” includes “deuterated analogs” of compounds described herein in which one or more hydrogens is/are replaced by deuterium, such as a hydrogen on a carbon atom. Such compounds exhibit increased resistance to metabolism and are thus useful for increasing the half-life of any compound when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F, ³H, ¹¹C labeled compound may be useful for PET or SPECT or other imaging studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in a compound described herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium. Further, in some embodiments, the corresponding deuterated analog is provided.

In many cases, the compounds of this disclosure are capable of forming acid and/or base salts by virtue of the presence of amino and/or carboxyl groups or groups similar thereto.

Provided also are a pharmaceutically acceptable salt, isotopically enriched analog, deuterated analog, isomer (such as a stereoisomer), mixture of isomers (such as a mixture of stereoisomers), prodrug, and metabolite of the compounds described herein.

“Pharmaceutically acceptable” or “physiologically acceptable” refer to compounds, salts, compositions, dosage forms and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include, e.g., hydrochloric acid, hydrobromic acid, sulfuric acid, nitric acid, phosphoric acid and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid and the like. Likewise, pharmaceutically acceptable base addition salts can be prepared from inorganic and organic bases. Salts derived from inorganic bases include, by way of example only, sodium, potassium, lithium, aluminum, ammonium, calcium and magnesium salts. Salts derived from organic bases include, but are not limited to, salts of primary, secondary and tertiary amines, such as alkyl amines (i.e., NH₂(alkyl)), dialkyl amines (i.e., HN(alkyl)₂), trialkyl amines (i.e., N(alkyl)₃), substituted alkyl amines (i.e., NH₂(substituted alkyl)), di(substituted alkyl) amines (i.e., HN(substituted alkyl)₂), tri(substituted alkyl) amines (i.e., N(substituted alkyl)₃), alkenyl amines (i.e., NH₂(alkenyl)), dialkenyl amines (i.e., HN(alkenyl)₂), trialkenyl amines (i.e., N(alkenyl)₃), substituted alkenyl amines (i.e., NH₂(substituted alkenyl)), di(substituted alkenyl) amines (i.e., HN(substituted alkenyl)₂), tri(substituted alkenyl) amines (i.e., N(substituted alkenyl)₃, mono-, di- or tri-cycloalkyl amines (i.e., NH₂(cycloalkyl), HN(cycloalkyl)₂, N(cycloalkyl)₃), mono-, di- or tri-arylamines (i.e., NH₂(aryl), HN(aryl)₂, N(aryl)₃) or mixed amines, etc. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine and the like.

The term “hydrate” refers to the complex formed by the combining of a compound described herein and water.

A “solvate” refers to an association or complex of one or more solvent molecules and a compound of the disclosure. Examples of solvents that form solvates include, but are not limited to, water, isopropanol, ethanol, methanol, dimethylsulfoxide, ethylacetate, acetic acid and ethanolamine.

Some of the compounds exist as tautomers. Tautomers are in equilibrium with one another. For example, amide containing compounds may exist in equilibrium with imidic acid tautomers. Regardless of which tautomer is shown and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

The compounds of the present disclosure, or their pharmaceutically acceptable salts include an asymmetric center and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)—, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centres of geometric asymmetry, and unless specified otherwise, it is intended that the compounds include both E and Z geometric isomers.

A “stereoisomer” refers to a compound made up of the same atoms bonded by the same bonds but having different three-dimensional structures, which are not interchangeable. The present disclosure contemplates various stereoisomers and mixtures thereof and includes “enantiomers,” which refers to two stereoisomers whose molecules are nonsuperimposeable mirror images of one another.

“Diastereomers” are stereoisomers that have at least two asymmetric atoms, but which are not mirror-images of each other.

Relative centers of the compounds as depicted herein are indicated graphically using the “thick bond” style (bold or parallel lines) and absolute stereochemistry is depicted using wedge bonds (bold or parallel lines).

“Prodrugs” means any compound which releases an active parent drug according to a structure described herein in vivo when such prodrug is administered to a mammalian subject. Prodrugs of a compound described herein are prepared by modifying functional groups present in the compound described herein in such a way that the modifications may be cleaved in vivo to release the parent compound. Prodrugs may be prepared by modifying functional groups present in the compounds in such a way that the modifications are cleaved, either in routine manipulation or in vivo, to the parent compounds. Prodrugs include compounds described herein wherein a hydroxy, amino, carboxyl, or sulfhydryl group in a compound described herein is bonded to any group that may be cleaved in vivo to regenerate the free hydroxy, amino, or sulfhydryl group, respectively. Examples of prodrugs include, but are not limited to esters (e.g., acetate, formate and benzoate derivatives), amides, guanidines, carbamates (e.g., N,N-dimethylaminocarbonyl) of hydroxy functional groups in compounds described herein and the like. Preparation, selection and use of prodrugs is discussed in T. Higuchi and V. Stella, “Pro-drugs as Novel Delivery Systems,” Vol. 14 of the A.C.S. Symposium Series; “Design of Prodrugs,” ed. H. Bundgaard, Elsevier, 1985; and in Bioreversible Carriers in Drug Design, ed. Edward B. Roche, American Pharmaceutical Association and Pergamon Press, 1987, each of which are hereby incorporated by reference in their entirety.

The term, “metabolite,” as used herein refers to a resulting product formed when a compound disclosed herein is metabolized. As used herein, the term “metabolized” refers to the sum of processes (including but not limited to hydrolysis reactions and reactions catalyzed by enzymes) by which a particular substance, such as a compound disclosed herein, is changed by an organism. For example, an aldehyde moiety (—C(O)H) of the compounds of the present disclosure may be reduced in vivo to a —CH₂OH moiety.

As used herein, the term “activator” refers to a compound of Formula I or a pharmaceutically acceptable salt thereof that increases the activity of pyruvate kinase R (PKR) or pyruvate kinase M2 (PKM2), unless specified otherwise. By “activate” herein is meant to increase the activity of PKR or PKM2 activity to a level that is greater than the basal levels of activity for PKR or PKM2 in the presence of the compound. In some embodiments, the term “activate” means an increase in the activity of PKR or PKM2 of at least about 5%, at least about 10%, at least about 20%, at least about 25%, at least about 50%, at least about 60%, at least about 70%, at least about 80%, at least about 90%, or at least about 95%. In other embodiments, activate means an increase in PKR or PKM2 activity of about 5% to about 25%, about 25% to about 50%, about 50% to about 75%, or about 75% to 100%. In some embodiments, activate means an increase in PKR or PKM2 activity of about 95% to 100%, e.g., an increase in activity of 95%, 96%, 97%, 98%, 99%, or 100%. Such increases can be measured using a variety of techniques that would be recognizable by one of skill in the art, including in vitro assays.

As used herein, the term “pyruvate kinase activator” and the like refers to a compound that activates, increases, or modulates one or more of the biological activities of pyruvate kinase. The activity could increase, for example, at least about 5%, 10%, 15%, 20%, 25%, 30%, 35%, 40%, 45%, 50%, 55%, 60%, 65%, 70%, 75%, 80%, 85%, 95% or 100% of the activity of pyruvate kinase compared to an appropriate control. The increase can be a statistically significant increase.

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy and/or veterinary applications. In some embodiments, the subject is a mammal. In one embodiment, the subject is a human.

The term “therapeutically effective amount” or “effective amount” of a compound described herein or a pharmaceutically acceptable salt, tautomer, stereoisomer, mixture of stereoisomers, prodrug, or deuterated analog thereof means an amount sufficient to effect treatment when administered to a subject, to provide a therapeutic benefit such as amelioration of symptoms or slowing of disease progression. For example, a therapeutically effective amount may be an amount sufficient to decrease a symptom of a pyruvate kinase deficiency (PKD). The therapeutically effective amount may vary depending on the subject, and disease or condition being treated, the weight and age of the subject, the severity of the disease or condition, and the manner of administering, which can readily be determined by one of ordinary skill in the art.

Additional definitions may also be provided below as appropriate.

### II. Compounds

In certain embodiments, the subject matter described herein is directed to compounds of Formula I, or pharmaceutically acceptable salts thereof:

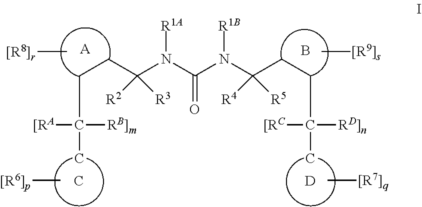

wherein:

m and n are each independently 0, 1, 2, or 3;

r and s are each independently 0, 1, 2, or 3;

p and q are each independently 0, 1, 2, 3, 4, or 5;

R^{A}, R^{B}, R^{C}, and R^{D}, in each instance, are independently selected from the group consisting of hydrogen, halogen, C₁-C₆ alkyl, hydroxy, hydroxy-C₁-C₆ alkyl, and C₁-C₆ alkoxy;

R⁶ and R⁷, in each instance, are independently selected from the group consisting of halogen, C₁-C₆ alkoxy, C₃-C₇ cycloalkyl, C₁-C₆ alkyl, —C(O)OR¹⁰, hydroxy, hydroxy-C₁-C₆ alkyl, halo-C₁-C₆ alkoxy, halo-C₁-C₆ alkyl, oxo, NR^{E1}R^{G1}, and —C₁-C₆ alkyl-NR^{x}R^{y};
wherein, R¹⁰, R^{E1}, R^{G1}, R^{x}, and R^{y} are each independently hydrogen or C₁-C₆ alkyl;

Ring C and Ring D are each independently selected from the group consisting of C₄-C₇ cycloalkyl, 4- to 10-membered monocyclic or bicyclic fused heterocyclyl, 6- to 10-membered aryl, and 5- to 10-membered heteroaryl,
wherein said 4- to 10-membered monocyclic or bicyclic fused heterocyclyl or 5- to 10-membered heteroaryl each independently contains 1, 2, 3, or 4 ring heteroatoms selected from N, O, and S;

R^{1A} and R^{1B} are each independently selected from the group consisting of hydrogen, C₁-C₆ alkyl, halogen, halo-C₁-C₆ alkyl, hydroxy-C₁-C₆ alkyl, hydroxy, C₁-C₆ alkoxy, and C₁-C₆ alkoxy-C₁-C₆ alkyl;

or, R^{1A} and R^{1B}, together with the nitrogen atom to which each is attached form a 5- to 7-membered heterocyclyl;

R², R³, R⁴, and R⁵ are each independently hydrogen, halogen, or C₁-C₆ alkyl;

Ring A and Ring B are each independently a 7- to 10-membered spirocyclic heterocyclyl, 5- or 6-membered partially unsaturated monocyclic heterocyclyl, or a 5- or 6-membered heteroaryl;
wherein, Ring A and Ring B each independently contains 1, 2, 3, or 4 ring heteroatoms selected from N, O, and S; and,

R⁸ and R⁹, in each instance, are independently selected from the group consisting of halogen, halo-C₁-C₆ alkyl, an oxo group formed by an oxygen double bonded to a ring carbon, C₁-C₆ alkyl, C₃-C₈ cycloalkyl, hydroxy, hydroxy-C₁-C₆ alkyl, C₁-C₃ alkoxy-C₁-C₆ alkyl, NR^{E2}R^{G2}, —C(o)NR⁴⁰R⁵⁰, and —CH₂C(O)OR⁶⁰;
wherein, R^{E2}, R^{G2}, R⁴⁰, R⁵⁰ and R⁶⁰ are each independently hydrogen or C₁-C₆ alkyl.

Useful compounds include all those having variables as described above.

The straight line in each of Ring A and Ring B represents a single bond, such that Ring A and Ring B are each substituted in an ortho position, and can additionally be substituted with [R⁸]ᵣ or [R⁹]ₛ, respectively.

Ring C and Ring D are always individually connected through a carbon atom in each ring to the remainder of the compound of Formula I.

In certain embodiments, compounds include those where WA and R^{1B} are each independently hydrogen, —CH₃, —CH₂CH₃, —CH₂CH₂CH₃, or —CH(CH₃)₂. In certain embodiments, compounds include those where WA and R^{1B} are each hydrogen.

In certain embodiments, compounds include those where R², R³, R⁴, and R⁵ are each hydrogen.

In certain embodiments, compounds include those where Ring A and Ring B each contain at least one N. In certain embodiments, compounds include those Ring A and Ring B are each independently a 5-membered heteroaryl or 5-membered partially unsaturated monocyclic heterocyclyl. In certain embodiments, compounds include those where Ring A and Ring B are each independently triazolyl or tetrazolyl. Ring A can be further substituted optionally with R⁸ whe r is a value other than zero. Ring B can be further substituted optionally with R⁹ whe s is a value other than zero.

In certain embodiments, compounds include those where R⁸ and R⁹, if present, in each instance, are selected from the group consisting of —CH₃, —CH₂CH₃, —CH₂CH₂CH₃, —CH(CH₃)₂, cyclopropyl, —CH₂OH, —CH₂CH₂OH, —CH₂CH₂OCH₃, —CH₂F, and an oxo group formed by an oxygen double bonded to a ring carbon. In certain embodiments, compounds include those where R⁸ and R⁹, if present, in each instance, are —CH₃.

In certain embodiments, compounds include those where r is 1. In certain embodiments, compounds include those where r is 0. In certain embodiments, compounds include those where s is 1. In certain embodiments, compounds include those where s is 0. In certain embodiments, compounds include those where Ring C

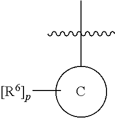

### and Ring D

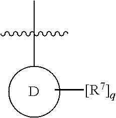

are each independently selected from the group consisting of 5- to 10-membered monocyclic or bicyclic fused heterocyclyl, 6- to 10-membered aryl, and 5- to 10-membered heteroaryl. Optionally substituted Ring C refers to unsubstituted Ring C when the value of p is zero or Ring C substituted with one to four R⁶ groups, when the value of p is not zero. Optionally substituted Ring D refers to unsubstituted Ring D when the value of q is zero or Ring D substituted with one to four R⁷ groups, when the value of q is not zero. Alternatively, a Ring C or Ring D shown as a structure having

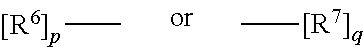

can be referred to as a specific ring, for example, a phenyl, which includes a phenyl substituted by R⁶ or R⁷, or unsubstituted phenyl.

In certain embodiments, compounds include those where Ring C

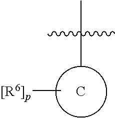

### and Ring D

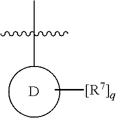

are each independently selected from the group consisting of optionally substituted phenyl, cyclohexyl, tetrahydrofuranyl, quinolinyl, pyrimidinyl, pyridinyl, benzothiazolyl, dihydrobenzofuranyl, quinoxalinyl, benzoimidazolyl, benzodioxolyl, naphthyridinyl, and imidazopyridinyl. In certain embodiments, compounds include those where Ring C

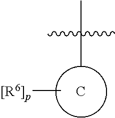

### and Ring D

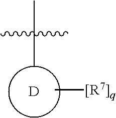

are each independently selected from the group consisting of optionally substituted phenyl, tetrahydrofuranyl, quinolinyl, benzothiazolyl, quinoxalinyl, and pyridinyl. In certain embodiments, compounds include those where Ring C

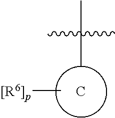

is selected from the group consisting of optionally substituted phenyl, tetrahydrofuranyl, pyridinyl, and quinolinyl. In certain embodiments, compounds include those where Ring D

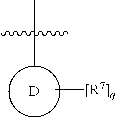

is selected from the group consisting of optionally substituted phenyl, tetrahydrofuranyl, quinolinyl, benzothiazolyl, and quinoxalinyl. In certain embodiments, compounds include those where Ring C

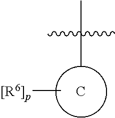

### and Ring D

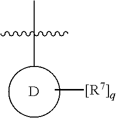

are each optionally substituted phenyl. In certain embodiments, compounds include those where Ring C

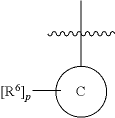

is optionally substituted phenyl and Ring D

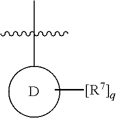

is optionally substituted tetrahydrofuranyl. In certain embodiments, compounds include those where Ring C

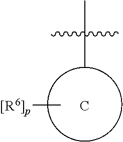

is optionally substituted tetrahydrofuranyl and ring D

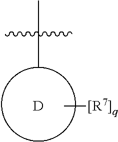

is optionally substituted quinolinyl. In certain embodiments, compounds include those where Ring C

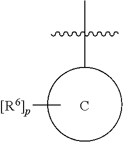

is optionally substituted phenyl and Ring D

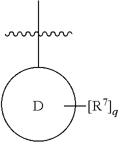

is optionally substituted benzothiazolyl. In certain embodiments, compounds include those where Ring C

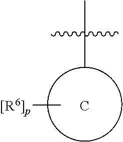

is optionally substituted phenyl and Ring D

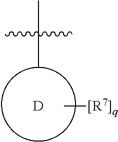

is optionally substituted quinoxalinyl. In certain embodiments, compounds include those where Ring C

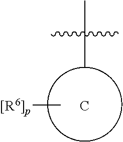

### and Ring D

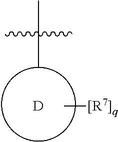

are each optionally substituted quinolinyl. In certain embodiments, compounds include those where Ring C

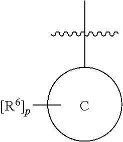

is optionally substituted pyridinyl and Ring D

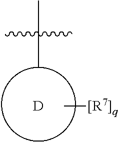

is optionally substituted phenyl.

In certain embodiments, compounds include those where R⁶ and R⁷, if present, in each instance are independently selected from the group consisting of chloro, fluoro, —CH₃, hydroxy, —CH₂OH, —CF₃, —OCH₃, —CH₂CH₃, cyclobutyl, and cyclopropyl.

In certain embodiments, compounds include those where m and n are each independently selected from the group consisting of 0, 1, and 2.

In certain embodiments, compounds include those where at least one of said Ring C

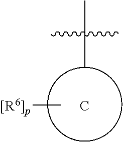

### and Ring D

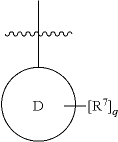

is selected from the group consisting of optionally substituted phenyl, tetrahydrofuranyl, and pyridinyl. In certain embodiments, compounds include those where at least one of said Ring C

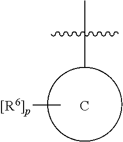

### and Ring D

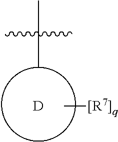

is optionally substituted phenyl. In certain embodiments, compounds include those where at least one of said Ring C

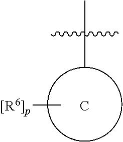

### and Ring D

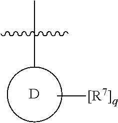

is optionally substituted tetrahydrofuranyl. In certain embodiments, compounds include those where at least one of said Ring C

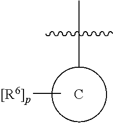

### and Ring D

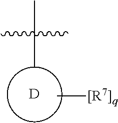

is optionally substituted pyridinyl.

In certain embodiments, compounds include those where said optionally substituted phenyl is selected from the group consisting of

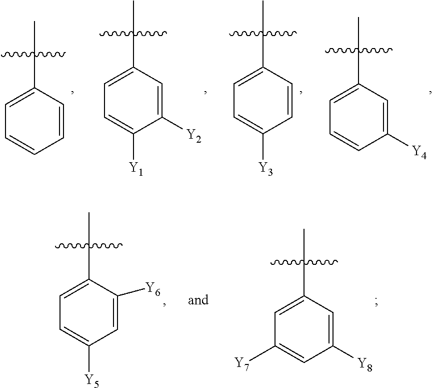

wherein Y₁, Y₂, Y₃, Y₄, Y₅, Y₆, Y₇, and Y₈ are each independently selected from the group consisting of fluoro, chloro, —OCH₃, hydroxy, —CH₂OH, and —CH₃. In certain embodiments, compounds include those where said optionally substituted phenyl is

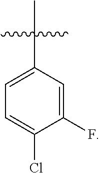 [SMILES: *c1ccc(Cl)c(F)c1]

In certain embodiments, compounds include those where said optionally substituted tetrahydrofuranyl is selected from the group consisting of

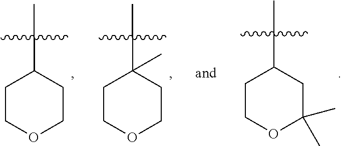

In certain embodiments, compounds include those where said optionally substituted pyridinyl is selected from the group consisting of

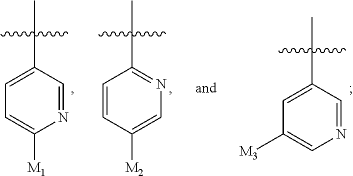

wherein M₁, M₂, and M₃ are each independently selected from the group consisting of hydroxy, cyclopropyl, cyclobutyl, —OCH₃, and —CH₃.

In certain embodiments, compounds include those where R^{A}, R^{B}, R^{C}, and R^{D}, if present, are each hydrogen.

In certain embodiments, compounds include those where m is 1. In certain embodiments, compounds include those where m is 0.

In certain embodiments, compounds include those where n is 0.

In certain embodiments, compounds include those where Ring A is a 7- to 10-membered spirocyclic heterocyclyl and ring B is 5-membered heteroaryl. In certain embodiments of this embodiment, compounds include those where R⁸ is oxo. In certain embodiments of this embodiment, compounds include those where r is 1. In certain embodiments of this embodiment, compounds include those where ring B is a triazolyl. In certain embodiments of this embodiment, compounds include those where R⁹ is —CH₃. In certain embodiments of this embodiment, compounds include those where s is 1. In certain embodiments of this embodiment, compounds include those where s is 0. In certain embodiments of this embodiment, compounds include those where Ring C

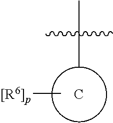

### and Ring D

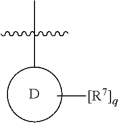

are each independently optionally substituted phenyl or optionally substituted quinolinyl. In certain embodiments, compounds include those where Ring C

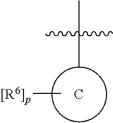

### and Ring D

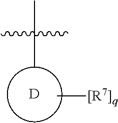

are each optionally substituted phenyl. In certain embodiments, compounds include those where one of Ring C

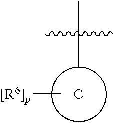

### and Ring D

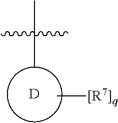

is optionally substituted phenyl and the other is optionally substituted quinolinyl. In certain embodiments of this embodiment, compounds include those where R⁶ and R⁷, if present, in each instance are independently selected from the group consisting of fluoro and chloro. In certain embodiments of this embodiment, compounds include those where p and q are each independently selected from the group consisting of 0, 1, and 2. In certain embodiments of this embodiment, compounds include those where said optionally substituted phenyl has a structure selected from

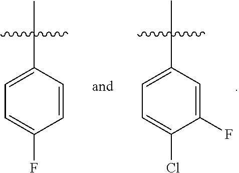 [SMILES: *c1ccc(F)cc1 ; *c1ccc(Cl)c(F)c1]

In certain embodiments of this embodiment, compounds include those where said optionally substituted quinolinyl has the structure

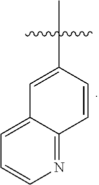 [SMILES: *c1ccc2ncccc2c1]

In certain embodiments of this embodiment, compounds include those where R^{A}, R^{B}, R^{C}, and R^{D}, if present, are each hydrogen. In certain embodiments of this embodiment, compounds include those where m is 1. In certain embodiments, compounds include those where m is 0. In certain embodiments of this embodiment, compounds include those where n is 0.

The subject matter described herein includes the following compounds in Table 1, or pharmaceutically acceptable salts thereof:

TABLE 1

| | | | Mass |
|---|---|---|---|
| Compd. | | | Found |
| No | Structure ^{a} | Nomenclature ^{a} | (M + 1) |
| 1 | 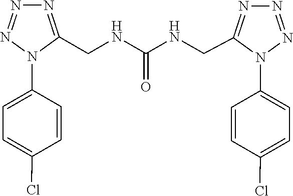 [SMILES: O=C(NCc1nnnn1-c1ccc(Cl)cc1)NCc1nnnn1-c1ccc(Cl)cc1] | 1,3-bis({[1-(4- chlorophenyl)-1H- 1,2,3,4-tetrazol-5- yl]methyl})urea | 443.1 |
| 2 | 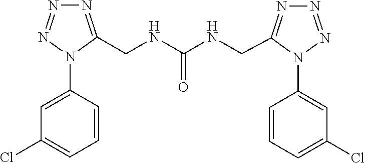 [SMILES: O=C(NCc1nnnn1-c1cccc(Cl)c1)NCc1nnnn1-c1cccc(Cl)c1] | 1,3-bis({[1-(3- chlorophenyl)-1H- 1,2,3,4-tetrazol-5- yl]methyl})urea | 443.1 |
| 3 | 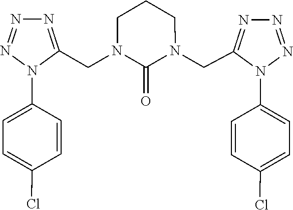 [SMILES: O=C1N(Cc2nnnn2-c2ccc(Cl)cc2)CCCN1Cc1nnnn1-c1ccc(Cl)cc1] | 1,3-bis({[1-(4- chlorophenyl)-1H- 1,2,3,4-tetrazol-5- yl]methyl})-1,3- diazinan-2-one | 485.0 |
| 4 | 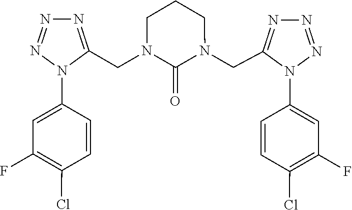 [SMILES: O=C1N(Cc2nnnn2-c2ccc(Cl)c(F)c2)CCCN1Cc1nnnn1-c1ccc(Cl)c(F)c1] | 1,3-bis({[1-(4-chloro- 3-fluorophenyl)-1H- 1,2,3,4-tetrazol-5- yl]methyl})-1,3- diazinan-2-one | 521.0 |
| 5 | 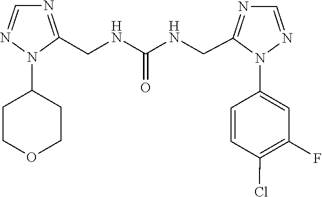 [SMILES: O=C(NCc1ncnn1-c1ccc(Cl)c(F)c1)NCc1ncnn1C1CCOCC1] | 3-{[1-(4-chloro-3- fluorophenyl)-1H- 1,2,4-triazol-5- yl]methyl}-1-{[1- (oxan-4-yl)-1H-1,2,4- triazol-5- yl]methyl}urea | 435.0 |
| 6 | 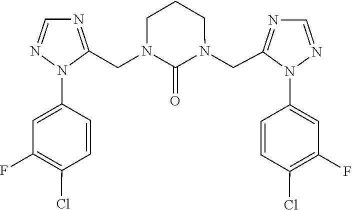 [SMILES: O=C1N(Cc2ncnn2-c2ccc(Cl)c(F)c2)CCCN1Cc1ncnn1-c1ccc(Cl)c(F)c1] | 1,3-bis({[1-(4-chloro- 3-fluorophenyl)-1H- 1,2,4-triazol-5- yl]methyl})-1,3- diazinan-2-one | 519.0 |
| 7 | 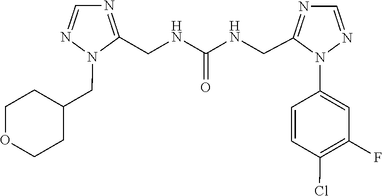 [SMILES: O=C(NCc1ncnn1CC1CCOCC1)NCc1ncnn1-c1ccc(Cl)c(F)c1] | 3-{[1-(4-chloro-3- fluorophenyl)-1H- 1,2,4-triazol-5- yl]methyl}-1-({1- [(oxan-4-yl)methyl]- 1H-1,2,4-triazol-5- yl}methyl)urea | 449.0 |
| 8 | 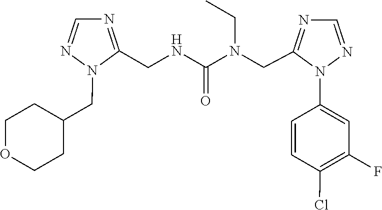 [SMILES: CCN(Cc1ncnn1-c1ccc(Cl)c(F)c1)C(=O)NCc1ncnn1CC1CCOCC1] | 3-{[1-(4-chloro-3- fluorophenyl)-1H- 1,2,4-triazol-5- yl]methyl}-3-ethyl-1- ({1-[(oxan-4- yl)methyl]-1H-1,2,4- triazol-5- yl}methyl)urea | 477.1 |
| 9 | 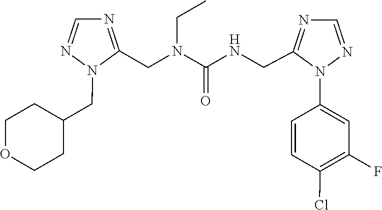 [SMILES: CCN(Cc1ncnn1CC1CCOCC1)C(=O)NCc1ncnn1-c1ccc(Cl)c(F)c1] | 3-{[1-(4-chloro-3- fluorophenyl)-1H- 1,2,4-triazol-5- yl]methyl}-1-ethyl-1- ({1-[(oxan-4- yl)methyl]-1H-1,2,4- triazol-5- yl}methyl)urea | 477.1 |
| 10 | 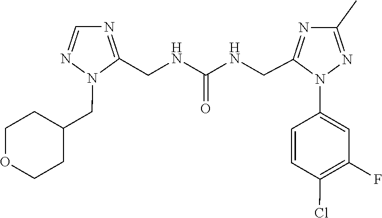 [SMILES: Cc1nc(CNC(=O)NCc2ncnn2CC2CCOCC2)n(-c2ccc(Cl)c(F)c2)n1] | 3-{[1-(4-chloro-3- fluorophenyl)-3- methyl-1H-1,2,4- triazol-5-yl]methyl}- 1-({1-[(oxan-4- yl)methyl]-1H-1,2,4- triazol-5- yl}methyl)urea | 463.0 |
| 11 | 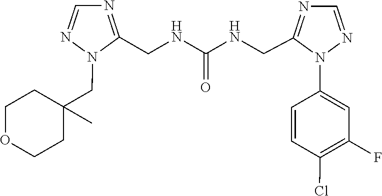 [SMILES: CC1(Cn2ncnc2CNC(=O)NCc2ncnn2-c2ccc(Cl)c(F)c2)CCOCC1] | 3-{[1-(4-chloro-3- fluorophenyl)-1H- 1,2,4-triazol-5- yl]methyl}-1-({1-[(4- methyloxan-4- yl)methyl]-1H-1,2,4- triazol-5- yl}methyl)urea | 463.0 |
| 12 | 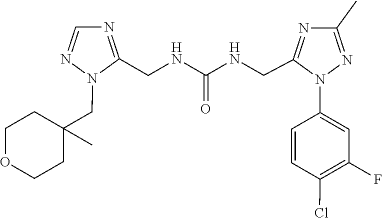 [SMILES: Cc1nc(CNC(=O)NCc2ncnn2CC2(C)CCOCC2)n(-c2ccc(Cl)c(F)c2)n1] | 3-{[1-(4-chloro-3- fluorophenyl)-3- methyl-1H-1,2,4- triazol-5-yl]methyl}- 1-({1-[(4-methyloxan- 4-yl)methyl]-1H- 1,2,4-triazol-5- yl}methyl)urea | 477.1 |
| 13 | 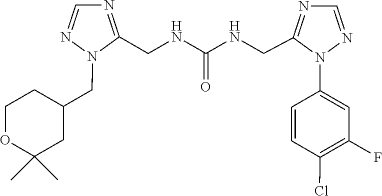 [SMILES: CC1(C)CC(Cn2ncnc2CNC(=O)NCc2ncnn2-c2ccc(Cl)c(F)c2)CCO1] | 3-{[1-(4-chloro-3- fluorophenyl)-1H- 1,2,4-triazol-5- yl]methyl}-1-({1- [(2,2-dimethyloxan-4- yl)methyl]-1H-1,2,4- triazol-5- yl}methyl)urea | 477.1 |
| 14 | 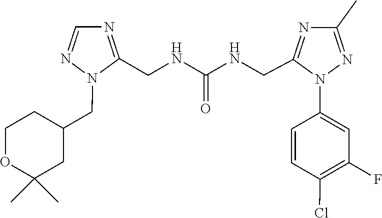 [SMILES: Cc1nc(CNC(=O)NCc2ncnn2CC2CCOC(C)(C)C2)n(-c2ccc(Cl)c(F)c2)n1] | 3-{[1-(4-chloro-3- fluorophenyl)-3- methyl-1H-1,2,4- triazol-5-yl]methyl}- 1-({1-[(2,2- dimethyloxan-4- yl)methyl]-1H-1,2,4- triazol-5- yl}methyl)urea | 491.0 |
| 15 | 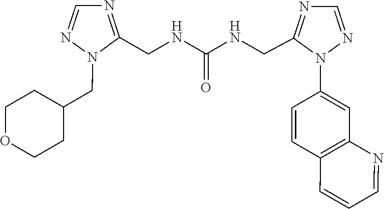 [SMILES: O=C(NCc1ncnn1CC1CCOCC1)NCc1ncnn1-c1ccc2cccnc2c1] | 3-({1-[(oxan-4- yl)methyl]-1H-1,2,4- triazol-5-yl}methyl)- 1-{[1-(quinolin-7-yl)- 1H-1,2,4-triazol-5- yl]methyl}urea | 447.9 |
| 16 | 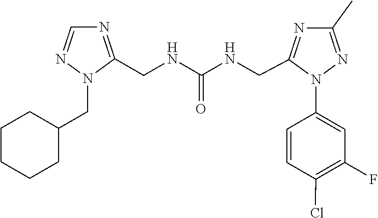 [SMILES: Cc1nc(CNC(=O)NCc2ncnn2CC2CCCCC2)n(-c2ccc(Cl)c(F)c2)n1] | 3-{[1-(4-chloro-3- fluorophenyl)-3- methyl-1H-1,2,4- triazol-5-yl]methyl}- 1-{[1- (cyclohexylmethyl)- 1H-1,2,4-triazol-5- yl]methyl}urea | 461.1 |
| 17 | 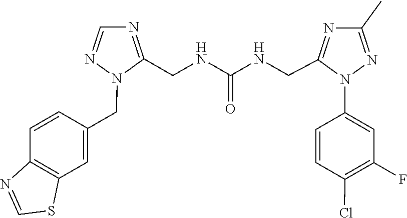 [SMILES: Cc1nc(CNC(=O)NCc2ncnn2Cc2ccc3ncsc3c2)n(-c2ccc(Cl)c(F)c2)n1] | 1-{[1-(1,3- benzothiazol-5-yl)- 1H-1,2,4-triazol-5- yl]methyl}-3-{[1-(4- chloro-3- fluorophenyl)-3- methyl-1H-1,2,4- triazol-5- yl]methyl}urea | 497.9 |
| 18 | 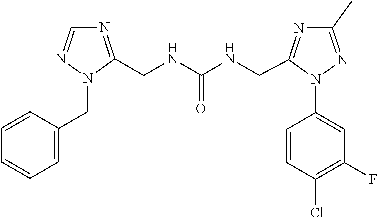 [SMILES: Cc1nc(CNC(=O)NCc2ncnn2Cc2ccccc2)n(-c2ccc(Cl)c(F)c2)n1] | 1-[(1-benzyl-1H-1,2,4- triazol-5-yl)methyl]-3- {[1-(4-chloro-3- fluorophenyl)-3- methyl-1H-1,2,4- triazol-5- yl]methyl}urea | 455.0 |
| 19 | 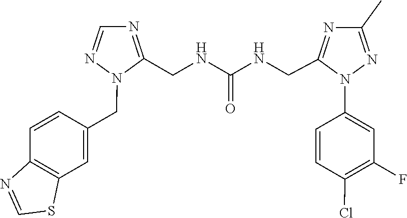 [SMILES: Cc1nc(CNC(=O)NCc2ncnn2Cc2ccc3ncsc3c2)n(-c2ccc(Cl)c(F)c2)n1] | 1-{[1-(1,3- benzothiazol-5-yl)- 1H-1,2,4-triazol-5- yl]methyl}-3-{[1-(4- chloro-3- fluorophenyl)-3- methyl-1H-1,2,4- triazol-5- yl]methyl}urea | 497.9 |
| 20 | 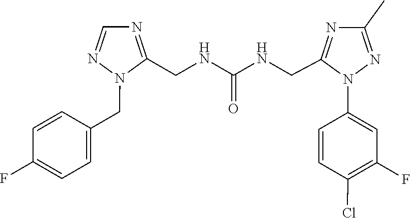 [SMILES: Cc1nc(CNC(=O)NCc2ncnn2Cc2ccc(F)cc2)n(-c2ccc(Cl)c(F)c2)n1] | 3-{[1-(4-chloro-3- fluorophenyl)-3- methyl-1H-1,2,4- triazol-5-yl]methyl}- 1-({1-[(4- fluorophenyl)methyl]- 1H-1,2,4-triazol-5- yl}methyl)urea | 473.1 |
| 21 | 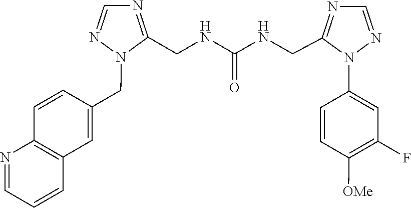 [SMILES: COc1ccc(-n2ncnc2CNC(=O)NCc2ncnn2Cc2ccc3ncccc3c2)cc1F] | 1-{[1-(3-fluoro-4- methoxyphenyl)-1H- 1,2,4-triazol-5- yl]methyl}-3-{[1- (quinolin-7-yl)-1H- 1,2,4-triazol-5- yl]methyl}urea | 473.9 |
| 22 | 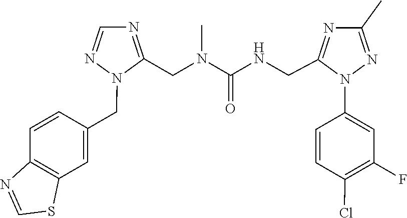 [SMILES: Cc1nc(CNC(=O)N(C)Cc2ncnn2Cc2ccc3ncsc3c2)n(-c2ccc(Cl)c(F)c2)n1] | 1-{[1-(1,3- benzothiazol-5-yl)- 1H-1,2,4-triazol-5- yl]methyl}-3-{[1-(4- chloro-3- fluorophenyl)-3- methyl-1H-1,2,4- triazol-5-yl]methyl}- 1-methylurea | 512.1 |
| 23 | 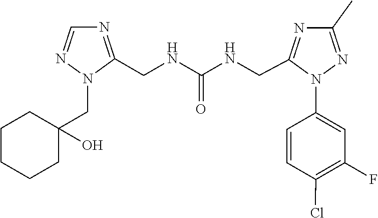 [SMILES: Cc1nc(CNC(=O)NCc2ncnn2CC2(O)CCCCC2)n(-c2ccc(Cl)c(F)c2)n1] | 3-{[1-(4-chloro-3- fluorophenyl)-3- methyl-1H-1,2,4- triazol-5-yl]methyl}- 1-({1-[(1- hydroxycyclohexyl)m ethyl]-1H-1,2,4- triazol-5- yl}methyl)urea | 477.0 |
| 24 | 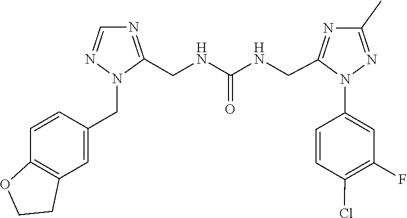 [SMILES: Cc1nc(CNC(=O)NCc2ncnn2Cc2ccc3c(c2)CCO3)n(-c2ccc(Cl)c(F)c2)n1] | 3-{[1-(4-chloro-3- fluorophenyl)-3- methyl-1H-1,2,4- triazol-5-yl]methyl}- 1-{[1-(2,3-dihydro-1- benzofuran-6-yl)-1H- 1,2,4-triazol-5- yl]methyl}urea | 483.3 |
| 25 | 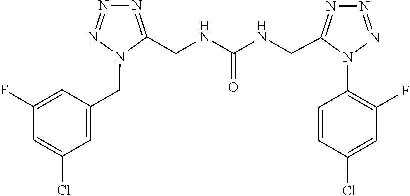 [SMILES: O=C(NCc1nnnn1Cc1cc(F)cc(Cl)c1)NCc1nnnn1-c1ccc(Cl)cc1F] | 1,3-bis({[1-(4-chloro- 2-fluorophenyl)-1H- 1,2,3,4-tetrazol-5- yl]methyl})urea | 481.05- 483.35 |
| 26 | 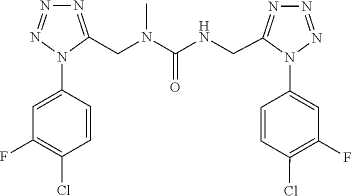 [SMILES: CN(Cc1nnnn1-c1ccc(Cl)c(F)c1)C(=O)NCc1nnnn1-c1ccc(Cl)c(F)c1] | 1,3-bis({[1-(4-chloro- 3-fluorophenyl)-1H- 1,2,3,4-tetrazol-5- yl]methyl})-1- methylurea | 496.8 |
| 27 | 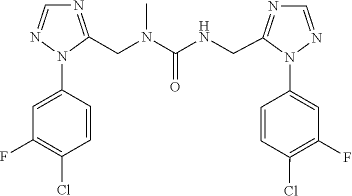 [SMILES: CN(Cc1ncnn1-c1ccc(Cl)c(F)c1)C(=O)NCc1ncnn1-c1ccc(Cl)c(F)c1] | 1,3-bis({[1-(4-chloro- 3-fluorophenyl)-1H- 1,2,4-triazol-5- yl]methyl})-1- methylurea | 493.4 |
| 28 | 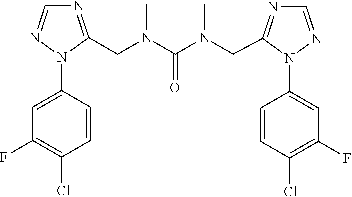 [SMILES: CN(Cc1ncnn1-c1ccc(Cl)c(F)c1)C(=O)N(C)Cc1ncnn1-c1ccc(Cl)c(F)c1] | 1,3-bis({[1-(4-chloro- 3-fluorophenyl)-1H- 1,2,4-triazol-5- yl]methyl})-1,3- dimethylurea | 507.2/ 509.1 |
| 29 | 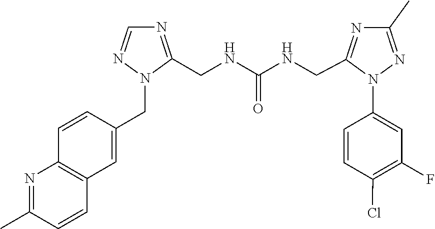 [SMILES: Cc1ccc2cc(Cn3ncnc3CNC(=O)NCc3nc(C)nn3-c3ccc(Cl)c(F)c3)ccc2n1] | 3-{[1-(4-chloro-3- fluorophenyl)-3- methyl-1H-1,2,4- triazol-5-yl]methyl}- 1-{[1-(2- methylquinolin-7-yl)- 1H-1,2,4-triazol-5- yl]methyl}urea | 506 |
| 30 | 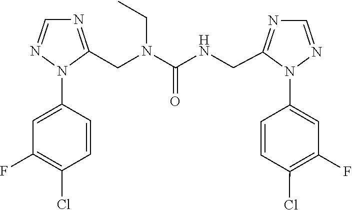 [SMILES: CCN(Cc1ncnn1-c1ccc(Cl)c(F)c1)C(=O)NCc1ncnn1-c1ccc(Cl)c(F)c1] | 1,3-bis({[1-(4-chloro- 3-fluorophenyl)-1H- 1,2,4-triazol-5- yl]methyl})-1- ethylurea | 507.1 |
| 31 | 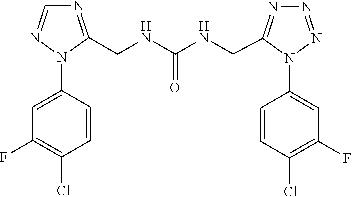 [SMILES: O=C(NCc1ncnn1-c1ccc(Cl)c(F)c1)NCc1nnnn1-c1ccc(Cl)c(F)c1] | 3-{[1-(4-chloro-3- fluorophenyl)-1H- 1,2,3,4-tetrazol-5- yl]methyl}-1-{[1-(4- chloro-3- fluorophenyl)-1H- 1,2,4-triazol-5- yl]methyl}urea | 480.1 |
| 32 | 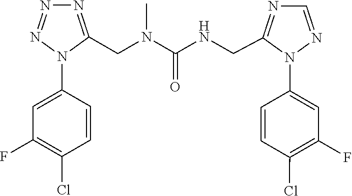 [SMILES: CN(Cc1nnnn1-c1ccc(Cl)c(F)c1)C(=O)NCc1ncnn1-c1ccc(Cl)c(F)c1] | 1-{[1-(4-chloro-3- fluorophenyl)-1H- 1,2,3,4-tetrazol-5- yl]methyl}-3-{[1-(4- chloro-3- fluorophenyl)-1H- 1,2,4-triazol-5- yl]methyl}-1- methylurea | 494.31 |
| 33 | 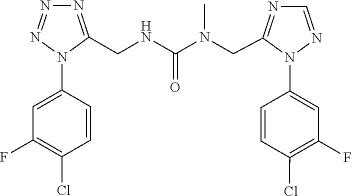 [SMILES: CN(Cc1ncnn1-c1ccc(Cl)c(F)c1)C(=O)NCc1nnnn1-c1ccc(Cl)c(F)c1] | 1-{[1-(4-chloro-3- fluorophenyl)-1H- 1,2,3,4-tetrazol-5- yl]methyl}-3-{[1-(4- chloro-3- fluorophenyl)-1H- 1,2,4-triazol-5- yl]methyl}-3- methylurea | 495.97 |
| 34 | 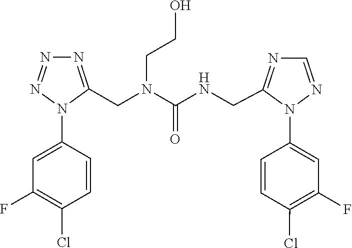 [SMILES: O=C(NCc1ncnn1-c1ccc(Cl)c(F)c1)N(CCO)Cc1nnnn1-c1ccc(Cl)c(F)c1] | 1-{[1-(4-chloro-3- fluorophenyl)-1H- 1,2,3,4-tetrazol-5- yl]methyl}-3-{[1-(4- chloro-3- fluorophenyl)-1H- 1,2,4-triazol-5- yl]methyl}-1-(2- hydroxyethyl)urea | 524.1 |
| 35 | 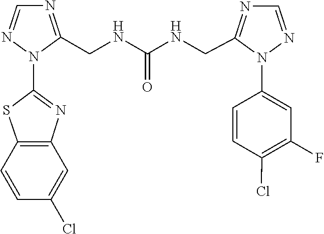 [SMILES: O=C(NCc1ncnn1-c1ccc(Cl)c(F)c1)NCc1ncnn1-c1nc2cc(Cl)ccc2s1] | 1-{[1-(5-chloro-1,3- benzothiazol-2-yl)- 1H-1,2,4-triazol-5- yl]methyl}-3-{[1-(4- chloro-3- fluorophenyl)-1H- 1,2,4-triazol-5- yl]methyl}urea | 518.2 |
| 36 | 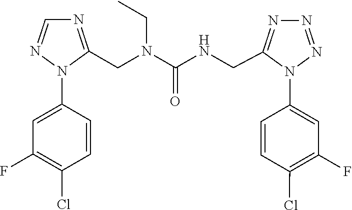 [SMILES: CCN(Cc1ncnn1-c1ccc(Cl)c(F)c1)C(=O)NCc1nnnn1-c1ccc(Cl)c(F)c1] | 3-{[1-(4-chloro-3- fluorophenyl)-1H- 1,2,3,4-tetrazol-5- yl]methyl}-1-{[1-(4- chloro-3- fluorophenyl)-1H- 1,2,4-triazol-5- yl]methyl}-1- ethylurea | 508.23 |
| 37 | 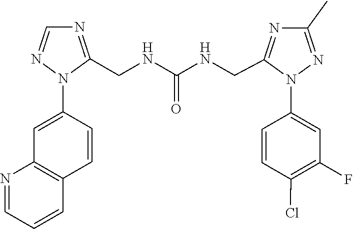 [SMILES: Cc1nc(CNC(=O)NCc2ncnn2-c2ccc3cccnc3c2)n(-c2ccc(Cl)c(F)c2)n1] | 3-{[1-(4-chloro-3- fluorophenyl)-3- methyl-1H-1,2,4- triazol-5-yl]methyl}- 1-{[1-(quinolin-7-yl)- 1H-1,2,4-triazol-5- yl]methyl}urea | 492.22 |
| 38 | 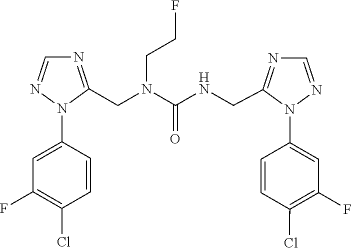 [SMILES: O=C(NCc1ncnn1-c1ccc(Cl)c(F)c1)N(CCF)Cc1ncnn1-c1ccc(Cl)c(F)c1] | 1,3-bis({[1-(4-chloro- 3-fluorophenyl)-1H- 1,2,4-triazol-5- yl]methyl})-1-(2- fluoroethyl)urea | 527.03 |
| 39 | 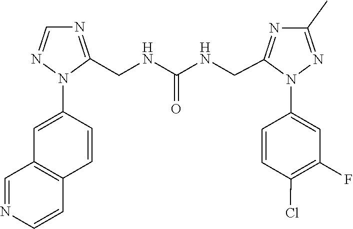 [SMILES: Cc1nc(CNC(=O)NCc2ncnn2-c2ccc3ccncc3c2)n(-c2ccc(Cl)c(F)c2)n1] | 3-{[1-(4-chloro-3- fluorophenyl)-3- methyl-1H-1,2,4- triazol-5-yl]methyl}- 1-{[1-(isoquinolin-7- yl)-1H-1,2,4-triazol-5- yl]methyl}urea | 492.17 |
| 40 | 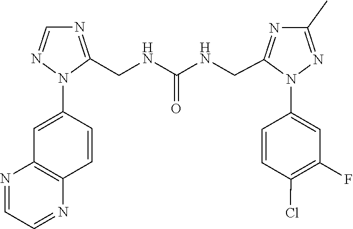 [SMILES: Cc1nc(CNC(=O)NCc2ncnn2-c2ccc3nccnc3c2)n(-c2ccc(Cl)c(F)c2)n1] | 3-{[1-(4-chloro-3- fluorophenyl)-3- methyl-1H-1,2,4- triazol-5-yl]methyl}- 1-{[1-(quinoxalin-6- yl)-1H-1,2,4-triazol-5- yl]methyl}urea | 493.37 |
| 41 | 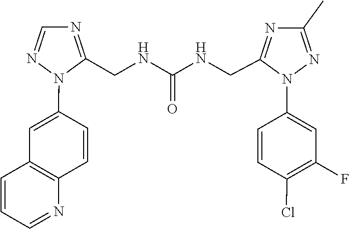 [SMILES: Cc1nc(CNC(=O)NCc2ncnn2-c2ccc3ncccc3c2)n(-c2ccc(Cl)c(F)c2)n1] | 3-{[1-(4-chloro-3- fluorophenyl)-3- methyl-1H-1,2,4- triazol-5-yl]methyl}- 1-{[1-(quinolin-6-yl)- 1H-1,2,4-triazol-5- yl]methyl}urea | 491.78 |
| 42 | 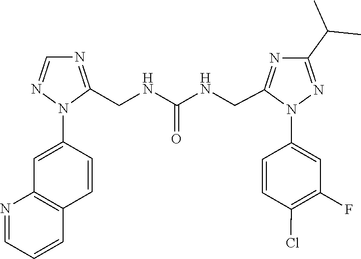 [SMILES: CC(C)c1nc(CNC(=O)NCc2ncnn2-c2ccc3cccnc3c2)n(-c2ccc(Cl)c(F)c2)n1] | 3-{[1-(4-chloro-3- fluorophenyl)-3- (propan-2-yl)-1H- 1,2,4-triazol-5- yl]methyl}-1-{[1- (quinolin-7-yl)-1H- 1,2,4-triazol-5- yl]methyl}urea | 520.36 |
| 43 | 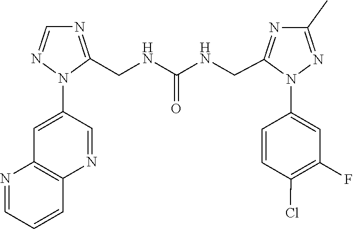 [SMILES: Cc1nc(CNC(=O)NCc2ncnn2-c2cnc3cccnc3c2)n(-c2ccc(Cl)c(F)c2)n1] | 3-{[1-(4-chloro-3- fluorophenyl)-3- methyl-1H-1,2,4- triazol-5-yl]methyl}- 1-{[1-(1,5- naphthyridin-3-yl)- 1H-1,2,4-triazol-5- yl]methyl}urea | 493.24 |
| 44 | 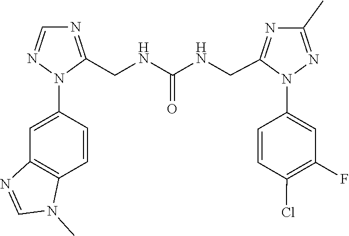 [SMILES: Cc1nc(CNC(=O)NCc2ncnn2-c2ccc3c(c2)ncn3C)n(-c2ccc(Cl)c(F)c2)n1] | 3-{[1-(4-chloro-3- fluorophenyl)-3- methyl-1H-1,2,4- triazol-5-yl]methyl}- 1-{[1-(1-methyl-1H- 1,3-benzodiazol-5-yl)- 1H-1,2,4-triazol-5- yl]methyl}urea | 494.90 |
| 45 | 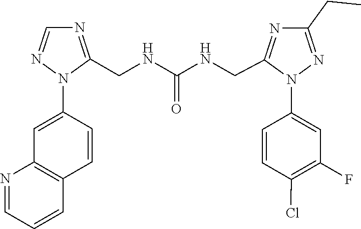 [SMILES: CCc1nc(CNC(=O)NCc2ncnn2-c2ccc3cccnc3c2)n(-c2ccc(Cl)c(F)c2)n1] | 3-{[1-(4-chloro-3- fluorophenyl)-3-ethyl- 1H-1,2,4-triazol-5- yl]methyl}-1-{[1- (quinolin-7-yl)-1H- 1,2,4-triazol-5- yl]methyl}urea | 505.83 |
| 46 | 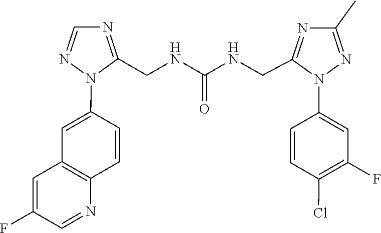 [SMILES: Cc1nc(CNC(=O)NCc2ncnn2-c2ccc3ncc(F)cc3c2)n(-c2ccc(Cl)c(F)c2)n1] | 3-{[1-(4-chloro-3- fluorophenyl)-3- methyl-1H-1,2,4- triazol-5-yl]methyl}- 1-{[1-(3- fluoroquinolin-6-yl)- 1H-1,2,4-triazol-5- yl]methyl}urea | 505.83 |
| 47 | 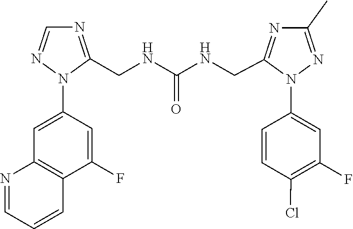 [SMILES: Cc1nc(CNC(=O)NCc2ncnn2-c2cc(F)c3cccnc3c2)n(-c2ccc(Cl)c(F)c2)n1] | 3-{[1-(4-chloro-3- fluorophenyl)-3- methyl-1H-1,2,4- triazol-5-yl]methyl}- 1-{[1-(5- fluoroquinolin-7-yl)- 1H-1,2,4-triazol-5- yl]methyl}urea | 511.10 |
| 48 | 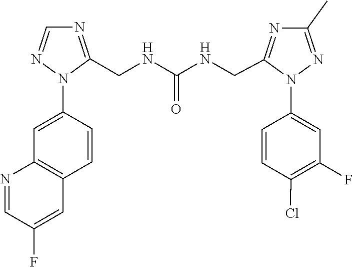 [SMILES: Cc1nc(CNC(=O)NCc2ncnn2-c2ccc3cc(F)cnc3c2)n(-c2ccc(Cl)c(F)c2)n1] | 3-{[1-(4-chloro-3- fluorophenyl)-3- methyl-1H-1,2,4- triazol-5-yl]methyl}- 1-{[1-(3- fluoroquinolin-7-yl)- 1H-1,2,4-triazol-5- yl]methyl}urea | 510.03 |
| 49 | 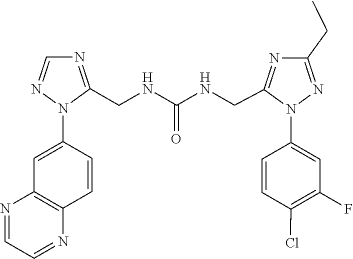 [SMILES: CCc1nc(CNC(=O)NCc2ncnn2-c2ccc3nccnc3c2)n(-c2ccc(Cl)c(F)c2)n1] | 3-{[1-(4-chloro-3- fluorophenyl)-3-ethyl- 1H-1,2,4-triazol-5- yl]methyl}-1-{[1- (quinoxalin-6-yl)-1H- 1,2,4-triazol-5- yl]methyl}urea | 507.43 |
| 50 | 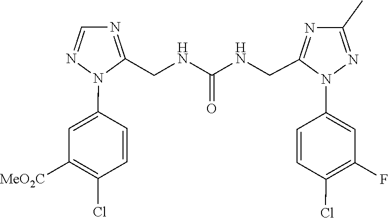 [SMILES: COC(=O)c1cc(-n2ncnc2CNC(=O)NCc2nc(C)nn2-c2ccc(Cl)c(F)c2)ccc1Cl] | methyl2-chloro-5-(5- {[({[1-(4-chloro-3- fluorophenyl)-3- methyl-1H-1,2,4- triazol-5- yl]methyl}carbamoyl) amino]methyl}-1H- 1,2,4-triazol-1- yl)benzoate | 533.35 |
| 51 | 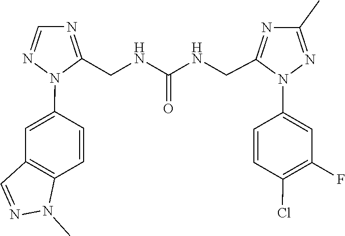 [SMILES: Cc1nc(CNC(=O)NCc2ncnn2-c2ccc3c(cnn3C)c2)n(-c2ccc(Cl)c(F)c2)n1] | 3-{[1-(4-chloro-3- fluorophenyl)-3- methyl-1H-1,2,4- triazol-5-yl]methyl}- 1-{[1-(1-methyl-1H- indazol-5-yl)-1H- 1,2,4-triazol-5- yl]methyl}urea | 495.04 |
| 52 | 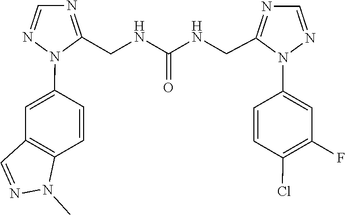 [SMILES: Cn1ncc2cc(-n3ncnc3CNC(=O)NCc3ncnn3-c3ccc(Cl)c(F)c3)ccc21] | 3-{[1-(4-chloro-3- fluorophenyl)-1H- 1,2,4-triazol-5- yl]methyl}-1-{[1-(1- methyl-1H-indazol-5- yl)-1H-1,2,4-triazol-5- yl]methyl}urea | 481.05 |
| 53 | 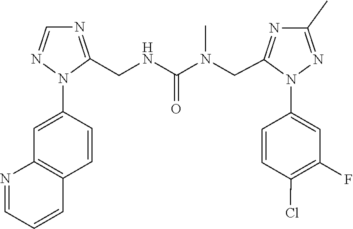 [SMILES: Cc1nc(CN(C)C(=O)NCc2ncnn2-c2ccc3cccnc3c2)n(-c2ccc(Cl)c(F)c2)n1] | 3-{[1-(4-chloro-3- fluorophenyl)-3- methyl-1H-1,2,4- triazol-5-yl]methyl}- 3-methyl-1-{[1- (quinolin-7-yl)-1H- 1,2,4-triazol-5- yl]methyl}urea | |
| 54 | 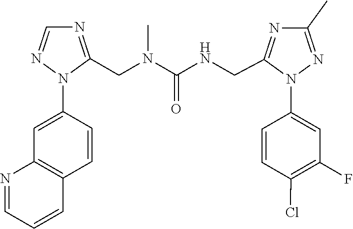 [SMILES: Cc1nc(CNC(=O)N(C)Cc2ncnn2-c2ccc3cccnc3c2)n(-c2ccc(Cl)c(F)c2)n1] | 3-{[1-(4-chloro-3- fluorophenyl)-3- methyl-1H-1,2,4- triazol-5-yl]methyl}- 1-methyl-1-{[1- (quinolin-7-yl)-1H- 1,2,4-triazol-5- yl]methyl}urea | 506.16 |
| 55 | 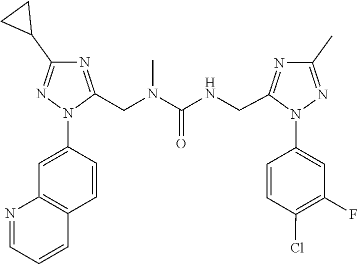 [SMILES: Cc1nc(CNC(=O)N(C)Cc2nc(C3CC3)nn2-c2ccc3cccnc3c2)n(-c2ccc(Cl)c(F)c2)n1] | 3-{[1-(4-chloro-3- fluorophenyl)-3- methyl-1H-1,2,4- triazol-5-yl]methyl}- 1-{[3-cyclopropyl-1- (quinolin-7-yl)-1H- 1,2,4-triazol-5- yl]methyl}-1- methylurea | 532.9 |
| 56 | 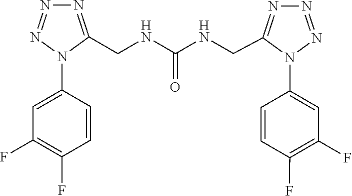 [SMILES: O=C(NCc1nnnn1-c1ccc(F)c(F)c1)NCc1nnnn1-c1ccc(F)c(F)c1] | 1,3-bis({[1-(3,4- difluorophenyl)-1H- 1,2,3,4-tetrazol-5- yl]methyl})urea | 449.2 |
| 57 | 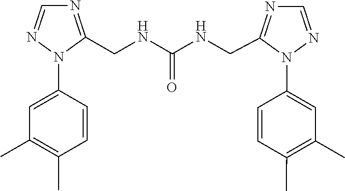 | 1,3-bis({[1-(3,4- dimethylphenyl)-1H- 1,2,4-triazol-5- yl]methyl})urea | 431.2 |
| 58 | 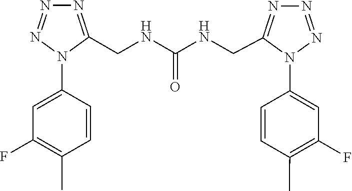 [SMILES: Cc1ccc(-n2nnnc2CNC(=O)NCc2nnnn2-c2ccc(C)c(F)c2)cc1F] | 1,3-bis({[1-(3-fluoro- 4-methylphenyl)-1H- 1,2,3,4-tetrazol-5- yl]methyl})urea | 441.2 |
| 59 | 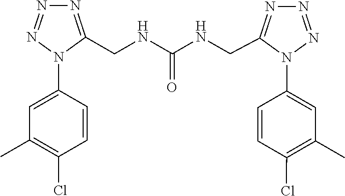 | 1,3-bis({[1-(4-chloro- 3-methylphenyl)-1H- 1,2,3,4-tetrazol-5- yl]methyl})urea | 473.1 |
| 60 | 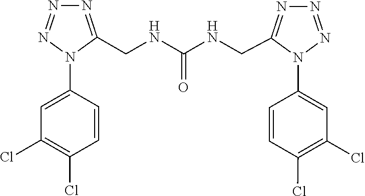 [SMILES: O=C(NCc1nnnn1-c1ccc(Cl)c(Cl)c1)NCc1nnnn1-c1ccc(Cl)c(Cl)c1] | 1,3-bis({[1-(3,4- dichlorophenyl)-1H- 1,2,3,4-tetrazol-5- yl]methyl})urea | 515.0 |
| 61 | 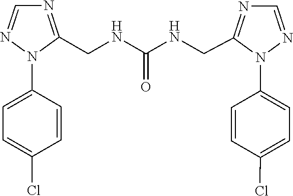 [SMILES: O=C(NCc1ncnn1-c1ccc(Cl)cc1)NCc1ncnn1-c1ccc(Cl)cc1] | 1,3-bis({[1-(4- chlorophenyl)-1H- 1,2,4-triazol-5- yl]methyl})urea | 443.2 |
| 62 | 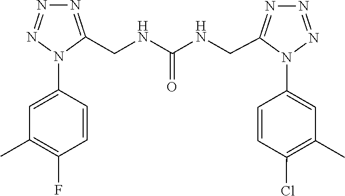 | 1,3-bis({[1-(4-fluoro- 3-methylphenyl)-1H- 1,2,3,4-tetrazol-5- yl]methyl})urea | 441.2 |
| 63 | 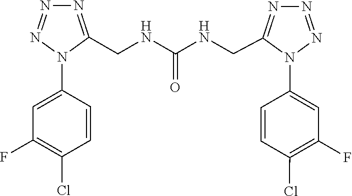 [SMILES: O=C(NCc1nnnn1-c1ccc(Cl)c(F)c1)NCc1nnnn1-c1ccc(Cl)c(F)c1] | 1,3-bis({[1-(4-chloro- 3-fluorophenyl)-1H- 1,2,3,4-tetrazol-5- yl]methyl})urea | 481.1 |
| 64 | 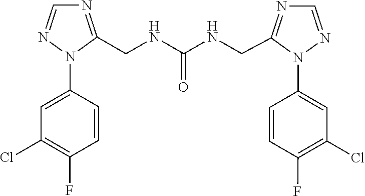 [SMILES: O=C(NCc1ncnn1-c1ccc(F)c(Cl)c1)NCc1ncnn1-c1ccc(F)c(Cl)c1] | 1,3-bis({[1-(3-chloro- 4-fluorophenyl)-1H- 1,2,4-triazol-5- yl]methyl})urea | 479 |
| 65 | 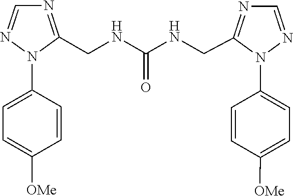 [SMILES: COc1ccc(-n2ncnc2CNC(=O)NCc2ncnn2-c2ccc(OC)cc2)cc1] | 1,3-bis({[1-(4- methoxyphenyl)-1H- 1,2,4-triazol-5- yl]methyl})urea | 435.2 |
| 66 | 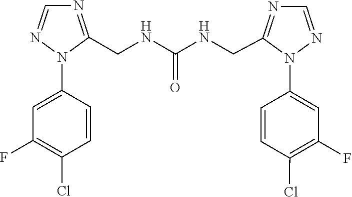 [SMILES: O=C(NCc1ncnn1-c1ccc(Cl)c(F)c1)NCc1ncnn1-c1ccc(Cl)c(F)c1] | 1,3-bis({[1-(4-chloro- 3-fluorophenyl)-1H- 1,2,4-triazol-5- yl]methyl})urea | 479.1 |
| 67 | 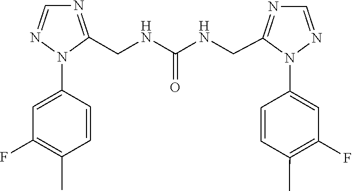 [SMILES: Cc1ccc(-n2ncnc2CNC(=O)NCc2ncnn2-c2ccc(C)c(F)c2)cc1F] | 1,3-bis({[1-(3-fluoro- 4-methylphenyl)-1H- 1,2,4-triazol-5- yl]methyl})urea | 439.2 |
| 68 | 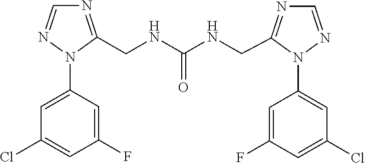 [SMILES: O=C(NCc1ncnn1-c1cc(F)cc(Cl)c1)NCc1ncnn1-c1cc(F)cc(Cl)c1] | 1,3-bis({[1-(3-chloro- 5-fluorophenyl)-1H- 1,2,4-triazol-5- yl]methyl})urea | 479 |
| 69 | 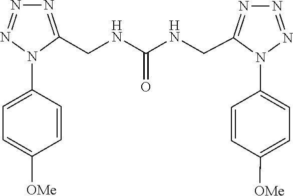 [SMILES: COc1ccc(-n2nnnc2CNC(=O)NCc2nnnn2-c2ccc(OC)cc2)cc1] | 1,3-bis({[1-(4- methoxyphenyl)-1H- 1,2,3,4-tetrazol-5- yl]methyl})urea | 437.3 |
| 70 | 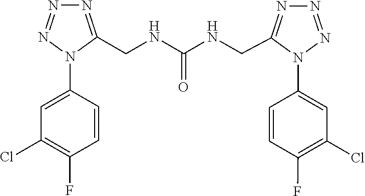 [SMILES: O=C(NCc1nnnn1-c1ccc(F)c(Cl)c1)NCc1nnnn1-c1ccc(F)c(Cl)c1] | 1,3-bis({[1-(3-chloro- 4-fluorophenyl)-1H- 1,2,3,4-tetrazol-5- yl]methyl})urea | 481.1 |
| 71 | 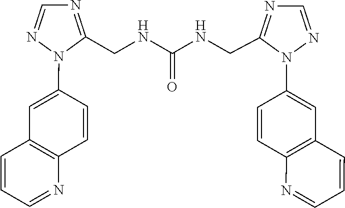 [SMILES: O=C(NCc1ncnn1-c1ccc2ncccc2c1)NCc1ncnn1-c1ccc2ncccc2c1] | 1,3-bis({[1-(quinolin- 6-yl)-1H-1,2,4-triazol- 5-yl]methyl})urea | 477.3 |
| 72 | 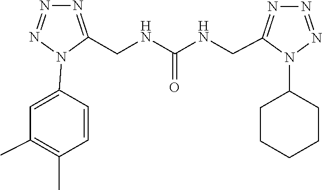 [SMILES: Cc1ccc(-n2nnnc2CNC(=O)NCc2nnnn2C2CCCCC2)cc1C] | 1-[(1-cyclohexyl-1H- 1,2,3,4-tetrazol-5- yl)methyl]-3-{[1-(3,4- dimethylphenyl)-1H- 1,2,3,4-tetrazol-5- yl]methyl}urea | 411 |
| 73 | 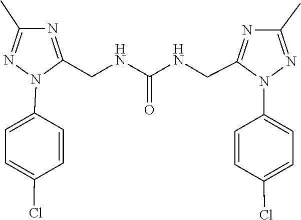 | 1,3-bis({[1-(4- chlorophenyl)-3- methyl-1H-1,2,4- triazol-5- yl]methyl})urea | 471 |
| 74 | 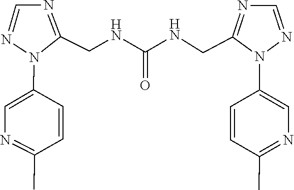 [SMILES: Cc1ccc(-n2ncnc2CNC(=O)NCc2ncnn2-c2ccc(C)nc2)cn1] | 1,3-bis({[1-(6- methylpyridin-3-yl)- 1H-1,2,4-triazol-5- yl]methyl})urea | 405.3 |
| 75 | 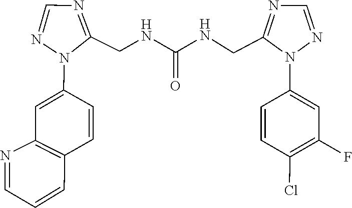 [SMILES: O=C(NCc1ncnn1-c1ccc(Cl)c(F)c1)NCc1ncnn1-c1ccc2cccnc2c1] | 1-{[1-(4-chloro-3- fluorophenyl)-1H- 1,2,4-triazol-5- yl]methyl}-3-{[1- (quinolin-7-yl)-1H- 1,2,4-triazol-5- yl]methyl}urea | 478.3 |
| 76 | 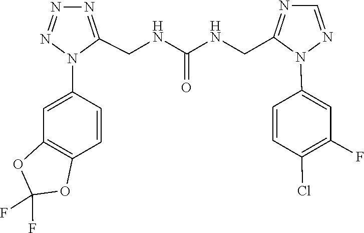 [SMILES: O=C(NCc1ncnn1-c1ccc(Cl)c(F)c1)NCc1nnnn1-c1ccc2c(c1)OC(F)(F)O2] | 1-{[1-(4-chloro-3- fluorophenyl)-1H- 1,2,4-triazol-5- yl]methyl}-3-{[1-(2,2- difluoro-2H-1,3- benzodioxol-5-yl)-1H- 1,2,3,4-tetrazol-5- yl]methyl}urea | 508.2 |
| 77 | 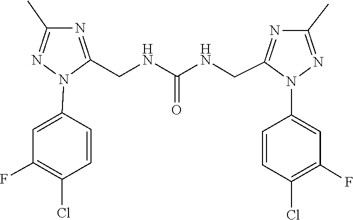 [SMILES: Cc1nc(CNC(=O)NCc2nc(C)nn2-c2ccc(Cl)c(F)c2)n(-c2ccc(Cl)c(F)c2)n1] | 1,3-bis({[1-(4-chloro- 3-fluorophenyl)-3- methyl-1H-1,2,4- triazol-5- yl]methyl})urea | 507 |
| 78 | 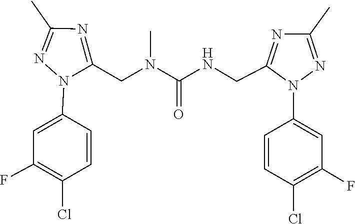 [SMILES: Cc1nc(CNC(=O)N(C)Cc2nc(C)nn2-c2ccc(Cl)c(F)c2)n(-c2ccc(Cl)c(F)c2)n1] | 1,3-bis({[1-(4-chloro- 3-fluorophenyl)-3- methyl-1H-1,2,4- triazol-5-yl]methyl})- 1-methylurea | 521.2 |
| 79 | 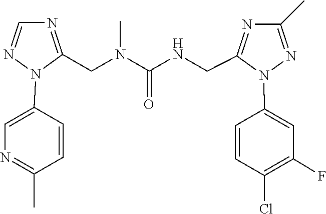 [SMILES: Cc1ccc(-n2ncnc2CN(C)C(=O)NCc2nc(C)nn2-c2ccc(Cl)c(F)c2)cn1] | 3-{[1-(4-chloro-3- fluorophenyl)-3- methyl-1H-1,2,4- triazol-5-yl]methyl}- 1-methyl-1-{[1-(6- methylpyridin-3-yl)- 1H-1,2,4-triazol-5- yl]methyl}urea | 470.2 |
| 80 | 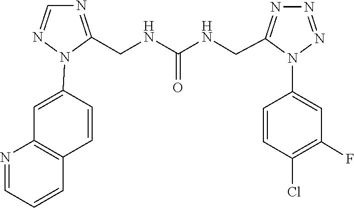 [SMILES: O=C(NCc1ncnn1-c1ccc2cccnc2c1)NCc1nnnn1-c1ccc(Cl)c(F)c1] | 1-{[1-(4-chloro-3- fluorophenyl)-1H- 1,2,3,4-tetrazol-5- yl]methyl}-3-{[1- (quinolin-7-yl)-1H- 1,2,4-triazol-5- yl]methyl}urea | 479.4 |
| 81 | 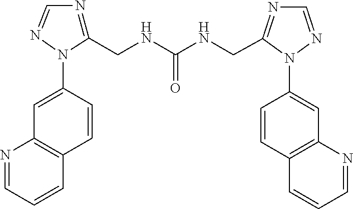 [SMILES: O=C(NCc1ncnn1-c1ccc2cccnc2c1)NCc1ncnn1-c1ccc2cccnc2c1] | 1,3-bis({[1-(quinolin- 7-yl)-1H-1,2,4-triazol- 5-yl]methyl})urea | 477.5 |
| 82 | 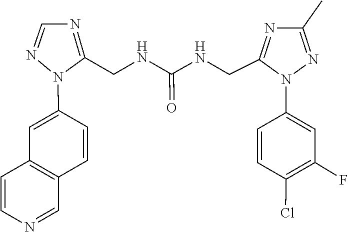 [SMILES: Cc1nc(CNC(=O)NCc2ncnn2-c2ccc3cnccc3c2)n(-c2ccc(Cl)c(F)c2)n1] | 1-{[1-(4-chloro-3- fluorophenyl)-3- methyl-1H-1,2,4- triazol-5-yl]methyl}- 3-{[1-(isoquinolin-6- yl)-1H-1,2,4-triazol-5- yl]methyl}urea | 492.4 |
| 83 | 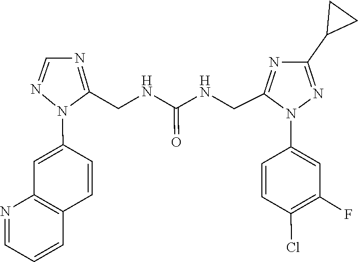 [SMILES: O=C(NCc1ncnn1-c1ccc2cccnc2c1)NCc1nc(C2CC2)nn1-c1ccc(Cl)c(F)c1] | 1-{[1-(4-chloro-3- fluorophenyl)-3- cyclopropyl-1H-1,2,4- triazol-5-yl]methyl}- 3-{[1-(quinolin-7-yl)- 1H-1,2,4-triazol-5- yl]methyl}urea | 518.4 |
| 84 | 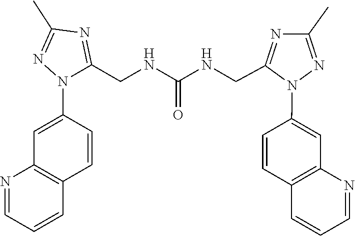 [SMILES: Cc1nc(CNC(=O)NCc2nc(C)nn2-c2ccc3cccnc3c2)n(-c2ccc3cccnc3c2)n1] | 1,3-bis({[3-methyl-1- (quinolin-7-yl)-1H- 1,2,4-triazol-5- yl]methyl})urea | 505 |
| 85 | 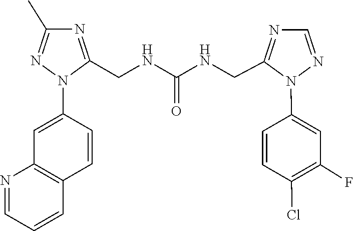 [SMILES: Cc1nc(CNC(=O)NCc2ncnn2-c2ccc(Cl)c(F)c2)n(-c2ccc3cccnc3c2)n1] | 1-{[1-(4-chloro-3- fluorophenyl)-1H- 1,2,4-triazol-5- yl]methyl}-3-{[3- methyl-1-(quinolin-7- yl)-1H-1,2,4-triazol-5- yl]methyl}urea | 492 |
| 86 | 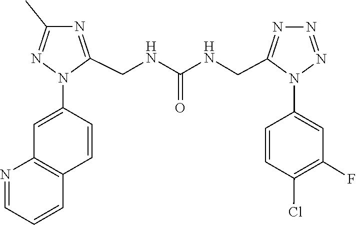 [SMILES: Cc1nc(CNC(=O)NCc2nnnn2-c2ccc(Cl)c(F)c2)n(-c2ccc3cccnc3c2)n1] | 1-{[1-(4-chloro-3- fluorophenyl)-1H- 1,2,3,4-tetrazol-5- yl]methyl}-3-{[3- methyl-1-(quinolin-7- yl)-1H-1,2,4-triazol-5- yl]methyl}urea | 493.1 |
| 87 | 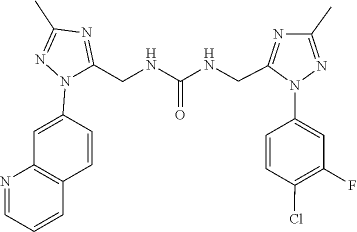 [SMILES: Cc1nc(CNC(=O)NCc2nc(C)nn2-c2ccc3cccnc3c2)n(-c2ccc(Cl)c(F)c2)n1] | 1-{[1-(4-chloro-3- fluorophenyl)-3- methyl-1H-1,2,4- triazol-5-yl]methyl}- 3-{[3-methyl-1- (quinolin-7-yl)-1H- 1,2,4-triazol-5- yl]methyl}urea | 506 |
| 88 | 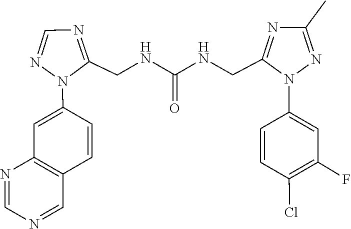 [SMILES: Cc1nc(CNC(=O)NCc2ncnn2-c2ccc3cncnc3c2)n(-c2ccc(Cl)c(F)c2)n1] | 1-{[1-(4-chloro-3- fluorophenyl)-3- methyl-1H-1,2,4- triazol-5-yl]methyl}- 3-{[1-(quinazolin-7- yl)-1H-1,2,4-triazol-5- yl]methyl}urea | 493.4 |
| 89 | 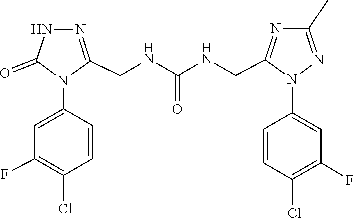 [SMILES: Cc1nc(CNC(=O)NCc2n[nH]c(=O)n2-c2ccc(Cl)c(F)c2)n(-c2ccc(Cl)c(F)c2)n1] | 1-{[1-(4-chloro-3- fluorophenyl)-3- methyl-1H-1,2,4- triazol-5-yl]methyl}- 3-{[4-(4-chloro-3- fluorophenyl)-5-oxo- 4,5-dihydro-1H-1,2,4- triazol-3- yl]methyl}urea | 509.1 |
| 90 | 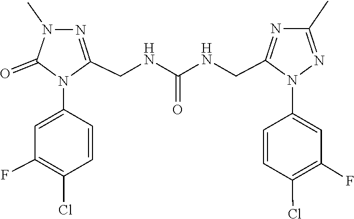 [SMILES: Cc1nc(CNC(=O)NCc2nn(C)c(=O)n2-c2ccc(Cl)c(F)c2)n(-c2ccc(Cl)c(F)c2)n1] | 3-{[4-(4-chloro-3- fluorophenyl)-1- methyl-5-oxo-4,5- dihydro-1H-1,2,4- triazol-3-yl]methyl}- 1-{[1-(4-chloro-3- fluorophenyl)-3- methyl-1H-1,2,4- triazol-5- yl]methyl}urea | 523.2 |
| 91 | 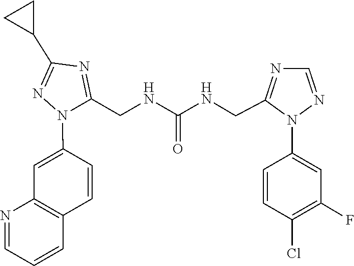 [SMILES: O=C(NCc1ncnn1-c1ccc(Cl)c(F)c1)NCc1nc(C2CC2)nn1-c1ccc2cccnc2c1] | 1-{[1-(4-chloro-3- fluorophenyl)-1H- 1,2,4-triazol-5- yl]methyl}-3-{[3- cyclopropyl-1- (quinolin-7-yl)-1H- 1,2,4-triazol-5- yl]methyl}urea | 518 |
| 92 | 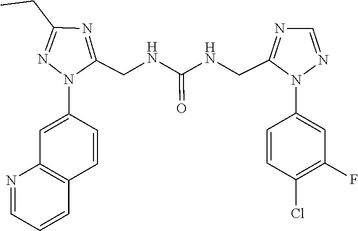 [SMILES: CCc1nc(CNC(=O)NCc2ncnn2-c2ccc(Cl)c(F)c2)n(-c2ccc3cccnc3c2)n1] | 1-{[1-(4-chloro-3- fluorophenyl)-1H- 1,2,4-triazol-5- yl]methyl}-3-{[3- ethyl-1-(quinolin-7- yl)-1H-1,2,4-triazol-5- yl]methyl}urea | 506.2 |
| 93 | 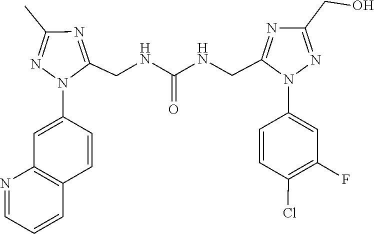 [SMILES: Cc1nc(CNC(=O)NCc2nc(CO)nn2-c2ccc(Cl)c(F)c2)n(-c2ccc3cccnc3c2)n1] | 1-{[1-(4-chloro-3- fluorophenyl)-3- (hydroxymethyl)-1H- 1,2,4-triazol-5- yl]methyl}-3-{[3- methyl-1-(quinolin-7- yl)-1H-1,2,4-triazol-5- yl]methyl}urea | 522 |
| 94 | 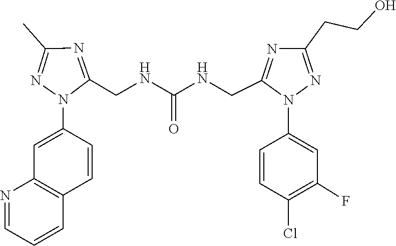 [SMILES: Cc1nc(CNC(=O)NCc2nc(CCO)nn2-c2ccc(Cl)c(F)c2)n(-c2ccc3cccnc3c2)n1] | 1-{[1-(4-chloro-3- fluorophenyl)-3-(2- hydroxyethyl)-1H- 1,2,4-triazol-5- yl]methyl}-3-{[3- methyl-1-(quinolin-7- yl)-1H-1,2,4-triazol-5- yl]methyl}urea | 536 |
| 95 | 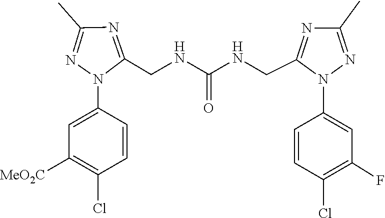 [SMILES: COC(=O)c1cc(-n2nc(C)nc2CNC(=O)NCc2nc(C)nn2-c2ccc(Cl)c(F)c2)ccc1Cl] | methyl2-chloro-5-(5- {[({[1-(4-chloro-3- fluorophenyl)-3- methyl-1H-1,2,4- triazol-5- yl]methyl}carbamoyl) amino]methyl}-3- methyl-1H-1,2,4- triazol-1-yl)benzoate | 547 |
| 96 | 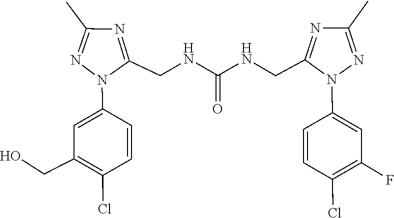 [SMILES: Cc1nc(CNC(=O)NCc2nc(C)nn2-c2ccc(Cl)c(CO)c2)n(-c2ccc(Cl)c(F)c2)n1] | 1-({1-[4-chloro-3- (hydroxymethyl)phenyl]- 3-methyl-1H-1,2,4- triazol-5-yl}methyl)- 3-{[1-(4-chloro-3- fluorophenyl)-3- methyl-1H-1,2,4- triazol-5- yl]methyl}urea | 519 |
| 97 | 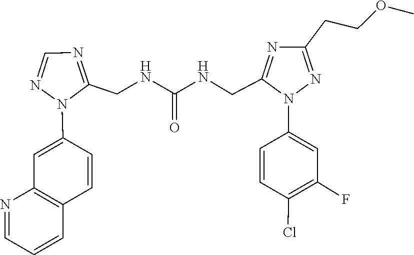 [SMILES: COCCc1nc(CNC(=O)NCc2ncnn2-c2ccc3cccnc3c2)n(-c2ccc(Cl)c(F)c2)n1] | 1-{[1-(4-chloro-3- fluorophenyl)-3-(2- methoxyethyl)-1H- 1,2,4-triazol-5- yl]methyl}-3-{[1- (quinolin-7-yl)-1H- 1,2,4-triazol-5- yl]methyl}urea | 536 |
| 98 | 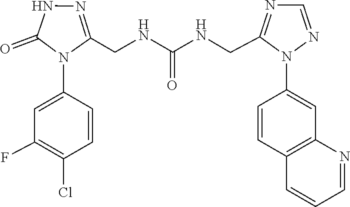 [SMILES: O=C(NCc1ncnn1-c1ccc2cccnc2c1)NCc1n[nH]c(=O)n1-c1ccc(Cl)c(F)c1] | 3-{[4-(4-chloro-3- fluorophenyl)-5-oxo- 4,5-dihydro-1H-1,2,4- triazol-3-yl]methyl}- 1-{[1-(quinolin-7-yl)- 1H-1,2,4-triazol-5- yl]methyl}urea | 494.1. |
| 99 | 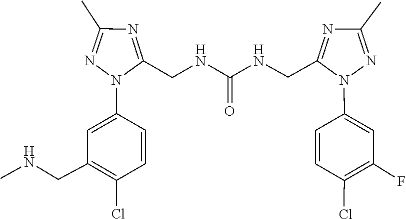 [SMILES: CNCc1cc(-n2nc(C)nc2CNC(=O)NCc2nc(C)nn2-c2ccc(Cl)c(F)c2)ccc1Cl] | 1-[(1-{4-chloro-3- [(methylamino)methyl] phenyl}-3-methyl- 1H-1,2,4-triazol-5- yl)methyl]-3-{[1-(4- chloro-3- fluorophenyl)-3- methyl-1H-1,2,4- triazol-5- yl]methyl}urea | 532 |
| 100 | 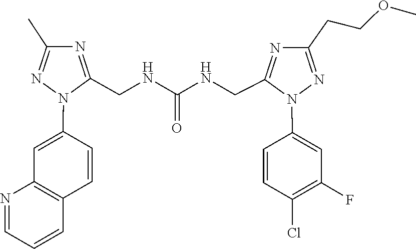 [SMILES: COCCc1nc(CNC(=O)NCc2nc(C)nn2-c2ccc3cccnc3c2)n(-c2ccc(Cl)c(F)c2)n1] | 1-{[1-(4-chloro-3- fluorophenyl)-3-(2- methoxyethyl)-1H- 1,2,4-triazol-5- yl]methyl}-3-{[3- methyl-1-(quinolin-7- yl)-1H-1,2,4-triazol-5- yl]methyl}urea | 550.1 |
| 101 | 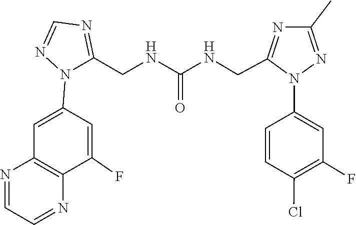 [SMILES: Cc1nc(CNC(=O)NCc2ncnn2-c2cc(F)c3nccnc3c2)n(-c2ccc(Cl)c(F)c2)n1] | 1-{[1-(4-chloro-3- fluorophenyl)-3- methyl-1H-1,2,4- triazol-5-yl]methyl}- 3-{[1-(8- fluoroquinoxalin-6- yl)-1H-1,2,4-triazol-5- yl]methyl}urea | 511 |
| 102 | 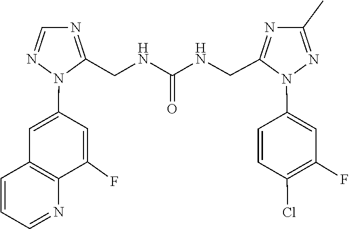 [SMILES: Cc1nc(CNC(=O)NCc2ncnn2-c2cc(F)c3ncccc3c2)n(-c2ccc(Cl)c(F)c2)n1] | 1-{[1-(4-chloro-3- fluorophenyl)-3- methyl-1H-1,2,4- triazol-5-yl]methyl}- 3-{[1-(8- fluoroquinolin-6-yl)- 1H-1,2,4-triazol-5- yl]methyl}urea | 510 |
| 103 | 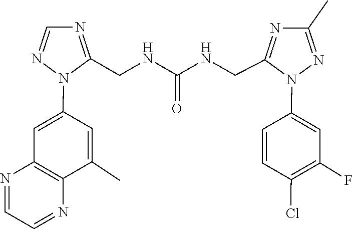 [SMILES: Cc1nc(CNC(=O)NCc2ncnn2-c2cc(C)c3nccnc3c2)n(-c2ccc(Cl)c(F)c2)n1] | 1-{[1-(4-chloro-3- fluorophenyl)-3- methyl-1H-1,2,4- triazol-5-yl]methyl}- 3-{[1-(8- methylquinoxalin-6- yl)-1H-1,2,4-triazol-5- yl]methyl}urea | 507 |
| 104 | 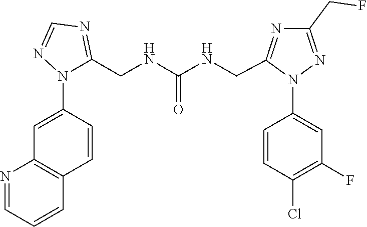 [SMILES: O=C(NCc1ncnn1-c1ccc2cccnc2c1)NCc1nc(CF)nn1-c1ccc(Cl)c(F)c1] | 1-{[1-(4-chloro-3- fluorophenyl)-3- (fluoromethyl)-1H- 1,2,4-triazol-5- yl]methyl}-3-{[1- (quinolin-7-yl)-1H- 1,2,4-triazol-5- yl]methyl}urea | 510 |
| 105 | 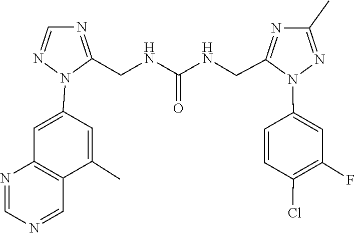 [SMILES: Cc1nc(CNC(=O)NCc2ncnn2-c2cc(C)c3cncnc3c2)n(-c2ccc(Cl)c(F)c2)n1] | 1-{[1-(4-chloro-3- fluorophenyl)-3- methyl-1H-1,2,4- triazol-5-yl]methyl}- 3-{[1-(5- methylquinazolin-7- yl)-1H-1,2,4-triazol-5- yl]methyl}urea | 507 |
| 106 | 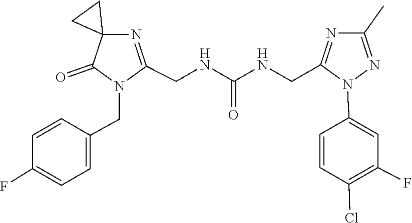 [SMILES: Cc1nc(CNC(=O)NCC2=NC3(CC3)C(=O)N2Cc2ccc(F)cc2)n(-c2ccc(Cl)c(F)c2)n1] | 1-{[1-(4-chloro-3- fluorophenyl)-3- methyl-1H-1,2,4- triazol-5-yl]methyl}- 3-({6-[(4- fluorophenyl)methyl]- 7-oxo-4,6- diazaspiro[2.4]hept-4- en-5-yl}methyl)urea | 514.1 |
| 107 | 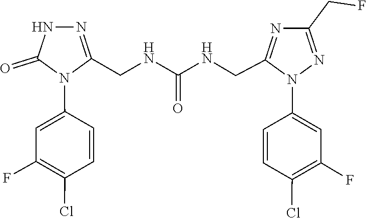 [SMILES: O=C(NCc1nc(CF)nn1-c1ccc(Cl)c(F)c1)NCc1n[nH]c(=O)n1-c1ccc(Cl)c(F)c1] | 1-{[1-(4-chloro-3- fluorophenyl)-3- (fluoromethyl)-1H- 1,2,4-triazol-5- yl]methyl}-3-{[4-(4- chloro-3- fluorophenyl)-5-oxo- 4,5-dihydro-1H-1,2,4- triazol-3- yl]methyl}urea | 527 |
| 108 | 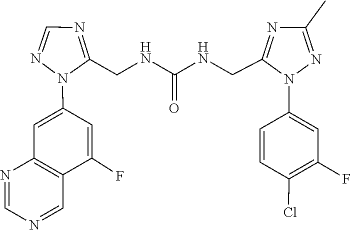 [SMILES: Cc1nc(CNC(=O)NCc2ncnn2-c2cc(F)c3cncnc3c2)n(-c2ccc(Cl)c(F)c2)n1] | 1-{[1-(4-chloro-3- fluorophenyl)-3- methyl-1H-1,2,4- triazol-5-yl]methyl}- 3-{[1-(5- fluoroquinazolin-7- yl)-1H-1,2,4-triazol-5- yl]methyl}urea | 511 |
| 109 | 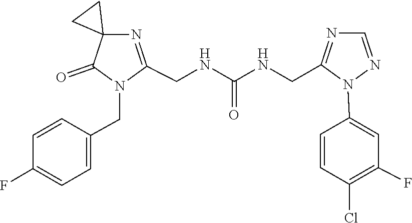 [SMILES: O=C(NCC1=NC2(CC2)C(=O)N1Cc1ccc(F)cc1)NCc1ncnn1-c1ccc(Cl)c(F)c1] | 1-{[1-(4-chloro-3- fluorophenyl)-1H- 1,2,4-triazol-5- yl]methyl}-3-({6-[(4- fluorophenyl)methyl]- 7-oxo-4,6- diazaspiro[2.4]hept-4- en-5-yl}methyl)urea | 500 |
| 110 | 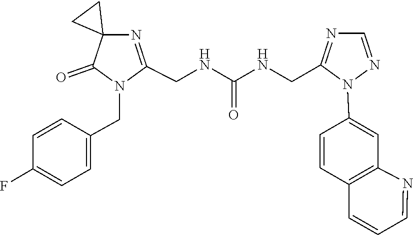 [SMILES: O=C(NCC1=NC2(CC2)C(=O)N1Cc1ccc(F)cc1)NCc1ncnn1-c1ccc2cccnc2c1] | 3-({6-[(4- fluorophenyl)methyl]- 7-oxo-4,6- diazaspiro[2.4]hept-4- en-5-yl}methyl)-1- {[1-(quinolin-7-yl)- 1H-1,2,4-triazol-5- yl]methyl}urea | 499.2 |
| 111 | 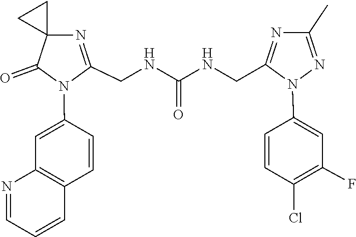 [SMILES: Cc1nc(CNC(=O)NCC2=NC3(CC3)C(=O)N2c2ccc3cccnc3c2)n(-c2ccc(Cl)c(F)c2)n1] | 1-{[1-(4-chloro-3- fluorophenyl)-3- methyl-1H-1,2,4- triazol-5-yl]methyl}- 3-{[7-oxo-6-(quinolin- 7-yl)-4,6- diazaspiro[2.4]hept-4- en-5-yl]methyl}urea | 533 |
| 112 | 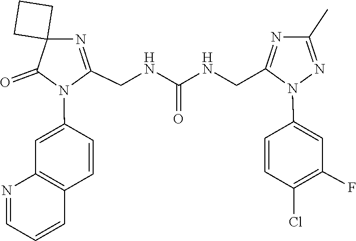 [SMILES: Cc1nc(CNC(=O)NCC2=NC3(CCC3)C(=O)N2c2ccc3cccnc3c2)n(-c2ccc(Cl)c(F)c2)n1] | 1-{[1-(4-chloro-3- fluorophenyl)-3- methyl-1H-1,2,4- triazol-5-yl]methyl}- 3-{[8-oxo-7-(quinolin- 7-yl)-5,7- diazaspiro[3.4]oct-5- en-6-yl]methyl}urea | 547 |
| 113 | 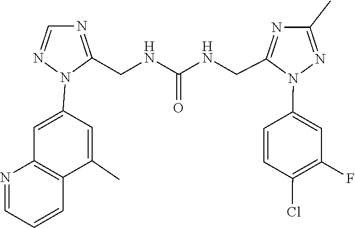 [SMILES: Cc1nc(CNC(=O)NCc2ncnn2-c2cc(C)c3cccnc3c2)n(-c2ccc(Cl)c(F)c2)n1] | 1-{[1-(4-chloro-3- fluorophenyl)-3- methyl-1H-1,2,4- triazol-5-yl]methyl}- 3-{[1-(5- methylquinolin-7-yl)- 1H-1,2,4-triazol-5- yl]methyl}urea | 506 |
| 114 | 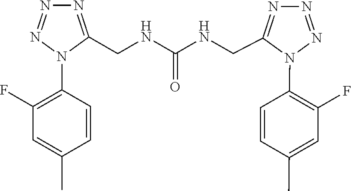 [SMILES: Cc1ccc(-n2nnnc2CNC(=O)NCc2nnnn2-c2ccc(C)cc2F)c(F)c1] | 1,3-bis({[1-(2-fluoro- 4-methylphenyl)-1H- 1,2,3,4-tetrazol-5- yl]methyl})urea | 441 |
| 115 | 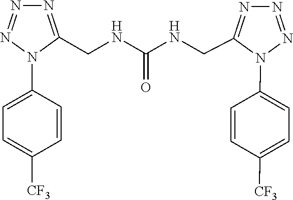 [SMILES: O=C(NCc1nnnn1-c1ccc(C(F)(F)F)cc1)NCc1nnnn1-c1ccc(C(F)(F)F)cc1] | 1,3-bis({1-[4- (trifluoromethyl)phenyl]- 1H-1,2,3,4-tetrazol- 5-yl}methyl)urea | 513 |
| 116 | 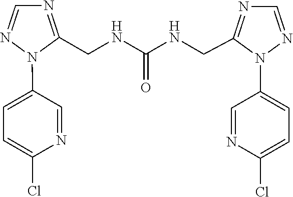 [SMILES: O=C(NCc1ncnn1-c1ccc(Cl)nc1)NCc1ncnn1-c1ccc(Cl)nc1] | 1,3-bis({[1-(6- chloropyridin-3-yl)- 1H-1,2,4-triazol-5- yl]methyl})urea | 446 |
| 117 | 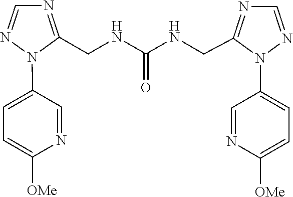 [SMILES: COc1ccc(-n2ncnc2CNC(=O)NCc2ncnn2-c2ccc(OC)nc2)cn1] | 1,3-bis({[1-(6- methoxypyridin-3-yl)- 1H-1,2,4-triazol-5- yl]methyl})urea | 437 |
| 118 | 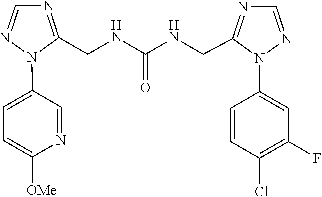 [SMILES: COc1ccc(-n2ncnc2CNC(=O)NCc2ncnn2-c2ccc(Cl)c(F)c2)cn1] | 3-{[1-(4-chloro-3- fluorophenyl)-1H- 1,2,4-triazol-5- yl]methyl}-1-{[1-(6- methoxypyridin-3-yl)- 1H-1,2,4-triazol-5- yl]methyl}urea | 458 |
| 119 | 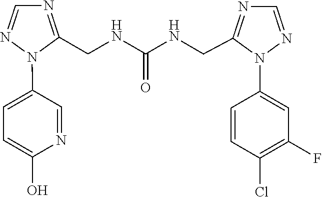 [SMILES: O=C(NCc1ncnn1-c1ccc(O)nc1)NCc1ncnn1-c1ccc(Cl)c(F)c1] | 3-{[1-(4-chloro-3- fluorophenyl)-1H- 1,2,4-triazol-5- yl]methyl}-1-{[1-(6- hydroxypyridin-3-yl)- 1H-1,2,4-triazol-5- yl]methyl}urea | 444 |
| 120 | 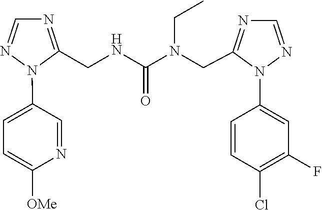 [SMILES: CCN(Cc1ncnn1-c1ccc(Cl)c(F)c1)C(=O)NCc1ncnn1-c1ccc(OC)nc1] | 3-{[1-(4-chloro-3- fluorophenyl)-1H- 1,2,4-triazol-5- yl]methyl}-3-ethyl-1- {[1-(6- methoxypyridin-3-yl)- 1H-1,2,4-triazol-5- yl]methyl}urea | 486 |
| 121 | 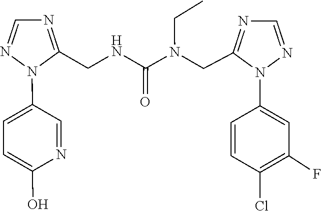 [SMILES: CCN(Cc1ncnn1-c1ccc(Cl)c(F)c1)C(=O)NCc1ncnn1-c1ccc(O)nc1] | 3-{[1-(4-chloro-3- fluorophenyl)-1H- 1,2,4-triazol-5- yl]methyl}-3-ethyl-1- {[1-(6- hydroxypyridin-3-yl)- 1H-1,2,4-triazol-5- yl]methyl}urea | 472 |
| 122 | 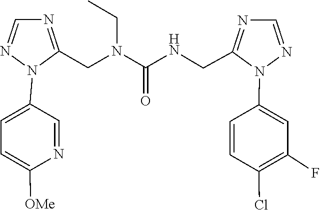 [SMILES: CCN(Cc1ncnn1-c1ccc(OC)nc1)C(=O)NCc1ncnn1-c1ccc(Cl)c(F)c1] | 3-{[1-(4-chloro-3- fluorophenyl)-1H- 1,2,4-triazol-5- yl]methyl}-1-ethyl-1- {[1-(6- methoxypyridin-3-yl)- 1H-1,2,4-triazol-5- yl]methyl}urea | 486 |
| 123 | 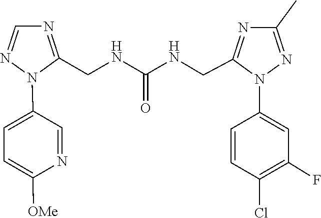 [SMILES: COc1ccc(-n2ncnc2CNC(=O)NCc2nc(C)nn2-c2ccc(Cl)c(F)c2)cn1] | 3-{[1-(4-chloro-3- fluorophenyl)-3- methyl-1H-1,2,4- triazol-5-yl]methyl}- 1-{[1-(6- methoxypyridin-3-yl)- 1H-1,2,4-triazol-5- yl]methyl}urea | 472 |
| 124 | 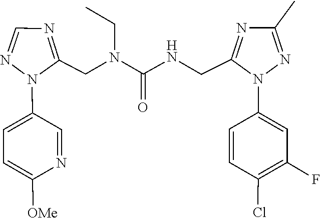 [SMILES: CCN(Cc1ncnn1-c1ccc(OC)nc1)C(=O)NCc1nc(C)nn1-c1ccc(Cl)c(F)c1] | 3-{[1-(4-chloro-3- fluorophenyl)-3- methyl-1H-1,2,4- triazol-5-yl]methyl}- 1-ethyl-1-{[1-(6- methoxypyridin-3-yl)- 1H-1,2,4-triazol-5- yl]methyl}urea | 500 |
| 125 | 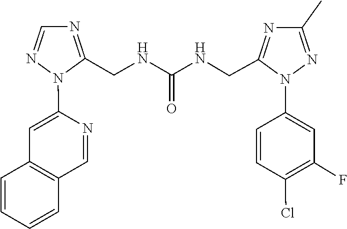 [SMILES: Cc1nc(CNC(=O)NCc2ncnn2-c2cc3ccccc3cn2)n(-c2ccc(Cl)c(F)c2)n1] | 1-{[1-(4-chloro-3- fluorophenyl)-3- methyl-1H-1,2,4- triazol-5-yl]methyl}- 3-{[1-(isoquinolin-3- yl)-1H-1,2,4-triazol-5- yl]methyl}urea | 492 |
| 126 | 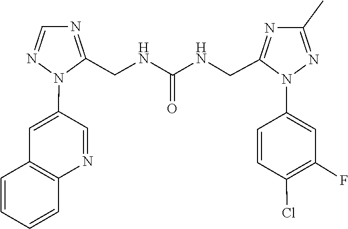 [SMILES: Cc1nc(CNC(=O)NCc2ncnn2-c2cnc3ccccc3c2)n(-c2ccc(Cl)c(F)c2)n1] | 1-{[1-(4-chloro-3- fluorophenyl)-3- methyl-1H-1,2,4- triazol-5-yl]methyl}- 3-{[1-(quinolin-3-yl)- 1H-1,2,4-triazol-5- yl]methyl}urea | 492 |
| 127 | 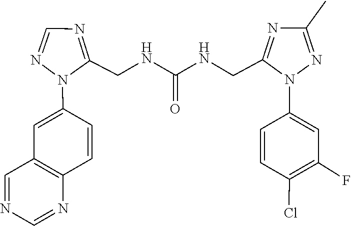 [SMILES: Cc1nc(CNC(=O)NCc2ncnn2-c2ccc3ncncc3c2)n(-c2ccc(Cl)c(F)c2)n1] | 1-{[1-(4-chloro-3- fluorophenyl)-3- methyl-1H-1,2,4- triazol-5-yl]methyl}- 3-{[1-(quinazolin-6- yl)-1H-1,2,4-triazol-5- yl]methyl}urea | 493 |
| 128 | 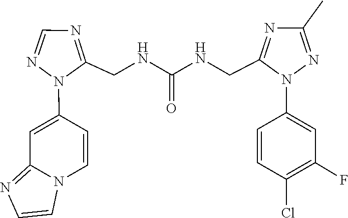 [SMILES: Cc1nc(CNC(=O)NCc2ncnn2-c2ccn3ccnc3c2)n(-c2ccc(Cl)c(F)c2)n1] | 1-{[1-(4-chloro-3- fluorophenyl)-3- methyl-1H-1,2,4- triazol-5-yl]methyl}- 3-[(1-{imidazo[1,2- a]pyridin-7-yl}-1H- 1,2,4-triazol-5- yl)methyl]urea | 481 |
| 129 | 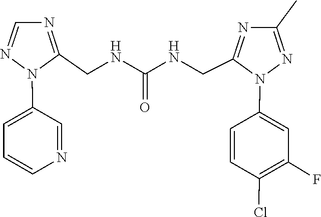 [SMILES: Cc1nc(CNC(=O)NCc2ncnn2-c2cccnc2)n(-c2ccc(Cl)c(F)c2)n1] | 1-{[1-(4-chloro-3- fluorophenyl)-3- methyl-1H-1,2,4- triazol-5-yl]methyl}- 3-{[1-(pyridin-3-y])- 1H-1,2,4-triazol-5- yl]methyl}urea | 442 |
| 130 | 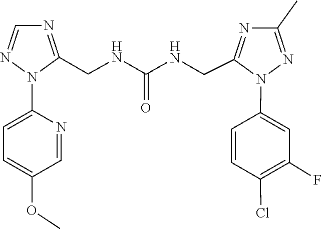 [SMILES: COc1ccc(-n2ncnc2CNC(=O)NCc2nc(C)nn2-c2ccc(Cl)c(F)c2)nc1] | 1-{[1-(4-chloro-3- fluorophenyl)-3- methyl-1H-1,2,4- triazol-5-yl]methyl}- 3-{[1-(5- methoxypyridin-2-yl)- 1H-1,2,4-triazol-5- yl]methyl}urea | 472 |
| 131 | 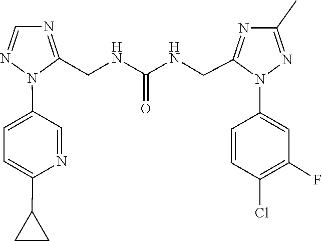 [SMILES: Cc1nc(CNC(=O)NCc2ncnn2-c2ccc(C3CC3)nc2)n(-c2ccc(Cl)c(F)c2)n1] | 1-{[1-(4-chloro-3- fluorophenyl)-3- methyl-1H-1,2,4- triazol-5-yl]methyl}- 3-{[1-(6- cyclopropylpyridin-3- yl)-1H-1,2,4-triazol-5- yl]methyl}urea | 482. |
| 132 | 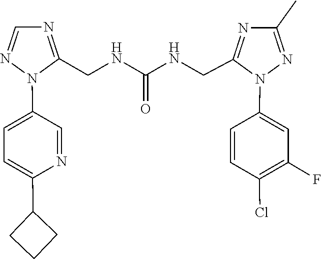 [SMILES: Cc1nc(CNC(=O)NCc2ncnn2-c2ccc(C3CCC3)nc2)n(-c2ccc(Cl)c(F)c2)n1] | 1-{[1-(4-chloro-3- fluorophenyl)-3- methyl-1H-1,2,4- triazol-5-yl]methyl}- 3-{[1-(6- cyclobutylpyridin-3- yl)-1H-1,2,4-triazol-5- yl]methyl}urea | 496 |
| 133 | 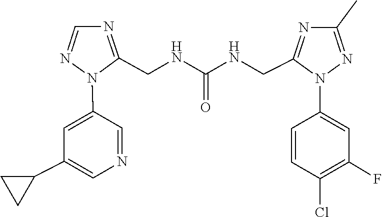 [SMILES: Cc1nc(CNC(=O)NCc2ncnn2-c2cncc(C3CC3)c2)n(-c2ccc(Cl)c(F)c2)n1] | 1-{[1-(4-chloro-3- fluorophenyl)-3- methyl-1H-1,2,4- triazol-5-yl]methyl}- 3-{[1-(5- cyclopropylpyridin-3- yl)-1H-1,2,4-triazol-5- yl]methyl}urea | 482 |
| 134 | 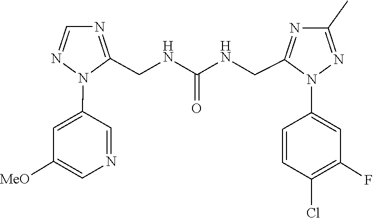 [SMILES: COc1cncc(-n2ncnc2CNC(=O)NCc2nc(C)nn2-c2ccc(Cl)c(F)c2)c1] | 1-{[1-(4-chloro-3- fluorophenyl)-3- methyl-1H-1,2,4- triazol-5-yl]methyl}- 3-{[1-(5- methoxypyridin-3-yl)- 1H-1,2,4-triazol-5- yl]methyl}urea | 472 |
| 135 | 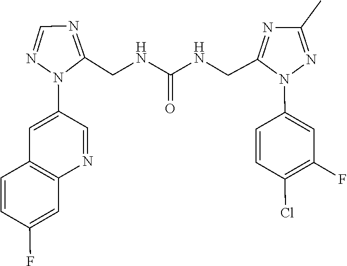 [SMILES: Cc1nc(CNC(=O)NCc2ncnn2-c2cnc3cc(F)ccc3c2)n(-c2ccc(Cl)c(F)c2)n1] | 1-{[1-(4-chloro-3- fluorophenyl)-3- methyl-1H-1,2,4- triazol-5-yl]methyl}- 3-{[1-(7- fluoroquinolin-3-yl)- 1H-1,2,4-triazol-5- yl]methyl}urea | 510 |
| 136 | 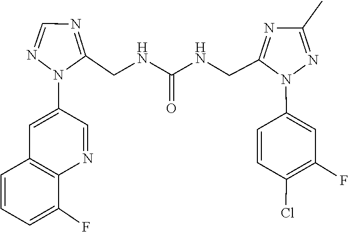 [SMILES: Cc1nc(CNC(=O)NCc2ncnn2-c2cnc3c(F)cccc3c2)n(-c2ccc(Cl)c(F)c2)n1] | 3-{[1-(4-chloro-3- fluorophenyl)-3- methyl-1H-1,2,4- triazol-5-yl]methyl}- 1-{[1-(8- fluoroquinolin-3-yl)- 1H-1,2,4-triazol-5- yl]methyl}urea | 510 |
| 137 | 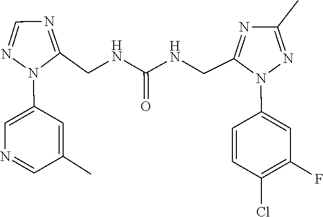 [SMILES: Cc1cncc(-n2ncnc2CNC(=O)NCc2nc(C)nn2-c2ccc(Cl)c(F)c2)c1] | 1-{[1-(4-chloro-3- fluorophenyl)-3- methyl-1H-1,2,4- triazol-5-yl]methyl}- 3-{[1-(5- methylpyridin-3-yl)- 1H-1,2,4-triazol-5- yl]methyl}urea | 456 |
| 138 | 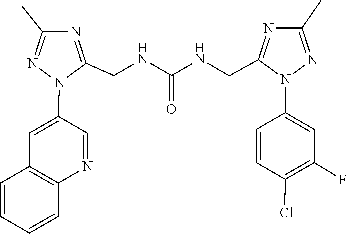 [SMILES: Cc1nc(CNC(=O)NCc2nc(C)nn2-c2cnc3ccccc3c2)n(-c2ccc(Cl)c(F)c2)n1] | 1-{[1-(4-chloro-3- fluorophenyl)-3- methyl-1H-1,2,4- triazol-5-yl]methyl}- 3-{[3-methyl-1- (quinolin-3-yl)-1H- 1,2,4-triazol-5- yl]methyl}urea | 506 |
| 139 | 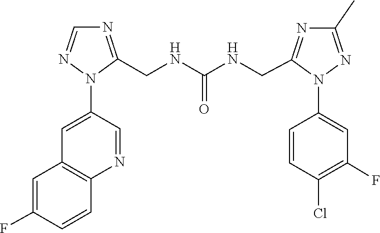 [SMILES: Cc1nc(CNC(=O)NCc2ncnn2-c2cnc3ccc(F)cc3c2)n(-c2ccc(Cl)c(F)c2)n1] | 1-{[1-(4-chloro-3- fluorophenyl)-3- methyl-1H-1,2,4- triazol-5-yl]methyl}- 3-{[1-(6- fluoroquinolin-3-yl)- 1H-1,2,4-triazol-5- yl]methyl}urea | 510 |
| 140 | 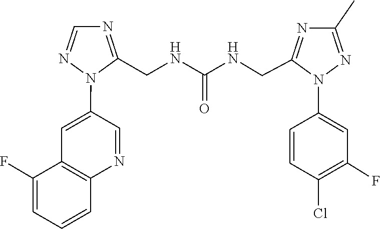 [SMILES: Cc1nc(CNC(=O)NCc2ncnn2-c2cnc3cccc(F)c3c2)n(-c2ccc(Cl)c(F)c2)n1] | 1-{[1-(4-chloro-3- fluorophenyl)-3- methyl-1H-1,2,4- triazol-5-yl]methyl}- 3-{[1-(5- fluoroquinolin-3-yl)- 1H-1,2,4-triazol-5- yl]methyl}urea | 510 |
| 141 | 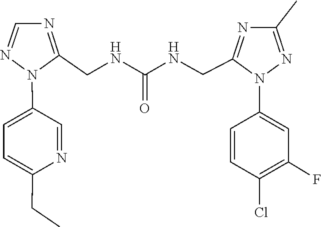 [SMILES: CCc1ccc(-n2ncnc2CNC(=O)NCc2nc(C)nn2-c2ccc(Cl)c(F)c2)cn1] | 1-{[1-(4-chloro-3- fluorophenyl)-3- methyl-1H-1,2,4- triazol-5-yl]methyl}- 3-{[1-(6-ethylpyridin- 3-yl)-1H-1,2,4-triazol- 5-yl]methyl}urea | 470 |
| 142 | 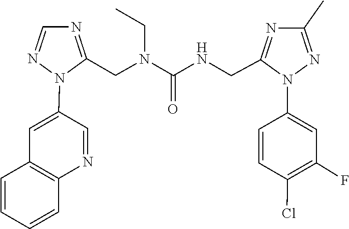 [SMILES: CCN(Cc1ncnn1-c1cnc2ccccc2c1)C(=O)NCc1nc(C)nn1-c1ccc(Cl)c(F)c1] | 1-{[1-(4-chloro-3- fluorophenyl)-3- methyl-1H-1,2,4- triazol-5-yl]methyl}- 3-ethyl-3-{[1- (quinolin-3-yl)-1H- 1,2,4-triazol-5- yl]methyl}urea | 520 |
| 143 | 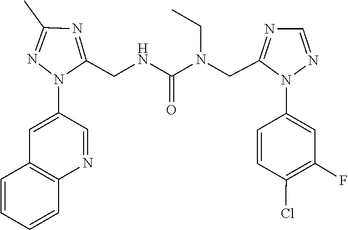 [SMILES: CCN(Cc1ncnn1-c1ccc(Cl)c(F)c1)C(=O)NCc1nc(C)nn1-c1cnc2ccccc2c1] | 1-{[1-(4-chloro-3- fluorophenyl)-1H- 1,2,4-triazol-5- yl]methyl}-1-ethyl-3- {[3-methyl-1- (quinolin-3-yl)-1H- 1,2,4-triazol-5- yl]methyl}urea | 520 |
| 144 | 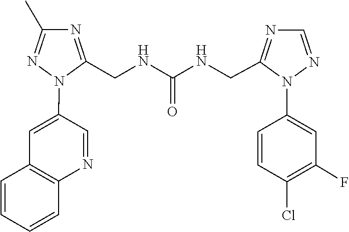 [SMILES: Cc1nc(CNC(=O)NCc2ncnn2-c2ccc(Cl)c(F)c2)n(-c2cnc3ccccc3c2)n1] | 1-{[1-(4-chloro-3- fluorophenyl)-1H- 1,2,4-triazol-5- yl]methyl}-3-{[3- methyl-1-(quinolin-3- yl)-1H-1,2,4-triazol-5- yl]methyl}urea | 492 |
| 145 | 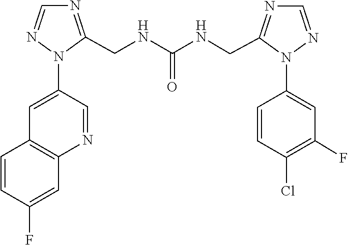 [SMILES: O=C(NCc1ncnn1-c1ccc(Cl)c(F)c1)NCc1ncnn1-c1cnc2cc(F)ccc2c1] | 1-{[1-(4-chloro-3- fluorophenyl)-1H- 1,2,4-triazol-5- yl]methyl}-3-{[1-(7- fluoroquinolin-3-yl)- 1H-1,2,4-triazol-5- yl]methyl}urea | 496 |
| 146 | 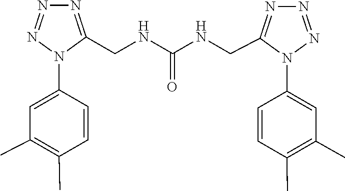 | 1,3-bis({[1-(3,4- dimethylphenyl)-1H- 1,2,3,4-tetrazol-5- yl]methyl})urea | 433.1 |

^{a} Unless made from an optically pure commercial starting material, absolute stereochemistry was not determined for each individual enantiomer or diastereomer.

### III. Pharmaceutical Compositions and Modes of Administration

Compounds provided herein are usually administered in the form of pharmaceutical compositions. Thus, provided herein are also pharmaceutical compositions that comprise one or more of the compounds described herein or a pharmaceutically acceptable salt, a stereoisomer, or a mixture of stereoisomers thereof and one or more pharmaceutically acceptable vehicles selected from carriers, adjuvants and excipients. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The pharmaceutical compositions may be administered in either single or multiple doses. The pharmaceutical composition may be administered by various methods including, for example, rectal, buccal, intranasal and transdermal routes. In certain embodiments, the pharmaceutical composition may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compounds described herein. Administration may be via, for example, capsule or enteric coated tablets. In making the pharmaceutical compositions that include at least one compound described herein or a pharmaceutically acceptable salt, a stereoisomer, or a mixture of stereoisomers thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The compositions that include at least one compound described herein or a pharmaceutically acceptable salt, a stereoisomer, or a mixture of stereoisomers thereof can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods disclosed herein employ transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein or a pharmaceutically acceptable salt, a stereoisomer, or a mixture of stereoisomers thereof. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described herein. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

The specific dose level of a compound of the present application for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject. A dose may be administered once a day (QD), twice per day (BID), or more frequently, depending on the pharmacokinetic and pharmacodynamic properties, including absorption, distribution, metabolism, and excretion of the particular compound. In addition, toxicity factors may influence the dosage and administration regimen. When administered orally, the pill, capsule, or tablet may be ingested daily or less frequently for a specified period of time. The regimen may be repeated for a number of cycles of therapy.

### IV. Methods of Treatment

The methods described herein may be applied to cell populations in vivo or ex vivo. “In vivo” means within a living individual, as within an animal or human. In this context, the methods described herein may be used therapeutically in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. In this context, the compounds and compositions described herein may be used for a variety of purposes, including therapeutic and experimental purposes. For example, the compounds and compositions described herein may be used ex vivo to determine the optimal schedule and/or dosing of administration of a compound of the present disclosure for a given indication, cell type, individual, and other parameters. Information gleaned from such use may be used for experimental purposes or in the clinic to set protocols for in vivo treatment. Other ex vivo uses for which the compounds and compositions described herein may be suited are described below or will become apparent to those skilled in the art. The selected compounds may be further characterized to examine the safety or tolerance dosage in human or non-human subjects. Such properties may be examined using commonly known methods to those skilled in the art.

In certain embodiments, the subject matter disclosed herein is directed to a method of activating PKR and/or PKM2, including methods of treating a disease or disorder in a subject by administering a therapeutically effective amount of a compound of Formula I, a pharmaceutically acceptable salt thereof, or a pharmaceutical composition comprising a compound of Formula I. In certain embodiments, the disease or disorder is selected from the group consisting of PKD (pyruvate kinase deficiency), SCD (e.g., sickle cell anemia), and thalassemia (e.g., beta-thalassemia).

In certain embodiments, the subject matter disclosed herein is directed to a method of treating a subject afflicted with a disease associated with decreased activity of PKR and/or PKM2, comprising administering to the subject an effective amount of a compound of Formula I, a pharmaceutically acceptable salt thereof, or a pharmaceutical composition comprising a compound of Formula I. In certain embodiments, the disease associated with decreased activity of PKR is selected from the group consisting of sickle cell disease, thalassemia, hereditary non-spherocytic hemolytic anemia, hemolytic anemia (e.g., chronic hemolytic anemia caused by phosphoglycerate kinase deficiency), hereditary spherocytosis, hereditary elliptocytosis, abetalipoproteinemia (or Bassen-Kornzweig syndrome), paroxysmal nocturnal hemoglobinuria, acquired hemolytic anemia (e.g., congenital anemias (e.g., enzymopathies)), and anemia of chronic diseases. In certain embodiments, the sickle cell disease is sickle cell anemia.

In certain embodiments, the subject matter described herein is directed to a method of treating a disease or disorder associated with modulation of a pyruvate kinase in a subject, comprising administering to the subject an effective amount of a compound of Formula I, a pharmaceutically acceptable salt thereof, or a pharmaceutical composition comprising a compound of Formula I. In certain embodiments, the pyruvate kinase is pyruvate kinase R or PKM2.

In certain embodiments, the subject matter described herein is directed to a method for regulating 2,3-diphosphoglycerate levels in blood, comprising contacting the blood with an effective amount of a compound of Formula I, a pharmaceutically acceptable salt thereof, or a pharmaceutical composition comprising a compound of Formula I.

In certain embodiments, the subject matter described herein is directed to a method for activating mutant pyruvate kinase R (PKR) in red blood cells in a subject in need thereof comprising administering to the subject an effective amount of a compound of Formula I, a pharmaceutically acceptable salt thereof, or a pharmaceutical composition comprising a compound of Formula I.

In certain embodiments, the subject matter described herein is directed to a method for activating wild-type pyruvate kinase R (PKR) in red blood cells in a subject in need thereof comprising administering to the subject an effective amount of a compound of Formula I, a pharmaceutically acceptable salt thereof, or a pharmaceutical composition comprising a compound of Formula I.

In certain embodiments, the subject matter described herein is directed to a method of treating cancer in a subject in need thereof, comprising administering an effective amount of a compound of Formula I, a pharmaceutically acceptable salt thereof, or a pharmaceutical composition comprising a compound of Formula I. In certain embodiments, the cancer is selected from the group consisting of bladder cancer, breast cancer (e.g., ductal carcinoma), cervical cancer (e.g., squamous cell carcinoma), colorectal cancer (e.g., adenocarcinoma), esophageal cancer (e.g., squamous cell carcinoma), gastric cancer (e.g., adenocarcinoma, medulloblastoma, colon cancer, choriocarcinoma, squamous cell carcinoma), head and neck cancer, hematologic cancer (e.g., acute lymphocytic anemia, acute myeloid leukemia, acute lymphoblastic B cell leukemia, anaplastic large cell lymphoma, B-cell lymphoma, Burkitt's lymphoma, chronic lymphocytic leukemia, chronic eosinophillic leukemia/hypereosinophillic syndrome, chronic myeloid leukemia, Hodgkin's lymphoma, mantle cell lymphoma, multiple myeloma, T-cell acute lymphoblastic leukemia), lung cancer (e.g., bronchioloalveolar adenocarcinoma, mesothelioma, mucoepidermoid carcinoma, small-cell lung cancer, non-small cell lung cancer, adenocarcinoma, squamous cell carcinoma), liver cancer (e.g., hepatocellular carcinoma), lymphoma, neurological cancer (e.g., glioblastoma, neuroblastoma, neuroglioma), ovarian (e.g., adenocarcinoma), pancreatic cancer (e.g., ductal carcinoma), prostate cancer (e.g., adenocarcinoma), renal cancer (e.g., renal cell carcinoma, clear cell renal carcinoma), sarcoma (e.g., chondrosarcoma, Ewings sarcoma, fibrosarcoma, multipotential sarcoma, osteosarcoma, rhabdomyosarcoma, synovial sarcoma), skin cancer (e.g., melanoma, epidermoid carcinoma, squamous cell carcinoma), thyroid cancer (e.g., medullary carcinoma), and uterine cancer. In a preferred embodiment, the cancer is lung cancer.

In another embodiment, the present subject matter relates to the use of a compound of Formula I or a pharmaceutically acceptable salt thereof for the manufacture of a medicament for the methods and treatments of diseases described herein.

In certain embodiments, the methods of administering and treating described herein further comprise co-administration of one or more additional pharmaceutically active compounds.

In a combination therapy, the pharmaceutically active compounds can be administered at the same time, in the same formulation, or at different times. Such combination therapy comprises co-administration of a compound of Formula I or a pharmaceutically acceptable salt thereof with at least one additional pharmaceutically active compound. Combination therapy in a fixed dose combination therapy comprises co-administration of a compound of Formula I or a pharmaceutically acceptable salt thereof with at least one additional pharmaceutically active compound in a fixed-dose formulation. Combination therapy in a free dose combination therapy comprises co-administration of a compound of Formula I or a pharmaceutically acceptable salt thereof and at least one additional pharmaceutically active compound in free doses of the respective compounds, either by simultaneous administration of the individual compounds or by sequential use of the individual compounds over a period of time.

### V. Methods of Preparing Compounds of Formula I and Pharmaceutically Acceptable Salts Thereof

The starting materials and reagents used in preparing the compounds described herein are either available from commercial suppliers such as Sigma-Aldrich Chemical Co., (Milwaukee, Wis.), Bachem (Torrance, Calif.), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this disclosure can be synthesized, and various modifications to these schemes can be made and will be suggested to one skilled in the art reading this disclosure. The starting materials and the intermediates, and the final products of the reaction may be isolated and purified if desired using conventional techniques, including but not limited to filtration, distillation, crystallization, chromatography and the like. Such materials may be characterized using conventional means, including physical constants and spectral data.

Synthetic chemistry transformations and protecting group methodologies (protection and deprotection) useful in synthesizing compounds and necessary reagents and intermediates are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); and L. Paquette, ed., Encyclopedia of Reagents for Organic Synthesis, John Wiley and Sons (1995) and subsequent editions thereof.

Compounds may be prepared singly or as compound libraries comprising at least 2, for example 5 to 1,000 compounds, or 10 to 100 compounds. Libraries of compounds of Formula I may be prepared by a combinatorial ‘split and mix’ approach or by multiple parallel syntheses using either solution phase or solid phase chemistry, by procedures known to those skilled in the art. Thus, according to a further aspect, there is provided a compound library comprising at least 2 compounds, or pharmaceutically acceptable salts thereof.

Unless specified to the contrary, the reactions described herein take place at atmospheric pressure over a temperature range from about −78° C. to about 150° C., such as from about 0° C. to about 125° C. and further such as at about room (or ambient) temperature, e.g., about 20° C. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

### General Synthetic Schemes

Compounds of Formula I can be prepared by the following General Procedure described in Scheme 1 below:

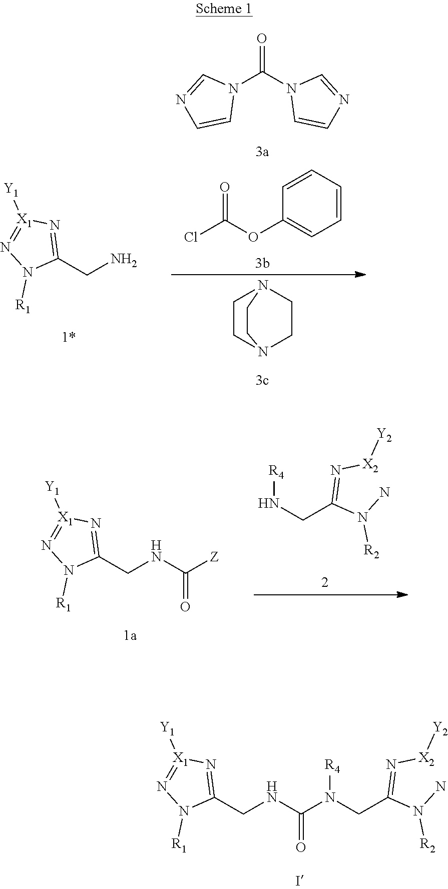

Reaction of compound 1* (synthesized as described herein) with carbonyl di-imidazole (3a; CDI henceforth) or phenyl chloroformate (3b) in the presence of base (e.g. Et₃N), or with 1,4-diazabicyclo[2,2,2]octane (3c; DABCO henceforth) in the presence of di-tertbutyl dicarbonate (Boc₂O) gives intermediate compound 1a, which, without purification can be further treated with compound 2 (synthesized as described herein) to provide asymmetric final compound I′.

Compounds of Formula II can be prepared by the following General Procedure described in Scheme 2 below:

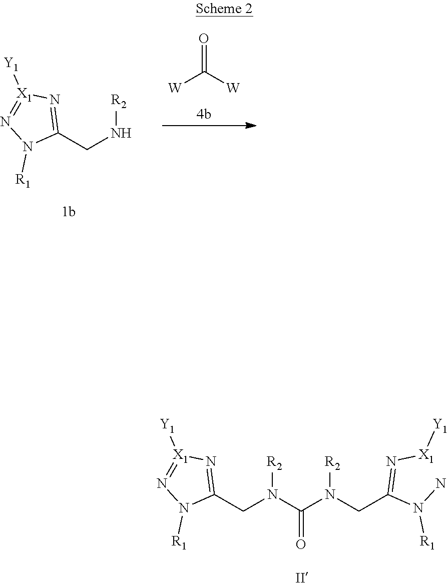

Reaction of 2 equivalents of compound 1b (synthesized as described herein) with CDI (4b where W is imidazolyl) or triphosgene (4b where W is trichloromethoxyl) in the presence of base (e.g. Et₃N) or with DABCO (3c in scheme 1) in the presence of Boc₂O provides symmetric final compound II′.

The subject matter described herein includes but is not limited to the following embodiments:
1. A compound of Formula I

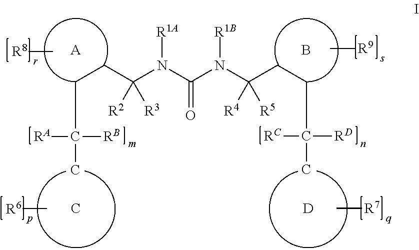

or a pharmaceutically acceptable salt thereof, wherein,

m and n are each independently 0, 1, 2, or 3;

r and s are each independently 0, 1, 2, or 3;

p and q are each independently 0, 1, 2, 3, 4, or 5;

R^{A}, R^{B}, R^{C}, and R^{D}, in each instance, are independently selected from the group consisting of hydrogen, halogen, C₁-C₆ alkyl, hydroxy, hydroxy-C₁-C₆ alkyl, and C₁-C₆ alkoxy;

R⁶ and R⁷, in each instance, are independently selected from the group consisting of halogen, C₁-C₆ alkoxy, C₃-C₇ cycloalkyl, C₁-C₆ alkyl, —C(O)OR¹⁰, hydroxy, hydroxy-C₁-C₆ alkyl, halo-C₁-C₆ alkoxy, halo-C₁-C₆ alkyl, oxo, NR^{E1}R^{G1}, and —C₁-C₆ alkyl-NR^{x}R^{y};
wherein, R¹⁰, R^{E1}, R^{G1}, R^{x} and R^{y} are each independently hydrogen or C₁-C₆ alkyl;

Ring C and Ring D are each independently selected from the group consisting of C₄-C₇ cycloalkyl, 4- to 10-membered monocyclic or bicyclic fused heterocyclyl, 6- to 10-membered aryl, and 5- to 10-membered heteroaryl, each optionally substituted with R⁶ or R⁷;
wherein said 4- to 10-membered monocyclic or bicyclic fused heterocyclyl or 5- to 10-membered heteroaryl each independently contains 1, 2, 3, or 4 ring heteroatoms selected from N, O, and S;

R^{1A} and R^{1B} are each independently selected from the group consisting of hydrogen, C₁-C₆ alkyl, halogen, halo-C₁-C₆ alkyl, hydroxy-C₁-C₆ alkyl, hydroxy, C₁-C₆ alkoxy, and C₁-C₆ alkoxy-C₁-C₆ alkyl;

or, R^{1A} and R^{1B}, together with the nitrogen atom to which each is attached form a 5- to 7-membered heterocyclyl;

R², R³, R⁴, and R⁵ are each independently hydrogen, halogen, or C₁-C₆ alkyl;

Ring A and Ring B are each independently a 7- to 10-membered spirocyclic heterocyclyl, 5- or 6-membered partially unsaturated monocyclic heterocyclyl, or a 5- or 6-membered heteroaryl, each optionally substituted with R⁸ or R⁹;
wherein, Ring A and Ring B each independently contains 1, 2, 3, or 4 ring heteroatoms selected from N, O, and S;
   and,

R⁸ and R⁹, in each instance, are independently selected from the group consisting of halogen, halo-C₁-C₆ alkyl, an oxo group formed by an oxygen double bonded to a ring carbon, C₁-C₆ alkyl, C₃-C₅ cycloalkyl, hydroxy, hydroxy-C₁-C₆ alkyl, C₁-C₃ alkoxy-C₁-C₆ alkyl, NR^{E2}R^{G2}, —C(O)NR⁴⁰R⁵⁰, and —CH₂C(O)OR⁶⁰;
wherein, R^{E2}, R^{G2}, R⁴⁰, R⁵⁰ and R⁶⁰ are each independently hydrogen or C₁-C₆ alkyl.
2. The compound of embodiment 1, wherein R^{1A} and R^{1B} are each independently hydrogen, —CH₃, —CH₂CH₃, —CH₂CH₂CH₃, or —CH(CH₃)₂.
3. The compound of embodiment 1 or 2, wherein WA and R^{1B} are each hydrogen.
4. The compound of any one of embodiments 1-3, wherein R², R³, R⁴, and R⁵ are each hydrogen.
5. The compound of any one of embodiments 1-4, wherein Ring A and Ring B each contain at least one N.
6. The compound of any one of embodiments 1-5, wherein Ring A and Ring B are each independently a 5-membered heteroaryl or 5-membered partially unsaturated monocyclic heterocyclyl.
7. The compound of any one of embodiments 1-6, wherein Ring A and Ring B are each independently triazolyl or tetrazolyl, optionally substituted with R⁸ or R⁹.
8. The compound of any one of embodiments 1-7, wherein R⁸ and R⁹, if present, in each instance, are selected from the group consisting of —CH₃, —CH₂CH₃, —CH₂CH₂CH₃, —CH(CH₃)₂, cyclopropyl, —CH₂OH, —CH₂CH₂OH, —CH₂CH₂OCH₃, —CH₂F, and an oxo group formed by an oxygen double bonded to a ring carbon.
9. The compound of any one of embodiments 1-8, wherein R⁸ and R⁹, if present, in each instance, are —CH₃.
10. The compound of any one of embodiments 1-9, wherein r is 1.
11. The compound of any one of embodiments 1-9, wherein r is 0.
12. The compound of any one of embodiments 1-11, wherein s is 1.
13. The compound of any one of embodiments 1-11, wherein s is 0.
14. The compound of any one of embodiments 1-13, wherein Ring C

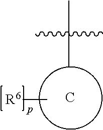

### and Ring D

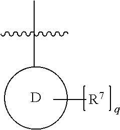

are each independently selected from the group consisting of 5- to 10-membered monocyclic or bicyclic fused heterocyclyl, 6- to 10-membered aryl, and 5- to 10-membered heteroaryl, each optionally substituted with R⁶ or R⁷.
15. The compound of any one of embodiments 1-14, wherein Ring C

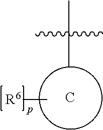

### and Ring D

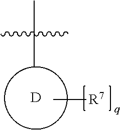

are each independently selected from the group consisting of optionally substituted phenyl, cyclohexyl, tetrahydrofuranyl, quinolinyl, pyrimidinyl, pyridinyl, benzothiazolyl, dihydrobenzofuranyl, quinoxalinyl, benzoimidazolyl, benzodioxolyl, naphthyridinyl, and imidazopyridinyl.
16. The compound of any one of embodiments 1-15, wherein Ring C

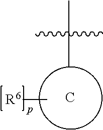

### and Ring D

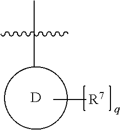

are each independently selected from the group consisting of optionally substituted phenyl, tetrahydrofuranyl, quinolinyl, benzothiazolyl, quinoxalinyl, and pyridinyl.
17. The compound of any one of embodiments 1-16, wherein Ring C

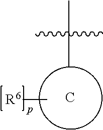

is selected from the group consisting of optionally substituted phenyl, tetrahydrofuranyl, quinolinyl, and pyridinyl, and Ring D

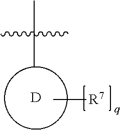

is selected from the group consisting of optionally substituted phenyl, tetrahydrofuranyl, quinolinyl, benzothiazolyl, quinoxalinyl, and quinolinyl.
18. The compound of any one of embodiments 1-17, wherein Ring C

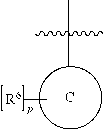

### and Ring D

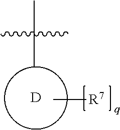

are each optionally substituted phenyl.
19. The compound of any one of embodiments 1-17, wherein Ring C

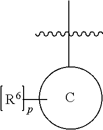

is optionally substituted phenyl and Ring

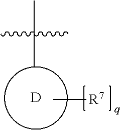

is optionally substituted tetrahydrofuranyl.
20. The compound of any one of claims embodiments 1-17, wherein Ring C

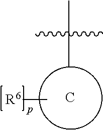

is optionally substituted tetrahydrofuranyl and ring D

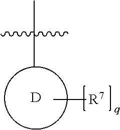

optionally substituted quinolinyl.
21. The compound of any one of embodiments 1-17, wherein Ring C

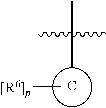

is optionally substituted phenyl and Ring D

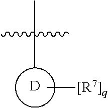

is optionally substituted benzothiazolyl.
22. The compound of any one of embodiments 1-17, wherein Ring C

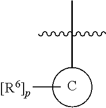

is optionally substituted phenyl and Ring D

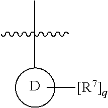

is optionally substituted quinoxalinyl.
23. The compound of any one of embodiments 1-17, wherein Ring C

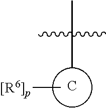

### and Ring D

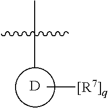

are each optionally substituted quinolinyl.
24. The compound of any one of embodiments 1-17, wherein Ring C

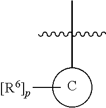

is optionally substituted pyridinyl and Ring D

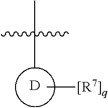

is optionally substituted phenyl.
25. The compound of any one of embodiments 1-24, wherein R⁶ and R⁷, if present, in each instance are independently selected from the group consisting of chloro, fluoro, —CH₃, hydroxy, —CH₂OH, —CF₃, —OCH₃, —CH₂CH₃, cyclobutyl, and cyclopropyl.
26. The compound of embodiment 25, wherein m and n are each independently selected from the group consisting of 0, 1, and 2.
27. The compound of embodiment 25 or 26, wherein at least one of said Ring C

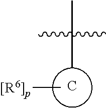

### and Ring D

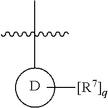

is selected from the group consisting of optionally substituted phenyl, tetrahydrofuranyl, and pyridinyl.
28. The compound of any one of embodiments 25-27, wherein at least one of said Ring C

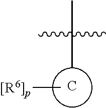

### and Ring D

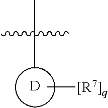

is optionally substituted phenyl.
29. The compound of any one of embodiments 25-27, wherein at least one of said Ring C

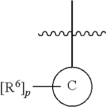

### and Ring D

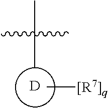

is optionally substituted tetrahydrofuranyl.
30. The compound of any one of embodiments 25-27, wherein at least one of said Ring C

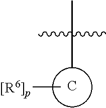

### and Ring D

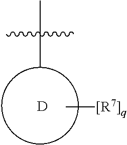

is optionally substituted pyridinyl.
31. The compound of any one of embodiments 15, 16, 17, 18, 19, 21, 22, 24, 27, or 28, wherein said optionally substituted phenyl is selected from the group consisting of

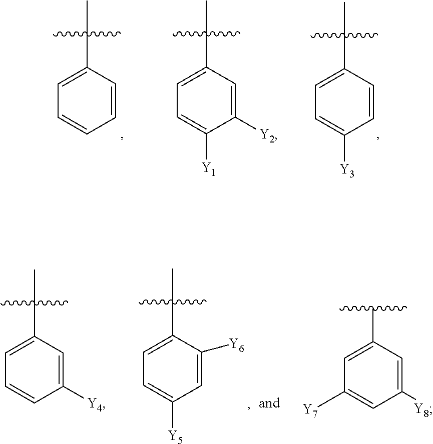

wherein Y₁, Y₂, Y₃, Y₄, Y₅, Y₆, Y₇, and Y₈ are each independently selected from the group consisting of fluoro, chloro, —OCH₃, hydroxy, —CH₂OH, and —CH₃.
32. The compound of embodiment 31, wherein said optionally substituted phenyl is

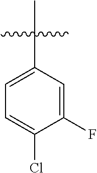 [SMILES: *c1ccc(Cl)c(F)c1]

33. The compound of any one of embodiments 15, 16, 17, 19, 20, 27, or 29, wherein said optionally substituted tetrahydrofuranyl is selected from the group consisting of

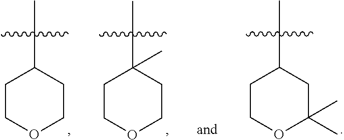

34. The compound of any one of embodiments 15, 16, 17, 24, 27, or 30, wherein said optionally substituted pyridinyl is selected from the group consisting of

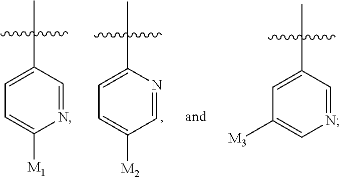

wherein M₁, M₂, and M₃ are each independently selected from the group consisting of hydroxy, cyclopropyl, cyclobutyl, —OCH₃, and —CH₃.
35. The compound of any one of embodiments 1-34, wherein R^{A}, R^{B}, R^{C}, and R^{D}, if present, are each hydrogen.
36. The compound of any one of embodiments 1-35, wherein m is 1.
37. The compound of any one of embodiments 1-35, wherein m is 0.
38. The compound of any one of embodiments 1-37, wherein n is 0.
39. The compound of embodiment 5, wherein Ring A is a 7- to 10-membered spirocyclic heterocyclyl and ring B is 5-membered heteroaryl.
40. The compound of embodiment 39, wherein R⁸ is oxo.
41. The compound of embodiment 39 or 40, wherein r is 1.
42. The compound of any one of embodiments 39-41, wherein ring B is an optionally substituted triazolyl.
43. The compound of embodiment 42, wherein R⁹ is —CH₃.
44. The compound of embodiment 43, wherein s is 1.
45. The compound of embodiment 42, wherein s is 0.
46. The compound of any one of embodiments 39-45, wherein Ring C

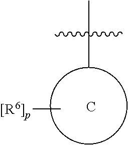

### and Ring D

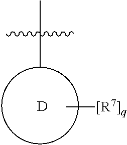

are each independently optionally substituted phenyl or optionally substituted quinolinyl.
47. The compound of embodiment 46, wherein Ring C

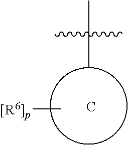

### and Ring D

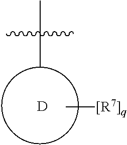

are each optionally substituted phenyl.
48. The compound of embodiment 46, wherein one of Ring C

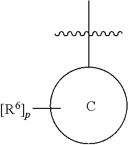

### and Ring D

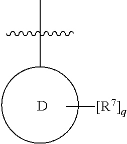

is optionally substituted phenyl and the other is optionally substituted quinolinyl.
49. The compound of any one of embodiments 46-48, wherein R⁶ and R⁷, if present, in each instance are independently selected from the group consisting of fluoro and chloro.
50. The compound of embodiment 49, wherein p and q are each independently selected from the group consisting of 0, 1, and 2.
51. The compound of any one of embodiments 46-50, wherein said optionally substituted phenyl has a structure selected from

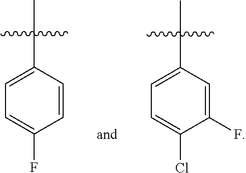 [SMILES: *c1ccc(F)cc1 ; *c1ccc(Cl)c(F)c1]

52. The compound of any one of embodiments 46 and 48-50, wherein said optionally substituted quinolinyl has the structure

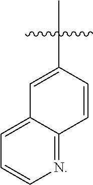 [SMILES: *c1ccc2ncccc2c1]

53. The compound of any one of embodiments 39-52, wherein R^{A}, R^{B}, R^{C}, and R^{D}, if present, are each hydrogen.
54. The compound of any one of embodiments 39-53, wherein m is 1.
55. The compound of any one of embodiments 39-53, wherein m is 0.
56. The compound of any one of embodiments 39-55, wherein n is 0.
57. The compound of embodiment 1, selected from a structure in Table 1, or a pharmaceutically acceptable salt thereof
58. A pharmaceutical composition comprising a compound of any one of embodiments 1-57 or a pharmaceutically acceptable salt thereof, and a pharmaceutically acceptable excipient.
59. A method of treating a disease or disorder associated with modulation of a pyrukate kinase in a subject, comprising administering to the subject an effective amount of a compound of any one of embodiments 1-57 or the pharmaceutical composition of embodiment 58.
60. A method of activating PKR and/or PKM2 in a subject, comprising administering to the subject an effective amount of a compound of any one of embodiments 1-57 or the pharmaceutical composition of embodiment 58.
61. A method of treating a subject afflicted with a disease associated with decreased activity of PKR and/or PKM2, comprising administering to the subject an effective amount of a compound of any one of embodiments 1-57 or the pharmaceutical composition of embodiment 58.
62. The method of embodiment 61, wherein the disease is selected from the group consisting of sickle cell disease, thalassemia, hereditary non-spherocytic hemolytic anemia, hemolytic anemia, hereditary spherocytosis, hereditary elliptocytosis, abetalipoproteinemia, paroxysmal nocturnal hemoglobinuria, acquired hemolytic, and anemia of chronic diseases.
63. The method of embodiment 62, wherein said sickle cell disease is sickle cell anemia.
64. A method for regulating 2,3-diphosphoglycerate levels in blood comprising contacting the blood with an effective amount of a compound of any one of embodiments 1-57 or the pharmaceutical composition of embodiment 58.
65. A method for activating mutant pyruvate kinase R (PKR) in red blood cells in a subject in need thereof comprising administering to the subject an effective amount of a compound of any one of embodiments 1-57 or the pharmaceutical composition of embodiment 58.
66. A method for activating wild-type pyruvate kinase R (PKR) in red blood cells in a subject in need thereof comprising administering to the subject an effective amount of a compound of any one of embodiments 1-57 or the pharmaceutical composition of embodiment 58.

The following examples are offered by way of illustration and not by way of limitation.

### EXAMPLES

### I. Synthetic Examples

Several intermediates used in the synthetic preparations of the compounds described herein are provided below:

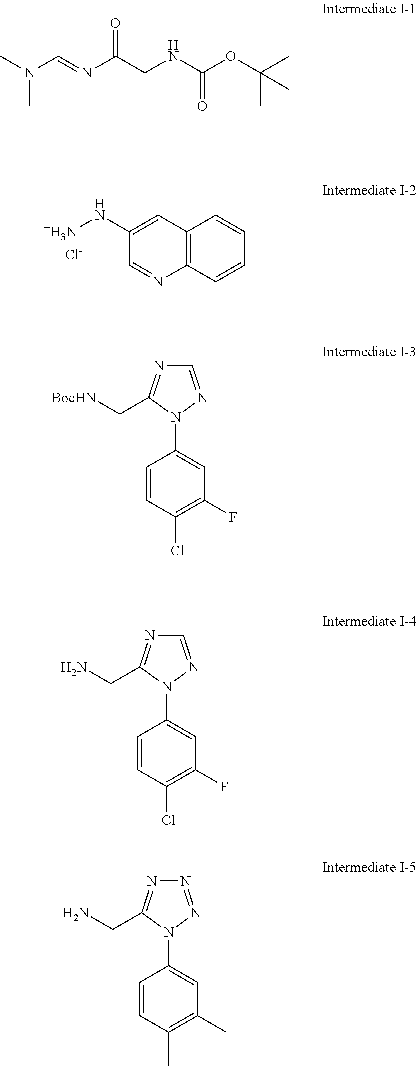 [SMILES: CN(C)/C=N/C(=O)CNC(=O)OC(C)(C)C ; [Cl-] ; [NH3+]Nc1cnc2ccccc2c1 ; CC(C)(C)OC(=O)NCc1ncnn1-c1ccc(Cl)c(F)c1 ; NCc1ncnn1-c1ccc(Cl)c(F)c1 ; Cc1ccc(-n2nnnc2CN)cc1C]

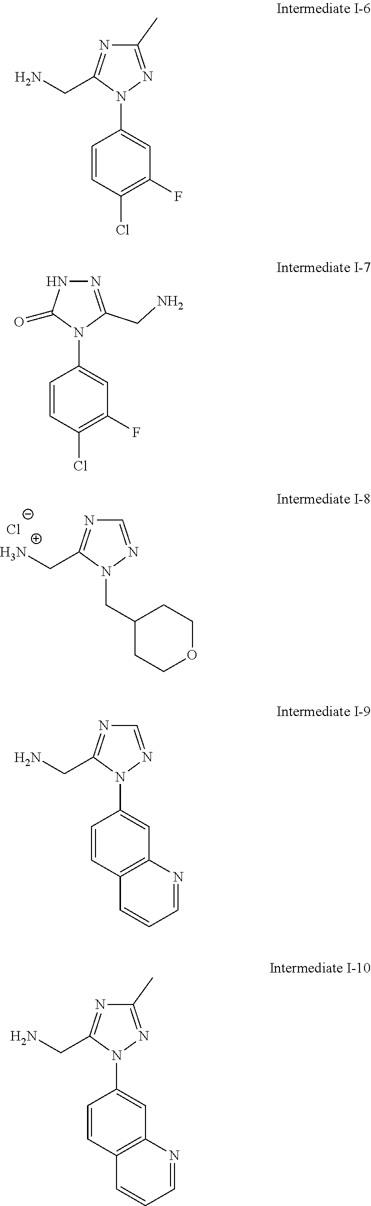 [SMILES: Cc1nc(CN)n(-c2ccc(Cl)c(F)c2)n1 ; NCc1n[nH]c(=O)n1-c1ccc(Cl)c(F)c1 ; [Cl-] ; [NH3+]Cc1ncnn1CC1CCOCC1 ; NCc1ncnn1-c1ccc2cccnc2c1 ; Cc1nc(CN)n(-c2ccc3cccnc3c2)n1]

### Example 1.1

### Synthesis of tert-butyl N-({[(1E)(dimethylamino)methylidene]arbamoyl}methyl) carbamate (Intermediate I-1)

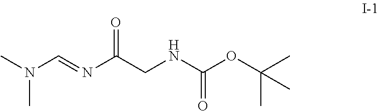 [SMILES: CN(C)/C=N/C(=O)CNC(=O)OC(C)(C)C]

A mixture of dimethoxy-N,N-dimethylmethanamine (38.13 mL; 0.29 mol; 2.00 eq.) and tert-butyl N-(carbamoylmethyl)carbamate (25.00 g; 0.14 mol; 1.00 eq.) in tetrahydrofuran (500.00 mL) was stirred under reflux for 4 h. It was then allowed to stir at ambient temperature overnight. Most of the solvent was evaporated, and hexane was added to the residue while stirring. The resulting mixture was cooled to 0° C. and the white precipitate formed was collected by gravity filtration and then air-dried. This crude material (32 g, 97.3% yield), was directly used in the next step without further purification. 1-H NMR (400 MHz, DMSO-d6) δ 8.38 (s, 1H), 6.72 (t, J=6.2 Hz, 1H), 3.63 (d, J=6.1 Hz, 2H), 3.10 (d, J=1.8 Hz, 3H), 2.96 (d, J=1.7 Hz, 3H), 1.36 (s, 9H).

### Example 1.2

### General Procedure 1: Synthesis of Hydrazines from Amines

### Synthesis of 2-(quinolin-3-yl)hydrazinium chloride (Intermediate I-2)

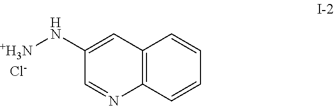 [SMILES: [Cl-] ; [NH3+]Nc1cnc2ccccc2c1]

To a suspension of 3-quinolinamine (3 000.00 mg; 20.81 mmol; 1.00 eq.) in concentrated HCl (5 V; 15 mL) at 0° C. was added an aqueous solution (2.5 mL; 8 M) of sodium nitrite (1 435.67 mg; 20.81 mmol; 1.00 eq.) drop-wise. After 1 h, tin(II)chloride dihydrate (9 390.64 mg; 41.62 mmol; 2.00 eq.) as a solution in 0.6 V (6 mL) of concentrated HCl was added and the resulting mixture was stirred while warming to ambient temperature. After 2.5 h, the mixture was filtered and the filter cake was washed with EtOH and Et₂O (once each), and air-dried to provide 2-(quinolin-3-yl)hydrazinium chloride (3.78 g; 92%) as a tan solid. This crude material was directly used in the next step without further purification. 1-H NMR (400 MHz, DMSO-d6) δ 11.26 (br. s, 3H), 9.22 (br. s, 1H), 8.85 (d, J=2.6 Hz, 1H), 8.07 (dd, J=8.3, 1.5 Hz, 1H), 8.03-7.97 (m, 1H), 7.94 (dd, J=7.7, 1.9 Hz, 1H), 7.69 (dqd, J=8.3, 6.9, 1.5 Hz, 2H). LCMS (ES) [M+1]⁺ m/z 160.

### Example 1.3

### General Procedure 2: Synthesis of Triazole-BOC-methanamines from Hydrazines

### Synthesis of tert-butyl N-{[1-(4-chloro-3-fluorophenyl)-1H-1,2,4-triazol-5-yl]methyl}carbamate (Intermediate I-3)

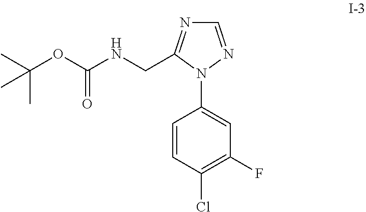 [SMILES: CC(C)(C)OC(=O)NCc1ncnn1-c1ccc(Cl)c(F)c1]

A mixture of (tert-butyl (E)-(2-(((dimethylamino)methylene)amino)-2-oxoethyl)carbamate) (10.00 g; 43.62 mmol; 1.00 eq.) and (4-chloro-3-fluorophenyl)hydrazine (7 704.00 mg; 47.98 mmol; 1.10 eq.) in acetic acid (300.00 mL; 30.00 V) was stirred at 80° C. for 50 min. The reaction mixture was then cooled to ambient temperature and concentrated in vacuo. The residue was poured into saturated NaHCO₃. Water and EtOAc were added, and the mixture was extracted twice with EtOAc. The combined organic layers were concentrated and the residue was purified on a silica gel column eluting with a gradient of 0-35% EtOAc in heptanes to provide tert-butyl N-{[1-(4-chloro-3-fluorophenyl)-1H-1,2,4-triazol-5-yl]methyl}carbamate as a light brown syrup, which solidified upon drying. 1-H NMR (400 MHz, DMSO-d6) δ 8.09 (s, 1H), 7.87-7.69 (m, 2H), 7.41 (dd, J=38.3, 7.3 Hz, 2H), 4.38 (d, J=5.7 Hz, 2H), 1.29 (s, 9H). LCMS (ES) [M+1]⁺ m/z: 327

### Example 1.4

### General Procedure 3: Synthesis of Triazole-methanamines

### Synthesis of 1-[1-(4-chloro-3-fluorophenyl)-1H-1,2,4-triazol-5-yl]methanamine (Intermediate I-4)

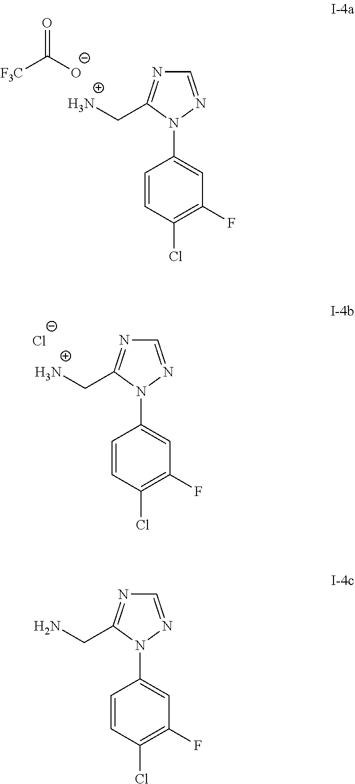 [SMILES: O=C([O-])C(F)(F)F ; [NH3+]Cc1ncnn1-c1ccc(Cl)c(F)c1 ; [Cl-] ; [NH3+]Cc1ncnn1-c1ccc(Cl)c(F)c1 ; NCc1ncnn1-c1ccc(Cl)c(F)c1]

Trifluoroacetic acid (40.43 mL; 527.92 mmol; 15.00 eq.) was added drop-wise to a solution of tert-butyl N-{[1-(4-chloro-3-fluorophenyl)-1H-1,2,4-triazol-5-yl]methyl}carbamate (11.50 g; 35.19 mmol; 1.00 eq.) in dichloromethane (230.00 mL). The mixture was stirred at ambient temperature for 3 h. The solvent was then removed under vacuum and the residue was treated with saturated NaHCO₃ and extracted 3 times with a mixture of CHCl₃: isopropanol (3:1). The combined organics were dried over MgSO4 and concentrated. The residue was purified on a silica gel column eluting with a gradient of 0-50% of a (DCM:MeOH:NH₄OH; 90:9:1) in DCM. The product thus isolated was a TFA salt (I-4a).

Alternatively, HCl (4.0 N in dioxane) can be used in place of trifluoroacetic acid, resulting in the corresponding chloride salt (I-4b). Both these materials (I-4a and I-4b) can be directly used in subsequent steps (i.e. General Procedure 7) without further purification.

This material (I-4a) was dissolved in a 3:1 mixture of CHCl₃: isopropanol and treated with a saturated solution of NaHCO₃. The organic layer was washed with brine, dried over MgSO4, filtered, and concentrated to provide 1-[1-(4-chloro-3-fluorophenyl)-1H-1,2,4-triazol-5-yl]methanamine as the free amine I-4c. 1H NMR (400 MHz, DMSO-d6) δ 8.10 (d, J=1.5 Hz, 1H), 7.90 (dt, J=10.2, 1.9 Hz, 1H), 7.79 (td, J=8.4, 1.5 Hz, 1H), 7.58 (ddd, J=8.6, 2.4, 1.2 Hz, 1H), 3.88 (s, 2H), 1.98 (s, 2H). LCMS (ES) [M+1]+m/z: 227.

### Example 1.5

### General Procedure 4: Synthesis of Tetrazole-methanamines

### Synthesis of 1-[1-(3,4-dimethylphenyl)-1H-1,2,3,4-tetrazol-5-yl]methanamine (Intermediate I-5)

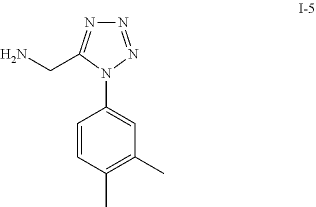

Step 1

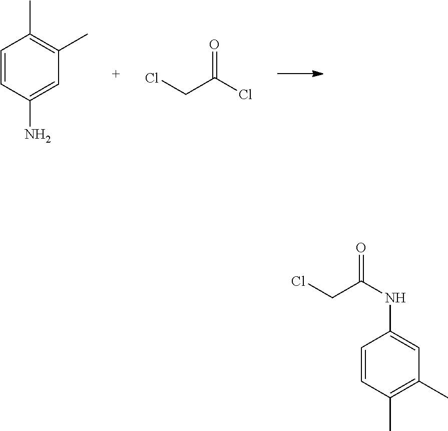

Into a 250-mL three neck round-bottom flask, 2-chloroacetyl chloride (5.6 g, 49.56 mmol, 1.2 eq.) was added drop-wise to a solution of 3,4-dimethylaniline (5 g, 41.3 mmol, 1.00 eq.) and Et₃N (4.17 g, 41.3 mmol, 1.00 eq.) in dry DMF (100 mL) at 0° C. The resulting solution was stirred for 2 h at ambient temperature. The reaction was then poured into ice water and the formed precipitate was filtered off and dried to obtain 6.5 g (80%) of 2-chloro-N-(3,4-dimethylphenyl)acetamide as an off-white solid. LCMS (ES) [M+1]⁺ m/z: 198.

Step 2

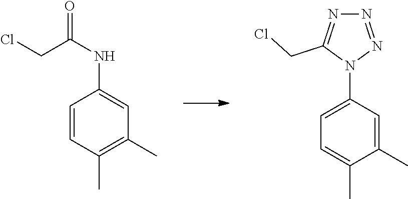

Into a 250-mL three neck round-bottom flask, was placed 2-chloro-N-(3,4-dimethylphenyl)acetamide (3.0 g, 15.2 mmol, 1.00 equiv), TMSN₃ (7.0 g, 60.8 mmol, 4.00 equiv) and Na₂CO₃ (4.5 g, 42.56 mmol, 2.80 equiv) in DCE (180 mL). Then (OTf)₂O (4.72 g, 16.72 mmol, 1.10 equiv) was added at −40° C. under N₂. The resulting mixture was slowly warmed to ambient temperature and stirred for 20 h at 80° C. The resulting mixture was then filtered, and the filtrate was concentrated under vacuum. The residue was purified by a silica gel column eluted with ethyl acetate/petroleum ether (⅛). This resulted in 1.2 g (36%) of 5-(chloromethyl)-1-(3,4-dimethylphenyl)-1H-tetrazole as a yellow solid. LCMS (ES) [M+1]⁺ m/z: 223.

Step 3

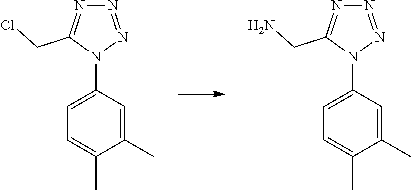

Into a 40-mL tube was placed methyl 5-(chloromethyl)-1-(3,4-dimethylphenyl)-1H-tetrazole (500 mg, 2.25 mmol, 1.00 equiv), dioxane (6 mL) and NH₄OH (2 mL) and the resulting solution was stirred for 16 h at 40° C. The solution was then concentrated under vacuum and the residue was purified by a silica gel column eluted with methanol/DCM (1/15). This resulted in 400 mg (87.5%) of 1-[1-(3,4-dimethylphenyl)-1H-1,2,3,4-tetrazol-5-yl]methanamine as a white solid. LCMS (ES) [M+1]⁺ m/z: 204.

### Example 1.6

### General Procedure 5: Synthesis of Methyltriazole-methanamines

### Synthesis of 1-[1-(4-chloro-3-fluorophenyl)-3-methyl-1H-1,2,4-triazol-5-yl]methanamine (Intermediate I-6)

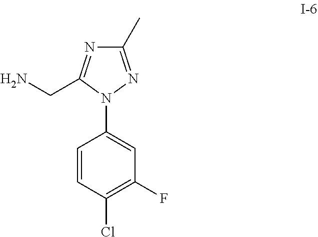 [SMILES: Cc1nc(CN)n(-c2ccc(Cl)c(F)c2)n1]

Step 1

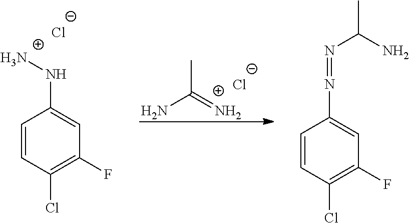

Into a 10-L 3-necked round-bottom flask, was placed (4-chloro-3-fluorophenyl)hydrazine hydrochloride (200.00 g, 1.015 mol, 1.00 eq.), methanol (4.0 L), TEA (307.61 g, 3.045 mol, 3 eq.), acetamidine hydrochloride (114.52 g, 1.218 mmol, 1.20 eq.) and the resulting solution was stirred for 2 days at 60° C. The reaction mixture was then cooled with a water/ice bath and concentrated under vacuum. The residue was purified by silica gel column eluted with chloroform/methanol (10:1) providing 203.00 g (99.02%) of (1-aminoethyl)(4-chloro-3-fluorophenyl)diazene as an off-white solid. LCMS (ES) [M+1]⁺ m/z:202.

Step 2

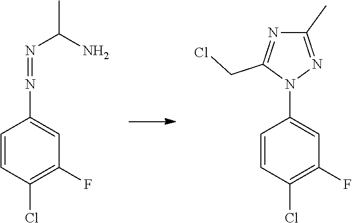 [SMILES: CC(N)N=Nc1ccc(Cl)c(F)c1 ; Cc1nc(CCl)n(-c2ccc(Cl)c(F)c2)n1]

Into a 5-L 3-necked round-bottom flask, was placed (1-aminoethyl)(4-chloro-3-fluorophenyl)diazene (75.00 g, 0.371 mol, 1.00 eq.) DCM (1.50 L) and pyridine (88.43 g, 1.119 mol, 3.00 eq.). Chloroacetyl chloride (62.93 g, 0.557 mol, 1.5 eq.) was then added dropwise at 0° C. over 40 min and the resulting mixture was stirred at 25° C. for 1 hr. Dioxane (1.5 L) was then added into the mixture and the resulting mixture was reacted at 100° C. with a Dean-Stark trap to remove the DCM for 2 hr. The reaction mixture was then cooled and quenched by the addition of water/ice. The resulting solution was extracted with 3×800 mL of dichloromethane and the organic layers were combined, dried over anhydrous sodium sulfate and concentrated under vacuum. The residue was purified by silica gel column eluted with 100% petroleum ether to 15% ethyl acetate in petroleum ether providing 37.00 g (38.33%) of 1-(4-chloro-3-fluorophenyl)-5-(chloromethyl)-3-methyl-1,2,4-triazole as off-white oil. LCMS (ES) [M+1]⁺ m/z: 260.

Step 3

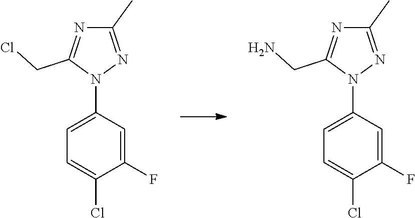 [SMILES: Cc1nc(CCl)n(-c2ccc(Cl)c(F)c2)n1 ; Cc1nc(CN)n(-c2ccc(Cl)c(F)c2)n1]

Into a 3-L 4-necked round-bottom flask, was placed 1-(4-chloro-3-fluorophenyl)-5-(chloromethyl)-3-methyl-1,2,4-triazole (37.00 g, 1.00 eq.), dioxane (1.11 L), and ammonium hydroxide (1.11 L) and the resulting solution was stirred for 48 h at ambient temperature. The reaction mixture was then concentrated under vacuum. The solids were then slurried by MTBE providing 19.3 g (56.33%) of 1-[1-(4-chloro-3-fluorophenyl)-3-methyl-1H-1,2,4-triazol-5-yl]methanamine as a light yellow solid. ¹H NMR (300 MHz, DMSO-d6) δ 7.87 (dd, J=10.3, 2.4 Hz, 1H), 7.78 (t, J=8.4 Hz, 1H), 7.57 (ddd, J=8.7, 2.4, 1.2 Hz, 1H), 3.85 (s, 2H), 2.31 (s, 3H), 1.96 (br, 2H). LCMS (ES) [M+1]⁺ m/z: 241.

### Example 1.7

### General Procedure 6: Synthesis of Symmetric Compounds

### Method A

### Synthesis of 1,3-bis({[1-(3,4-dimethylphenyl)-1H-1,2,3,4-tetrazol-5-yl]methyl})urea (Compound 146)

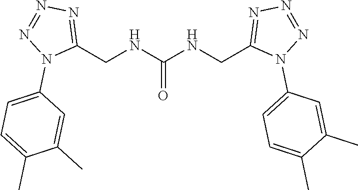 [SMILES: Cc1ccc(-n2nnnc2CNC(=O)NCc2nnnn2-c2ccc(C)c(C)c2)cc1C]

Into a 50-mL three neck round-bottom flask, was placed (1-(3,4-dimethylphenyl)-1H-tetrazol-5-yl)methanamine (Intermediate I-5; 200 mg, 0.985 mmol, 1.00 eq.) and triphosgene (350 mg, 1.182 mmol, 1.2 eq.) in dichloromethane (10 mL) and the mixture was cooled 0° C. Et₃N (298 mg, 2.96 mmol, 3.00 eq.) was then added drop-wise and the resulting solution was stirred while allowing to reach ambient temperature. Additional portions of reagents can be added to the cooled solution to drive the reaction to completion. After 1 h the reaction was quenched with water (5 mL), extracted with dichloromethane (2×10 mL) and concentrated. The crude residue was subjected to reverse preparative HPLC (Atlantis HILIC OBD Column 19×150 mm×5 um; gradient elution of 15% MeCN in water to 65% MeCN in water over a 20 min period, where both solvents contain 0.05% ammonia) to provide 92.5 mg (43%) of 1,3-bis({[1-(3,4-dimethylphenyl)-1H-1,2,3,4-tetrazol-5-yl]methyl})urea as a white solid. ¹H NMR (300 MHz, DMSO-d6) δ 7.41-7.34 (m, 6H), 6.89 (dd, J=5.7 Hz, 2H), 4.47 (d, J=5.7 Hz, 4H), 2.10 (s, 6H), 2.08 (s, 6H). LCMS (ES) [M+1]⁺ m/z: 433.1.

### Example 1.8

### General Procedure 6: Synthesis of Symmetric Compounds

### Method B

### Synthesis of 1,3-bis({[1-(4-chlorophenyl)-3-methyl-1H-1,2,4-triazol-5-yl]methyl})urea (Compound 73)

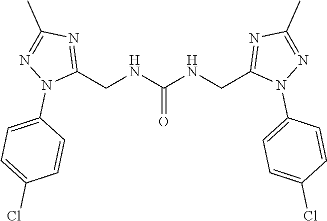 [SMILES: Cc1nc(CNC(=O)NCc2nc(C)nn2-c2ccc(Cl)cc2)n(-c2ccc(Cl)cc2)n1]

Into a 40-mL vial, was placed 1-[1-(4-chlorophenyl)-3-methyl-1H-1,2,4-triazol-5-yl]methanamine (Intermediate I-6; 150.00 mg, 0.67 mmol, 1.00 equiv) in THF (6.00 mL) followed by the addition of a solution of CDI (43.69 mg, 0.27 mmol, 0.40 equiv) in THF (2.00 mL) drop-wise with stirring at ambient temperature. The resulting solution was stirred overnight at ambient temperature then it was concentrated. The residue (150 mg) was purified by Prep-HPLC with the following conditions: Column,) (Bridge Prep C18 OBD Column, 19 cm, 150 mm, Sum; mobile phase, Water (0.1% HCOOH) and CAN (30% Phase B up to 60% in 11 min); Detector, 254 providing 33.7 mg (10.61%) of 1,3-bis({[1-(4-chlorophenyl)-3-methyl-1H-1,2,4-triazol-5-yl]methyl})urea as a white solid. ¹H NMR (300 MHz, DMSO-d6): δ 7.60 (s, 8H), 6.72 (t, J=5.6 Hz, 2H), 4.33 (d, J=5.5 Hz, 4H), 2.29 (s, 6H). LCMS (ES, m/z): [M+H]⁺: 471.

### Example 1.9

### General Procedure 6: Synthesis of Symmetric Compounds

### Method C

### Synthesis of 1,3-bis({[1-(3-chloro-4-fluorophenyl)-1H-1,2,4-triazol-5-yl]methyl})urea (Compound 64)

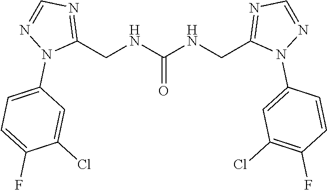 [SMILES: O=C(NCc1ncnn1-c1ccc(F)c(Cl)c1)NCc1ncnn1-c1ccc(F)c(Cl)c1]

Into a 50-mL round-bottom flask, was placed 1-[1-(3-chloro-4-fluorophenyl)-1H-1,2,4-triazol-5-yl]methanamine (200.00 mg, 0.882 mmol, 1.00 eq.; synthesized according to General Procedures 2 and 3 starting from commercial 2-(3-chloro-4-fluorophenyl)hydrazinium chloride), DCM (4.00 mL), DABCO (19.80 mg, 0.176 mmol, 0.20 eq.), and di-tert-butyl dicarbonate (96.30 mg, 0.441 mmol, 0.50 eq.) and the resulting solution was stirred for 2 h at 25° C. The reaction mixture was then concentrated, filtered and subjected to reverse phase preparative HPLC (Prep-C18, 20-45M, 120 g, Tianjin Bonna-Agela Technologies; gradient elution of 30% MeCN in water to 40% MeCN in water over a 10 min period, water contains 0.05% NH₃H₂O) to provide 1,3-bis({[1-(3-chloro-4-fluorophenyl)-1H-1,2,4-triazol-5-yl]methyl})urea as a white solid (110.4 mg, 26.1%). ¹H NMR (300 MHz, DMSO-d6) δ 8.10 (s, 2H), 7.96-7.87 (m, 2H), 7.70-7.55 (m, 4H), 6.72 (t, J=5.7 Hz, 2H), 4.36 (d, J=5.6 Hz, 4H). LCMS (ES) [M+1]+m/z 479.

### Example 1.10

### General Procedure 7: Synthesis of Asymmetric Compounds

### Method A

### Synthesis of 3-{[1-(4-chloro-3-fluorophenyl)-3-methyl-1H-1,2,4-triazol-5-yl]methyl}-1-{[1-(8-fluoroquinolin-3-yl)-1H-1,2,4-triazol-5-yl]methyl}urea (Compound 136)

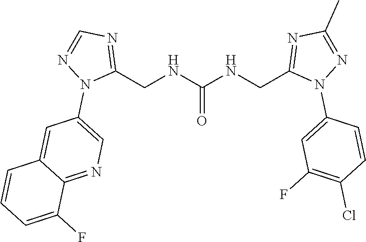 [SMILES: Cc1nc(CNC(=O)NCc2ncnn2-c2cnc3c(F)cccc3c2)n(-c2ccc(Cl)c(F)c2)n1]

Carbonyl diimidazole (110.15 mg; 0.68 mmol; 1.00 eq.) was added in one portion to a solution of [1-(8-fluoroquinolin-3-yl)-1H-1,2,4-triazol-5-yl]methanaminium chloride (190.00 mg; 0.68 mmol; 1.00 eq.; synthesized from 8-fluoroquinolin-3-amine according to General Procedures 1, 2 and 3) in 30 V dichloromethane (5.70 mL) and Hunig's base (0.33 mL; 2.04 mmol; 3.00 eq.) at 0° C. and the resulting mixture was stirred at 0° C. After 1.5 h aliquot analysis of the reacting mixture by LCMS showed complete conversion to the CDI-urea intermediate (Intermediate 1a in scheme 1). This reaction mixture was then added drop-wise to a solution of [1-(4-chloro-3-fluorophenyl)-3-methyl-1H-1,2,4-triazol-5-yl]methanamine (Intermediate I-6; 163.48 mg; 0.68 mmol; 1.00 eq.) in 15 V (3.0 mL) dichloromethane and 3 eq. (0.3 mL) Hunig's base and the resulting mixture was stirred at ambient temperature. After 3 h the mixture was concentrated in vacuo and the residue was dissolved in DMF/H₂O/1N HCl, filtered and purified by reverse phase preparative HPLC (Shimadzu eluting with a gradient of 8-70% MeCN (containing 0.1% formic acid) in water, 16 minutes 10 injections) to provide 3-{{[1-)4-chloro-3-yl)-1H-1,2,4-triazol-5-yl]methyl}-1-{[1-(8-fluoroquinolin-3-yl)-1H-1,2,4-triazol-5-yl]methyl}urea as a white solid (98.1 mg; 28%). ¹H NMR (400 MHz, DMSO-d6) δ 9.12 (d, J=2.4 Hz, 1H), 8.73 (t, J=2.0 Hz, 1H), 8.20 (s, 1H), 7.86 (dt, J=7.8, 3.9 Hz, 1H), 7.78-7.62 (m, 4H), 7.42 (dd, J=8.7, 2.4 Hz, 1H), 6.74 (t, J=5.7 Hz, 1H), 6.66 (t, J=5.6 Hz, 1H), 4.50 (d, J=5.6 Hz, 2H), 4.26 (d, J=5.6 Hz, 2H), 2.24 (s, 3H). LCMS (ES) [M+1]⁺ m/z 510.1.

Alternatively, the CDI-urea intermediate could be isolated as a crude material and/or purified by silica gel column chromatography eluting with a gradient of 0-60% (DCM:MeOH:NH₄OH; 90:9:1) in DCM and used as a dry solid in the procedure described above.

### Example 1.11

### General Procedure 7: Synthesis of Asymmetric Compounds

### Method B

### Synthesis of 1-{[1-(4-chloro-3-fluorophenyl)-1H-1,2,3,4-tetrazol-5-yl]methyl}-3-{[3-methyl-1-(quinolin-7-yl)-1H-1,2,4-triazol-5-yl]methyl}urea (Compound 86)

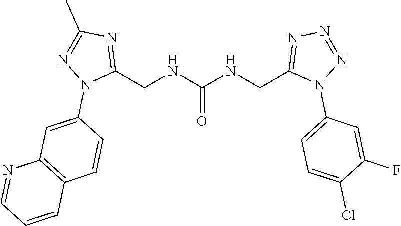 [SMILES: Cc1nc(CNC(=O)NCc2nnnn2-c2ccc(Cl)c(F)c2)n(-c2ccc3cccnc3c2)n1]

Into a 100-mL round-bottom flask, was placed 1-[1-(4-chloro-3-fluorophenyl)-1,2,3,4-tetrazol-5-yl]methanamine (100.00 mg, 0.439 mmol, 1.00 eq.; synthesized according to General Procedure 4 using commercial 4-fluoro-3-chloroaniline), 1-[5-methyl-2-(quinolin-7-yl)-1,2,4-triazol-3-yl]methanamine (105.12 mg, 0.439 mmol, 1.00 eq.; Intermediate I-10), DCM (15 mL), 1,4-diazabicyclo[2,2,2]octane (49.28 mg, 0.439 mmol, 1.00 equiv). This was followed by the addition of a solution of Boc₂O (191.76 mg, 0.879 mmol, 2.00 equiv) in DCE (1 mL) drop-wise with stirring at room temperature over 10 min. The resulting solution was stirred for 0.5 h at ambient temperature. The crude product was purified by Flash-Prep-HPLC (Prep-C18, 20-45M, 120 g, Tianjin Bonna-Agela Technologies; gradient elution of 45% MeCN in water to 65% MeCN in water over a 10 min period, where both solvents contain 0.1% NH₃H₂O) providing 64 mg (29.56%) of 1-{[1-(4-chloro-3-fluorophenyl)-1H-1,2,3,4-tetrazol-5-yl]methyl}-3-{[3-methyl-1-(quinolin-7-yl)-1H-1,2,4-triazol-5-yl]methyl}urea as a white solid. 1H NMR (300 MHz, DMSO-d6) δ 9.00 (dd, J=4.2, 1.7 Hz, 1H), 8.48 (dd, J=8.7, 1.6 Hz, 1H), 8.21-8.13 (m, 2H), 7.94-7.76 (m, 3H), 7.67-7.54 (m, 2H), 6.81 (t, J=5.4 Hz, 2H), 4.52 (d, J=5.6 Hz, 2H), 4.42 (d, J=5.6 Hz, 2H), 2.35 (s, 3H). LCMS (ES) [M+1]⁺ m/z 493.1.

### Example 1.12

### General Procedure 7: Synthesis of Asymmetric Compounds

### Method C

### Synthesis of 1-{[1-(1,3-benzothiazol-5-yl)-1H-1,2,4-triazol-5-yl]methyl}-3-{[1-(4-chloro-3-fluorophenyl)-3-methyl-1H-1,2,4-triazol-5-yl]methyl}urea (Compound 17)

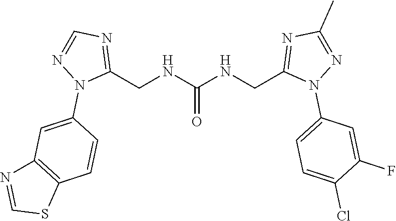 [SMILES: Cc1nc(CNC(=O)NCc2ncnn2-c2ccc3scnc3c2)n(-c2ccc(Cl)c(F)c2)n1]

Phenyl chloroformate (235 mg; 1.50 mmol) was added to a solution of [1-(1,3-benzothiazol-5-yl)-1H-1,2,4-triazol-5-yl]methanamine (400 mg; 1.50 mmol; synthesized according to General Procedures 1, 2 and 3 starting from commercial 5-aminobenzothiazole) and triethylamine (0.42 mL; 3.0 mol) in dichloromethane (3 mL) and the resulting mixture was stirred for 1 hour at ambient temperature. The reaction mixture was then diluted with water and extracted with ethyl acetate. The combined organics were dried with MgSO4, filtered, concentrated under reduced pressure and purified by silica gel column chromatography using 10% MeOH in DCM as the eluting solvents to afford phenyl N-{[1-(1,3-benzothiazol-5-yl)-1H-1,2,4-triazol-5-yl]methyl}carbamate. LCMS (ES) [M+1]⁺ m/z: 351.9

This material (200 mg; 0.57 mmol) was combined in a capped microwave vial with [1-(4-chloro-3-fluorophenyl)-3-methyl-1H-1,2,4-triazol-5-yl]methanamine (Intermediate I-6; 157 mg; 0.57 mmol), and triethylamine (0.24 mL; 1.71 mmol) in DMF (3 mL) and the resulting mixture was heated to 120° C. for 20 minutes in a microwave reactor. The cooled reaction mixture was directly purified by reverse phase preparative HPLC using 5% MeCN to 70% MeCN in 0.1% formic acid in water as the eluting solvents to afford 1-{[1-(1,3-benzothiazol-5-yl)-1H-1,2,4-triazol-5-yl]methyl}-3-[{1-(4-chloro-3-fluorophenyl)-3-methyl-1H-1,2,4-triazol-5-yl]methyl}urea (47 mg; 17%). 1-H NMR (400 MHz, DMSO-d6) δ 9.53 (s, 1H), 8.37-8.29 (m, 2H), 8.11 (s, 1H), 7.77-7.64 (m, 3H), 7.48-7.40 (m, 1H), 6.71 (q, J=6.1 Hz, 2H), 4.40 (d, J=5.6 Hz, 2H), 4.32 (d, J=5.6 Hz, 2H), 2.27 (s, 3H). LCMS (ES) [M+1]⁺ m/z: 497.9.

### Example 1.13

### Synthesis of 3-(aminomethyl)-4-(4-chloro-3-fluorophenyl)-4,5-dihydro-1H-1,2,4-triazol-5-one (Intermediate I-7)

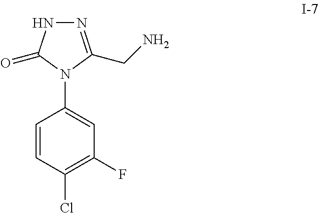 [SMILES: NCc1n[nH]c(=O)n1-c1ccc(Cl)c(F)c1]

Step 1

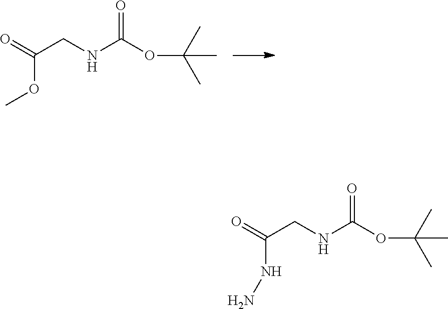

Into a 250-mL round-bottom flask purged and maintained with an inert atmosphere of nitrogen, was placed methyl 2-[(tert-butoxycarbonyl)amino]acetate (10.00 g, 52.851 mmol, 1.00 eq.), EtOH (50.00 mL) and NH₂NH₂·H₂O (12.50 mL). The resulting solution was stirred for 5 h at 80° C. in an oil bath. The mixture was then cooled to ambient temperature, diluted with 100 mL of water, extracted with 5×200 mL of DCM:MeOH=10:1, dried over anhydrous sodium sulfate and concentrated under vacuum providing 8 g (80.00%) of tert-butyl N-[(hydrazinecarbonyl)methyl]carbamate as a white solid. LCMS (ES) [M+1]⁺ m/z 190.1.

Step 2

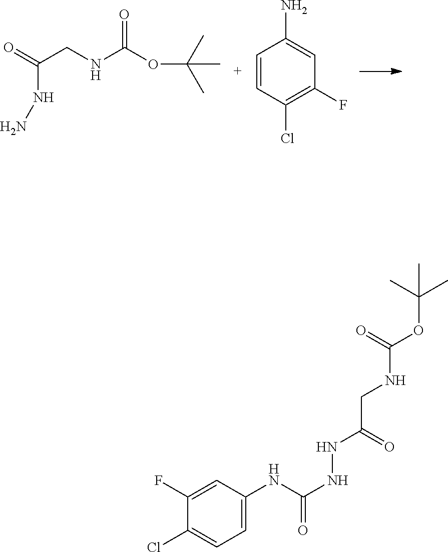

Into a 1-L round-bottom flask purged and maintained with an inert atmosphere of nitrogen, was placed triphosgene (6.9 g, 23.27 mmol, 0.55 eq.) and DCM (500 mL). The resulting solution was stirred for 15 min at 0° C., then 4-chloro-3-fluoroaniline (6.15 g, 42.25 mmol, 1.00 eq.) was added and the resulting solution was stirred for 2 h at 0° C. TEA (12.8 g, 127 mmol, 3.00 equiv) was then added at 0° C. followed by a solution of tert-butyl N-[(hydrazinecarbonyl)methyl]carbamate (6.14 g, 42.3 mmol, 1.00 eq.) in DCM (50 mL). The resulting solution was stirred overnight at ambient temperature. The solution was then diluted with 100 mL of water, extracted with 3×200 mL of dichloromethane, dried over anhydrous sodium sulfate and concentrated under vacuum. The residue was applied onto a silica gel column and eluted with PE/THF (30%-100% THF in PE) providing 3 g (19.67%) of tert-butyl N-[([[(4-chloro-3-fluorophenyl)carbamoyl]amino]carbamoyl)methyl]carbamate as a white solid. LCMS (ES) [M−1]⁻ m/z 359.

Step 3

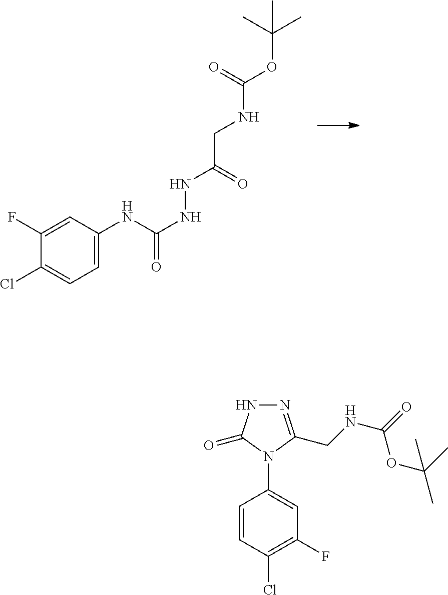 [SMILES: CC(C)(C)OC(=O)NCC(=O)NNC(=O)Nc1ccc(Cl)c(F)c1 ; CC(C)(C)OC(=O)NCc1n[nH]c(=O)n1-c1ccc(Cl)c(F)c1]

Into a 250-mL round-bottom flask purged and maintained with an inert atmosphere of nitrogen, was placed tert-butyl N-[([[(4-chloro-3-fluorophenyl)carbamoyl]amino]carbamoyl)methyl]carbamate (3.00 g, 8.316 mmol, 1.00 eq.) and a solution of NaOH (1.20 g, 0.030 mmol) in H₂O (60 mL). The resulting solution was stirred overnight at 80° C. in an oil bath then it was cooled to ambient temperature and concentrated. The residue was purified by Flash-Prep-HPLC providing 1.5 g (52.63%) of tert-butyl N-[[4-(4-chloro-3-fluorophenyl)-5-oxo-1H-1,2,4-triazol-3-yl]methyl]carbamate as a light yellow solid. LCMS (ES) [M+1]⁺ m/z 343.

Step 4

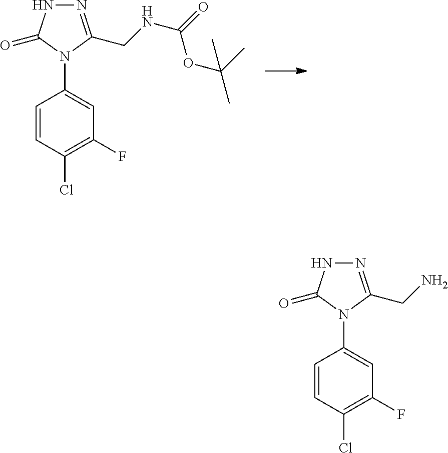 [SMILES: CC(C)(C)OC(=O)NCc1n[nH]c(=O)n1-c1ccc(Cl)c(F)c1 ; NCc1n[nH]c(=O)n1-c1ccc(Cl)c(F)c1]

Into a 100-mL round-bottom flask purged and maintained with an inert atmosphere of nitrogen, was placed tert-butyl N-[[4-(4-chloro-3-fluorophenyl)-5-oxo-1H-1,2,4-triazol-3-yl]methyl]carbamate (1.20 g, 3.501 mmol, 1.00 equiv), dioxane (15.00 mL) and HCl(gas)in 1,4-dioxane (4N) (10.00 mL). The resulting solution was stirred overnight at ambient temperature then it was concentrated under vacuum providing 700 mg (82.40%) of 3-(aminomethyl)-4-(4-chloro-3-fluorophenyl)-4,5-dihydro-1H-1,2,4-triazol-5-one as a light yellow solid. LCMS (ES) [M+1]⁺ m/z 243.

### Example 1.14

### General Procedure 8: Synthesis of Alkyl-triazole-methanamines

### Synthesis of 1-{1-[(oxan-4-yl)methyl]-1H-1,2,4-triazol-5-yl}methanamine hydrochloride (Intermediate I-8)

Step 1

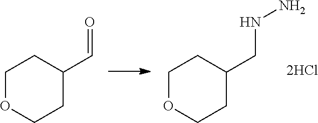 [SMILES: O=CC1CCOCC1 ; NNCC1CCOCC1]

Sodium cyanoborohydride (671 mg; 10.7 mmol) was added to a solution containing tetrahydro-2H-pyran-4-carbaldehyde (610 mg; 5.3 mmol) and benzyl carbazate (888 mg; 5.3 mmol) in methanol (18 mL) and formic acid (0.18 mL). The reaction was stirred overnight. The reaction mixture was concentrated under reduced pressure and diluted with water. The aqueous layer was extracted with ethyl acetate. The combined organics were dried with MgSO4, filtered, concentrated under reduced pressure and purified by silica gel column using 10% MeOH in DCM to afford 1 g benzyl 2-((tetrahydro-2H-pyran-4-yl)methyl)hydrazine-1-carboxylate. MS (ESI, pos. ion) m/z: 265.0 (M+1).

This carbamate intermediate was diluted with 1M hydrogen chloride (20 mL) and MeCN (2 mL). The reaction mixture was stirred and heated to 90° C. for 3 hours and then it was concentrated to afford 1-(tetrahydro-2H-pyran-4-ylmethyl)hydrazine dihydrochloride (700 mg; 65%). MS (ESI, pos. ion) m/z: 131.1 (M+1).

### Step 2

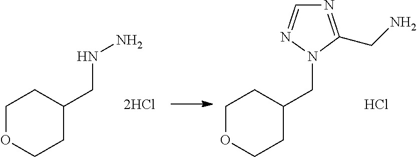 [SMILES: NNCC1CCOCC1 ; NCc1ncnn1CC1CCOCC1 ; Cl]

A mixture of 1-(tetrahydro-2H-pyran-4-ylmethyl)hydrazine dihydrochloride (700 mg; 4.2 mmol) and tert-butyl N-({[(1E)-(dimethylamino)methylidene]carbamoyl}methyl)carbamate (Intermediate I-1; 969 mg; 4.2 mmol) in 1,4-dioxane (14 mL) and acetic acid (7 mL) was heated to 90° C. for 1 hour. The reaction mixture was then cooled to ambient temperature and concentrated under reduced pressure. The resulting residue was purified by silica gel column using 10% MeOH in DCM to afford tert-butyl ((1-((tetrahydro-2H-pyran-4-yl)methyl)-1H-1,2,4-triazol-5-yl)methyl)carbamate (860 mg; 69%).

This carbamate intermediate was diluted with 1M hydrogen chloride (20 mL) and MeCN (2 mL). The reaction mixture was stirred and heated to 50° C. for 3 hours and it was then concentrated to afford 1-{1-[(oxan-4-yl)methyl]-1H-1,2,4-triazol-5-yl}methanamine hydrochloride (680 mg; 69%).

### Example 1.15

### Synthesis of 1-[1-(quinolin-7-yl)-1H-1,2,4-triazol-5-yl]methanamine (Intermediate I-9)

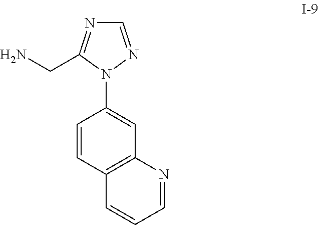 [SMILES: NCc1ncnn1-c1ccc2cccnc2c1]

The title compound was synthesized according to General Procedures 2 and 3 starting from commercial 7-hydrazinylquinoline) to provide 1-[1-(quinolin-7-yl)-1H-1,2,4-triazol-5-yl]methanamine.

### Example 1.16

### Synthesis of 1-[3-methyl-1-(quinolin-7-yl)-1H-1,2,4-triazol-5-yl]methanamine (Intermediate I-10)

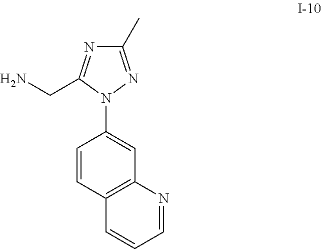 [SMILES: Cc1nc(CN)n(-c2ccc3cccnc3c2)n1]

The title compound was synthesized according to General Procedure 5 using 7-hydrazinylquinoline (commercially sourced or synthesized according to General Procedure 1 using commercial quinoline-7-amine) to provide 1-[3-methyl-1-(quinolin-7-yl)-1H-1,2,4-triazol-5-yl]methanamine. 1H NMR (300 MHz, DMSO-d6) δ 9.00 (dd, J=4.2, 1.7 Hz, 1H), 8.49 (dt, J=8.3, 1.5 Hz, 1H), 8.31 (d, J=2.2 Hz, 1H), 8.17 (d, J=8.8 Hz, 1H), 7.89 (dd, J=8.8, 2.2 Hz, 1H), 7.63 (dd, J=8.3, 4.2 Hz, 1H), 3.93 (s, 2H), 2.36 (s, 3H), 2.01 (br, 2H). LCMS (ES) [M+1]⁺ m/z:240.

### Example 1.17

### Synthesis of 1,3-bis({[1-(4-chlorophenyl)-1H-1,2,3,4-tetrazol-5-yl]methyl})urea (Compound 1)

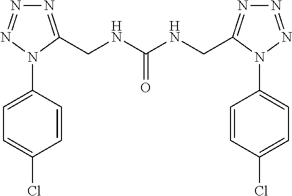 [SMILES: O=C(NCc1nnnn1-c1ccc(Cl)cc1)NCc1nnnn1-c1ccc(Cl)cc1]

The title compound was synthesized according to General Procedure 6, Method A using 1-[1-(4-chlorophenyl)-1H-1,2,3,4-tetrazol-5-yl]methanamine (550 mg; synthesized according to General Procedure 4 starting from commercial 4-chloroaniline) to provide 1,3-bis({[1-(4-chlorophenyl)-1H-1,2,3,4-tetrazol-5-yl]methyl})urea as a white solid (80 mg: 14% yield). 1-H NMR (400 MHz, DMSO-d6) δ 7.68 (d, J=1.6 Hz, 8H), 6.87 (t, J=5.7 Hz, 2H), 4.50-4.37 (m, 4H). LCMS (ES) [M+1]⁺ m/z: 443.1.

### Example 1.18

### Synthesis of 1,3-bis({[1-(3-chlorophenyl)-1H-1,2,3,4-tetrazol-5-yl]methyl})urea (Compound 2)

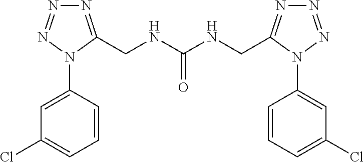 [SMILES: O=C(NCc1nnnn1-c1cccc(Cl)c1)NCc1nnnn1-c1cccc(Cl)c1]

The title compound was synthesized according to General Procedure 6, Method A using 1-[1-(3-chlorophenyl)-1H-1,2,3,4-tetrazol-5-yl]methanamine (200 mg; synthesized according to General Procedure 4 starting from commercial 3-chloroaniline) to provide 1,3-bis({[1-(3-chlorophenyl)-1H-1,2,3,4-tetrazol-5-yl]methyl})urea as a white solid (110 mg: 52% yield). ¹H NMR (400 MHz, DMSO-d6) δ 7.82 (d, J=2.2 Hz, 2H), 7.72-7.65 (m, 2H), 7.65-7.59 (m, 4H), 6.88 (t, J=5.7 Hz, 2H), 4.46 (d, J=5.6 Hz, 4H). LCMS (ES) [M+1]⁺ m/z: 443.1.

### Example 1.19

### Synthesis of 1,3-bis({[1-(4-chlorophenyl)-1H-1,2,3,4-tetrazol-5-yl]methyl})-1,3-diazinan-2-one (Compound 3)

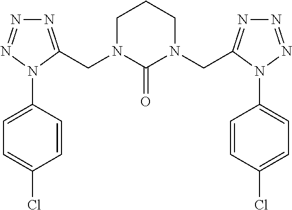 [SMILES: O=C1N(Cc2nnnn2-c2ccc(Cl)cc2)CCCN1Cc1nnnn1-c1ccc(Cl)cc1]

Sodium hydride (60% wt) (99.87 mg; 2.50 mmol) was added to a solution containing tetrahydro-2(1H)-pyrimidinone (50.00 mg; 0.50 mmol) in tetrahydrofuran (5 mL). After stirring for 5 minutes, 5-(chloromethyl)-1-(4-chlorophenyl)-1H-1,2,3,4-tetrazole (0.23 g; 1.00 mmol; synthesized according to General Procedure 4, steps 1 and 2 starting from commercial 4-chloroaniline) was added to the heterogeneous solution and heated to 60° C. for 15 minutes. The reaction mixture was then cooled to ambient temperature and water was added to quench the reaction. Ethyl acetate was added and both layers were filtered. The organic layer was collected, dried with MgSO4, and concentrated. The residue was purified by reverse phase preparative HPLC using 5% to 70% MeCN in 0.1% formic acid in water to provide 1,3-bis({[1-(4-chlorophenyl)-1H-1,2,3,4-tetrazol-5-yl]methyl})-1,3-diazinan-2-one (66 mg; 27%) as a white solid. ¹H NMR (400 MHz, DMSO-d6) δ 7.66 (d, J=1.5 Hz, 8H), 4.65 (s, 4H), 3.29 (s, 4H), 1.78 (p, J=5.8 Hz, 2H). LCMS (ES) [M+1]⁺ m/z: 485.0.

### Example 1.20

### Synthesis of 1,3-bis({[1-(4-chloro-3-fluorophenyl)-1H-1,2,3,4-tetrazol-5-yl]methyl})-1,3-diazinan-2-one (Compound 4)

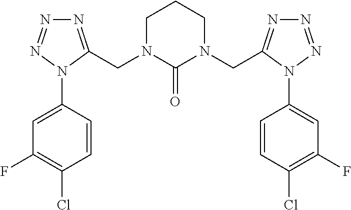 [SMILES: O=C1N(Cc2nnnn2-c2ccc(Cl)c(F)c2)CCCN1Cc1nnnn1-c1ccc(Cl)c(F)c1]

The title compound was synthesized according to the procedure described for Example 1.19 for the preparation of Compound 3 using 1-(4-chloro-3-fluorophenyl)-5-(chloromethyl)-1H-1,2,3,4-tetrazole (synthesized according to General Procedure 4, steps 1 and 2 starting from commercial 4-chloro-3-fluoroaniline). ¹H NMR (400 MHz, DMSO-d6) δ 7.92-7.85 (m, 2H), 7.83 (d, J=8.2 Hz, 2H), 7.54 (dd, J=8.6, 2.4 Hz, 2H), 4.67 (s, 4H), 3.29 (d, J=11.7 Hz, 4H), 1.80 (p, J=5.9 Hz, 2H). LCMS (ES) [M+1]⁺ m/z: 521.0.

### Example 1.21

### Synthesis of 3-{[1-(4-chloro-3-fluorophenyl)-1H-1,2,4-triazol-5-yl]methyl}-1-{[1-(oxan-4-yl)-1H-1,2,4-triazol-5-yl]methyl}urea (Compound 5)

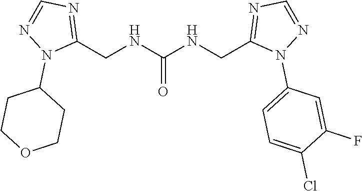 [SMILES: O=C(NCc1ncnn1-c1ccc(Cl)c(F)c1)NCc1ncnn1C1CCOCC1]

Triphosgene (36 mg; 0.12 mmol) was added to a solution containing 1-[1-(oxan-4-yl)-1H-1,2,4-triazol-5-yl]methanamine hydrochloride (107 mg; 0.5 mmol; synthesized according to General Procedure 8, starting from commercial 1-[tetrahydro-2H-pyran-4-yl]hydrazine hydrochloride) and Hunig's base (0.26 mL) in DCM (5 mL). After 5 minutes, 1-[1-(4-chloro-3-fluorophenyl)-1H-1,2,4-triazol-5-yl]methanamine (Intermediate I-4; 44 mg; 0.2 mmol) was added to the reaction and the resulting mixture was stirred at ambient temperature for 1 hour and then it was concentrated under reduced pressure. The residue was purified by reverse phase preparative HPLC using 5% MeCN to 70% MeCN in 0.1% formic acid in water as the eluting solvents to provide 3-{[1-(4-chloro-3-fluorophenyl)-1H-1,2,4-triazol-5-yl]methyl}-1-{[1-(oxan-4-yl)-1H-1,2,4-triazol-5-yl]methyl}urea (3 mg; 1% yield) as a white solid. ¹H NMR (400 MHz, DMSO-d6) δ 8.10 (s, 1H), 7.89-7.73 (m, 3H), 7.52 (d, J=8.7 Hz, 1H), 6.79 (t, J=6.0 Hz, 1H), 6.73 (t, J=5.8 Hz, 1H), 4.63 (td, J=11.2, 5.4 Hz, 1H), 4.38 (dd, J=14.8, 5.7 Hz, 4H), 3.88 (dd, J=11.4, 4.5 Hz, 2H), 3.30 (m, 2H), 1.91 (qd, J=12.3, 4.7 Hz, 2H), 1.76-1.64 (m, 2H). LCMS (ES) [M+1]⁺ m/z: 435.0.

### Example 1.22

### Synthesis of 1,3-bis({[1-(4-chloro-3-fluorophenyl)-1H-1,2,4-triazol-5-yl]methyl})-1,3-diazinan-2-one (Compound 6)

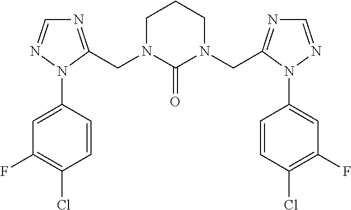 [SMILES: O=C1N(Cc2ncnn2-c2ccc(Cl)c(F)c2)CCCN1Cc1ncnn1-c1ccc(Cl)c(F)c1]

Step 1

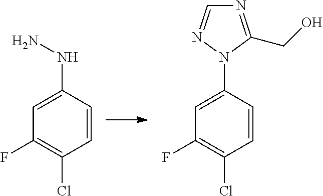 [SMILES: NNc1ccc(Cl)c(F)c1 ; OCc1ncnn1-c1ccc(Cl)c(F)c1]

A mixture of 2-(benzyloxy)acetamide (2.1 g; 12.71 mmol) and dimethoxy-N,N-dimethylmethanamine (25 mL) was heated to 95° C. for 3 hours. The reaction mixture was then cooled to ambient temperature and concentrated under reduced pressure. To the residue was added (4-chloro-3-fluorophenyl)hydrazine (2.0 g; 12.71 mmol), 1,4-dioxane (32 mL) and acetic acid (32 mL) and the resulting mixture was heated to 95° C. for 1 hour. The reaction mixture was then cooled to ambient temperature and concentrated under reduced pressure. The residue was purified by silica gel column chromatography using 10% MeOH in DCM to afford 5-((benzyloxy)methyl)-1-(4-chloro-3-fluorophenyl)-1H-1,2,4-triazole. This intermediate was treated with trifluoroacetic acid (30.00 mL) at ambient temperature for 4 hours. The reaction mixture was then concentrated under reduced pressure, and purified by silica gel column chromatography using 60% ethyl acetate in heptanes to afford (1-(4-chloro-3-fluorophenyl)-1H-1,2,4-triazol-5-yl)methanol (660.00 mg; 23%).

Step 2

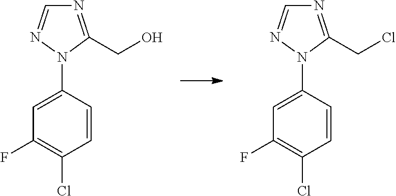 [SMILES: OCc1ncnn1-c1ccc(Cl)c(F)c1 ; Fc1cc(-n2ncnc2CCl)ccc1Cl]

Oxalyl chloride (0.25 mL; 2.90 mmol.) was added to a solution containing [1-(4-chloro-3-fluorophenyl)-1H-1,2,4-triazol-5-yl]methanol (660.00 mg; 2.90 mmol) and triethylamine (1.21 mL; 8.70 mmol) in dichloromethane (10 mL) and the resulting mixture was stirred for 30 minutes at ambient temperature. The reaction mixture was then concentrated under reduced pressure and purified by silica gel column chromatography using 75% ethyl acetate in hepatanes to afford 1-(4-chloro-3-fluorophenyl)-5-(chloromethyl)-1H-1,2,4-triazole (300.00 mg; 42%). LCMS (ES) [M+1]⁺ m/z: 245.8.

Step 3

The title compound was synthesized according to the procedure described for Example 1.19 using 1-(4-chloro-3-fluorophenyl)-5-(chloromethyl)-1H-1,2,4-triazole (from step 2 above). 1H NMR (400 MHz, DMSO-d6) δ 8.10 (d, J=1.6 Hz, 2H), 7.82-7.68 (m, 4H), 7.51-7.39 (m, 2H), 4.58 (s, 4H), 3.31 (d, J=11.6 Hz, 4H), 1.82 (p, J=5.8 Hz, 2H). LCMS (ES) [M+1]⁺ m/z: 519.0.

### Example 1.23

### Synthesis of 3-{[1-(4-chloro-3-fluorophenyl)-1H-1,2,4-triazol-5-yl]methyl}-1-({1-[(oxan-4-yl)methyl]-1H-1,2,4-triazol-5-yl}methyl)urea (Compound 7)

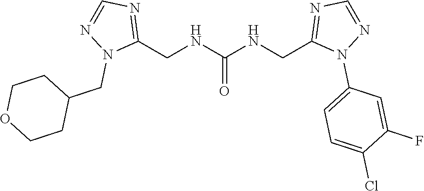 [SMILES: O=C(NCc1ncnn1CC1CCOCC1)NCc1ncnn1-c1ccc(Cl)c(F)c1]

The title compound was synthesized according to General Procedure 7, Method C, using phenyl N-({1-[(oxan-4-yl)methyl]-1H-1,2,4-triazol-5-yl}methyl)carbamate (144 mg; synthesized according to General Procedure 8, starting from commercial tetrahydro-2H-pyran-4-carbaldehyde) and 1-[1-(4-chloro-3-fluorophenyl)-1H-1,2,4-triazol-5-yl]methanamine (Intermediate I-4; 1 eq.) to provide 3-{[1-(4-chloro-3-fluorophenyl)-1H-1,2,4-triazol-5-yl]methyl}-1-({1-[(oxan-4-yl)methyl]-1H-1,2,4-triazol-5-yl}methyl)urea (55 mg; 27% yield) as a white solid. 1-H NMR (400 MHz, DMSO-d6) δ 8.10 (d, J=1.6 Hz, 1H), 7.86-7.74 (m, 3H), 7.50 (dd, J=8.7, 2.3 Hz, 1H), 6.71 (t, J=5.9 Hz, 2H), 4.43 (d, J=5.5 Hz, 2H), 4.32 (d, J=5.7 Hz, 2H), 3.99 (d, J=7.1 Hz, 2H), 3.83-3.70 (m, 2H), 3.18 (td, J=11.6, 2.1 Hz, 2H), 1.98 (dtd, J=11.3, 7.9, 7.5, 4.1 Hz, 1H), 1.41-1.29 (m, 2H), 1.20 (qd, J=12.3, 4.3 Hz, 2H). LCMS (ES) [M+1]⁺ m/z: 449.0.

### Example 1.24

### 3-{[1-(4-chloro-3-fluorophenyl)-1H-1,2,4-triazol-5-yl]methyl}-3-ethyl-1-({1-[(oxan-4-yl)methyl]-1H-1,2,4-triazol-5-yl}methyl)urea (Compound 8)

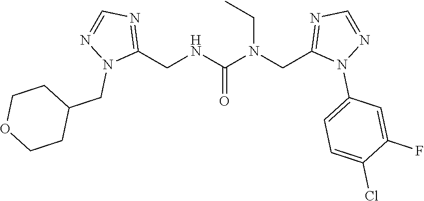 [SMILES: CCN(Cc1ncnn1-c1ccc(Cl)c(F)c1)C(=O)NCc1ncnn1CC1CCOCC1]

The title compound was synthesized according to General Procedure 7, Method C, using phenyl N-({1-[(oxan-4-yl)methyl]-1H-1,2,4-triazol-5-yl}methyl)carbamate (50 mg; synthesized according to General Procedure 8, starting from commercial tetrahydro-2H-pyran-4-carbaldehyde) and {[1-(4-chloro-3-fluorophenyl)-3-methyl-1H-1,2,4-triazol-5-yl]methyl}(ethyl)amine (Example 1.46 for the preparation of Compound 30, steps 1 and 2; 40 mg) to provide 3-{[1-(4-chloro-3-fluorophenyl)-1H-1,2,4-triazol-5-yl]methyl}-3-ethyl-1-({1-[(oxan-4-yl)methyl]-1H-1,2,4-triazol-5-yl}methyl)urea (12 mg; 16% yield) as a white solid. ¹H NMR (400 MHz, Methanol-d4) δ 8.04 (s, 1H), 7.85 (s, 1H), 7.70-7.56 (m, 2H), 7.42 (ddd, J=8.7, 2.5, 1.3 Hz, 1H), 4.70 (s, 2H), 4.45 (d, J=4.2 Hz, 2H), 4.10 (d, J=7.2 Hz, 2H), 3.91 (ddd, J=11.7, 4.5, 1.8 Hz, 2H), 3.43-3.24 (m, 4H), 2.16 (ttt, J=11.3, 7.5, 3.9 Hz, 1H), 1.47 (ddd, J=12.9, 4.3, 2.1 Hz, 2H), 1.42-1.25 (m, 2H), 1.12 (t, J=7.1 Hz, 3H). LCMS (ES) [M+1]⁺ m/z: 477.1.

### Example 1.25

### Synthesis of 3-{[1-(4-chloro-3-fluorophenyl)-1H-1,2,4-triazol-5-yl]methyl}-1-ethyl-1-({1-[(oxan-4-yl)methyl]-1H-1,2,4-triazol-5-yl}methyl)urea (Compound 9)

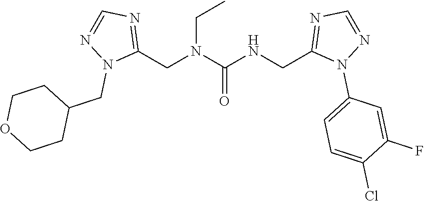 [SMILES: CCN(Cc1ncnn1CC1CCOCC1)C(=O)NCc1ncnn1-c1ccc(Cl)c(F)c1]

Step 1

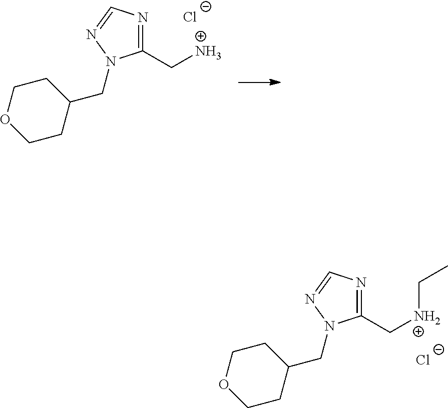 [SMILES: [NH3+]Cc1ncnn1CC1CCOCC1 ; [Cl-] ; CC[NH2+]Cc1ncnn1CC1CCOCC1 ; [Cl-]]

A mixture of 1-{1-[(oxan-4-yl)methyl]-1H-1,2,4-triazol-5-yl}methanamine hydrochloride (Intermediate I-8; 500 mg; 2.1 mmol), Hunig's base (1 mL) and di-tert-butyl dicarbonate (600 mg; 2.7 mmol) in THF (10 mL) was stirred at ambient temperature for 3 hours. The reaction mixture was then diluted with water and extracted with ethyl acetate. The combined organics were dried with MgSO4, filtered, concentrated under reduced pressure, and purified by silica gel column using 10% MeOH in DCM as the eluting solvents to afford tert-butyl N-({1-[(oxan-4-yl)methyl]-1H-1,2,4-triazol-5-yl}methyl)carbamate (400 mg; 62% yield)

Sodium hydride 60% wt (103.43 mg; 2.6 mmol) as added to a solution containing tert-butyl N-({1-[(oxan-4-yl)methyl]-1H-1,2,4-triazol-5-yl}methyl)carbamate (400 mg; 1.3 mol) in N,N-dimethylformamide (7 mL). After 30 minutes, iodoethane (0.22 mL; 2.6 mmol) was added and the reaction mixture was stirred for 3 hours. The mixture was then diluted with water and extracted with ethyl acetate. The combined organics were dried with MgSO4, filtered, concentrated under reduced pressure, and purified by silica gel column using 90% ethyl acetate in heptanes to afford tert-butyl N-ethyl-N-({1-[(oxan-4-yl)methyl]-1H-1,2,4-triazol-5-yl}methyl)carbamate (150 mg; 34%). LCMS (ES) [M+1]⁺ m/z: 325.1. This intermediate was treated with 4M HCl in dioxanes (4 mL) at 50° C. for 1 hour. The reaction mixture was then concentrated under reduced pressure to provide ethyl({1-[(oxan-4-yl)methyl]-1H-1,2,4-triazol-5-yl}methyl)amine hydrochloride (120 mg; 99% crude yield) as a colorless oil.

Step 2

The title compound was synthesized according to General Procedure 7, Method C, using [1-(4-chloro-3-fluorophenyl)-1H-1,2,4-triazol-5-yl]methanamine (Intermediate I-4; 160 mg) and ethyl({1-[(oxan-4-yl)methyl]-1H-1,2,4-triazol-5-yl}methyl)amine hydrochloride (120 mg; from step 1 above) to provide 3-{[1-(4-chloro-3-fluorophenyl)-1H-1,2,4-triazol-5-yl]methyl}1-[(oxan-4-yl)methyl]-1H-1,2,4-triazol-5-yl}methyl)urea (47 mg; 21% yield) as a white solid. ¹H NMR (400 MHz, Methanol-d4) δ 8.05 (s, 1H), 7.87 (s, 1H), 7.74-7.62 (m, 2H), 7.48 (ddd, J=8.6, 2.4, 1.2 Hz, 1H), 4.64 (s, 2H), 4.55 (d, J=4.0 Hz, 2H), 4.07 (d, J=7.3 Hz, 2H), 3.89 (ddd, J=11.6, 4.5, 1.8 Hz, 2H), 3.41-3.21 (m, 4H), 2.12 (th, J=11.3, 3.7 Hz, 1H), 1.42 (ddd, J=12.9, 4.2, 2.0 Hz, 2H), 1.36-1.22 (m, 2H), 1.04 (t, J=7.1 Hz, 3H). LCMS (ES) [M+1]⁺ m/z: 477.1.

### Example 1.26

### 3-{[1-(4-chloro-3-fluorophenyl)-3-methyl-1H-1,2,4-triazol-5-yl]methyl}-1-({1-[(oxan-4-yl)methyl]-1H-1,2,4-triazol-5-yl}methyl)urea (Compound 10)

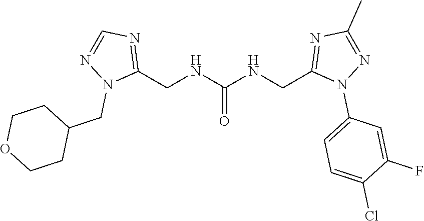 [SMILES: Cc1nc(CNC(=O)NCc2ncnn2CC2CCOCC2)n(-c2ccc(Cl)c(F)c2)n1]

The title compound was synthesized according to General Procedure 7, Method C, using 1-{1-[(oxan-4-yl)methyl]-1H-1,2,4-triazol-5-yl}methanamine hydrochloride (Intermediate I-8; 160 mg) and 1-[1-(4-chloro-3-fluorophenyl)-3-methyl-1H-1,2,4-triazol-5-yl]methanamine (Intermediate I-6; 1 eq.) to provide 3-{[1-(4-chloro-3-fluorophenyl)-1H-1,2,4-triazol-5-yl]methyl}-1-({1-[(oxan-4-yl)methyl]-1H-1,2,4-triazol-5-yl}methyl)urea (55 mg; 27% yield) as a white solid. 1-H NMR (400 MHz, Acetonitrile-d3) δ 7.75 (s, 1H), 7.63 (t, J=8.3 Hz, 1H), 7.57 (dd, J=9.9, 2.4 Hz, 1H), 7.39 (ddd, J=8.6, 2.4, 1.2 Hz, 1H), 5.90 (q, J=5.4 Hz, 2H), 4.41 (dd, J=9.2, 5.8 Hz, 4H), 4.02 (d, J=7.3 Hz, 2H), 3.86 (ddd, J=11.4, 5.0, 1.9 Hz, 2H), 3.28 (td, J=11.7, 2.2 Hz, 2H), 2.33 (s, 3H), 2.11 (tp, J=11.3, 3.6 Hz, 1H), 1.43 (ddd, J=13.0, 4.2, 2.1 Hz, 2H), 1.36-1.22 (m, 2H). LCMS (ES) [M+1]⁺ m/z: 463.0.

### Example 1.27

### Synthesis of 3-{[1-(4-chloro-3-fluorophenyl)-1H-1,2,4-triazol-5-yl]methyl}-1-({1-[(4-methyloxan-4-yl)methyl]-1H-1,2,4-triazol-5-yl}methyl)urea (Compound 11)

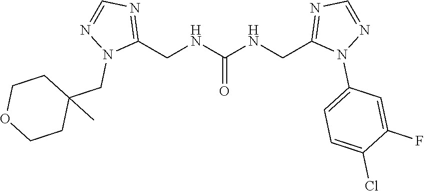 [SMILES: CC1(Cn2ncnc2CNC(=O)NCc2ncnn2-c2ccc(Cl)c(F)c2)CCOCC1]

The title compound was synthesized according to General Procedure 7, Method C, using {1-[(4-methyloxan-4-yl)methyl]-1H-1,2,4-triazol-5-yl}methanaminium chloride (65 mg; synthesized according to General Procedure 8, starting from commercial tetrahydro-4-methyl-2h-pyran-4-carboxaldehyde) and 1-[1-(4-chloro-3-fluorophenyl)-1H-1,2,4-triazol-5-yl]methanamine (Intermediate I-4; 1 eq.) to provide 3-{[1-(4-chloro-3-fluorophenyl)-1H-1,2,4-triazol-5-yl]methyl}-1-({1-[(oxan-4-yl)methyl]-1H-1,2,4-triazol-5-yl}methyl)urea (58 mg; 48% yield) as a white solid. 1-H NMR (400 MHz, DMSO-d6) δ 7.84 (s, 1H), 7.83-7.74 (m, 3H), 7.50 (ddd, J=8.6, 2.4, 1.1 Hz, 1H), 6.72 (t, J=5.7 Hz, 2H), 4.43 (d, J=5.7 Hz, 2H), 4.33 (d, J=5.7 Hz, 2H), 4.01 (s, 2H), 3.62 (dt, J=11.7, 4.4 Hz, 2H), 3.47 (ddd, J=12.0, 9.7, 2.8 Hz, 2H), 1.51 (ddd, J=13.8, 9.7, 4.3 Hz, 2H), 1.21 (dd, J=13.2, 3.8 Hz, 2H), 0.88 (s, 3H). LCMS (ES) [M+1]⁺ m/z: 463.0.

### Example 1.28

### Synthesis of 3-{[1-(4-chloro-3-fluorophenyl)-3-methyl-1H-1,2,4-triazol-5-yl]methyl}-1-({1-[(4-methyloxan-4-yl)methyl]-1H-1,2,4-triazol-5-yl}methyl)urea (Compound 12)

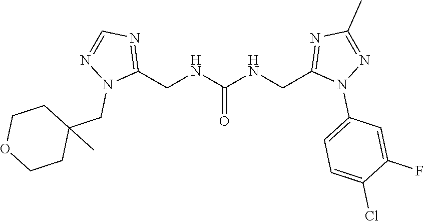 [SMILES: Cc1nc(CNC(=O)NCc2ncnn2CC2(C)CCOCC2)n(-c2ccc(Cl)c(F)c2)n1]

The title compound was synthesized according to General Procedure 7, Method C, using {1-[(4-methyloxan-4-yl)methyl]-1H-1,2,4-triazol-5-yl}methanaminium chloride (65 mg; synthesized according to General Procedure 8, starting from commercial tetrahydro-4-methyl-2h-pyran-4-carboxaldehyde) and 1-[1-(4-chloro-3-fluorophenyl)-3-methyl-1H-1,2,4-triazol-5-yl]methanamine (Intermediate I-6; 1 eq.) to provide 3-{[1-(4-chloro-3-fluorophenyl)-3-methyl-1H-1,2,4-triazol-5-yl]methyl}-1-({1-[(4-methyloxan-4-yl)methyl]-1H-1,2,4-triazol-5-yl}methyl)urea (37 mg; 29% yield) as a white solid. 1H NMR (400 MHz, DMSO-d6) δ 7.84 (s, 1H), 7.80-7.71 (m, 2H), 7.47 (ddd, J=8.7, 2.5, 1.2 Hz, 1H), 6.71 (t, J=5.7 Hz, 2H), 4.38 (d, J=5.6 Hz, 2H), 4.33 (d, J=5.8 Hz, 2H), 4.02 (s, 2H), 3.62 (dt, J=11.7, 4.4 Hz, 2H), 3.47 (ddd, J=12.0, 9.7, 2.8 Hz, 2H), 2.28 (s, 3H), 1.51 (ddd, J=13.8, 9.7, 4.3 Hz, 2H), 1.28-1.15 (m, 2H), 0.88 (s, 3H). LCMS (ES) [M+1]⁺ m/z: 477.1.

### Example 1.29

### Synthesis of 3-{[1-(4-chloro-3-fluorophenyl)-1H-1,2,4-triazol-5-yl]methyl}-1-({1-[(2,2-dimethyloxan-4-yl)methyl]-1H-1,2,4-triazol-5-yl}methyl)urea (Compound 13)

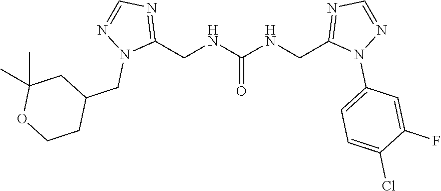 [SMILES: CC1(C)CC(Cn2ncnc2CNC(=O)NCc2ncnn2-c2ccc(Cl)c(F)c2)CCO1]

The title compound was synthesized according to General Procedure 7, Method C, using {1-[(2,2-dimethyloxan-4-yl)methyl]-1H-1,2,4-triazol-5-yl}methanaminium chloride (114 mg; synthesized according to General Procedure 8 starting from commercial 2,2-dimethyltetrahydro-2H-pyran-4-carbaldehyde) and 1-[1-(4-chloro-3-fluorophenyl)-1H-1,2,4-triazol-5-yl]methanamine (Intermediate I-4; 1 eq.) to provide 3-{[1-(4-chloro-3-fluorophenyl)-1H-1,2,4-triazol-5-yl]methyl}-1-({1-[(2,2-dimethyloxan-4-yl)methyl]-1H-1,2,4-triazol-5-yl}methyl)urea (41 mg; 20% yield) as a white solid. 1-H NMR (400 MHz, DMSO-d6) δ 8.08 (s, 1H), 7.85-7.71 (m, 3H), 7.49 (ddd, J=8.7, 2.5, 1.2 Hz, 1H), 6.70 (td, J=5.8, 3.1 Hz, 2H), 4.47-4.34 (m, 2H), 4.30 (d, J=5.8 Hz, 2H), 4.00-3.84 (m, 2H), 3.51 (ddd, J=11.9, 5.1, 1.6 Hz, 1H), 3.41 (td, J=12.1, 2.3 Hz, 1H), 2.14 (dd, J=9.3, 5.7 Hz, 1H), 1.36-1.21 (m, 2H), 1.13-0.92 (m, 8H). LCMS (ES) [M+1]⁺ m/z: 477.1.

### Example 1.30

### Synthesis of 3-{[1-(4-chloro-3-fluorophenyl)-3-methyl-1H-1,2,4-triazol-5-yl]methyl}-1-({1-[(2,2-dimethyloxan-4-yl)methyl]-1H-1,2,4-triazol-5-yl}methyl)urea (Compound 14)

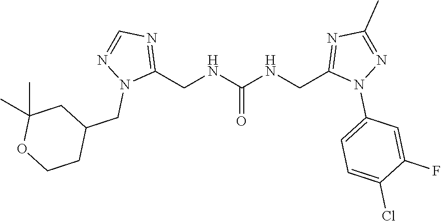 [SMILES: Cc1nc(CNC(=O)NCc2ncnn2CC2CCOC(C)(C)C2)n(-c2ccc(Cl)c(F)c2)n1]

The title compound was synthesized according to General Procedure 7, Method C, using {1-[(2,2-dimethyloxan-4-yl)methyl]-1H-1,2,4-triazol-5-yl}methanaminium chloride (114 mg; synthesized according to General Procedure 8 starting from commercial 2,2-dimethyltetrahydro-2H-pyran-4-carbaldehyde) and 1-[1-(4-chloro-3-fluorophenyl)-3-methyl-1H-1,2,4-triazol-5-yl]methanamine (Intermediate I-6; 1 eq.) to provide 3-{[1-(4-chloro-3-fluorophenyl)-1H-1,2,4-triazol-5-yl]methyl}-1-({1-[(2,2-dimethyloxan-4-yl)methyl]-1H-1,2,4-triazol-5-yl}methyl)urea (41 mg; 20% yield) as a white solid. ¹H NMR (400 MHz, DMSO-d6) δ 7.83-7.69 (m, 3H), 7.46 (ddd, J=8.7, 2.5, 1.2 Hz, 1H), 6.67 (td, J=5.7, 3.4 Hz, 2H), 4.36 (dd, J=5.7, 0.9 Hz, 2H), 4.31 (d, J=5.8 Hz, 2H), 4.00-3.85 (m, 2H), 3.56-3.46 (m, 1H), 3.41 (td, J=12.1, 2.3 Hz, 1H), 2.26 (s, 3H), 2.15 (td, J=8.0, 4.5 Hz, 1H), 1.34-1.23 (m, 2H), 1.12-0.93 (m, 8H). LCMS (ES) [M+1]⁺ m/z: 491.0.

### Example 1.31

### Synthesis of 3-({1-[(oxan-4-yl)methyl]-1H-1,2,4-triazol-5-yl}methyl)-1-{[1-(quinolin-7-yl)-1H-1,2,4-triazol-5-yl]methyl}urea (Compound 15)

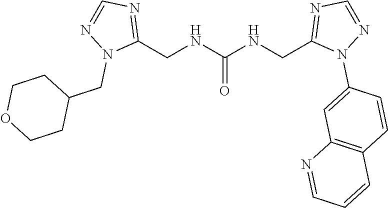 [SMILES: O=C(NCc1ncnn1CC1CCOCC1)NCc1ncnn1-c1ccc2cccnc2c1]

The title compound was synthesized according to General Procedure 7, Method C, using 1-{1-[(oxan-4-yl)methyl]-1H-1,2,4-triazol-5-yl}methanamine hydrochloride (Intermediate I-8; 83 mg) and 1-[1-(quinolin-7-yl)-1H-1,2,4-triazol-5-yl]methanamine (Intermediate I-9; 1 eq.) to provide 3-({1-[(oxan-4-yl)methyl]-1H-1,2,4-triazol-5-yl}methyl)-1-{[1-(quinolin-7-yl)-1H-1,2,4-triazol-5-yl]methyl}urea (33 mg; 15% yield) as a white solid. 1-H NMR (400 MHz, Acetonitrile-d3) δ 9.01 (dd, J=4.2, 1.7 Hz, 1H), 8.39 (dd, J=8.1, 1.8 Hz, 1H), 8.25 (d, J=2.1 Hz, 1H), 8.14-8.06 (m, 1H), 8.02 (s, 1H), 7.77 (dd, J=8.7, 2.2 Hz, 1H), 7.73 (s, 1H), 7.59 (dd, J=8.4, 4.2 Hz, 1H), 5.89 (dt, J=29.9, 5.8 Hz, 2H), 4.57 (d, J=5.7 Hz, 2H), 4.37 (d, J=5.8 Hz, 2H), 3.99 (d, J=7.3 Hz, 2H), 3.84 (ddd, J=11.6, 5.0, 2.0 Hz, 2H), 3.32-3.19 (m, 2H), 2.08 (ddh, J=15.2, 7.4, 3.8 Hz, 1H), 1.41 (ddd, J=13.0, 4.2, 2.1 Hz, 2H), 1.36-1.17 (m, 2H). LCMS (ES) [M+1]⁺ m/z: 447.9.

### Example 1.32

### Synthesis of 3-{[1-(4-chloro-3-fluorophenyl)-3-methyl-1H-1,2,4-triazol-5-yl]methyl}-1-{[1-(cyclohexylmethyl)-1H-1,2,4-triazol-5-yl]methyl}urea (Compound 16)

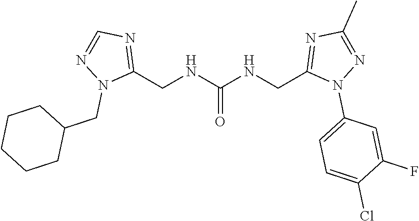 [SMILES: Cc1nc(CNC(=O)NCc2ncnn2CC2CCCCC2)n(-c2ccc(Cl)c(F)c2)n1]

The title compound was synthesized according to General Procedure 7, Method C, using [1-(cyclohexylmethyl)-1H-1,2,4-triazol-5-yl]methanaminium chloride (147 mg; synthesized according to General Procedure 8 starting from commercial cyclohexylmethyl-hydrazine hydrochloride) and 1-[1-(4-chloro-3-fluorophenyl)-3-methyl-1H-1,2,4-triazol-5-yl]methanamine (Intermediate I-6; 1 eq.) to provide 3-{[1-(4-chloro-3-fluorophenyl)-3-methyl-1H-1,2,4-triazol-5-yl]methyl}-1-{[1-(cyclohexylmethyl)-1H-1,2,4-triazol-5-yl]methyl}urea (150 mg; 51% yield) as a white solid. 1H NMR (400 MHz, Acetonitrile-d3) δ 7.73 (s, 1H), 7.62 (t, J=8.3 Hz, 1H), 7.57 (dd, J=9.9, 2.4 Hz, 1H), 7.39 (ddd, J=8.6, 2.4, 1.2 Hz, 1H), 5.98 (q, J=6.4 Hz, 2H), 4.42 (d, J=5.7 Hz, 2H), 4.38 (d, J=5.7 Hz, 2H), 3.95 (d, J=7.3 Hz, 2H), 2.32 (s, 3H), 1.85 (dtq, J=15.1, 7.5, 3.6 Hz, 1H), 1.73-1.59 (m, 3H), 1.58-1.49 (m, 2H), 1.27-1.12 (m, 3H), 0.98 (qd, J=11.8, 3.4 Hz, 2H). LCMS (ES) [M+1]⁺ m/z: 461.1.

### Example 1.33

### Synthesis of 1-{[1-(1,3-benzothiazol-5-yl)-1H-1,2,4-triazol-5-yl]methyl}-3-{[1-(4-chloro-3-fluorophenyl)-3-methyl-1H-1,2,4-triazol-5-yl]methyl}urea (Compound 17)

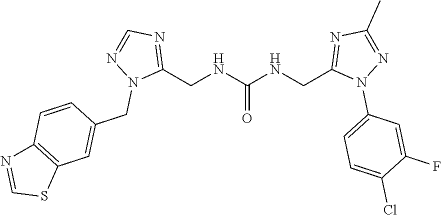 [SMILES: Cc1nc(CNC(=O)NCc2ncnn2Cc2ccc3ncsc3c2)n(-c2ccc(Cl)c(F)c2)n1]

The synthesis of this compound is described in General Procedure 7, Method C.

### Example 1.34

### Synthesis of 1-[(1-benzyl-1H-1,2,4-triazol-5-yl)methyl]-3-{[1-(4-chloro-3-fluorophenyl)-3-methyl-1H-1,2,4-triazol-5-yl]methyl}urea (Compound 18)

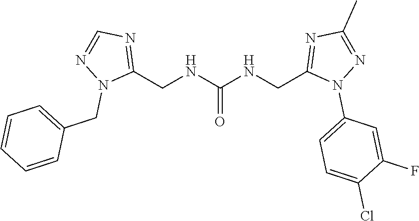 [SMILES: Cc1nc(CNC(=O)NCc2ncnn2Cc2ccccc2)n(-c2ccc(Cl)c(F)c2)n1]

The title compound was synthesized according to General Procedure 7, Method C, using (1-benzyl-1H-1,2,4-triazol-5-yl)methanaminium chloride (212 mg; synthesized according to General Procedure 8, step 2 starting from commercially available benzylhydrazine) and 1-[1-(4-chloro-3-fluorophenyl)-3-methyl-1H-1,2,4-triazol-5-yl]methanamine (Intermediate I-6; 1 eq.) to provide 1-[(1-benzyl-1H-1,2,4-triazol-5-yl)methyl]-3-{[1-(4-chloro-3-fluorophenyl)-3-methyl-1H-1,2,4-triazol-5-yl]methyl}urea as a white solid (243 mg; 57% yield). 1-H NMR (400 MHz, DMSO-d6) δ 7.85 (s, 1H), 7.79-7.70 (m, 2H), 7.46 (ddd, J=8.6, 2.5, 1.1 Hz, 1H), 7.33-7.23 (m, 3H), 7.21-7.13 (m, 2H), 6.75 (t, J=5.8 Hz, 1H), 6.70 (t, J=5.7 Hz, 1H), 5.38 (s, 2H), 4.36 (dd, J=11.8, 5.7 Hz, 4H), 2.27 (s, 3H). LCMS (ES) [M+1]⁺ m/z: 455.0.

### Example 1.35

### Synthesis of 1-{[1-(1,3-benzothiazol-6-yl)-1H-1,2,4-triazol-5-yl]methyl}-3-{[1-(4-chloro-3-fluorophenyl)-3-methyl-1H-1,2,4-triazol-5-yl]methyl}urea (Compound 19)

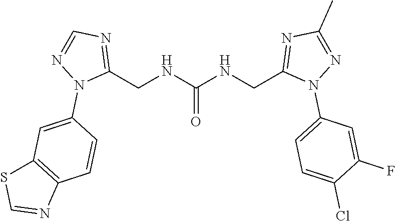 [SMILES: Cc1nc(CNC(=O)NCc2ncnn2-c2ccc3ncsc3c2)n(-c2ccc(Cl)c(F)c2)n1]

The title compound was synthesized according to General Procedure 7, Method C, using [1-(1,3-benzothiazol-6-yl)-1H-1,2,4-triazol-5-yl]methanaminium chloride (153 mg; synthesized according to General Procedures 1, 2 and 3 starting from commercial 6-aminobenzothiazole) and 1-[1-(4-chloro-3-fluorophenyl)-3-methyl-1H-1,2,4-triazol-5-yl]methanamine (Intermediate I-6; 1 eq.) to provide 1-{[1-(1,3-benzothiazol-6-yl)-1H-1,2,4-triazol-5-yl]methyl}-3-{[1-(4-chloro-3-fluorophenyl)-3-methyl-1H-1,2,4-triazol-5-yl]methyl}urea (190 mg; 67% yield) as a white solid. ¹H NMR (400 MHz, DMSO-d6) δ 9.50 (s, 1H), 8.43 (d, J=2.1 Hz, 1H), 8.21 (d, J=8.7 Hz, 1H), 8.11 (s, 1H), 7.78-7.69 (m, 3H), 7.44 (ddd, J=8.7, 2.4, 1.2 Hz, 1H), 6.72 (td, J=5.7, 2.3 Hz, 2H), 4.41 (d, J=5.6 Hz, 2H), 4.32 (d, J=5.6 Hz, 2H), 2.26 (s, 3H). LCMS (ES) [M+1]⁺ m/z: 497.9.

### Example 1.36

### Synthesis of 3-{[1-(4-chloro-3-fluorophenyl)-3-methyl-1H-1,2,4-triazol-5-yl]methyl}-1-({1-[(4-fluorophenyl)methyl]-1H-1,2,4-triazol-5-yl}methyl)urea (Compound 20)

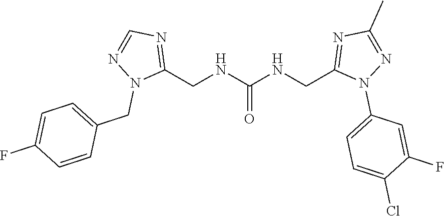 [SMILES: Cc1nc(CNC(=O)NCc2ncnn2Cc2ccc(F)cc2)n(-c2ccc(Cl)c(F)c2)n1]

The title compound was synthesized according to General Procedure 7, Method C, using {1-[(4-fluorophenyl)methyl]-1H-1,2,4-triazol-5-yl}methanaminium chloride (97 mg; synthesized according to General Procedure 8, step 2 starting from commercial 4-fluorobenzyl-hydrazine) and 1-[1-(4-chloro-3-fluorophenyl)-3-methyl-1H-1,2,4-triazol-5-yl]methanamine (Intermediate I-6; 1 eq.) to provide 3-{[1-(4-chloro-3-fluorophenyl)-3-methyl-1H-1,2,4-triazol-5-yl]methyl}-1-({1-[(4-fluorophenyl)methyl]-1H-1,2,4-triazol-5-yl}methyl)urea (108 mg; 57% yield) as a white solid. ¹H NMR (400 MHz, DMSO-d6) δ 7.85 (s, 1H), 7.79-7.70 (m, 2H), 7.46 (ddd, J=8.6, 2.5, 1.2 Hz, 1H), 7.30-7.21 (m, 2H), 7.16-7.07 (m, 2H), 6.74 (t, J=5.8 Hz, 1H), 6.68 (t, J=5.7 Hz, 1H), 5.36 (s, 2H), 4.36 (dd, J=7.9, 5.8 Hz, 4H), 2.27 (s, 3H). LCMS (ES) [M+1]⁺ m/z: 473.1.

### Example 1.37

### Synthesis of 1-{[1-(3-fluoro-4-methoxyphenyl)-3-methyl-1H-1,2,4-triazol-5-yl]methyl}-3-{[1-(quinolin-7-yl)-1H-1,2,4-triazol-5-yl]methyl}urea (Compound 21)

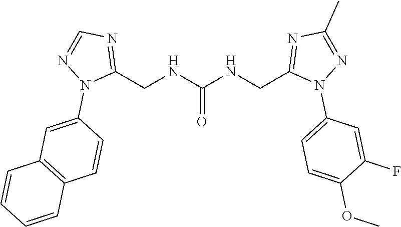 [SMILES: COc1ccc(-n2nc(C)nc2CNC(=O)NCc2ncnn2-c2ccc3ccccc3c2)cc1F]

The title compound was synthesized according to General Procedure 7, Method C, using [1-(3-fluoro-4-methoxyphenyl)-1H-1,2,4-triazol-5-yl]methanaminium chloride (167 mg; synthesized according to General Procedure 2 and 3 starting from commercial 3-fluoro-4-methoxyphenyl-hydrazine) and Intermediate I-9 (144 mg) to provide 1-{[1-(3-fluoro-4-methoxyphenyl)-3-methyl-1H-1,2,4-triazol-5-yl]methyl}-3-{[1-(116uinoline-7-yl)-1H-1,2,4-triazol-5-yl]methyl}urea (200 mg; 66% yield) as a white solid. 1H NMR (400 MHz, DMSO-d6) δ 8.99 (dd, J=4.2, 1.7 Hz, 1H), 8.52-8.43 (m, 1H), 8.23 (d, J=2.1 Hz, 1H), 8.19-8.12 (m, 2H), 8.03 (s, 1H), 7.81 (dd, J=8.7, 2.1 Hz, 1H), 7.62 (dd, J=8.3, 4.2 Hz, 1H), 7.51 (dd, J=11.8, 2.5 Hz, 1H), 7.35 (ddd, J=8.8, 2.5, 1.2 Hz, 1H), 7.28 (t, J=8.9 Hz, 1H), 6.77 (t, J=5.6 Hz, 1H), 6.75-6.69 (m, 1H), 4.49 (d, J=5.5 Hz, 2H), 4.30 (d, J=5.6 Hz, 2H), 3.88 (s, 3H). LCMS (ES) [M+1] ⁺m/z: 473.9.

### Example 1.38

### Synthesis of 1-{[1-(1,3-benzothiazol-5-yl)-1H-1,2,4-triazol-5-yl]methyl}-3-{[1-(4-chloro-3-fluorophenyl)-3-methyl-1H-1,2,4-triazol-5-yl]methyl}-1-methylurea (Compound 22)

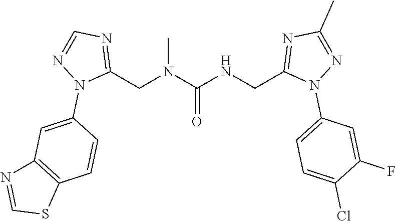 [SMILES: Cc1nc(CNC(=O)N(C)Cc2ncnn2-c2ccc3scnc3c2)n(-c2ccc(Cl)c(F)c2)n1]

Step 1

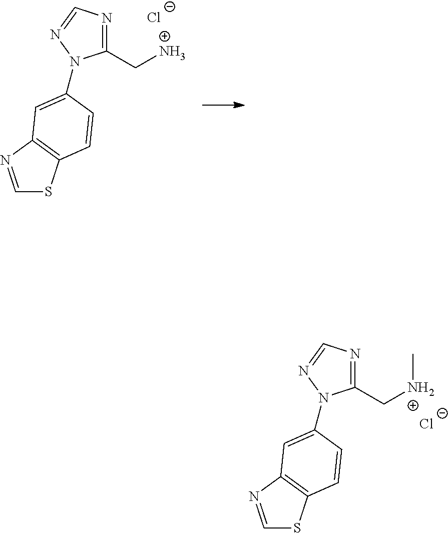 [SMILES: [NH3+]Cc1ncnn1-c1ccc2scnc2c1 ; [Cl-] ; C[NH2+]Cc1ncnn1-c1ccc2scnc2c1 ; [Cl-]]

Di-tert butyl dicarbonate was added to a solution of [1-(1,3-benzothiazol-5-yl)-1H-1,2,4-triazol-5-yl]methanamine (300 mg, synthesized according to General Procedures 1, 2 and 3 starting from commercial 5-aminobenzothiazole), and triethylamine (0.3 mL) in THF (6 mL) and the resulting mixture was stirred for 3 hours at ambient temperature. The mixture was then concentrated under reduced pressure and purified by silica gel column chromatography using ethyl acetate as the eluting solvent to afford tert-butyl N-{[1-(1,3-benzothiazol-5-yl)-1H-1,2,4-triazol-5-yl]methyl}carbamate (260 mg; 70% yield).

This material was dissolved in THF (4 mL) and sodium hydride 60% wt (62 mg; 1.6 mmol) was added to the reaction mixture over 10 minutes. 2 M iodomethane in TBME (0.78 mL, 1.6 mmol) was then added and the resulting mixture was stirred for 20 hours at ambient temperature. The reaction mixture was then diluted with water and extracted with ethyl acetate. The combined organics were dried with MgSO4, filtered, and concentrated. The residue was purified by silica gel column chromatography using 80% ethyl acetate in heptanes as the eluting solvents to afford tert-butyl N-{[1-(1,3-benzothiazol-5-yl)-1H-1,2,4-triazol-5-yl]methyl}-N-methylcarbamate (240 mg; 90% yield) as a white solid. This intermediate was treated with 4 M HCl in dioxanes (4 mL) and after 2 hours, the reaction mixture was concentrated under reduced pressure to provide {[1-(1,3-benzothiazol-5-yl)-1H-1,2,4-triazol-5-yl]methyl (methyl)amine hydrochloride (220 mg; 99% crude yield) as a white solid. LCMS (ES) [M+1] ⁺ m/z: 245.9.

Step 2

The title compound was synthesized according to General Procedure 7, Method C, using {[1-(1,3-benzothiazol-5-yl)-1H-1,2,4-triazol-5-yl]methyl}(methyl)amine hydrochloride (30 mg) and 1-[1-(4-chloro-3-fluorophenyl)-3-methyl-1H-1,2,4-triazol-5-yl]methanamine (Intermediate I-6; 1 eq.) to provide 1-{[1-(1,3-benzothiazol-5-yl)-1H-1,2,4-triazol-5-yl]methyl}-3-{[1-(4-chloro-3-fluorophenyl)-3-methyl-1H-1,2,4-triazol-5-yl]methyl}-1-methylurea (8 mg; 15% yield) as a white solid. 1H NMR (400 MHz, Acetonitrile-d3) δ 9.24 (s, 1H), 8.23 (d, J=2.0 Hz, 1H), 8.19 (d, J=8.6 Hz, 1H), 7.99 (s, 1H), 7.66-7.57 (m, 2H), 7.54 (dd, J=9.9, 2.4 Hz, 1H), 7.36 (ddd, J=8.7, 2.5, 1.3 Hz, 1H), 5.85 (t, J=5.5 Hz, 1H), 4.62 (s, 2H), 4.30 (d, J=5.5 Hz, 2H), 2.81 (s, 3H), 2.32 (s, 3H). LCMS (ES) [M+1]⁺ m/z: 512.1.

### Example 1.39

### Synthesis of 3-{[1-(4-chloro-3-fluorophenyl)-3-methyl-1H-1,2,4-triazol-5-yl]methyl}-1-({1-[(1-hydroxycyclohexyl)methyl]-1H-1,2,4-triazol-5-yl}methyl)urea (Compound 23)

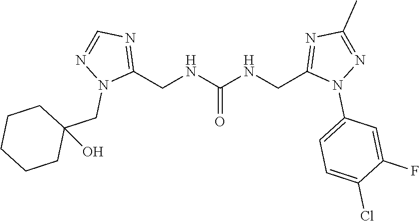 [SMILES: Cc1nc(CNC(=O)NCc2ncnn2CC2(O)CCCCC2)n(-c2ccc(Cl)c(F)c2)n1]

The title compound was synthesized according to General Procedure 7, Method C, using 1-[1-(4-chloro-3-fluorophenyl)-3-methyl-1H-1,2,4-triazol-5-yl]methanamine (Intermediate I-6; 130 mg) and 1-[1-({1-[(tert-butyldimethylsilyl)oxy]cyclohexyl}methyl)-1H-1,2,4-triazol-5-yl]methanamine hydrochloride (169 mg; synthesized according to General Procedure 8 starting from commercial 1-((tert-butyldimethylsilyl)oxy)cyclohexane-1-carbaldehyde) to provide 1-{[1-({1-[(tert-butyldimethylsilyl)oxy]cyclohexyl methyl)-1H-1,2,4-triazol-5-yl]methyl}-3-{[1-(4-chloro-3-fluorophenyl)-3-methyl-1H-1,2,4-triazol-5-yl]methyl}urea (277 mg, 99% yield) as a crude oil. LCMS (ES) [M+1]⁺ m/z: 591.3. This crude material was treated with 1M HCl (5 mL) and MeCN (1 mL) at 90° C. for 3 hours and then it was cooled to ambient temperature and concentrated under reduced pressure. The residue was purified by prep HPLC using 5% to 75% MeCN in 0.1% formic acid in water as the eluting solvents to provide 3-{[1-(4-chloro-3-fluorophenyl)-3-methyl-1H-1,2,4-triazol-5-yl]methyl}-1-({1-[(1-hydroxycyclohexyl)methyl]-1H-1,2,4-triazol-5-yl}methyl)urea (146 mg, 65%) as a white solid. ¹H NMR (400 MHz, DMSO-d6) δ 7.84-7.68 (m, 3H), 7.47 (ddd, J=8.6, 2.4, 1.1 Hz, 1H), 6.73 (t, J=5.7 Hz, 1H), 6.61 (t, J=5.7 Hz, 1H), 4.52 (s, 1H), 4.36 (t, J=6.0 Hz, 4H), 4.06 (s, 2H), 3.21-3.08 (m, 2H), 2.28 (s, 3H), 1.63-1.44 (m, 2H), 1.44-1.36 (m, 2H), 1.36-1.12 (m, 4H), 0.92 (t, J=7.3 Hz, 2H). LCMS (ES) [M+1]⁺ m/z: 477.0.

### Example 1.40

### Synthesis of 3-{[1-(4-chloro-3-fluorophenyl)-3-methyl-1H-1,2,4-triazol-5-yl]methyl}-1-{[1-(2,3-dihydro-1-benzofuran-6-yl)-1H-1,2,4-triazol-5-yl]methyl}urea (Compound 24)

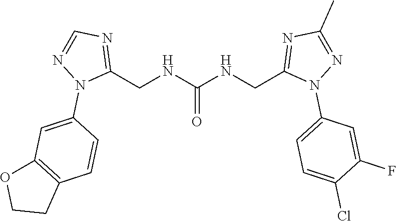 [SMILES: Cc1nc(CNC(=O)NCc2ncnn2-c2ccc3c(c2)OCC3)n(-c2ccc(Cl)c(F)c2)n1]

The title compound was synthesized according to General Procedure 7, Method C, using [1-(2,3-dihydro-1-benzofuran-6-yl)-1H-1,2,4-triazol-5-yl]m ethanaminium chloride (233 mg; synthesized according to General Procedures 1, 2 and 3, starting from commercial 2,3-dihydrobenzofuran-6-amine) and 1-[1-(4-chloro-3-fluorophenyl)-3-methyl-1H-1,2,4-triazol-5-yl]methanamine (Intermediate I-6; 1 eq.) to provide 3-{[1-(4-chloro-3-fluorophenyl)-3-methyl-1H-1,2,4-triazol-5-yl]methyl}-1-{[1-(2,3-dihydro-1-benzofuran-6-yl)-1H-1,2,4-triazol-5-yl]methyl}urea (317 mg; 71% yield) as a white solid. 1H NMR (400 MHz, DMSO-d6) δ 8.02 (s, 1H), 7.78-7.69 (m, 2H), 7.45 (ddd, J=8.7, 2.4, 1.2 Hz, 1H), 7.39-7.30 (m, 1H), 7.01-6.92 (m, 2H), 6.71 (dt, J=16.0, 5.6 Hz, 2H), 4.60 (t, J=8.8 Hz, 2H), 4.33 (dd, J=12.9, 5.6 Hz, 4H), 3.27-3.16 (m, 2H), 2.27 (s, 3H). LCMS (ES) [M+1]⁺ m/z: 483.3.

### Example 1.41

### Synthesis of 1,3-bis({[1-(4-chloro-2-fluorophenyl)-1H-1,2,3,4-tetrazol-5-yl]methyl})urea (Compound 25)

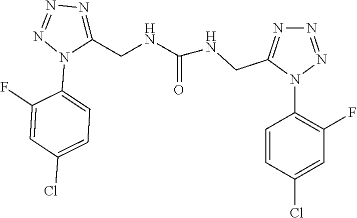 [SMILES: O=C(NCc1nnnn1-c1ccc(Cl)cc1F)NCc1nnnn1-c1ccc(Cl)cc1F]

The title compound was synthesized according to General Procedure 6, Method B using 1-[1-(4-chloro-2-fluorophenyl)-1H-1,2,3,4-tetrazol-5-yl]methanamine (55 mg; synthesized according to General Procedure 4 starting from commercial 2-fluoro-4-chloroaniline) to provide 1,3-bis({[1-(4-chloro-2-fluorophenyl)-1H-1,2,3,4-tetrazol-5-yl]methyl})urea (19 mg, 32.68%). 1H NMR (400 MHz, DMSO-d6) δ 7.83 (dd, J=10.0, 2.2 Hz, 2H), 7.75 (t, J=8.3 Hz, 2H), 7.54 (dd, J=8.8, 2.0 Hz, 2H), 6.78 (t, J=5.8 Hz, 2H), 4.36 (d, J=5.7 Hz, 4H). LCMS (ES) [M+1]⁺ m/z 481.05-483.35.

### Example 1.42

### Synthesis of 1,3-bis({[1-(4-chloro-3-fluorophenyl)-1H-1,2,3,4-tetrazol-5-yl]methyl})-1-methylurea (Compound 26)

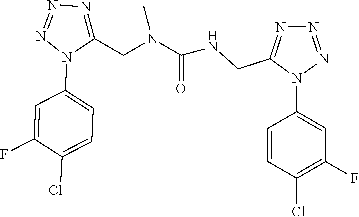 [SMILES: CN(Cc1nnnn1-c1ccc(Cl)c(F)c1)C(=O)NCc1nnnn1-c1ccc(Cl)c(F)c1]

Step 1

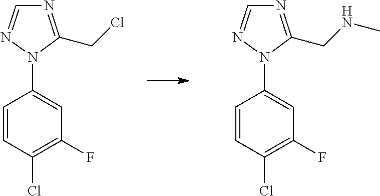 [SMILES: Fc1cc(-n2ncnc2CCl)ccc1Cl ; CNCc1ncnn1-c1ccc(Cl)c(F)c1]

A sealed tube containing 1-(4-chloro-3-fluorophenyl)-5-(chloromethyl)-1H-1,2,3,4-tetrazole (General Procedure 4, Step 2, 450.00 mg; 1.82 mmol; 1.00 eq.) and methylamine (9.11 mL; 2.00 mol/L; 18.21 mmol; 10.00 eq.) in ethanol (9.00 mL) was heated at 40° C. for 90 min and then left stirring at RT over the weekend The reaction mix was concentrated to dryness and the product was purified by silica gel column using 0-70% Magic (DCM:MEOH:NH₄OH; 90:9:1) in DCM to afford {[1-(4-chloro-3-fluorophenyl)-1H-1,2,3,4-tetrazol-5-yl]methyl}(methyl)amine (390 mg, 88.6%). 1-H NMR (400 MHz, DMSO-d6) δ 8.03 (dt, J=9.8, 1.7 Hz, 1H), 7.89 (td, J=8.3, 1.2 Hz, 1H), 7.68 (ddd, J=8.7, 2.4, 1.2 Hz, 1H), 3.98 (s, 2H), 2.40 (s, 1H), 2.22 (d, J=1.4 Hz, 3H). LCMS (ES) [M+1]⁺ m/z 242.21.

Step 2

The title compound was synthesized according to General Procedure 7, Method A using 1-[1-(4-chloro-3-fluorophenyl)-1H-1,2,3,4-tetrazol-5-yl]methanamine (125.00 mg; 0.55 mmol; 1.00 eq. synthesized from 3-chloro-4-fluoroaniline according to General Procedure 4), and {[1-(4-chloro-3-fluorophenyl)-1H-1,2,3,4-tetrazol-5-yl]methyl}(methyl)amine (132.70 mg; 0.55 mmol; 1.00 eq.), to give 1,3-bis({[1-(4-chloro-3-fluorophenyl)-1H-1,2,3,4-tetrazol-5-yl]methyl})-1-methylurea (210 mg, 77.21%). 1H NMR (400 MHz, DMSO-d6) δ 7.96-7.79 (m, 4H), 7.53 (d, J=8.2 Hz, 2H), 7.30 (t, J=5.2 Hz, 1H), 4.67 (s, 2H), 4.43 (d, J=5.1 Hz, 2H), 2.74 (s, 3H). LCMS (ES) [M+1]⁺ m/z 496.8.

### Example 1.43

### Synthesis of 1,3-bis({[1-(4-chloro-3-fluorophenyl)-1H-1,2,4-triazol-5-yl]methyl})-1-methylurea (Compound 27)

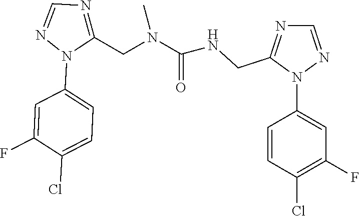 [SMILES: CN(Cc1ncnn1-c1ccc(Cl)c(F)c1)C(=O)NCc1ncnn1-c1ccc(Cl)c(F)c1]

Step 1

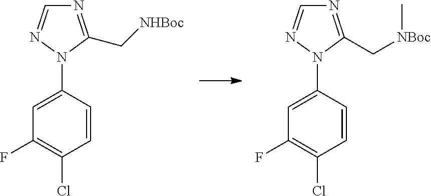 [SMILES: CC(C)(C)OC(=O)NCc1ncnn1-c1ccc(Cl)c(F)c1 ; CN(Cc1ncnn1-c1ccc(Cl)c(F)c1)C(=O)OC(C)(C)C]

To a stirred solution of tert-butyl N-{[1-(4-chloro-3-fluorophenyl)-1H-1,2,4-triazol-5-yl]methyl}carbamate (Intermediate I-3, 1 000.00 mg; 3.06 mmol; 1.00 eq.) in N,N-dimethylformamide cooled to 0° C. was added sodium hydride (306.01 mg; 7.65 mmol; 2.50 eq.) in one portion and the mixture was stirred at 0° C. for 30 min. Iodomethane (0.95 mL; 15.30 mmol; 5.00 eq.) was then added drop wise and the mixture was left to reach RT slowly. After 1 h, LCMS showed reaction completion. The reaction flask was cooled in an ice bath and the reaction mixture was quenched by drop wise addition of water while stirring and then diluted with water. A solid precipitated which was confirmed to be the desired tert-butyl N-{[1-(4-chloro-3-fluorophenyl)-1H-1,2,4-triazol-5-yl]methyl}-N-methylcarbamate which was isolated by filtration and dried under high vacuum (1035 g, 99%). ¹H NMR (400 MHz, DMSO-d6) δ 8.14 (s, 1H), 7.78 (dt, J=9.9, 2.1 Hz, 2H), 7.46 (d, J=8.6 Hz, 1H), 4.65 (d, J=10.3 Hz, 2H), 2.76 (d, J=25.1 Hz, 3H), 1.22 (d, J=57.2 Hz, 10H). LCMS (ES) [M+1]⁺ m/z 340.88. LCMS (ES) [M-100]+m/z 240.88.

Step 2

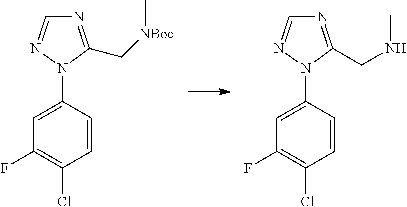 [SMILES: CN(Cc1ncnn1-c1ccc(Cl)c(F)c1)C(=O)OC(C)(C)C ; CNCc1ncnn1-c1ccc(Cl)c(F)c1]

Trifluoroacetic acid (3.47 mL; 45.34 mmol; 15.00 eq.) was added drop wise to a solution of tert-butyl N-{[1-(4-chloro-3-fluorophenyl)-1H-1,2,4-triazol-5-yl]methyl}-N-methylcarbamate (1 030.00 mg; 3.02 mmol; 1.00 eq.) in dichloromethane (20.60 mL) and the mixture was stirred at RT for 2 h. The solvent was removed under vacuum and the crude was partitioned in DCM/sat. sol. of NaHCO₃. The organic layer was isolated, and the aqueous layer was extracted two more times with DCM. The combined organics were dried over MgSO4 and concentrated to dryness. The crude {[1-(4-chloro-3-fluorophenyl)-1H-1,2,4-triazol-5-yl]methyl}(methyl)amine (670 mg, 92%) was used as is in the next step. ¹H NMR (400 MHz, DMSO-d6) δ 8.13 (d, J=1.8 Hz, 1H), 7.96 (dt, J=10.3, 2.2 Hz, 1H), 7.79 (td, J=8.5, 1.8 Hz, 1H), 7.62 (ddd, J=8.7, 2.4, 1.2 Hz, 1H), 3.81 (d, J=1.8 Hz, 2H), 2.44-2.33 (m, 1H), 2.25 (d, J=1.8 Hz, 3H). LCMS (ES) [M+1]+m/z 239.1/241.1.

Step 3

The title compound was synthesized according to General Procedure 7, Method A using [1-(4-chloro-3-fluorophenyl)-1H-1,2,4-triazol-5-yl]methanamine (Intermediate I-4, 30.00 mg; 0.13 mmol; 1.00 eq.) and {[1-(4-chloro-3-fluorophenyl)-1H-1,2,4-triazol-5-yl]methyl}(methyl)amine (31.86 mg; 0.13 mmol; 1.00 eq.) to give the desired 1,3-bis({[1-(4-chloro-3-fluorophenyl)-1H-1,2,4-triazol-5-yl]methyl 1)-1-methylurea (49 mg, 75%). 1H NMR (400 MHz, DMSO-d6) δ 8.08 (dd, J=6.9, 1.8 Hz, 2H), 7.82-7.69 (m, 4H), 7.46 (dd, J=12.5, 8.8 Hz, 2H), 7.12 (d, J=5.8 Hz, 1H), 4.59 (s, 2H), 4.32 (d, J=5.3 Hz, 2H), 2.78 (s, 3H). LCMS (ES) [M+1]⁺ m/z 493.4.

### Example 1.44

### Synthesis of 1,3-bis({[1-(4-chloro-3-fluorophenyl)-1H-1,2,4-triazol-5-yl]methyl})-1,3-dimethylurea (Compound 28)

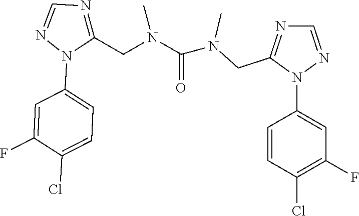 [SMILES: CN(Cc1ncnn1-c1ccc(Cl)c(F)c1)C(=O)N(C)Cc1ncnn1-c1ccc(Cl)c(F)c1]

The title compound was synthesized according to General Procedure 6, Method A using {[1-(4-chloro-3-fluorophenyl)-1H-1,2,4-triazol-5-yl]methyl}(methyl)amine (Example 1.43 used to prepare Compound 27 Step 2; 60 mg) to provide 11,3-bis({[1-(4-chloro-3-fluorophenyl)-1H-1,2,4-triazol-5-yl]methyl})-1,3-dimethylurea (17 mg, 26.88%). ¹H NMR (400 MHz, DMSO-d6) δ 8.11 (d, J=2.2 Hz, 2H), 7.83-7.67 (m, 4H), 7.50-7.36 (m, 2H), 4.45 (s, 4H), 2.60 (d, J=2.1 Hz, 6H). LCMS (ES) [M+1]⁺ m/z 507.2/509.1.

### Example 1.45

### Synthesis of 3-{[1-(4-chloro-3-fluorophenyl)-3-methyl-1H-1,2,4-triazol-5-yl]methyl}-1-{[1-(2-methylquinolin-7-yl)-1H-1,2,4-triazol-5-yl]methyl}urea (Compound 29)

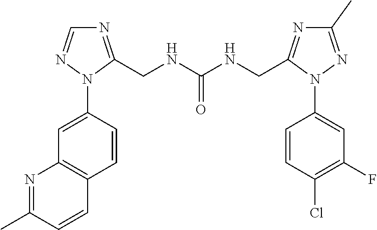 [SMILES: Cc1ccc2ccc(-n3ncnc3CNC(=O)NCc3nc(C)nn3-c3ccc(Cl)c(F)c3)cc2n1]

Step 1

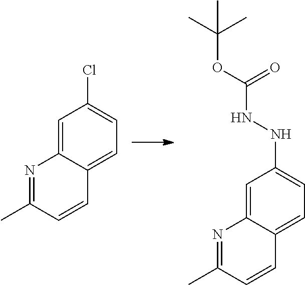 [SMILES: Cc1ccc2ccc(Cl)cc2n1 ; Cc1ccc2ccc(NNC(=O)OC(C)(C)C)cc2n1]

Into a 250-mL 3-necked round-bottom flask purged and maintained with an inert atmosphere of argon, was placed 7-chloro-2-methylquinoline (5.0 g, 28.1 mmol, 1.0 eq.), tert-butoxycarbohydrazide (7.44 g, 56.29 mmol, 2.0 eq.), Pd₂(dba)₃·CHCl₃ (1.46 g, 1.40 mmol, 0.05 eq), t-BuONa (5.41 g, 56.29 mmol, 2.0 eq.), XantPhos (1.62 g, 2.80 mmol, 0.10 eq.), and dioxane (50 mL) and the resulting solution was stirred for 16 h at 90° C. in an oil bath. The reaction mixture was then cooled to ambient temperature and the solids were separated by filtration. The filtrate was concentrated and the residue was applied onto a silica gel column and eluted with 100% petroleum ether to 50% ethyl acetate in petroleum ether providing 5.5 g (71.48%) of N′-(2-methylquinolin-7-yl)tert-butoxycarbohydrazide as an off white solid. LCMS (ES) [M+1]⁺ m/z: 274.

Step 2

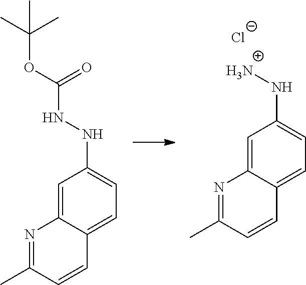 [SMILES: Cc1ccc2ccc(NNC(=O)OC(C)(C)C)cc2n1 ; Cc1ccc2ccc(N[NH3+])cc2n1 ; [Cl-]]

Into a 100-mL round-bottom flask, was placed N′-(2-methylquinolin-7-yl)tert-butoxycarbohydrazide (5.5 g, 20.12 mmol, 1.0 eq.) in MeOH (20 mL). To the above HCl(g)in MeOH (6.1 mL, 167.6 mmol, 10 eq.) was introduced at 25° C. and the resulting solution was stirred for 3 h at 25° C. The reaction mixture was then concentrated under vacuum providing 3.3 g (94.68%) of 7-hydrazinyl-2-methylquinoline hydrochloride as a yellow solid. LCMS (ES) [M+1-HCl] ⁺ m/z: 174.

Step 3

The title compound was synthesized according to General Procedure 7; Method A using 1-[2-(2-methylquinolin-7-yl)-1,2,4-triazol-3-yl]methanamine (300 mg, 1.25 mmol, 1.05 eq. synthesized according to General Procedures 2 and 3 using 7-hydrazinyl-2-methylquinoline hydrochloride from step 2 above) and 1-[1-(4-chloro-3-fluorophenyl)-3-methyl-1H-1,2,4-triazol-5-yl]methanamine (Intermediate I-6; 301 mg, 1.25 mmol, 1.00 eq.) providing 3-{[1-(4-chloro-3-fluorophenyl)-3-methyl-1H-1,2,4-triazol-5-yl]methyl}-1-{[1-(2-methylquinolin-7-yl)-1H-1,2,4-triazol-5-yl]methyl}urea (75 mg, 11.82%) as a white solid. 1H NMR (300 MHz, DMSO-d6): δ 8.36 (d, J=8.4 Hz, 1H), 8.17-8.10 (m, 3H), 7.78-7.71 (m, 3H), 7.54-7.48 (m, 2H), 6.76 (t, J=5.5 Hz, 1H), 6.74 (t, J=5.7 Hz, 1H), 4.50 (d, J=5.4 Hz, 2H), 4.35 (d, J=5.4 Hz, 2H), 2.69 (s, 3H), 2.29 (s, 3H). LCMS (ES) [M+1]⁺ m/z: 506.

### Example 1.46

### Synthesis of 1,3-bis({[1-(4-chloro-3-fluorophenyl)-1H-1,2,4-triazol-5-yl]methyl})-1-ethylurea (Compound 30)

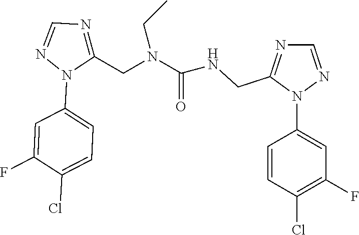 [SMILES: CCN(Cc1ncnn1-c1ccc(Cl)c(F)c1)C(=O)NCc1ncnn1-c1ccc(Cl)c(F)c1]

Step 1

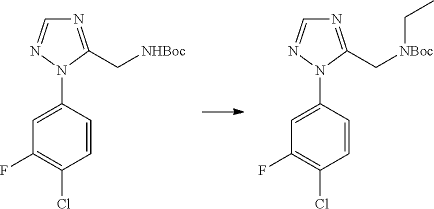 [SMILES: CC(C)(C)OC(=O)NCc1ncnn1-c1ccc(Cl)c(F)c1 ; CCN(Cc1ncnn1-c1ccc(Cl)c(F)c1)C(=O)OC(C)(C)C]

Tert-butyl N-{[1-(4-chloro-3-fluorophenyl)-1H-1,2,4-triazol-5-yl]methyl}-N-ethylcarbamate was synthesized from tert-butyl N-{[1-(4-chloro-3-fluorophenyl)-1H-1,2,4-triazol-5-yl]methyl}carbamate (Intermediate I-3, 300 mg; 0.92 mmol; 1.00 eq.) under the conditions reported in Step 1 of Example 1.43 used to prepare Compound 27, Step 1, but using ethyl iodide instead of methyl iodide. The crude was purified on a silica gel column using 0 to 40% EtOAc in heptanes (270 mg, 88%). ¹H NMR (400 MHz, DMSO-d6) δ 8.13 (s, 1H), 7.77 (dt, J=9.9, 2.2 Hz, 2H), 7.45 (ddd, J=8.6, 2.5, 1.3 Hz, 1H), 4.64 (s, 2H), 3.09 (s, 2H), 1.40-0.79 (m, 12H). LCMS (ES) [M-100]⁺ m/z 255.

Step 2

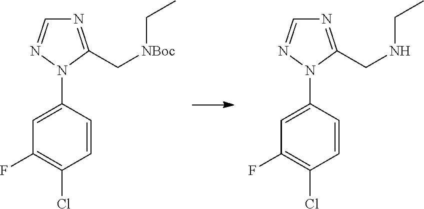 [SMILES: CCN(Cc1ncnn1-c1ccc(Cl)c(F)c1)C(=O)OC(C)(C)C ; CCNCc1ncnn1-c1ccc(Cl)c(F)c1]

Tert-butyl N-{[1-(4-chloro-3-fluorophenyl)-1H-1,2,4-triazol-5-yl]methyl}-N-ethylcarbamate (270.00 mg; 0.76 mmol; 1.00 eq.) was treated under the conditions reported in Example 1.43 used to prepare Compound 27, Step 2 to give {[1-(4-chloro-3-fluorophenyl)-1H-1,2,4-triazol-5-yl]methyl}(ethyl)amine (170 mg, 87.71%). ¹H NMR (400 MHz, DMSO-d6) δ 8.12 (s, 1H), 7.99 (dd, J=10.3, 2.4 Hz, 1H), 7.79 (t, J=8.4 Hz, 1H), 7.66-7.60 (m, 1H), 3.85 (s, 2H), 2.54 (t, J=7.1 Hz, 2H), 0.96 (t, J=7.1 Hz, 3H).

Step 3

The title compound was synthesized according to General Procedure 7, Method A using [1-(4-chloro-3-fluorophenyl)-1H-1,2,4-triazol-5-yl]methanamine (Intermediate I-4, 30.00 mg; 0.13 mmol; 1.00 eq.) and {[1-(4-chloro-3-fluorophenyl)-1H-1,2,4-triazol-5-yl]methyl}(ethyl)amine (45 mg; 0.18 mmol; 1.00 eq.) to give the desired 1,3-bis({[1-(4-chloro-3-fluorophenyl)-1H-1,2,4-triazol-5-yl]methyl})-1-ethylurea (56.80 mg, 63.44%). 1H NMR (400 MHz, DMSO-d6) δ 8.07 (d, J=1.9 Hz, 2H), 7.75 (ddt, J=12.7, 8.3, 2.4 Hz, 4H), 7.45 (dd, J=13.0, 8.9 Hz, 2H), 7.07 (t, J=5.7 Hz, 1H), 4.54 (s, 2H), 4.34 (d, J=5.2 Hz, 2H), 3.17 (q, J=7.0 Hz, 2H), 0.92 (t, J=7.1 Hz, 3H). LCMS (ES) [M+1]⁺ m/z 481.05-483.35. LCMS (ES) [M+1]⁺ m/z 507.1.

### Example 1.47

### Synthesis of 3-{[1-(4-chloro-3-fluorophenyl)-1H-1,2,3,4-tetrazol-5-yl]methyl}-1-{[1-(4-chloro-3-fluorophenyl)-1H-1,2,4-triazol-5-yl]methyl}urea (Compound 31)

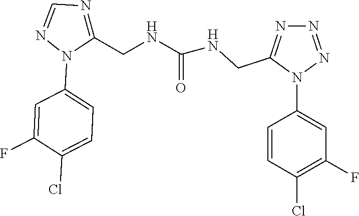 [SMILES: O=C(NCc1ncnn1-c1ccc(Cl)c(F)c1)NCc1nnnn1-c1ccc(Cl)c(F)c1]

The title compound was synthesized according to General Procedure 7, Method A using 1-[1-(4-chloro-3-fluorophenyl)-1H-1,2,4-triazol-5-yl]methanamine (Intermediate I-4; 32 mg, 0.14 mmol; 1.00 eq.) and [1-(4-chloro-3-fluorophenyl)-1H-1,2,3,4-tetrazol-5-yl]methanamine (31.94 mg; 0.14 mmol; 1.00 eq.; synthesized according to General Procedure 4 from commercial 2-fluoro-4-chloroaniline to give 3-{[1-(4-chloro-3-fluorophenyl)-1H-1,2,4-triazol-5-yl]methyl}urea (28 mg, 41.55%). 1H NMR (400 MHz, DMSO-d6) δ 8.10 (d, J=1.5 Hz, 1H), 7.94-7.81 (m, 2H), 7.81-7.73 (m, 2H), 7.59-7.52 (m, 1H), 7.47 (dd, J=9.1, 2.1 Hz, 1H), 6.79 (q, J=5.8 Hz, 2H), 4.50 (d, J=5.6 Hz, 2H), 4.35 (d, J=5.5 Hz, 2H). LCMS (ES) [M+1]⁺ m/z 480.1.

### Example 1.48

### Synthesis of 3-{[1-(4-chloro-3-fluorophenyl)-1H-1,2,3,4-tetrazol-5-yl]methyl}-1-{[1-(4-chloro-3-fluorophenyl)-1H-1,2,4-triazol-5-yl]methyl}urea (Compound 32)

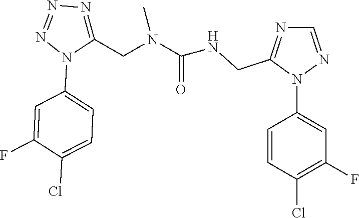 [SMILES: CN(Cc1nnnn1-c1ccc(Cl)c(F)c1)C(=O)NCc1ncnn1-c1ccc(Cl)c(F)c1]

The title compound was synthesized according to General Procedure 7, Method A using 1-[1-(4-chloro-3-fluorophenyl)-1H-1,2,4-triazol-5-yl]methanamine (Intermediate I-4; 37 mg; 0.17 mmol; 1.00 eq.) and {-[1-(4-chloro-3-fluorophenyl)-1H-1,2,3,4-tetrazol-5-yl]methyl}(methyl)amine (Example 1.43 used to prepare Compound 27, Step 1; 39.94 mg; 0.17 mmol; 1.00 eq) to give 1-{[1-(4-chloro-3-fluorophenyl)-1H-1,2,3,4-tetrazol-5-yl]methyl}-3-{[1-(4-chloro-3-fluorophenyl)-1H-1,2,4-triazol-5-yl]methyl}-1-methylurea (31 mg, 37.95%).1-H NMR (400 MHz, DMSO-d6) δ 8.07 (d, J=1.8 Hz, 1H), 7.92-7.70 (m, 4H), 7.50 (dd, J=27.5, 8.8 Hz, 2H), 7.19 (t, J=5.5 Hz, 1H), 4.70 (s, 2H), 4.30 (d, J=5.3 Hz, 2H), 2.78 (d, J=1.8 Hz, 3H). LCMS (ES) [M+1]+m/z 494.31.

### Example 1.49

### Synthesis of 1-{[1-(4-chloro-3-fluorophenyl)-1H-1,2,3,4-tetrazol-5-yl]methyl}-3-{[1-(4-chloro-3-fluorophenyl)-1H-1,2,4-triazol-5-yl]methyl}-3-methylurea (Compound 33)

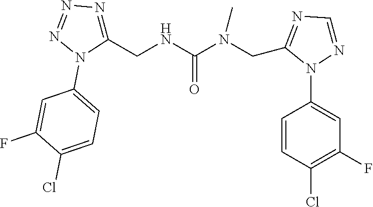 [SMILES: CN(Cc1ncnn1-c1ccc(Cl)c(F)c1)C(=O)NCc1nnnn1-c1ccc(Cl)c(F)c1]

The title compound was synthesized according to General Procedure 7, Method A using 1-[1-(4-chloro-3-fluorophenyl)-1H-1,2,3,4-tetrazol-5-yl]methanamine (64.00 mg; 0.28 mmol; 1.00 eq.; synthesized from 3-chloro-4-fluoroaniline according to General Procedure 4) and {-[1-(4-chloro-3-fluorophenyl)-1H-1,2,4-triazol-5-yl]methyl}(methyl)amine (67.67 mg; 0.28 mmol; 1.00 eq; Example 1.43 used to prepare Compound 27, Step 2) to give 1-{[1-(4-chloro-3-fluorophenyl)-1H-1,2,3,4-tetrazol-5-yl]methyl}-3-{[1-(4-chloro-3-fluorophenyl)-1H-1,2,4-triazol-5-yl]methyl-3-methylurea (98 mg, 72% yield). 1-H NMR (400 MHz, DMSO-d6) δ 8.08 (d, J=2.1 Hz, 1H), 7.92-7.81 (m, 2H), 7.79-7.68 (m, 2H), 7.53 (d, J=8.7 Hz, 1H), 7.43 (d, J=8.7 Hz, 1H), 7.20 (t, J=5.3 Hz, 1H), 4.55 (s, 2H), 4.46 (d, J=5.1 Hz, 2H), 2.74 (s, 3H). LCMS (ES) [M+1]⁺ m/z 495.97.

### Example 1.50

### Synthesis of 1-{[1-(4-chloro-3-fluorophenyl)-1H-1,2,3,4-tetrazol-5-yl]methyl}-3-{[1-(4-chloro-3-fluorophenyl)-1H-1,2,4-triazol-5-yl]methyl}-1-(2-hydroxyethyl)urea (Compound 34)

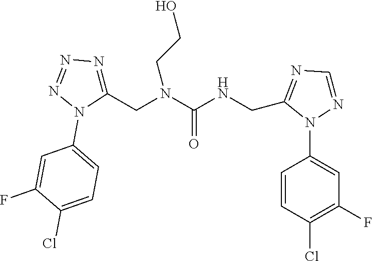 [SMILES: O=C(NCc1ncnn1-c1ccc(Cl)c(F)c1)N(CCO)Cc1nnnn1-c1ccc(Cl)c(F)c1]

Step 1

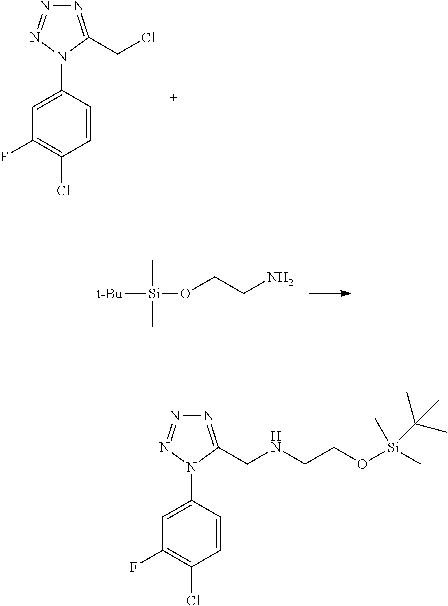 [SMILES: Fc1cc(-n2nnnc2CCl)ccc1Cl ; CC(C)(C)[Si](C)(C)OCCN ; CC(C)(C)[Si](C)(C)OCCNCc1nnnn1-c1ccc(Cl)c(F)c1]

A mixture of 1-(4-chloro-3-fluorophenyl)-5-(chloromethyl)-1H-1,2,3,4-tetrazole (291.00 mg; 1.18 mmol; 1.00 eq.; synthesized according to General Procedure 4, steps 1 and 2 starting from commercial 4-chloro-3-fluoroaniline) and 2-((tert-butyldimethylsilyl)oxy)ethan-1-amine (413.06 mg; 2.36 mmol; 2.00 eq.) in 1,4-dioxane (3 mL) and triethylamine (0.5 mL) in a sealed reaction vessel was allowed to stir at 70° C. for 120 min and then cooled to ambient temperature. After removal of volatiles under reduced pressure, the remaining residue was purified by flash chromatography on 25 g silica gel column using 0-100% EtOAC/Hexanes to provide {2-[(tert-butyldimethyl silyl)oxy]ethyl}({[1-(4-chloro-3-fluorophenyl)-1H-1,2,3,4-tetrazol-5-yl]methyl})amine as white solid (331 mg, 68%). LCMS (ES) [M+1]⁺ m/z: 386.3.

Step 2

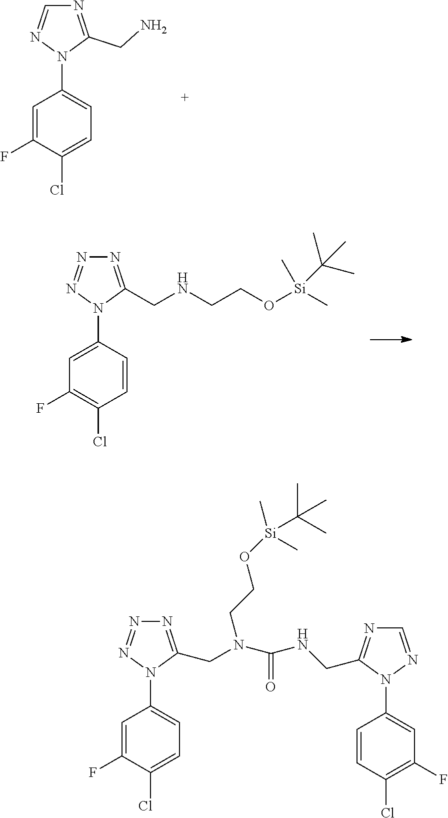 [SMILES: NCc1ncnn1-c1ccc(Cl)c(F)c1 ; CC(C)(C)[Si](C)(C)OCCNCc1nnnn1-c1ccc(Cl)c(F)c1 ; CC(C)(C)[Si](C)(C)OCCN(Cc1nnnn1-c1ccc(Cl)c(F)c1)C(=O)NCc1ncnn1-c1ccc(Cl)c(F)c1]

Intermediate compound (1-{2-[(tert-butyldimethylsilyl)oxy]ethyl}-1-{[1-(4-chloro-3-fluorophenyl)-1H-1,2,3,4-tetrazol-5-yl]methyl}-3-{[1-(4-chloro-3-fluorophenyl)-1H-1,2,4-triazol-5-yl]methyl}urea) was synthesized according to General Procedure 7, Method A using (1-(4-chloro-3-fluorophenyl)-1H-1,2,4-triazol-5-yl)methanamine (intermediate I-4, 61.17 mg; 0.27 mmol; 1.00 eq.) and {2-[(tert-butyldimethylsilyl)oxy]ethyl}({[1-(4-chloro-3-fluorophenyl)-1H-1,2,3,4-tetrazol-5-yl]methyl})amine (103.49 mg; 0.27 mmol; 1.00 eq. from step 1 above) to give 1-{2-[(tert-butyldimethylsilyl)oxy]ethyl}-1-{[1-(4-chloro-3-fluorophenyl)-1H-1,2,3,4-tetrazol-5-yl]methyl}-3-{[1-(4-chloro-3-fluorophenyl)-1H-1,2,4-triazol-5-yl]methyl}urea (80 mg, 46.72%). LCMS (ES) [M+1]⁺ m/z 638.37.

Step 3

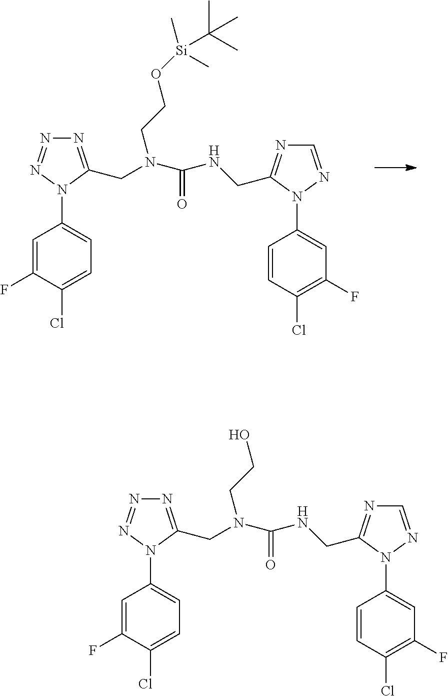 [SMILES: CC(C)(C)[Si](C)(C)OCCN(Cc1nnnn1-c1ccc(Cl)c(F)c1)C(=O)NCc1ncnn1-c1ccc(Cl)c(F)c1 ; O=C(NCc1ncnn1-c1ccc(Cl)c(F)c1)N(CCO)Cc1nnnn1-c1ccc(Cl)c(F)c1]

Tetrabutylammonium fluoride (0.14 mL; 1.00 mol/L; 0.14 mmol; 1.10 eq.) was added to a solution of 1-{2-[(tert-butyldimethylsilyl)oxy]ethyl}-1-{[1-(4-chloro-3-fluorophenyl)-1H-1,2,3,4-tetrazol-5-yl]methyl}-3-{[1-(4-chloro-3-fluorophenyl)-1H-1,2,4-triazol-5-yl]methyl}urea (80.00 mg; 0.13 mmol; 1.00 eq. from step 2 above) in tetrahydrofuran (2.40 mL) at 0° C. over a 5 min period and the resulting mixture was stirred at ambient temperature for 30 min. The reaction mixture was then concentrated and the residue, cooled in an ice bath, was diluted with water. The resulting mixture was extracted with EtOAc and the aqueous layer was back extracted twice more. The combined organics were washed with saturated aqueous NaHCO₃, dried over MgSO4 and the crude was purified on a silica gel column using 0-50% (DCM:MeOH:NH₄OH; 90:9:1) in DCM to give 1-{[1-(4-chloro-3-fluorophenyl)-1H-1,2,3,4-tetrazol-5-yl]methyl}-3-{[1-(4-chloro-3-fluorophenyl)-1H-1,2,4-triazol-5-yl]methyl}-1-(2-hydroxyethyl)urea (59 mg, 89%). 1-H NMR (400 MHz, DMSO-d6) δ 8.08 (s, 1H), 7.90-7.80 (m, 2H), 7.79-7.71 (m, 2H), 7.56-7.50 (m, 1H), 7.49-7.42 (m, 1H), 7.17 (t, J=5.4 Hz, 1H), 4.81 (t, J=5.0 Hz, 1H), 4.72 (s, 2H), 4.32 (d, J=4.9 Hz, 2H), 3.44 (q, J=5.5 Hz, 2H), 3.25 (dd, J=10.4, 5.0 Hz, 2H); LCMS (ES) [M+1]⁺ m/z 524.1.

### Example 1.51

### Synthesis of 1-{[1-(5-chloro-1,3-benzothiazol-2-yl)-1H-1,2,4-triazol-5-yl]methyl}-3-{[1-(4-chloro-3-fluorophenyl)-1H-1,2,4-triazol-5-yl]methyl}urea (Compound 35)

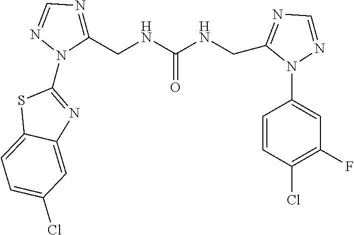 [SMILES: O=C(NCc1ncnn1-c1ccc(Cl)c(F)c1)NCc1ncnn1-c1nc2cc(Cl)ccc2s1]

The title compound was synthesized according to General Procedure 7, Method A using (1-(4-chloro-3-fluorophenyl)-1H-1,2,4-triazol-5-yl)methanamine (intermediate I-4, 43.02 mg; 0.27 mmol; 1.00 eq.) and [1-(5-chloro-1,3-benzothiazol-2-yl)-1H-1,2,4-triazol-5-yl]methanamine (49.40 mg; 0.19 mmol; 1.00 eq.; synthesized according to General Procedure 2 and 3 from 5-chloro-2-hydrazinyl-1,3-benzothiazole) to provide 1-{[1-(5-chloro-1,3-benzothiazol-2-yl)-1H-1,2,4-triazol-5-yl]methyl}-3-{[1-(4-chloro-3-fluorophenyl)-1H-1,2,4-triazol-5-yl]methyl}urea as white solid (49 mg; 50.85%). ¹H NMR (400 MHz, DMSO-d6) δ 8.26 (d, J=1.7 Hz, 1H), 8.18 (d, J=8.7 Hz, 1H), 8.11 (d, J=2.2 Hz, 2H), 7.83-7.73 (m, 2H), 7.57-7.47 (m, 2H), 7.00-6.93 (m, 1H), 6.75 (t, J=5.9 Hz, 1H), 4.86 (d, J=5.8 Hz, 2H), 4.43 (d, J=5.6 Hz, 2H); LCMS (ES) [M+1]⁺ m/z 518.2.

### Example 1.52

### Synthesis of 3-{[1-(4-chloro-3-fluorophenyl)-1H-1,2,3,4-tetrazol-5-yl]methyl}-1-{[1-(4-chloro-3-fluorophenyl)-1H-1,2,4-triazol-5-yl]methyl}-1-ethylurea (Compound 36)

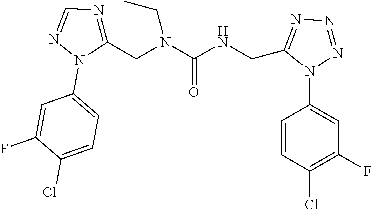 [SMILES: CCN(Cc1ncnn1-c1ccc(Cl)c(F)c1)C(=O)NCc1nnnn1-c1ccc(Cl)c(F)c1]

The title compound was synthesized according to General Procedure 7, Method A using 1-[1-(4-chloro-3-fluorophenyl)-1H-1,2,3,4-tetrazol-5-yl]methanamine (50.00 mg; 0.22 mmol; 1.00 eq.); synthesized from 3-chloro-4-fluoroaniline according to General Procedure 4) and {[1-(4-chloro-3-fluorophenyl)-1H-1,2,4-triazol-5-yl]methyl}(ethyl)amine (Example 1.46 used to prepare Compound 30; Step 2, 55.95 mg; 0.22 mmol; 1.00 eq.), to give 3-{[1-(4-chloro-3-fluorophenyl)-1H-1,2,3,4-tetrazol-5-yl]methyl}-1-{[1-(4-chloro-3-fluorophenyl)-1H-1,2,4-triazol-5-yl]methyl}-1-ethylurea (71 mg, 63.59%). 1-H NMR (400 MHz, DMSO-d6) δ 8.07 (s, 1H), 7.91-7.80 (m, 2H), 7.78-7.67 (m, 2H), 7.52 (ddd, J=8.6, 2.4, 1.2 Hz, 1H), 7.43 (ddd, J=8.6, 2.4, 1.1 Hz, 1H), 7.14 (t, J=5.3 Hz, 1H), 4.57-4.41 (m, 4H), 3.14 (q, J=7.0 Hz, 2H), 0.88 (t, J=7.0 Hz, 3H). LCMS (ES) [M+1]⁺ m/z 508.23.

### Example 1.53

### Synthesis of 3-{[1-(4-chloro-3-fluorophenyl)-3-methyl-1H-1,2,4-triazol-5-yl]methyl}-1-{[1-(quinolin-7-yl)-1H-1,2,4-triazol-5-yl]methyl}urea (Compound 37)

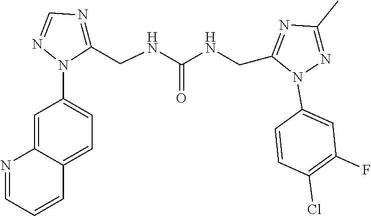 [SMILES: Cc1nc(CNC(=O)NCc2ncnn2-c2ccc3cccnc3c2)n(-c2ccc(Cl)c(F)c2)n1]

The title compound was synthesized according to General Procedure 7, Method A using 1-[1-(4-chloro-3-fluorophenyl)-3-methyl-1H-1,2,4-triazol-5-yl]methanamine (Intermediate I-6; 1.20 g; 4.99 mmol; 1.00 eq.) and 1-[1-(quinolin-7-yl)-1H-1,2,4-triazol-5-yl]methanamine (Intermediate I-9; 1.12 g; 4.99 mmol; 1.00 eq.) to give 3-{[1-(4-chloro-3-fluorophenyl)-3-methyl-1H-1,2,4-triazol-5-yl]methyl}-1-{[1-(quinolin-7-yl)-1H-1,2,4-triazol-5-yl]methyl}urea (1.10 g, 44.85%). 1H NMR (400 MHz, DMSO-d6) δ 8.99 (dd, J=4.2, 1.8 Hz, 1H), 8.48 (dt, J=8.2, 1.5 Hz, 1H), 8.23 (d, J=2.1 Hz, 1H), 8.17 (d, J=8.9 Hz, 2H), 7.81 (dd, J=8.7, 2.2 Hz, 1H), 7.77-7.68 (m, 2H), 7.63 (dd, J=8.3, 4.2 Hz, 1H), 7.44 (ddd, J=8.6, 2.5, 1.2 Hz, 1H), 6.73 (dt, J=13.8, 5.6 Hz, 2H), 4.48 (d, J=5.6 Hz, 2H), 4.32 (d, J=5.7 Hz, 2H), 2.26 (s, 3H). LCMS (ES) [M+1]⁺ m/z 492.22.

### Example 1.54

### Synthesis of 1,3-bis({[1-(4-chloro-3-fluorophenyl)-1H-1,2,4-triazol-5-yl]methyl})-1-(2-fluoroethyl)urea (Compound 38)

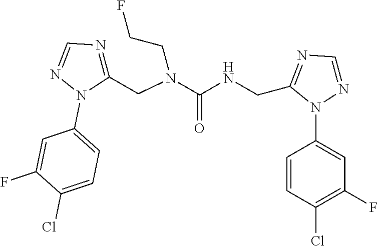 [SMILES: O=C(NCc1ncnn1-c1ccc(Cl)c(F)c1)N(CCF)Cc1ncnn1-c1ccc(Cl)c(F)c1]

The title compound was synthesized according to General Procedure 7, Method A using 1-[1-(4-chloro-3-fluorophenyl)-1H-1,2,4-triazol-5-yl]methanamine (Intermediate I-4; 43 mg; 0.17 mmol; 1.00 eq.) and {[1-(4-chloro-3-fluorophenyl)-1H-1,2,4-triazol-5-yl]methyl}(2-fluoroethyl)amine (51.86 mg; 0.19 mmol; 1.00 eq.; synthesized according to the procedure described in Example 1.43 used to prepare Compound 27, Steps 1 and 2, using 1-bromo-2-fluoroethane instead of iodomethane), to give 1,3-bis({[1-(4-chloro-3-fluorophenyl)-1H-1,2,4-triazol-5-yl]methyl})-1-(2-fluoroethyl)urea (74.50 mg, 74.56%). 1-H NMR (400 MHz, DMSO-d6) δ 8.08 (d, J=4.2 Hz, 2H), 7.83-7.67 (m, 4H), 7.45 (tdd, J=8.6, 2.5, 1.2 Hz, 2H), 7.18 (t, J=5.3 Hz, 1H), 4.62 (s, 2H), 4.48 (t, J=5.2 Hz, 1H), 4.36 (dd, J=6.7, 5.2 Hz, 3H), 3.51 (dt, J=24.2, 5.3 Hz, 2H). LCMS (ES) [M+1]⁺ m/z 527.03.

### Example 1.55

### Synthesis of 3-{[1-(4-chloro-3-fluorophenyl)-3-methyl-1H-1,2,4-triazol-5-yl]methyl}-1-{[1-(isoquinolin-7-yl)-1H-1,2,4-triazol-5-yl]methyl}urea (Compound 39)

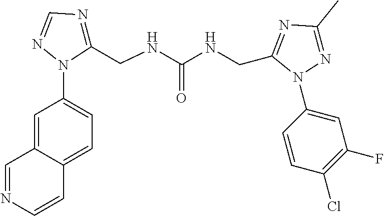 [SMILES: Cc1nc(CNC(=O)NCc2ncnn2-c2ccc3ccncc3c2)n(-c2ccc(Cl)c(F)c2)n1]

The title compound was synthesized according to General Procedure 7, Method A using 1-[1-(4-chloro-3-fluorophenyl)-3-methyl-1H-1,2,4-triazol-5-yl]methanamine (Intermediate I-6; 70.00 mg; 0.29 mmol; 1.00 eq.) and 1-[1-(isoquinolin-7-yl)-1H-1,2,4-triazol-5-yl]methanamine hydrochloride (76.12 mg; 0.29 mmol; 1.00 eq.; synthesized from 7-hydrazinylisoquinoline dihydrochloride according to General Procedures 2 and 3) to give 3-{[1-(4-chloro-3-fluorophenyl)-3-methyl-1H-1,2,4-triazol-5-yl]methyl}-1-{[1-(isoquinolin-7-yl)-1H-1,2,4-triazol-5-yl]methyl}urea (89 mg, 62.20%). 1-H NMR (400 MHz, DMSO-d6) δ 9.36 (s, 1H), 8.59 (d, J=5.7 Hz, 1H), 8.35 (d, J=2.1 Hz, 1H), 8.14 (d, J=8.5 Hz, 2H), 8.02-7.88 (m, 2H), 7.78-7.67 (m, 2H), 7.44 (ddd, J=8.7, 2.4, 1.1 Hz, 1H), 6.71 (dt, J=17.1, 5.7 Hz, 2H), 4.48 (d, J=5.6 Hz, 2H), 4.31 (d, J=5.6 Hz, 2H), 2.26 (s, 3H). LCMS (ES) [M+1]⁺ m/z 492.17.

### Example 1.56

### 3-{[1-(4-chloro-3-fluorophenyl)-3-methyl-1H-1,2,4-triazol-5-yl]methyl}-1-{[1-(quinoxalin-6-yl)-1H-1,2,4-triazol-5-yl]methyl}urea (Compound 40)

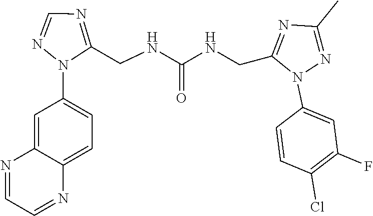 [SMILES: Cc1nc(CNC(=O)NCc2ncnn2-c2ccc3nccnc3c2)n(-c2ccc(Cl)c(F)c2)n1]

The title compound was synthesized according to General Procedure 7, Method A using 1-[1-(4-chloro-3-fluorophenyl)-3-methyl-1H-1,2,4-triazol-5-yl]methanamine (Intermediate I-6; 77.00 mg; 0.32 mmol; 1.00 eq.) and 1-[1-(quinoxalin-6-yl)-1H-1,2,4-triazol-5-yl]methanamine hydrochloride (84.05 mg; 0.32 mmol; 1.00 eq.; synthesized from 6-hydrazinylquinoxaline according to General Procedures 2 and 3), to give 3-{[1-(4-chloro-3-fluorophenyl)-3-methyl-1H-1,2,4-triazol-5-yl]methyl}-1-{[1-(quinoxalin-6-yl)-1H-1,2,4-triazol-5-yl]methyl}urea (84.20 mg, 53.39%). 1-H NMR (400 MHz, DMSO-d6) δ 9.03 (s, 2H), 8.36 (d, J=2.4 Hz, 1H), 8.25 (d, J=9.0 Hz, 1H), 8.18 (s, 1H), 8.06 (dd, J=9.0, 2.4 Hz, 1H), 7.77-7.67 (m, 2H), 7.44 (ddd, J=8.6, 2.5, 1.2 Hz, 1H), 6.72 (dt, J=21.2, 5.6 Hz, 2H), 4.51 (d, J=5.6 Hz, 2H), 4.31 (d, J=5.6 Hz, 2H), 2.26 (s, 3H). LCMS (ES) [M+1]⁺ m/z 493.37.

### Example 1.57

### Synthesis of 1-{[1-(4-chloro-3-fluorophenyl)-3-methyl-1H-1,2,4-triazol-5-yl]methyl}-3-{[1-(quinolin-6-yl)-1H-1,2,4-triazol-5-yl]methyl}urea (Compound 41)

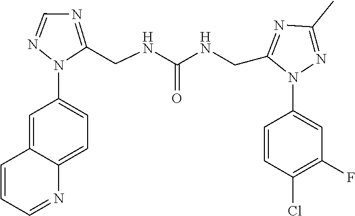 [SMILES: Cc1nc(CNC(=O)NCc2ncnn2-c2ccc3ncccc3c2)n(-c2ccc(Cl)c(F)c2)n1]

The title compound was synthesized according to General Procedure 7, Method A using [1-(4-chloro-3-fluorophenyl)-3-methyl-1H-1,2,4-triazol-5-yl]methanamine (intermediate I-6, 80.00 mg; 0.33 mmol; 1.00 eq.) and 1-[1-(quinolin-6-yl)-1H-1,2,4-triazol-5-yl]methanamine hydrochloride (87.00 mg; 0.33 mmol; 1.00 eq.; synthesized from 6-hydrazinylquinoline hydrochloride according to General Procedures 2 and 3), to give 1-{[1-(4-chloro-3-fluorophenyl)-3-methyl-1H-1,2,4-triazol-5-yl]methyl}-3-{[1-(quinolin-6-yl)-1H-1,2,4-triazol-5-yl]methyl}urea (71 mg, 43,42%). 1-H NMR (400 MHz, DMSO-d6) δ 8.98 (dd, J=4.2, 1.7 Hz, 1H), 8.45-8.36 (m, 1H), 8.22 (d, J=2.4 Hz, 1H), 8.16 (d, J=8.6 Hz, 2H), 7.94 (dd, J=9.0, 2.4 Hz, 1H), 7.76-7.67 (m, 2H), 7.61 (d, J=4.2 Hz, 1H), 7.44 (ddd, J=8.7, 2.4, 1.1 Hz, 1H), 6.71 (dt, J=11.8, 5.7 Hz, 2H), 4.48 (d, J=5.7 Hz, 2H), 4.31 (d, J=5.6 Hz, 2H), 2.26 (s, 3H). LCMS (ES) [M+1]⁺ m/z 491.78.

### Example 1.58

### Synthesis of 1-{[1-(4-chloro-3-fluorophenyl)-3-(propan-2-yl)-1H-1,2,4-triazol-5-yl]methyl}-3-{[1-(quinolin-7-yl)-1H-1,2,4-triazol-5-yl]methyl}urea (Compound 42)

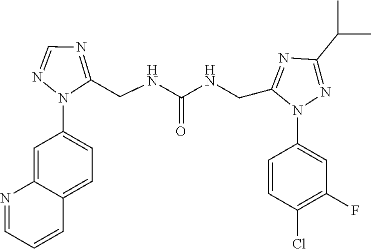 [SMILES: CC(C)c1nc(CNC(=O)NCc2ncnn2-c2ccc3cccnc3c2)n(-c2ccc(Cl)c(F)c2)n1]

The title compound was synthesized according to General Procedure 7, Method A using 1-[1-(quinolin-7-yl)-1H-1,2,4-triazol-5-yl]methanamine (intermediate I-9; 75.00 mg; 0.33 mmol; 1.00 eq.) and 1-[1-(4-chloro-3-fluorophenyl)-3-(propan-2-yl)-1H-1,2,4-triazol-5-yl]methanamine (89.47 mg; 0.33 mmol; 1.00 eq.; synthesized according to General Procedure 5 using isobutyrimidamide hydrochloride instead of acetamidine hydrochloride), to give 1-{[1-(4-chloro-3-fluorophenyl)-3-(propan-2-yl)-1H-1,2,4-triazol-5-yl]methyl}-3-{[1-(quinolin-7-yl)-1H-1,2,4-triazol-5-yl]methyl}urea (78.60 mg, 45.40%). 1H NMR (400 MHz, DMSO-d6) δ 8.99 (dd, J=4.2, 1.7 Hz, 1H), 8.48 (dt, J=8.0, 1.4 Hz, 1H), 8.23 (d, J=2.1 Hz, 1H), 8.20-8.10 (m, 2H), 7.81 (dd, J=8.7, 2.2 Hz, 1H), 7.76-7.67 (m, 2H), 7.63 (dd, J=8.3, 4.2 Hz, 1H), 7.44 (ddd, J=8.8, 2.4, 1.1 Hz, 1H), 6.74 (q, J=5.8 Hz, 2H), 4.48 (d, J=5.6 Hz, 2H), 4.34 (d, J=5.7 Hz, 2H), 2.95 (hept, J=7.0 Hz, 1H), 1.24 (d, J=6.9 Hz, 6H). LCMS (ES) [M+1]⁺ m/z 520.36.

### Example 1.59

### Synthesis of 1-{[1-(4-chloro-3-fluorophenyl)-3-methyl-1H-1,2,4-triazol-5-yl]methyl}-3-{[1-(1,5-naphthyridin-3-yl)-1H-1,2,4-triazol-5-yl]methyl}urea (Compound 43)

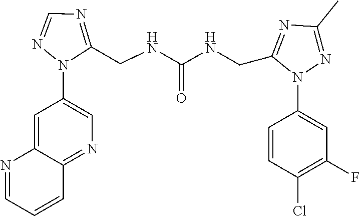 [SMILES: Cc1nc(CNC(=O)NCc2ncnn2-c2cnc3cccnc3c2)n(-c2ccc(Cl)c(F)c2)n1]

The title compound was synthesized according to General Procedure 7, Method A using [1-(4-chloro-3-fluorophenyl)-3-methyl-1H-1,2,4-triazol-5-yl]methanamine (intermediate I-6, 250.00 mg; 1.04 mmol; 1.00 eq.) and [1-(1,5-naphthyridin-3-yl)-1H-1,2,4-triazol-5-yl]methanamine hydrochloride (300.18 mg; 1.14 mmol; 1.10 eq.; synthesized from 1,5-naphthyridin-3-amine according to General Procedures 1, 2 and 3) to give 1-{[1-(4-chloro-3-fluorophenyl)-3-methyl-1H-1,2,4-triazol-5-yl]methyl}-3-{[1-(1,5-naphthyridin-3-yl)-1H-1,2,4-triazol-5-yl]methyl}urea (148 mg; 28.91%). ¹H NMR (400 MHz, DMSO-d6) δ 9.18 (d, J=2.4 Hz, 1H), 9.09 (dd, J=4.2, 1.7 Hz, 1H), 8.74 (dd, J=2.5, 0.9 Hz, 1H), 8.52 (dt, J=8.5, 1.3 Hz, 1H), 8.22 (s, 1H), 7.88 (dd, J=8.5, 4.2 Hz, 1H), 7.77-7.65 (m, 2H), 7.43 (ddd, J=8.7, 2.5, 1.1 Hz, 1H), 6.75 (t, J=5.6 Hz, 1H), 6.67 (t, J=5.6 Hz, 1H), 4.49 (d, J=5.7 Hz, 2H), 4.28 (d, J=5.6 Hz, 2H), 2.25 (s, 3H). LCMS (ES) [M+1]⁺ m/z 493.24.

### Example 1.60

### Synthesis of 1-{[1-(4-chloro-3-fluorophenyl)-3-methyl-1H-1,2,4-triazol-5-yl]methyl}-3-{[1-(1-methyl-1H-1,3-benzodiazol-5-yl)-1H-1,2,4-triazol-5-yl]methyl}urea (Compound 44)

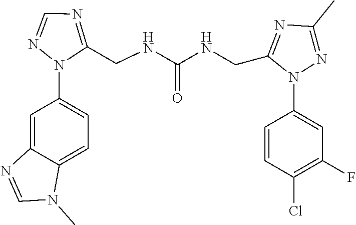 [SMILES: Cc1nc(CNC(=O)NCc2ncnn2-c2ccc3c(c2)ncn3C)n(-c2ccc(Cl)c(F)c2)n1]

The title compound was synthesized according to General Procedure 7, Method A using [1-(4-chloro-3-fluorophenyl)-3-methyl-1H-1,2,4-triazol-5-yl]methanamine (intermediate I-6, 250.00 mg; 1.04 mmol; 1.00 eq).and 1-[1-(1-methyl-1H-1,3-benzodiazol-5-yl)-1H-1,2,4-triazol-5-yl]methanamine hydrochloride (300.18 mg; 1.14 mmol; 1.10 eq.; synthesized from 1-methyl-1H-1,3-benzodiazol-5-amine according to General Procedures 1, 2 and 3) to give 1-{[1-(4-chloro-3-fluorophenyl)-3-methyl-1H-1,2,4-triazol-5-yl]methyl}-3-{[1-(1-methyl-1H-1,3-benzodiazol-5-yl)-1H-1,2,4-triazol-5-yl]methyl}urea (190 mg; 36.96%). ¹H NMR (400 MHz, DMSO-d6) δ 8.33 (s, 1H), 8.05 (s, 1H), 7.84 (d, J=1.9 Hz, 1H), 7.78-7.67 (m, 3H), 7.50-7.34 (m, 2H), 6.69 (dt, J=11.1, 5.6 Hz, 2H), 4.33 (dd, J=5.6, 4.2 Hz, 4H), 3.88 (s, 3H), 2.27 (s, 3H). LCMS (ES) [M+1]⁺ m/z 494.90.

### Example 1.61

### Synthesis of 1-{[1-(4-chloro-3-fluorophenyl)-3-ethyl-1H-1,2,4-triazol-5-yl]methyl}-3-{[1-(quinolin-7-yl)-1H-1,2,4-triazol-5-yl]methyl}urea (Compound 45)

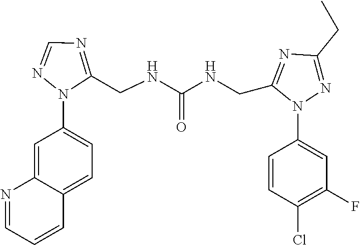 [SMILES: CCc1nc(CNC(=O)NCc2ncnn2-c2ccc3cccnc3c2)n(-c2ccc(Cl)c(F)c2)n1]

The title compound was synthesized according to General Procedure 7, Method A using 1-[1-(quinolin-7-yl)-1H-1,2,4-triazol-5-yl]methanamine (intermediate I-9; 97.28 mg; 0.43 mmol; 1.00 eq.) and 1-(4-chloro-3-fluorophenyl)-3-ethyl-1H-1,2,4-triazol-5-yl]methanamine (110.00 mg; 0.43 mmol; 1.00 eq.; synthesized according to General Procedure 5 using propionimidamide hydrochloride instead of acetamidine hydrochloride) to give 1-{[1-(4-chloro-3-fluorophenyl)-3-ethyl-1H-1,2,4-triazol-5-yl]methyl}-3-{[1-(quinolin-7-yl)-1H-1,2,4-triazol-5-yl]methyl}urea (74.70 mg, 34.19%). 1H NMR (400 MHz, DMSO-d6) δ 8.99 (dd, J=4.2, 1.7 Hz, 1H), 8.48 (dt, J=8.3, 1.4 Hz, 1H), 8.23 (d, J=2.1 Hz, 1H), 8.16 (d, J=9.3 Hz, 2H), 7.81 (dd, J=8.7, 2.1 Hz, 1H), 7.77-7.68 (m, 2H), 7.63 (dd, J=8.3, 4.2 Hz, 1H), 7.44 (ddd, J=8.7, 2.5, 1.2 Hz, 1H), 6.73 (dt, J=8.4, 5.6 Hz, 2H), 4.48 (d, J=5.6 Hz, 2H), 4.33 (d, J=5.6 Hz, 2H), 2.63 (q, J=7.6 Hz, 2H), 1.21 (t, J=7.6 Hz, 3H). LCMS (ES) [M+1]⁺ m/z 505.83.

### Example 1.62

### Synthesis of 3-{[1-(4-chloro-3-fluorophenyl)-3-methyl-1H-1,2,4-triazol-5-yl]methyl}-1-{[1-(3-fluoroquinolin-6-yl)-1H-1,2,4-triazol-5-yl]methyl}urea (Compound 46)

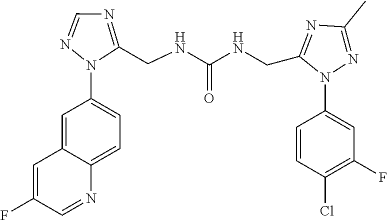 [SMILES: Cc1nc(CNC(=O)NCc2ncnn2-c2ccc3ncc(F)cc3c2)n(-c2ccc(Cl)c(F)c2)n1]

The title compound was synthesized according to General Procedure 7, Method A using [1-(4-chloro-3-fluorophenyl)-3-methyl-1H-1,2,4-triazol-5-yl]methanamine (intermediate I-6, 200.00 mg; 0.83 mmol; 1.00 eq.) and 1-[1-(3-fluoroquinolin-6-yl)-1H-1,2,4-triazol-5-yl]methanamine hydrochloride (255.68 mg; 0.91 mmol; 1.10 eq.; synthesized from 3-fluoroquinolin-6-amine according to General Procedures 1, 2 and 3) to give 1-{[1-(4-chloro-3-fluorophenyl)-3-methyl-1H-1,2,4-triazol-5-yl]methyl}-3-{[1-(3-fluoroquinolin-6-yl)-1H-1,2,4-triazol-5-yl]methyl}urea (183 mg; 43.19%). ¹H NMR (400 MHz, DMSO-d6) δ 9.02 (d, J=2.8 Hz, 1H), 8.29 (dd, J=9.4, 2.9 Hz, 1H), 8.25-8.12 (m, 3H), 7.95 (dd, J=9.0, 2.4 Hz, 1H), 7.78-7.66 (m, 2H), 7.44 (ddd, J=8.7, 2.6, 1.2 Hz, 1H), 6.71 (dt, J=12.1, 5.7 Hz, 2H), 4.49 (d, J=5.6 Hz, 2H), 4.31 (d, J=5.6 Hz, 2H), 2.25 (s, 3H). LCMS (ES) [M+1]⁺ m/z 505.83.

### Example 1.63

### Synthesis of 1-{[1-(4-chloro-3-fluorophenyl)-3-methyl-1H-1,2,4-triazol-5-yl]methyl}-3-{[1-(5-fluoroquinolin-7-yl)-1H-1,2,4-triazol-5-yl]methyl}urea (Compound 47)

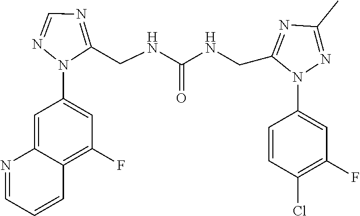 [SMILES: Cc1nc(CNC(=O)NCc2ncnn2-c2cc(F)c3cccnc3c2)n(-c2ccc(Cl)c(F)c2)n1]

The title compound was synthesized according to General Procedure 7, Method A using [1-(4-chloro-3-fluorophenyl)-3-methyl-1H-1,2,4-triazol-5-yl]methanamine (intermediate I-6; 90.00 mg; 0.37 mmol; 1.00 eq.) and [1-(5-fluoroquinolin-7-yl)-1H-1,2,4-triazol-5-yl]methanamine hydrochloride (104.60 mg; 0.37 mmol; 1.00 eq.; synthesized from 5-fluoroquinolin-7-amine according to General Procedures 1, 2 and 3) to give 1-{[1-(4-chloro-3-fluorophenyl)-3-methyl-1H-1,2,4-triazol-5-yl]methyl}-3-{[1-(5-fluoroquinolin-7-yl)-1H-1,2,4-triazol-5-yl]methyl}urea (60 mg, 31.47%). 1-H NMR (400 MHz, DMSO-d6) δ 9.07 (dd, J=4.2, 1.7 Hz, 1H), 8.55 (dt, J=8.4, 1.5 Hz, 1H), 8.20-8.08 (m, 2H), 7.83-7.66 (m, 4H), 7.44 (ddd, J=8.5, 2.4, 1.1 Hz, 1H), 6.72 (dt, J=18.0, 5.7 Hz, 2H), 4.50 (d, J=5.6 Hz, 2H), 4.31 (d, J=5.6 Hz, 2H), 2.26 (s, 3H). LCMS (ES) [M+1]⁺ m/z 511.10.

### Example 1.64

### Synthesis of 1-{[1-(4-chloro-3-fluorophenyl)-3-methyl-1H-1,2,4-triazol-5-yl]methyl}-3-{[1-(3-fluoroquinolin-7-yl)-1H-1,2,4-triazol-5-yl]methyl}urea (Compound 48)

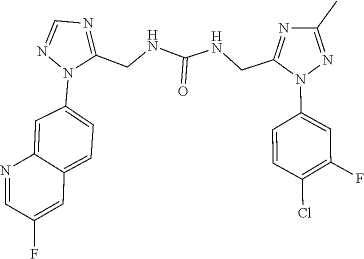 [SMILES: Cc1nc(CNC(=O)NCc2ncnn2-c2ccc3cc(F)cnc3c2)n(-c2ccc(Cl)c(F)c2)n1]

The title compound was synthesized according to General Procedure 7, Method A using [1-(4-chloro-3-fluorophenyl)-3-methyl-1H-1,2,4-triazol-5-yl]methanamine (intermediate I-6; 120.00 mg; 0.50 mmol; 1.00 eq.) and [1-(3-fluoroquinolin-7-yl)-1H-1,2,4-triazol-5-yl]methanamine hydrochloride (139.46 mg; 0.50 mmol; 1.00 eq.; synthesized from 3-fluoroquinolin-7-amine according to General Procedures 1, 2 and 3) to give 1-{[1-(4-chloro-3-fluorophenyl)-3-methyl-1H-1,2,4-triazol-5-yl]methyl}-3-{[1-(3-fluoroquinolin-7-yl)-1H-1,2,4-triazol-5-yl]methyl}urea (116 mg, 45.63%). 1-H NMR (400 MHz, DMSO-d6) δ 9.03 (d, J=2.9 Hz, 1H), 8.43-8.34 (m, 1H), 8.29 (d, J=2.1 Hz, 1H), 8.16 (d, J=8.8 Hz, 2H), 7.90-7.82 (m, 1H), 7.75-7.62 (m, 2H), 7.44 (ddd, J=8.6, 2.5, 1.2 Hz, 1H), 6.72 (dt, J=16.7, 5.6 Hz, 2H), 4.47 (d, J=5.6 Hz, 2H), 4.31 (d, J=5.6 Hz, 2H), 2.26 (s, 3H). LCMS (ES) [M+1]⁺ m/z 510.03.

### Example 1.65

### Synthesis of 1-{[1-(4-chloro-3-fluorophenyl)-3-ethyl-1H-1,2,4-triazol-5-yl]methyl}-3-{[1-(quinoxalin-6-yl)-1H-1,2,4-triazol-5-yl]methyl}urea (Compound 49)

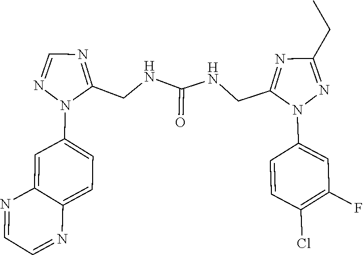 [SMILES: CCc1nc(CNC(=O)NCc2ncnn2-c2ccc3nccnc3c2)n(-c2ccc(Cl)c(F)c2)n1]

The title compound was synthesized according to General Procedure 7, Method A using 1-(4-chloro-3-fluorophenyl)-3-ethyl-1H-1,2,4-triazol-5-yl]methanamine (90.00 mg; 0.35 mmol; 1.00 eq.; synthesized according to General Procedure 5 using propionimidamide hydrochloride instead of acetamidine hydrochloride) and [1-(quinoxalin-6-yl)-1H-1,2,4-triazol-5-yl]methanamine hydrochloride (92.83 mg; 0.35 mmol; 1.00 eq.; synthesized from quinoxalin-6-amine according to General Procedures 1, 2 and 3) to give 1-{[1-(4-chloro-3-fluorophenyl)-3-ethyl-1H-1,2,4-triazol-5-yl]methyl}-3-{[1-(quinoxalin-6-yl)-1H-1,2,4-triazol-5-yl]methyl}urea (65 mg, 36.29%). ¹H NMR (400 MHz, DMSO-d6) δ 9.03 (s, 2H), 8.36 (d, J=2.3 Hz, 1H), 8.25 (d, J=8.9 Hz, 1H), 8.18 (s, 1H), 8.06 (dd, J=9.0, 2.4 Hz, 1H), 7.82-7.62 (m, 2H), 7.52-7.35 (m, 1H), 6.73 (dt, J=15.0, 5.7 Hz, 2H), 4.50 (d, J=5.5 Hz, 2H), 4.32 (d, J=5.6 Hz, 2H), 2.63 (q, J=7.6 Hz, 2H), 1.21 (t, J=7.6 Hz, 3H). LCMS (ES) [M+1]⁺ m/z 507.43.

### Example 1.66

### Synthesis of methyl 2-chloro-5-(5-{[({[1-(4-chloro-3-fluorophenyl)-3-methyl-1H-1,2,4-triazol-5-yl]methyl}carbamoyl)amino]methyl}-1H-1,2,4-triazol-1-yl)benzoate (Compound 50)

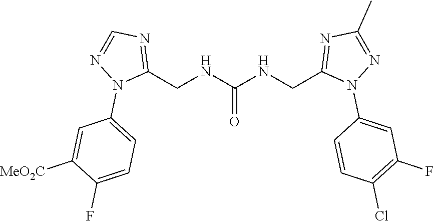 [SMILES: COC(=O)c1cc(-n2ncnc2CNC(=O)NCc2nc(C)nn2-c2ccc(Cl)c(F)c2)ccc1F]

The title compound was synthesized according to General Procedure 7, Method A using [1-(4-chloro-3-fluorophenyl)-3-methyl-1H-1,2,4-triazol-5-yl]methanamine (intermediate I-6; 480.00 mg; 1.99 mmol; 1.00 eq.) and methyl 5-[5-(aminomethyl)-1H-1,2,4-triazol-1-yl]-2-chlorobenzoate hydrochloride (604.61 mg; 1.99 mmol; 1.00 eq.; synthesized from methyl 5-amino-2-chlorobenzoate according to General Procedures 1, 2 and 3) to give methyl 2-chloro-5-(5-{[({[1-(4-chloro-3-fluorophenyl)-3-methyl-1H-1,2,4-triazol-5-yl]methyl}carbamoyl)amino]methyl}-1H-1,2,4-triazol-1-yl)benzoate. 1H NMR (400 MHz, DMSO-d6) δ 8.10 (s, 1H), 7.99 (d, J=2.6 Hz, 1H), 7.82 (dd, J=8.6, 2.6 Hz, 1H), 7.74 (ddd, J=8.5, 5.9, 1.7 Hz, 3H), 7.45 (ddd, J=8.7, 2.4, 1.2 Hz, 1H), 6.70 (t, J=5.7 Hz, 2H), 4.35 (dd, J=18.5, 5.6 Hz, 4H), 3.85 (s, 3H), 2.27 (s, 3H). LCMS (ES) [M+1]⁺ m/z 533.35.

### Example 1.67

### Synthesis of 1-{[1-(4-chloro-3-fluorophenyl)-3-methyl-1H-1,2,4-triazol-5-yl]methyl}-3-{[1-(1-methyl-1H-indazol-5-yl)-1H-1,2,4-triazol-5-yl]methyl}urea (Compound 51)

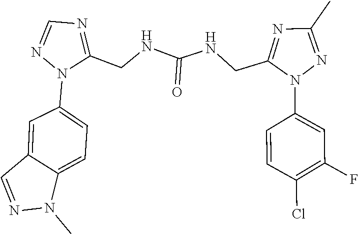 [SMILES: Cc1nc(CNC(=O)NCc2ncnn2-c2ccc3c(cnn3C)c2)n(-c2ccc(Cl)c(F)c2)n1]

The title compound was synthesized according to General Procedure 7, Method A using [1-(4-chloro-3-fluorophenyl)-3-methyl-1H-1,2,4-triazol-5-yl]methanamine (intermediate I-6; 100.00 mg; 0.42 mmol; 1.00 eq.) and 1-[1-(1-methyl-1H-indazol-5-yl)-1H-1,2,4-triazol-5-yl]methanamine hydrochloride (109.99 mg; 0.42 mmol; 1.00 eq.; synthesized from 1-methyl-1H-indazol-5-amine according to General Procedures 1, 2 and 3) to give 1-{[1-(4-chloro-3-fluorophenyl)-3-methyl-1H-1,2,4-triazol-5-yl]methyl}-3-{[1-(1-methyl-1H-indazol-5-yl)-1H-1,2,4-triazol-5-yl]methyl}urea (41 mg, 1994%). 1H NMR (400 MHz, DMSO-d6) δ 8.13 (d, J=1.0 Hz, 1H), 8.06 (s, 1H), 7.95 (dd, J=2.0, 0.7 Hz, 1H), 7.82-7.68 (m, 3H), 7.52 (dd, J=8.9, 2.0 Hz, 1H), 7.44 (ddd, J=8.7, 2.4, 1.2 Hz, 1H), 6.68 (dt, J=7.5, 5.6 Hz, 2H), 4.33 (dd, J=5.6, 1.9 Hz, 4H), 4.08 (s, 3H), 2.27 (s, 3H). LCMS (ES) [M+1]⁺ m/z 495.04.

### Example 1.68

### Synthesis of 1-{[1-(4-chloro-3-fluorophenyl)-1H-1,2,4-triazol-5-yl]methyl}-3-{[1-(1-methyl-1H-indazol-5-yl)-1H-1,2,4-triazol-5-yl]methyl}urea (Compound 52)

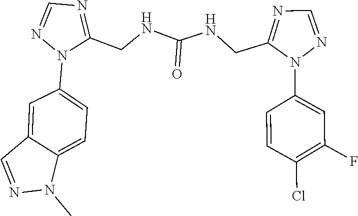 [SMILES: Cn1ncc2cc(-n3ncnc3CNC(=O)NCc3ncnn3-c3ccc(Cl)c(F)c3)ccc21]

The title compound was synthesized according to General Procedure 7, Method A using (1-(4-chloro-3-fluorophenyl)-1H-1,2,4-triazol-5-yl)methanamine (intermediate I-4, 70.26 mg; 0.31 mmol; 1.00 eq.) and N-{[1-(4-chloro-3-fluorophenyl)-1H-1,2,4-triazol-5-yl]methyl}-1H-imidazole-1-carboxamide (100.00 mg; 0.31 mmol; 1.00 eq.; synthesized from 1-methyl-1H-indazol-5-amine according to General Procedures 1, 2 and 3) to give 1-{[1-(4-chloro-3-fluorophenyl)-1H-1,2,4-triazol-5-yl]methyl}-3-{[1-(1-methyl-1H-indazol-5-yl)-1H-1,2,4-triazol-5-yl]methyl}urea (64 mg, 42.68%). 1H NMR (400 MHz, DMSO-d6) δ 8.13 (d, J=1.0 Hz, 1H), 8.10 (s, 1H), 8.06 (s, 1H), 7.95 (dd, J=2.1, 0.7 Hz, 1H), 7.81-7.72 (m, 3H), 7.54-7.44 (m, 2H), 6.70 (dt, J=11.2, 5.6 Hz, 2H), 4.35 (dd, J=19.9, 5.6 Hz, 4H), 4.08 (s, 3H). LCMS (ES) [M+1]+m/z 481.05.

### Example 1.69

### Synthesis of 1-{[1-(4-chloro-3-fluorophenyl)-3-methyl-1H-1,2,4-triazol-5-yl]methyl}-1-methyl-3-{[1-(quinolin-7-yl)-1H-1,2,4-triazol-5-yl]methyl}urea (Compound 53)

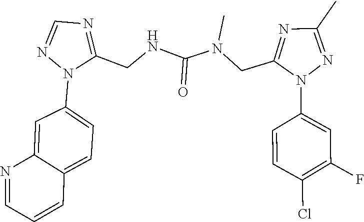 [SMILES: Cc1nc(CN(C)C(=O)NCc2ncnn2-c2ccc3cccnc3c2)n(-c2ccc(Cl)c(F)c2)n1]

The title compound was synthesized according to General Procedure 7, Method A using [1-(isoquinolin-7-yl)-1H-1,2,4-triazol-5-yl]methanamine (intermediate I-9; 100.00 mg; 0.44 mmol; 1.00 eq.) and {[1-(4-chloro-3-fluorophenyl)-3-methyl-1H-1,2,4-triazol-5-yl]methyl}(methyl)amine (113.07 mg; 0.44 mmol; 1.00 eq.; synthesized as described in Example 1.94 used to Compound 78, step 1) to give 1-{[1-(4-chloro-3-fluorophenyl)-3-methyl-1H-1,2,4-triazol-5-yl]methyl}-1-methyl-3-{[1-(quinolin-7-yl)-1H-1,2,4-triazol-5-yl]methyl}urea (63 mg, 28.05%). 1H NMR (400 MHz, DMSO-d6) δ 8.98 (dd, J=4.2, 1.7 Hz, 1H), 8.47 (dt, J=8.6, 1.2 Hz, 1H), 8.26 (d, J=2.1 Hz, 1H), 8.19-8.08 (m, 2H), 7.80 (dd, J=8.7, 2.1 Hz, 1H), 7.76-7.57 (m, 3H), 7.40 (ddd, J=8.7, 2.5, 1.2 Hz, 1H), 7.12 (t, J=5.4 Hz, 1H), 4.49 (s, 2H), 4.42 (d, J=5.3 Hz, 2H), 2.75 (s, 3H), 2.25 (s, 3H). LCMS (ES) [M+Na]+ m/z 528.09.

### Example 1.70

### Synthesis of 3-{[1-(4-chloro-3-fluorophenyl)-3-methyl-1H-1,2,4-triazol-5-yl]methyl}-1-methyl-1-{[1-(quinolin-7-yl)-1H-1,2,4-triazol-5-yl]methyl}urea (Compound 54)

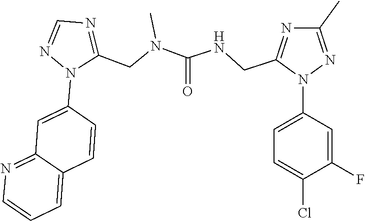 [SMILES: Cc1nc(CNC(=O)N(C)Cc2ncnn2-c2ccc3cccnc3c2)n(-c2ccc(Cl)c(F)c2)n1]

The title compound was synthesized according to General Procedure 7, Method A using [1-(4-chloro-3-fluorophenyl)-3-methyl-1H-1,2,4-triazol-5-yl]methanamine (intermediate I-6; 100.00 mg; 0.42 mmol; 1.00 eq.) and methyl({[1-(quinolin-7-yl)-1H-1,2,4-triazol-5-yl]methyl})amine hydrochloride (114.57 mg; 0.42 mmol; 1.00 eq.; synthesized following the procedures described for Example 1.43 used to prepare Compound 27, steps 1 and 2, starting from tert-butyl N-{[1-(quinolin-7-yl)-1H-1,2,4-triazol-5-yl]methyl}carbamate synthesized according to General Procedure 2 starting from commercial 7-hydrazineylquinoline) to give 3-{[1-(4-chloro-3-fluorophenyl)-3-methyl-1H-1,2,4-triazol-5-yl]methyl}-1-methyl-1-{[1-(quinolin-7-yl)-1H-1,2,4-triazol-5-yl]methyl}urea (110 mg, 52.33%). 1-H NMR (400 MHz, DMSO-d6) δ 8.98 (dd, J=4.2, 1.7 Hz, 1H), 8.47 (ddd, J=8.4, 1.8, 0.8 Hz, 1H), 8.20 (d, J=2.2 Hz, 1H), 8.17-8.11 (m, 2H), 7.78 (dd, J=8.7, 2.2 Hz, 1H), 7.75-7.67 (m, 2H), 7.62 (dd, J=8.3, 4.2 Hz, 1H), 7.43 (ddd, J=8.7, 2.4, 1.2 Hz, 1H), 7.09 (t, J=5.4 Hz, 1H), 4.70 (s, 2H), 4.25 (d, J=5.3 Hz, 2H), 2.83 (s, 3H), 2.25 (s, 3H). LCMS (ES) [M+1]⁺ m/z 506.16.

### Example 1.71

### Synthesis of 3-{[1-(4-chloro-3-fluorophenyl)-3-methyl-1H-1,2,4-triazol-5-yl]methyl}-1-{[3-cyclopropyl-1-(quinolin-7-yl)-1H-1,2,4-triazol-5-yl]methyl}urea (Compound 55)

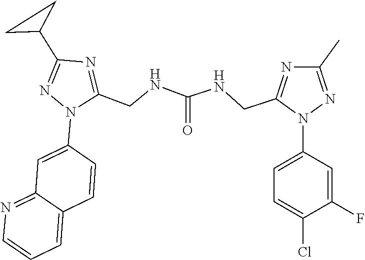 [SMILES: Cc1nc(CNC(=O)NCc2nc(C3CC3)nn2-c2ccc3cccnc3c2)n(-c2ccc(Cl)c(F)c2)n1]

The title compound was synthesized according to General Procedure 7, Method A using [1-(4-chloro-3-fluorophenyl)-3-methyl-1H-1,2,4-triazol-5-yl]methanamine (intermediate I-6, 150.00 mg; 0.62 mmol; 1.00 eq.) and [3-cyclopropyl-1-(quinolin-7-yl)-1H-1,2,4-triazol-5-yl]methanamine (165.36 mg; 0.62 mmol; 1.00 eq.; synthesized according to General Procedure 5 using 7-hydrazinylquinoline hydrochloride and cyclopropanecarboximidamide hydrochloride) to give 3-{[1-(4-chloro-3-fluorophenyl)-3-methyl-1H-1,2,4-triazol-5-yl]methyl}-1-{[3-cyclopropyl-1-(quinolin-7-yl)-1H-1,2,4-triazol-5-yl]methyl}urea (150 mg, 45.24%). ¹H NMR (400 MHz, DMSO-d6) δ 8.97 (dd, J=4.2, 1.7 Hz, 1H), 8.45 (dd, J=8.9, 1.6 Hz, 1H), 8.18 (d, J=2.1 Hz, 1H), 8.13 (d, J=8.8 Hz, 1H), 7.78 (dd, J=8.7, 2.2 Hz, 1H), 7.76-7.68 (m, 2H), 7.60 (dd, J=8.3, 4.2 Hz, 1H), 7.44 (ddd, J=8.6, 2.5, 1.2 Hz, 1H), 6.70 (td, J=5.6, 3.4 Hz, 2H), 4.40 (d, J=5.6 Hz, 2H), 4.32 (d, J=5.7 Hz, 2H), 2.27 (s, 3H), 2.03 (tt, J=8.3, 4.9 Hz, 1H), 1.02-0.91 (m, 2H), 0.90-0.82 (m, 2H). LCMS (ES) [M+1]⁺ m/z: 532.9

### Example 1.72

### Synthesis of 1,3-bis({[1-(3,4-difluorophenyl)-1H-1,2,3,4-tetrazol-5-yl]methyl})urea (Compound 56)

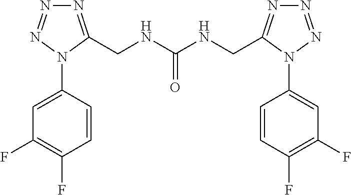 [SMILES: O=C(NCc1nnnn1-c1ccc(F)c(F)c1)NCc1nnnn1-c1ccc(F)c(F)c1]

The title compound was synthesized according to General Procedure 6, Method C using 1-[1-(3,4-di-fluorophenyl)-1H-1,2,3,4-tetrazol-5-yl]methanamine (synthesized according to General Procedure 4 starting from commercial 3,4-di-fluoro-4-chloroaniline) to provide 1,3-bis({[1-(3,4-difluorophenyl)-1H-1,2,3,4-tetrazol-5-yl]methyl})urea as a colorless solid (41.9 mg, 14.8%). 1H NMR (400 MHz, DMSO-d6) δ: 7.92 (ddd, J=10.4, 7.1, 2.5 Hz, 2H), 7.76-7.64 (m, 2H), 7.54 (dtd, J=9.0, 2.6, 1.4 Hz, 2H), 6.84 (t, J=5.8 Hz, 2H), 4.45 (t, J=2.9 Hz, 4H); LCMS (ES) [M+1]⁺ m/z: 449.2.

### Example 1.73

### Synthesis of 1,3-bis({[1-(3,4-dimethylphenyl)-1H-1,2,4-triazol-5-yl]methyl})urea (Compound 57)

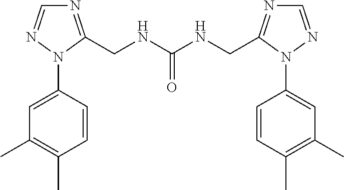

The title compound was synthesized according to General Procedure 6, Method A using 1-[1-(3,4-dimethylphenyl)-1H-1,2,4-triazol-5-yl]methanamine (100 mg; synthesized according to General Procedure 2 and 3 starting from commercial (3,4-dimethylphenyl)hydrazine) to provide 1,3-bis({[1-(3-chlorophenyl)-1H-1,2,3,4-tetrazol-5-yl]methyl})urea (17.9 mg, 8.41% yield) as a white solid. 1H NMR (300 MHz, DMSO-d6) δ 8.05 (s, 2H), 7.36-7.23 (m, 6H), 6.72 (t, J=5.6 Hz, 2H), 4.35 (d, J=5.5 Hz, 4H), 2.28 (s, 6H), 2.27 (s, 6H). LCMS (ES) [M+1]⁺ m/z 431.2.

### Example 1.74

### Synthesis of 1,3-bis({[1-(3-fluoro-4-methylphenyl)-1H-1,2,3,4-tetrazol-5-yl]methyl})urea (Compound 58)

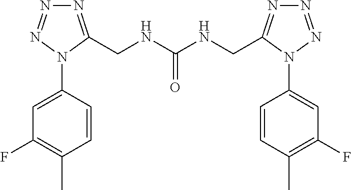 [SMILES: Cc1ccc(-n2nnnc2CNC(=O)NCc2nnnn2-c2ccc(C)c(F)c2)cc1F]

The title compound was synthesized according to General Procedure 6, Method B using 1-[1-(3-fluoro-4-methylphenyl)-1H-1,2,3,4-tetrazol-5-yl]methanamine (synthesized according to General Procedure 4 starting from commercial 3-fluoro-4-methylaniline) to provide 1,3-bis({[1-(3-fluoro-4-methylphenyl)-1H-1,2,3,4-tetrazol-5-yl]methyl})urea as a white solid (34.6 mg, 14.6%). 1H NMR (400 MHz, DMSO-d6) δ: 7.60-7.47 (m, 4H), 7.38 (dd, J=8.2, 2.1 Hz, 2H), 6.91 (t, J=5.7 Hz, 2H), 4.46 (d, J=5.5 Hz, 4H), 2.30 (d, J=2.0 Hz, 6H); LCMS (ES) [M+1]⁺ m/z: 441.2.

### Example 1.75

### Synthesis of 1,3-bis({[1-(4-chloro-3-methylphenyl)-1H-1,2,3,4-tetrazol-5-yl]methyl})urea (Compound 59)

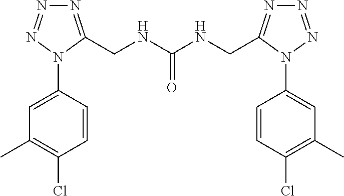

The title compound was synthesized according to General Procedure 6, Method C using 1-[1-(4-chloro-3-methylphenyl)-1H-1,2,3,4-tetrazol-5-yl]methanamine (synthesized according to General Procedure 4 starting from commercial 4-chloro-3-methylaniline) to provide 1,3-bis({[1-(4-chloro-3-methylphenyl)-1H-1,2,3,4-tetrazol-5-yl]methyl})urea as a white solid (93.6 mg, 21%). 111 NMR (400 MHz, DMSO-d6) δ: 7.68-7.61 (m, 4H), 7.50 (dd, J=8.6, 2.5 Hz, 2H), 6.87 (t, J=5.8 Hz, 2H), 4.47 (d, J=5.6 Hz, 4H), 2.37 (s, 6H); LCMS (ES) [M+1]⁺ m/z: 473.1.

### Example 1.76

### Synthesis of ,3-bis({[1-(3,4-dichlorophenyl)-1H-1,2,3,4-tetrazol-5-yl]methyl})urea (Compound 60)

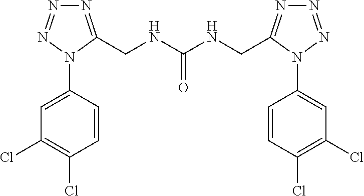 [SMILES: O=C(NCc1nnnn1-c1ccc(Cl)c(Cl)c1)NCc1nnnn1-c1ccc(Cl)c(Cl)c1]

The title compound was synthesized according to General Procedure 6, Method C using 1-[1-(3,4-dichlorophenyl)-1H-1,2,3,4-tetrazol-5-yl]methanamine (synthesized according to General Procedure 4 starting from commercial 3,4-dichloro aniline) to provide 1,3-bis({[1-(3,4-dichlorophenyl)-1H-1,2,3,4-tetrazol-5-yl]methyl})urea as a colorless solid (29.3 mg, 25%). 1H NMR (400 MHz, DMSO-d6): δ: 8.04 (d, J=2.4 Hz, 2H), 7.88 (d, J=8.6 Hz, 2H), 7.65 (dd, J=8.6, 2.5 Hz, 2H), 6.87 (t, J=5.7 Hz, 2H), 4.46 (d, J=5.7 Hz, 4H); LCMS (ES) [M+1]⁺ m/z 515.0.

### Example 1.77

### Synthesis of 1,3-bis({[1-(4-chlorophenyl)-1H-1,2,4-triazol-5-yl]methyl})urea (Compound 61)

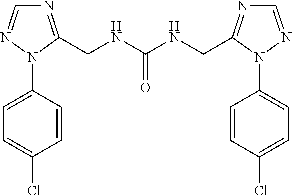 [SMILES: O=C(NCc1ncnn1-c1ccc(Cl)cc1)NCc1ncnn1-c1ccc(Cl)cc1]

The title compound was synthesized according to General Procedure 6, Method C using 1-[1-(4-chlorophenyl)-1H-1,2,4-triazol-5-yl]methanamine (synthesized according to General Procedures 2 and 3 starting from commercial 4-chloro-phenylhydrazinium chloride) to provide 1,3-bis({[1-(4-chlorophenyl)-1H-1,2,4-triazol-5-yl]methyl})urea as a white solid (40 mg, 14.5%). 1H NMR (400 MHz, DMSO-d6) δ: 8.08 (d, J=1.3 Hz, 2H), 7.60 (d, J=0.8 Hz, 2H), 7.60 (s, 6H), 6.72 (t, J=5.7 Hz, 2H), 4.35 (d, J=5.5 Hz, 4H); LCMS (ES) [M+1]⁺ m/z: 443.2.

### Example 1.78

### Synthesis of 1,3-bis({[1-(4-fluoro-3-methylphenyl)-1H-1,2,3,4-tetrazol-5-yl]methyl})urea (Compound 62)

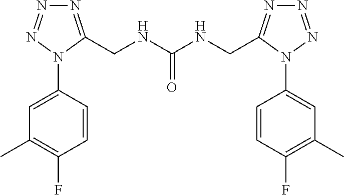

The title compound was synthesized according to General Procedure 6, Method C using 1-[1-(4-fluoro-3-methyl phenyl)-1H-1,2,3,4-tetrazol-5-yl]methanamine (synthesized according to General Procedure 4 starting from commercial 4-fluoro-3-methylaniline) to provide 1,3-bis({[1-(4-fluoro-3-methylphenyl)-1H-1,2,3,4-tetrazol-5-yl]methyl})urea as a white solid (25 mg, 11%). 1-H NMR (400 MHz, DMSO-d6) δ: 7.58 (dd, J=6.7, 2.5 Hz, 2H), 7.55-7.46 (m, 2H), 7.38 (t, J=9.0 Hz, 2H), 6.86 (t, J=5.7 Hz, 2H), 4.44 (d, J=5.6 Hz, 4H), 2.28 (d, J=2.0 Hz, 6H); LCMS (ES) [M+1]⁺ m/z: 441.2.

### Example 1.79

### Synthesis of 1,3-bis({[1-(4-chloro-3-fluorophenyl)-1H-1,2,3,4-tetrazol-5-yl]methyl})urea (Compound 63)

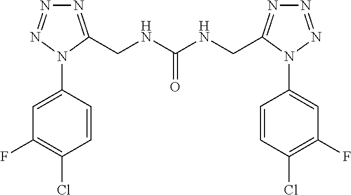 [SMILES: O=C(NCc1nnnn1-c1ccc(Cl)c(F)c1)NCc1nnnn1-c1ccc(Cl)c(F)c1]

The title compound was synthesized according to General Procedure 6, Method C using 1-[1-(4-chloro-3-fluorophenyl)-1H-1,2,3,4-tetrazol-5-yl]methanamine (synthesized according to General Procedure 4 starting from commercial 4-chloro-3-fluoroaniline) to provide 1,3-bis({[1-(4-chloro-3-fluorophenyl)-1H-1,2,3,4-tetrazol-5-yl]methyl})urea as a white solid (67 mg, 17.5%). 1H NMR (400 MHz, DMSO-d6) δ: 7.92-7.80 (m, 4H), 7.54 (d, J=8.6 Hz, 2H), 6.87 (t, J=5.8 Hz, 2H), 4.48 (d, J=5.5 Hz, 4H); LCMS (ES) [M+1]⁺ m/z: 481.1.

### Example 1.80

### Synthesis of 1,3-bis({[1-(3-chloro-4-fluorophenyl)-1H-1,2,4-triazol-5-yl]methyl})urea (Compound 64)

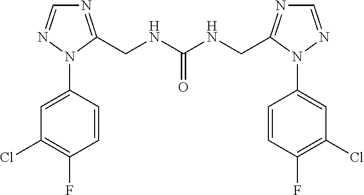 [SMILES: O=C(NCc1ncnn1-c1ccc(F)c(Cl)c1)NCc1ncnn1-c1ccc(F)c(Cl)c1]

The synthesis of the title compound is described in General Procedure 6; Method C.

### Example 1.81

### Synthesis of 1,3-bis({[1-(4-methoxyphenyl)-1H-1,2,4-triazol-5-yl]methyl})urea (Compound 65)

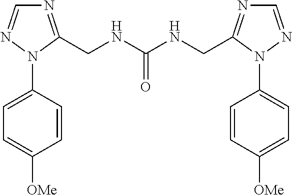 [SMILES: COc1ccc(-n2ncnc2CNC(=O)NCc2ncnn2-c2ccc(OC)cc2)cc1]

The title compound was synthesized according to General Procedure 6, Method C using 1-[1-(4-methoxyphenyl)-1H-1,2,4-triazol-5-yl]methanamine (synthesized according to General Procedures 2 and 3 starting from commercial 4-methoxy-phenylhydrazine) to provide 1,3-bis({[1-(4-methoxyphenyl)-1H-1,2,4-triazol-5-yl]methyl})urea as a white solid (40.2 mg, 14.2%). 1H NMR (400 MHz, DMSO-d6) δ: 8.06 (s, 2H), 7.44 (t, J=8.1 Hz, 2H), 7.17-7.08 (m, 4H), 7.05 (dd, J=8.4, 2.4 Hz, 2H), 6.74 (t, J=5.7 Hz, 2H), 4.37 (d, J=5.6 Hz, 4H), 3.77 (s, 6H); LCMS (ES) [M+1]⁺ m/z: 435.2.

### Example 1.82

### Synthesis of 1,3-bis({[1-(4-chloro-3-fluorophenyl)-1H-1,2,4-triazol-5-yl]methyl})urea (Compound 66)

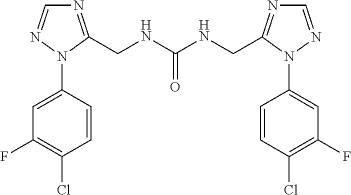 [SMILES: O=C(NCc1ncnn1-c1ccc(Cl)c(F)c1)NCc1ncnn1-c1ccc(Cl)c(F)c1]

The title compound was synthesized according to General Procedure 6; Method C using 1-[1-(4-chloro-3-fluorophenyl)-1H-1,2,4-triazol-5-yl]methanamine (270.00 mg, 1.191 mmol synthesized according to General Procedures 1, 2 and 3 starting from commercial 4-chloro-3-fluoroaniline), providing 1,3-bis({[1-(4-chloro-3-fluorophenyl)-1H-1,2,4-triazol-5-yl]methyl})urea (101.3 mg; 17.74%) as a white solid. 1H NMR (300 MHz, DMSO-d6) δ 8.12 (s, 2H), 7.85-7.73 (m, 4H), 7.50 (ddd, J=8.7, 2.4, 1.2 Hz, 2H), 6.74 (t, J=5.7 Hz, 2H), 4.40 (d, J=5.7 Hz, 4H). LCMS (ES) [M+1]⁺ m/z: 479.1.

### Example 1.83

### Synthesis of 1,3-bis({[1-(3-fluoro-4-methylphenyl)-1H-1,2,4-triazol-5-yl]methyl})urea (Compound 67)

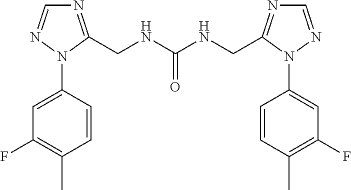 [SMILES: Cc1ccc(-n2ncnc2CNC(=O)NCc2ncnn2-c2ccc(C)c(F)c2)cc1F]

The title compound was synthesized according to General Procedure 6; Method C using 1-[1-(3-fluoro-4-methylphenyl)-1H-1,2,4-triazol-5-yl]methanamine (200.00 mg, 0.970 mmol; synthesized according to General Procedures 1, 2 and 3 starting from commercial 3-fluoro-4-methylaniline), providing 1,3-bis({[1-(3-fluoro-4-methylphenyl)-1H-1,2,4-triazol-5-yl]methyl})urea (120 mg, 28.22%) as a white solid. 1-H NMR (300 MHz, DMSO-d6) δ 8.08 (s, 2H), 7.53-7.41 (m, 4H), 7.39-7.31 (m, 2H), 6.74 (t, J=5.7 Hz 2H), 4.38 (d, J=5.7 Hz, 4H), 2.31 (s, 3H), 2.30 (s, 3H). LCMS (ES) [M+1]⁺ m/z: 439.2.

### Example 1.84

### Synthesis of 1,3-bis({[1-(3-chloro-5-fluorophenyl)-1H-1,2,4-triazol-5-yl]methyl})urea (Compound 68)

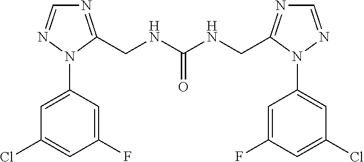 [SMILES: O=C(NCc1ncnn1-c1cc(F)cc(Cl)c1)NCc1ncnn1-c1cc(F)cc(Cl)c1]

The title compound was synthesized according to General Procedure 6; Method C using 1-[1-(3-chloro-5-fluorophenyl)-1H-1,2,4-triazol-5-yl]methanamine (208.00 mg, 0.92 mmol synthesized according to General Procedures 1, 2 and 3 starting from commercial 3-chloro-5-fluoroaniline), providing 1,3-bis({[1-(3-chloro-5-fluorophenyl)-1H-1,2,4-triazol-5-yl]methyl})urea (77 mg; 17%) as a white solid. 11-1 NMR (300 MHz, DMSO-d6): δ 8.13 (s, 2H), 7.65-7.59 (m, 6H), 6.76 (dd, J=5.7 Hz, 2H), 4.42 (d, J=5.7 Hz, 4H). LCMS (ES) [M+1]⁺ m/z: 479.

### Example 1.85

### Synthesis of 1,3-bis({[1-(4-methoxyphenyl)-1H-1,2,3,4-tetrazol-5-yl]methyl})urea (Compound 69)

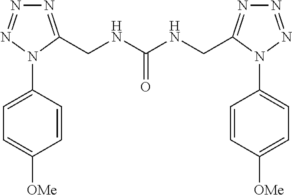 [SMILES: COc1ccc(-n2nnnc2CNC(=O)NCc2nnnn2-c2ccc(OC)cc2)cc1]

The title compound was synthesized according to General Procedure 6, Method C using 1-[1-(4-methoxyphenyl)-1H-1,2,3,4-tetrazol-5-yl]methanamine (synthesized according to General Procedure 4 starting from commercial 4-methoxyaniline) to provide 1,3-bis({[1-(4-methoxyphenyl)-1H-1,2,3,4-tetrazol-5-yl]methyl})urea as a white solid (57 mg, 12%). 1-H NMR (400 MHz, DMSO-d6) δ: 7.58-7.50 (m, 4H), 7.16-7.08 (m, 4H), 6.90 (t, J=5.7 Hz, 2H), 4.41 (d, J=5.6 Hz, 4H), 3.82 (s, 6H); LCMS (ES) [M+1]⁺ m/z: 437.3.

### Example 1.86

### Synthesis of 1,3-bis({[1-(3-chloro-4-fluorophenyl)-1H-1,2,3,4-tetrazol-5-yl]methyl})urea (Compound 70)

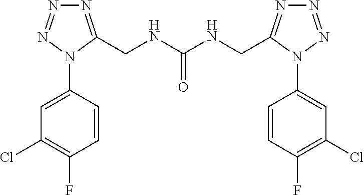 [SMILES: O=C(NCc1nnnn1-c1ccc(F)c(Cl)c1)NCc1nnnn1-c1ccc(F)c(Cl)c1]

The title compound was synthesized according to General Procedure 6, Method C using 1-(3-chloro-4-fluorophenyl)-1H-1,2,3,4-tetrazol-5-yl]methanamine (synthesized according to General Procedure 4 starting from commercial 3-chloro-4-fluoro aniline) to provide 1,3-bis({[1-(3-chloro-4-fluorophenyl)-1H-1,2,3,4-tetrazol-5-yl]methyl})urea as a white solid (46 mg, 20%). 111 NMR (400 MHz, DMSO-d6) δ: 8.01 (dd, J=6.9, 1.9 Hz, 2H), 7.68 (dd, J=7.7, 2.4 Hz, 4H), 6.85 (t, J=5.7 Hz, 2H), 4.44 (d, J=5.6 Hz, 4H); LCMS (ES) [M+1]⁺ m/z: 481.1.

### Example 1.87

### Synthesis of 1,3-bis({[1-(quinolin-6-yl)-1H-1,2,4-triazol-5-yl]methyl})urea (Compound 71)

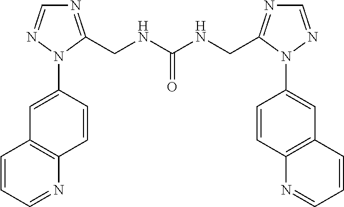 [SMILES: O=C(NCc1ncnn1-c1ccc2ncccc2c1)NCc1ncnn1-c1ccc2ncccc2c1]

The title compound was synthesized according to General Procedure 6, Method C using 1-(quinolin-6-yl)-1H-1,2,4-triazol-5-yl]methanamine (synthesized according to General Procedures 1, 2 and 3 starting from commercial quinolin-6-amine) to provide 1,3-bis({[1-(quinolin-6-yl)-1H-1,2,4-triazol-5-yl]methyl})urea as a white solid (10 mg, 11%). 1H NMR (400 MHz, DMSO-d6) δ: 8.97 (d, J=4.4 Hz, 2H), 8.41 (d, J=8.4 Hz, 2H), 8.24-8.11 (m, 6H), 7.93 (dd, J=8.9, 2.6 Hz, 2H), 7.60 (dd, J=8.3, 4.2 Hz, 2H), 6.75 (t, J=5.7 Hz, 2H), 4.45 (d, J=5.6 Hz, 4H); LCMS (ES) [M+1]⁺ m/z: 477.3.

### Example 1.88

### Synthesis of 1-[(1-cyclohexyl-1H-1,2,3,4-tetrazol-5-yl)methyl]-3-{[1-(3,4-dimethylphenyl)-1H-1,2,3,4-tetrazol-5-yl]methyl}urea (Compound 72)

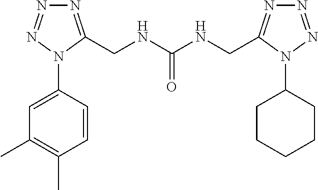 [SMILES: Cc1ccc(-n2nnnc2CNC(=O)NCc2nnnn2C2CCCCC2)cc1C]

The title compound was synthesized according to General Procedure 7; Method A using 1-[1-(3,4-dimethylphenyl)-1H-1,2,3,4-tetrazol-5-yl]methanamine (Intermediate I-5; 379 mg, 1.87 mmol, 1.0 eq.) and 1-(1-cyclohexyl-1H-1,2,3,4-tetrazol-5-yl)methanamine (338 mg, 1.87 mmol, 1.0 eq. synthesized according to General Procedure 5 starting from commercial cyclohexylamine), providing 1-[(1-cyclohexyl-1H-1,2,3,4-tetrazol-5-yl)methyl]-3-{[1-(3,4-dimethylphenyl)-1H-1,2,3,4-tetrazol-5-yl]methyl}urea (133.6 mg; 17%) as a white solid. 1H NMR (300 MHz, DMSO-d6, ppm): 6 7.44 (s, 1H), 7.39 (d, J=1.2 Hz, 2H), 6.93 (dt, J=13.2, 6.0 Hz, 2H), 4.55-4.49 (m, 5H), 2.33 (s, 3H), 2.31 (s, 3H), 1.94-1.91 (m, 2H), 1.82-1.63 (m, 5H), 1.40-1.16 (m, 3H). LCMS (ES) [M+1]⁺ m/z: 411.

### Example 1.89

### Synthesis of 1,3-bis({[1-(4-chlorophenyl)-3-methyl-1H-1,2,4-triazol-5-yl]methyl})urea (Compound 73)

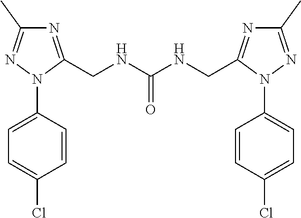

The synthesis of the title compound is described in General Procedure 6; Method B.

### Example 1.90

### Synthesis of 1,3-bis({[1-(6-methylpyridin-3-yl)-1H-1,2,4-triazol-5-yl]methyl})urea (Compound 74)

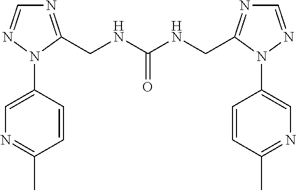 [SMILES: Cc1ccc(-n2ncnc2CNC(=O)NCc2ncnn2-c2ccc(C)nc2)cn1]

The title compound was synthesized according to General Procedure 6, Method C using 1-[1-(6-methylpyridin-3-yl)-1H-1,2,4-triazol-5-yl]methanamine (synthesized according to General Procedures 2 and 3 starting from commercial 5-hydrazinyl-2-methylpyridine hydrochloride) to provide 1,3-bis({[1-(6-methylpyridin-3-yl)-1H-1,2,4-triazol-5-yl]methyl})urea as a white solid (24 mg, 4.7%). 1-H NMR (400 MHz, DMSO-d6) δ: 8.63 (d, J=2.5 Hz, 2H), 8.10 (s, 2H), 7.90 (dd, J=8.3, 2.5 Hz, 2H), 7.43 (d, J=8.2 Hz, 2H), 6.70 (t, J=5.7 Hz, 2H), 4.32 (d, J=5.4 Hz, 4H), 2.53 (s, 6H); LCMS (ES) [M+1]⁺ m/z: 405.3.

### Example 1.91

### Synthesis of 1-{[1-(4-chloro-3-fluorophenyl)-1H-1,2,4-triazol-5-yl]methyl}-3-{[1-(quinolin-7-yl)-1H-1,2,4-triazol-5-yl]methyl}urea (Compound 75)

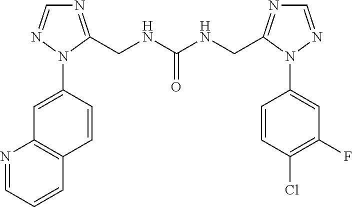 [SMILES: O=C(NCc1ncnn1-c1ccc(Cl)c(F)c1)NCc1ncnn1-c1ccc2cccnc2c1]

The title compound was synthesized according to General Procedure 7, Method A using 1-[1-(4-chloro-3-fluorophenyl)-1H-1,2,4-triazol-5-yl]methanamine (Intermediate I-4) and 1-[1-(quinolin-7-yl)-1H-1,2,4-triazol-5-yl]methanamine (Intermediate I-9) to provide 1-{[1-(4-chloro-3-fluorophenyl)-1H-1,2,4-triazol-5-yl]methyl}-3-{[1-(quinolin-7-yl)-1H-1,2,4-triazol-5-yl]methyl}urea as a white solid (64.9 mg, 40%). H NMR (400 MHz, DMSO-d6) δ: 9.27 (d, J=4.9 Hz, 1H), 9.02 (d, J=8.4 Hz, 1H), 8.52 (d, J=2.0 Hz, 1H), 8.43 (d, J=8.9 Hz, 1H), 8.23 (s, 1H), 8.09 (d, J=9.0 Hz, 2H), 7.99 (dd, J=8.4, 5.0 Hz, 1H), 7.80-7.71 (m, 2H), 7.46 (dd, J=8.7, 2.3 Hz, 1H), 6.85 (br, 2H); 4.53 (s, 2H), 4.36 (s, 2H); LCMS (ES) [M+1]⁺ m/z: 478.3.

### Example 1.92

### Synthesis of 1-{[1-(4-chloro-3-fluorophenyl)-1H-1,2,4-triazol-5-yl]methyl}-3-{[1-(2,2-difluoro-2H-1,3-benzodioxol-5-yl)-1H-1,2,3,4-tetrazol-5-yl]methyl}urea

### Example 76

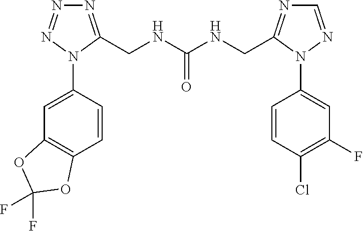 [SMILES: O=C(NCc1ncnn1-c1ccc(Cl)c(F)c1)NCc1nnnn1-c1ccc2c(c1)OC(F)(F)O2]

The title compound was synthesized according to General Procedure 7, Method A using 1-[1-(4-chloro-3-fluorophenyl)-1H-1,2,4-triazol-5-yl]methanamine (Intermediate I-4) and 1-[1-(2,2-difluoro-2H-1,3-benzodioxol-5-yl)-1H-1,2,3,4-tetrazol-5-yl]methanamine (synthesized according to General Procedures 1, 2 and 3 starting from commercial 2,2-difluoro-2H-1,3-benzodioxol-5-amine) to provide 1-{[1-(4-chloro-3-fluorophenyl)-1H-1,2,4-triazol-5-yl]methyl}-3-{[1-(2,2-difluoro-2H-1,3-benzodioxol-5-yl)-1H-1,2,3,4-tetrazol-5-yl]methyl}urea as a white solid (16 mg, 20%). ¹H NMR (400 MHz, DMSO-d6) δ: 8.11 (s, 1H), 7.85 (d, J=2.0 Hz, 1H), 7.81-7.72 (m, 2H), 7.63 (d, J=8.6 Hz, 1H), 7.54-7.38 (m, 2H), 6.84 (s, 2H), 4.46 (s, 2H), 4.35 (s, 2H); LCMS (ES) [M+1]⁺ m/z: 508.2.

### Example 1.93

### Synthesis of 1,3-bis({[1-(4-chloro-3-fluorophenyl)-3-methyl-1H-1,2,4-triazol-5-yl]methyl})urea (Compound 77)

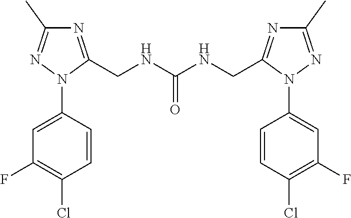 [SMILES: Cc1nc(CNC(=O)NCc2nc(C)nn2-c2ccc(Cl)c(F)c2)n(-c2ccc(Cl)c(F)c2)n1]

The title compound was synthesized according to General Procedure 6; Method C using 1-[1-(4-chloro-3-fluorophenyl)-3-methyl-1H-1,2,4-triazol-5-yl]methanamine (Intermediate I-6; 250.00 mg, 1.039 mmol), providing 1,3-bis({[1-(4-chloro-3-fluorophenyl)-3-methyl-1H-1,2,4-triazol-5-yl]methyl})urea (77.3 mg, 14.67%) as a white solid. ¹H NMR (300 MHz, DMSO-d6): δ 7.84-7.71 (m, 4H), 7.47 (ddd, J=8.7, 2.4, 1.2 Hz, 2H), 6.71 (t, J=5.7 Hz, 2H), 4.36 (d, J=5.6 Hz, 4H), 2.30 (s, 6H). LCMS (ES) [M+1]⁺ m/z: 507.

### Example 1.94

### Synthesis of 1,3-bis({[1-(4-chloro-3-fluorophenyl)-3-methyl-1H-1,2,4-triazol-5-yl]methyl})-1-methylurea (Compound 78)

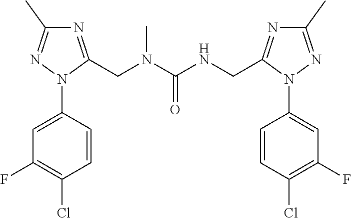 [SMILES: Cc1nc(CNC(=O)N(C)Cc2nc(C)nn2-c2ccc(Cl)c(F)c2)n(-c2ccc(Cl)c(F)c2)n1]

Step 1

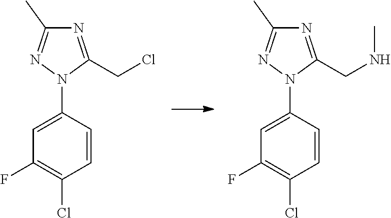 [SMILES: Cc1nc(CCl)n(-c2ccc(Cl)c(F)c2)n1 ; CNCc1nc(C)nn1-c1ccc(Cl)c(F)c1]

Into a 100-mL round-bottom flask purged and maintained with an inert atmosphere of nitrogen, was placed 1-(4-chloro-3-fluorophenyl)-5-(chloromethyl)-3-methyl-1,2,4-triazole (Intermediate I-6, Step 2; 500 mg, 1.922 mmol, 1.00 eq.) and methylamine (20 mL, 2 M) and the resulting solution was stirred for 2 h at 50° C. in an oil bath. The reaction mixture was then concentrated and the residue was applied onto a silica gel column eluted with 100% PE to 85% THF in PE providing 500 mg of crude of [[2-(4-chloro-3-fluorophenyl)-5-methyl-1,2,4-triazol-3-yl]methyl](methyl)amine as yellow oil which was used directly in the following step without further purification. LCMS (ES) [M+1]⁺ m/z: 255.

Step 2

The title compound was synthesized according to General Procedure 7; Method B using [[2-(4-chloro-3-fluorophenyl)-5-methyl-1,2,4-triazol-3-yl]methyl](methyl)amine (from step 1 above; 200.00 mg, 0.785 mmol, 1.00 eq.) and 1-[1-(4-chloro-3-fluorophenyl)-3-methyl-1H-1,2,4-triazol-5-yl]methanamine (Intermediate I-6; 188.99 mg, 0.785 mmol, 1.00 eq.), providing 1,3-bis({[1-(4-chloro-3-fluorophenyl)-3-methyl-1H-1,2,4-triazol-5-yl]methyl})-1-methylurea (114.6 mg, 27.99%) as a white solid. ¹H NMR (300 MHz, DMSO-d6): δ 7.82-7.67 (m, 4H), 7.46 (dddd, J=11.0, 8.6, 2.4, 1.1 Hz, 2H), 7.10 (t, J=5.4 Hz, 1H), 4.57 (s, 2H), 4.30 (d, J=5.3 Hz, 2H), 2.80 (s, 3H), 2.29 (s, 6H). LCMS (ES) [M+1]⁺ m/z: 521.2.

### Example 1.95

### Synthesis of 3-{[1-(4-chloro-3-fluorophenyl)-3-methyl-1H-1,2,4-triazol-5-yl]methyl}-1-methyl-1-{[1-(6-methylpyridin-3-yl)-1H-1,2,4-triazol-5-yl]methyl}urea (Compound 79)

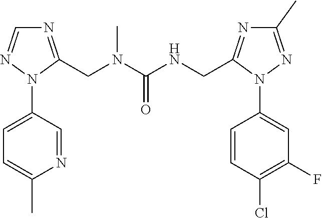 [SMILES: Cc1ccc(-n2ncnc2CN(C)C(=O)NCc2nc(C)nn2-c2ccc(Cl)c(F)c2)cn1]

Step 1

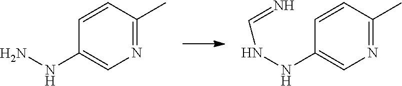

Into a 500-mL round-bottom flask, was placed 5-hydrazinyl-2-methylpyridine hydrochloride (2.00 g, 12.530 mmol, 1.00 eq.), formamidine hydrochloride (706 mg, 8.771 mmol, 0.70 eq.), MeOH (200.00 mL), and TEA (5.07 g, 50.12 mmol, 4.00 eq.) and the resulting solution was stirred for 2 days at ambient temperature. The crude product (3 g) was purified by Flash-Prep-HPLC (Prep-C18, 20-45M, 120 g, Tianjin Bonna-Agela Technologies; gradient elution of 10% MeCN in water to 30% MeCN in water over a 10 min period, where both solvents contain 0.1% NH₃H₂O) providing 1 g (41.00%) of N-[(6-methylpyridin-3-yl)amino]methanimidamide as a yellow semi-solid. LCMS (ES) [M+1]⁺ m/z 151.

Step 2

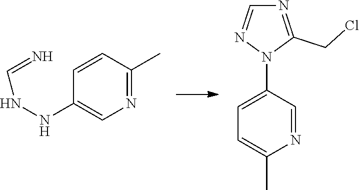

Into a 100-mL round-bottom flask purged and maintained with an inert atmosphere of nitrogen, was placed N-[(6-methylpyridin-3-yl)amino]methanimidamide (1.00 g, 6.658 mmol, 1.00 eq.), dioxane (20.00 mL) and pyridine (1.05 g, 13.317 mmol, 2.00 eq.) followed by the addition of chloroacetyl chloride (1.13 g, 9.988 mmol, 1.50 eq.) drop-wise with stirring at 0° C. over 2 min. The resulting solution was stirred for 2 h at 100° C. in an oil bath. The reaction mixture was then concentrated. The residue was applied onto a silica gel column and eluted with 100% PE to 50% THF in PE providing 250 mg (17.99%) of 5-[5-(chloromethyl)-1,2,4-triazol-1-yl]-2-methylpyridine as yellow oil. LCMS (ES) [M+1]⁺ m/z 209.

Step 3

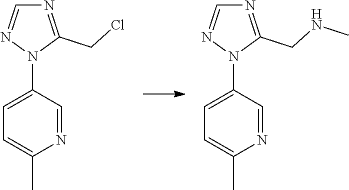

Into a 100-mL round-bottom flask, was placed 5-[5-(chloromethyl)-1,2,4-triazol-1-yl]-2-methylpyridine (250.00 mg, 1 equiv) and methylamine, 2 M in THF (20.00 mL) and the resulting solution was stirred for 2 h at 50° C. in an oil bath. The reaction mixture was then concentrated and the residue was applied onto a silica gel column and eluted with 100% PE to 80% THF in PE providing 150 mg (61.59%) of methyl([[2-(6-methylpyridin-3-yl)-1,2,4-triazol-3-yl]methyl])amine as yellow oil. LCMS (ES) [M+1]⁺ m/z 204.2.

Step 4

The title compound was synthesized according to General Procedure 7; Method B using 1-[2-(6-methylpyridin-3-yl)-1,2,4-triazol-3-yl]methanamine (from step 3 above; 150.00 mg, 0.738 mmol, 1.00 eq.) and 1-[1-(4-chloro-3-fluorophenyl)-3-methyl-1H-1,2,4-triazol-5-yl]methanamine (Intermediate I-6; 190.78 mg, 0.793 mmol, 1.00 eq.), providing 3-{[1-(4-chloro-3-fluorophenyl)-3-methyl-1H-1,2,4-triazol-5-yl]methyl}-1-methyl-14 [1-(6-methylpyridin-3-yl)-1H-1,2,4-triazol-5-yl]methyl}urea (59.3 mg, 17.10%) as a white solid. ¹H NMR (300 MHz, DMSO-d6): δ 8.62 (d, J=2.6 Hz, 1H), 8.11 (s, 1H), 7.88 (dd, J=8.3, 2.6 Hz, 1H), 7.81-7.70 (m, 2H), 7.52-7.39 (m, 2H), 7.09 (t, J=5.4 Hz, 1H), 4.57 (s, 2H), 4.30 (d, J=5.3 Hz, 2H), 2.82 (s, 3H), 2.55 (s, 3H), 2.29 (s, 3H). LCMS (ES) [M+1]⁺ m/z: 470.2.

### Example 1.96

### Synthesis of 1-{[1-(4-chloro-3-fluorophenyl)-1H-1,2,3,4-tetrazol-5-yl]methyl}-3-{[1-(quinolin-7-yl)-1H-1,2,4-triazol-5-yl]methyl}urea (Compound 80)

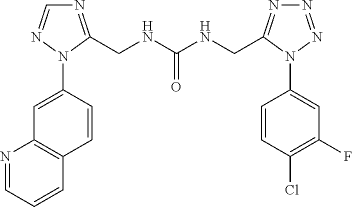 [SMILES: O=C(NCc1ncnn1-c1ccc2cccnc2c1)NCc1nnnn1-c1ccc(Cl)c(F)c1]

The title compound was synthesized according to General Procedure 7, Method A using 1-[1-(quinolin-7-yl)-1H-1,2,4-triazol-5-yl]methanamine (Intermediate I-9) and 1-[1-(4-chloro-3-fluorophenyl)-1H-1,2,3,4-tetrazol-5-yl]methanamine (synthesized according to General Procedure 4 starting from commercial 4-chloro-3-fluoroaniline) to provide 1-{[1-(4-chloro-3-fluorophenyl)-1H-1,2,3,4-tetrazol-5-yl]methyl}-3-{[1-(quinolin-7-yl)-1H-1,2,4-triazol-5-yl]methyl}urea as a white solid (133 mg, 89%). 1-H NMR (400 MHz, DMSO-d6) δ: 9.15 (dd, J=4.6, 1.6 Hz, 1H), 8.79 (d, J=8.3 Hz, 1H), 8.41-8.28 (m, 2H), 8.20 (s, 1H), 8.02-7.77 (m, 4H), 7.54 (ddd, J=8.6, 2.5, 1.2 Hz, 1H), 6.88-6.83 (m, 2H), 4.49 (m, 4H); LCMS (ES) [M+1]⁺ m/z: 479.4.

### Example 1.97

### Synthesis of 1,3-bis({[1-(quinolin-7-yl)-1H-1,2,4-triazol-5-yl]methyl})urea (Compound 81)

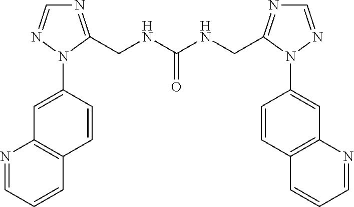 [SMILES: O=C(NCc1ncnn1-c1ccc2cccnc2c1)NCc1ncnn1-c1ccc2cccnc2c1]

The title compound was synthesized according to General Procedure 6, Method C using 1-[1-(quinolin-7-yl)-1H-1,2,4-triazol-5-yl]methanamine (Intermediate I-9) to provide 1,3-bis({[1-(quinolin-7-yl)-1H-1,2,4-triazol-5-yl]methyl})urea as a white solid (43 mg, 29%). 1H NMR (400 MHz, DMSO-d6) δ: 9.32 (ddt, J=5.6, 3.7, 1.8 Hz, 2H), 9.13 (dt, J=11.7, 6.0 Hz, 2H), 8.58 (dt, J=5.1, 2.3 Hz, 2H), 8.47 (ddd, J=9.0, 3.5, 2.0 Hz, 2H), 8.22 (s, 2H), 8.19-8.00 (m, 4H), 6.91 (s, 2H), 4.51 (s, 4H); LCMS (ES) [M+1]⁺ m/z: 477.5.

### Example 1.98

### Synthesis of 1-{[1-(4-chloro-3-fluorophenyl)-3-methyl-1H-1,2,4-triazol-5-yl]methyl}-3-{[1-(isoquinolin-6-yl)-1H-1,2,4-triazol-5-yl]methyl}urea (Compound 82)

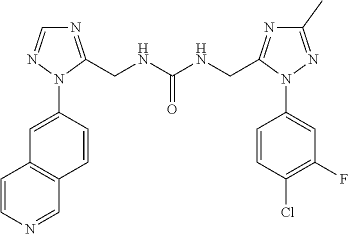 [SMILES: Cc1nc(CNC(=O)NCc2ncnn2-c2ccc3cnccc3c2)n(-c2ccc(Cl)c(F)c2)n1]

The title compound was synthesized according to General Procedure 7, Method A using 1-[1-(isoquinolin-6-yl)-1H-1,2,4-triazol-5-yl]methanamine (synthesized according to General Procedures 1, 2 and 3 starting from commercial isoquinolin-6-amine), and [1-(4-chloro-3-fluorophenyl)-3-methyl-1H-1,2,4-triazol-5-yl]methanamine (Intermediate I-6) to provide 1-{[1-(4-chloro-3-fluorophenyl)-3-methyl-1H-1,2,4-triazol-5-yl]methyl}-3-{[1-(isoquinolin-6-yl)-1H-1,2,4-triazol-5-yl]methyl}urea in form of hydrochloride salt as a white solid (30 mg, 16%). ¹H NMR (400 MHz, DMSO-d6) δ: 9.70 (s, 1H), 9.02 (s, 1H), 8.34-8.22 (m, 4H), 8.04 (dt, J=8.7, 1.6 Hz, 2H), 7.49 (m, 2H), 4.30 (m, 4H), 3.3 (s, 3H); LCMS (ES) [M+1]⁺ m/z: 492.4.

### Example 1.99

### Synthesis of 1-{[1-(4-chloro-3-fluorophenyl)-3-cyclopropyl-1H-1,2,4-triazol-5-yl]methyl}-3-{[1-(quinolin-7-yl)-1H-1,2,4-triazol-5-yl]methyl}urea (Compound 83)

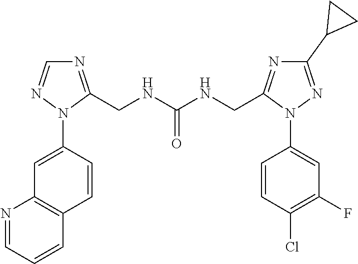 [SMILES: O=C(NCc1ncnn1-c1ccc2cccnc2c1)NCc1nc(C2CC2)nn1-c1ccc(Cl)c(F)c1]

The title compound was synthesized according to General Procedure 7, Method A using 1-[1-(4-chloro-3-fluorophenyl)-3-cyclopropyl-1H-1,2,4-triazol-5-yl]methanamine (synthesized according to General Procedure 5 using cyclopropanecarboximidamide dihydrochloride instead of acetamidine hydrochloride) and 1-(quinolin-7-yl)-1H-1,2,4-triazol-5-yl]methanamine (Intermediate I-9) to provide 1-{[1-(4-chloro-3-fluorophenyl)-3-cyclopropyl-1H-1,2,4-triazol-5-yl]methyl}-3-{[1-(quinolin-7-yl)-1H-1,2,4-triazol-5-yl]methyl}urea as a white solid (135 mg, 43%). 1-14 NMR (400 MHz, DMSO-d6) δ: 8.99 (dd, J=4.2, 1.8 Hz, 1H), 8.48 (dt, J=8.3, 1.3 Hz, 1H), 8.23 (d, J=2.1 Hz, 1H), 8.16 (d, J=9.2 Hz, 2H), 7.80 (dd, J=8.7, 2.2 Hz, 1H), 7.76-7.67 (m, 2H), 7.63 (dd, J=8.3, 4.2 Hz, 1H), 7.43 (ddd, J=8.7, 2.4, 1.2 Hz, 1H), 6.71 (dt, J=17.3, 5.6 Hz, 2H), 4.47 (d, J=5.6 Hz, 2H), 4.29 (d, J=5.6 Hz, 2H), 2.08-1.92 (m, 1H), 0.97-0.84 (m, 2H), 0.82 (dt, J=4.8, 3.0 Hz, 2H); LCMS (ES) [M+1]+m/z: 518.4.

### Example 1.100

### Synthesis of 1,3-bis({[3-methyl-1-(quinolin-7-yl)-1H-1,2,4-triazol-5-yl]methyl})urea (Compound 84)

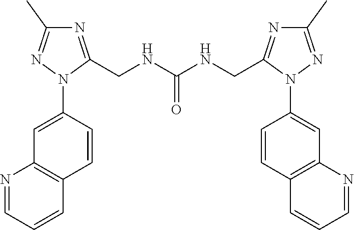 [SMILES: Cc1nc(CNC(=O)NCc2nc(C)nn2-c2ccc3cccnc3c2)n(-c2ccc3cccnc3c2)n1]

The title compound was synthesized according to General Procedure 6; Method C using 1-[3-methyl-1-(quinolin-7-yl)-1H-1,2,4-triazol-5-yl]methanamine (Intermediate I-10; 100.00 mg, 0.418 mmol), providing 1,3-bis({[3-methyl-1-(quinolin-7-yl)-1H-1,2,4-triazol-5-yl]methyl})urea (38.2 mg, 18.12%) as a white solid. 1H NMR (300 MHz, DMSO-d6): δ 8.99 (dd, J=4.3, 1.7 Hz, 2H), 8.47 (dt, J=8.1, 1.5 Hz, 2H), 8.22-8.11 (m, 4H), 7.81 (dd, J=8.7, 2.2 Hz, 2H), 7.62 (dd, J=8.3, 4.2 Hz, 2H), 6.76 (t, J=5.5 Hz, 2H), 4.46 (d, J=5.5 Hz, 4H), 2.34 (s, 6H). LCMS (ES) [M+1]⁺ m/z: 505.

### Example 1.101

### Synthesis of 1-{[1-(4-chloro-3-fluorophenyl)-1H-1,2,4-triazol-5-yl]methyl}-3-{[3-methyl-1-(quinolin-7-yl)-1H-1,2,4-triazol-5-yl]methyl}urea (Compound 85)

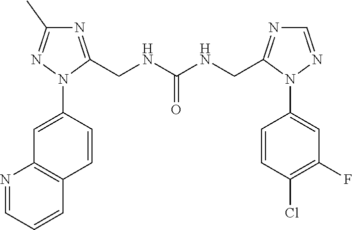 [SMILES: Cc1nc(CNC(=O)NCc2ncnn2-c2ccc(Cl)c(F)c2)n(-c2ccc3cccnc3c2)n1]

The title compound was synthesized according to General Procedure 7; Method B using 1-[1-(3-chloro-4-fluorophenyl)-1H-1,2,4-triazol-5-yl]methanamine (100.00 mg, 0.441 mmol, 1.00 eq.; synthesized according to General Procedures 2 and 3 starting from commercial 2-(3-chloro-4-fluorophenyl)hydrazinium chloride) and 1-[3-methyl-1-(quinolin-7-yl)-1H-1,2,4-triazol-5-yl]methanamine (Intermediate I-10; 105.58 mg, 0.441 mmol, 1.00 eq.), providing 1-{[1-(4-chloro-3-fluorophenyl)-1H-1,2,4-triazol-5-yl]methyl}-3-{[3-methyl-1-(quinolin-7-yl)-1H-1,2,4-triazol-5-yl]methyl}urea (37.2 mg, 17.14%) as a white solid. 1H NMR (300 MHz, DMSO-d6): δ 9.00 (dd, J=4.2, 1.7 Hz, 1H), 8.49 (dd, J=8.3, 1.0 Hz, 1H), 8.21 (d, J=2.1 Hz, 1H), 8.16 (d, J=8.8 Hz, 1H), 8.11 (s, 1H), 7.86-7.72 (m, 3H), 7.63 (dd, J=8.3, 4.2 Hz, 1H), 7.50 (dd, J=8.6, 2.9 Hz, 1H), 6.76 (q, J=5.4 Hz, 2H), 4.45 (d, J=5.4 Hz, 2H), 4.40 (d, J=5.6 Hz, 2H), 2.35 (s, 3H). LCMS (ES) [M+1]⁺ m/z: 492.

### Example 1.102

### Synthesis of 1-{[1-(4-chloro-3-fluorophenyl)-1H-1,2,3,4-tetrazol-5-yl]methyl}-3-{[3-methyl-1-(quinolin-7-yl)-1H-1,2,4-triazol-5-yl]methyl}urea (Compound 86)

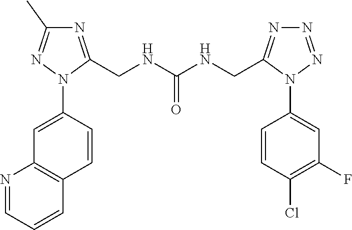 [SMILES: Cc1nc(CNC(=O)NCc2nnnn2-c2ccc(Cl)c(F)c2)n(-c2ccc3cccnc3c2)n1]

The synthesis of the title compound is described in General Procedure 7; Method B.

### Example 1.103

### Synthesis of 1-{[1-(4-chloro-3-fluorophenyl)-3-methyl-1H-1,2,4-triazol-5-yl]methyl}-3-{[3-methyl-1-(quinolin-7-yl)-1H-1,2,4-triazol-5-yl]methyl}urea (Compound 87)

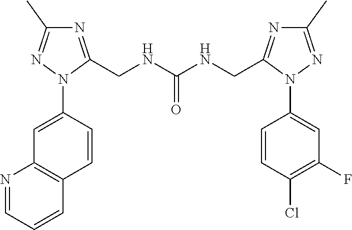 [SMILES: Cc1nc(CNC(=O)NCc2nc(C)nn2-c2ccc3cccnc3c2)n(-c2ccc(Cl)c(F)c2)n1]

The title compound was synthesized according to General Procedure 7; Method B using 1-[1-(4-chloro-3-fluorophenyl)-3-methyl-1H-1,2,4-triazol-5-yl]methanamine (Intermediate I-6; 60.00 mg, 0.249 mmol, 1.00 eq.) and 1-[3-methyl-1-(quinolin-7-yl)-1H-1,2,4-triazol-5-yl]methanamine (Intermediate I-10; 59.65 mg, 0.249 mmol, 1.00 eq.) providing 1-{[1-(4-chloro-3-fluorophenyl)-3-methyl-1H-1,2,4-triazol-5-yl]methyl}-3-{[3-methyl-1-(quinolin-7-yl)-1H-1,2,4-triazol-5-yl]methyl}urea (29.6 mg, 23.47%) as a white solid. 1-H NMR (300 MHz, DMSO-d6): δ 9.00 (dd, J=4.1, 1.7 Hz, 1H), 8.49 (d, J=7.6 Hz, 1H), 8.24-8.12 (m, 2H), 7.86-7.70 (m, 3H), 7.63 (dd, J=8.3, 4.3 Hz, 1H), 7.47 (d, J=8.9 Hz, 1H), 6.77-6.71 (m, 2H), 4.46 (d, J=5.5 Hz, 2H), 4.35 (d, J=5.6 Hz, 2H), 2.35 (s, 3H), 2.29 (s, 3H). LCMS (ES) [M+1]⁺ m/z: 506.

### Example 1.104

### Synthesis of 1-{[1-(4-chloro-3-fluorophenyl)-3-methyl-1H-1,2,4-triazol-5-yl]methyl}-3-{[1-(quinazolin-7-yl)-1H-1,2,4-triazol-5-yl]methyl}urea (Compound 88)

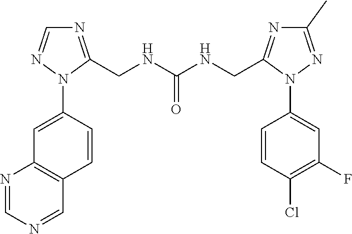 [SMILES: Cc1nc(CNC(=O)NCc2ncnn2-c2ccc3cncnc3c2)n(-c2ccc(Cl)c(F)c2)n1]

The title compound was synthesized according to General Procedure 7, Method A using 1-[1-(quinazolin-7-yl)-1H-1,2,4-triazol-5-yl]methanamine (synthesized according to General Procedures 2 and 3 starting from commercial 7-hydrazinylquinazoline), and [1-(4-chloro-3-fluorophenyl)-3-methyl-1H-1,2,4-triazol-5-yl]methanamine (Intermediate I-6) to provide 1-{[1-(4-chloro-3-fluorophenyl)-3-methyl-1H-1,2,4-triazol-5-yl]methyl}-3-{[1-(quinazolin-7-yl)-1H-1,2,4-triazol-5-yl]methyl}urea as a white solid (45 mg, 30%). 1-14 NMR (DMSO-d6) δ: 9.74 (s, 1H), 9.37 (s, 1H), 8.72 (m, 2H), 8.37 (d, J=8.7 Hz, 1H), 8.30-8.20 (m, 2H), 8.16 (s, 1H), 7.99 (dd, J=8.7, 2.1 Hz, 1H), 6.22 (d, J=2.5 Hz, 2H), 4.52-4.31 (m, 4H), 2.52 (s, 3H); LCMS (ES) [M+1]⁺ m/z: 493.4.

### Example 1.105

### Synthesis of 1-{[1-(4-chloro-3-fluorophenyl)-3-methyl-1H-1,2,4-triazol-5-yl]methyl}-3-{[4-(4-chloro-3-fluorophenyl)-5-oxo-4,5-dihydro-1H-1,2,4-triazol-3-yl]methyl}urea (Compound 89)

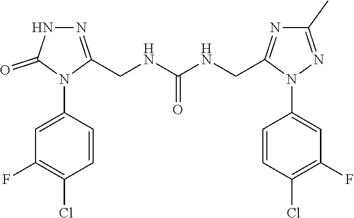 [SMILES: Cc1nc(CNC(=O)NCc2n[nH]c(=O)n2-c2ccc(Cl)c(F)c2)n(-c2ccc(Cl)c(F)c2)n1]

The title compound was synthesized according to General Procedure 7; Method A using 1-[1-(4-chloro-3-fluorophenyl)-3-methyl-1H-1,2,4-triazol-5-yl]methanamine (Intermediate I-6; 99.19 mg, 0.412 mmol, 1.00 eq.) and 3-(aminomethyl)-4-(4-chloro-3-fluorophenyl)-4,5-dihydro-1H-1,2,4-triazol-5-one (Intermediate I-7; 100.00 mg, 0.412 mmol, 1.00 eq.) providing 1-{[1-(4-chloro-3-fluorophenyl)-3-methyl-1H-1,2,4-triazol-5-yl]methyl}-3-{[4-(4-chloro-3-fluorophenyl)-5-oxo-4,5-dihydro-1H-1,2,4-triazol-3-yl]methyl}urea (92.9 mg; 44.26%) as a white solid. 1H NMR (300 MHz, DMSO-d6): δ 11.86 (br, 1H), 7.84-7.64 (m, 3H), 7.59 (dd, J=10.1, 2.3 Hz, 1H), 7.47 (ddd, J=8.6, 2.5, 1.2 Hz, 1H), 7.29 (ddd, J=8.6, 2.4, 1.1 Hz, 1H), 6.60 (t, J=5.6 Hz, 1H), 6.41 (t, J=5.6 Hz, 1H), 4.30 (d, J=5.6 Hz, 2H), 4.08 (d, J=5.5 Hz, 2H), 2.30 (s, 3H). LCMS (ES) [M+1]⁺ m/z: 509.1.

### Example 1.106

### Synthesis of 3-{[4-(4-chloro-3-fluorophenyl)-1-methyl-5-oxo-4,5-dihydro-1H-1,2,4-triazol-3-yl]methyl}-1-{[1-(4-chloro-3-fluorophenyl)-3-methyl-1H-1,2,4-triazol-5-yl]methyl}urea (Compound 90)

Step 1

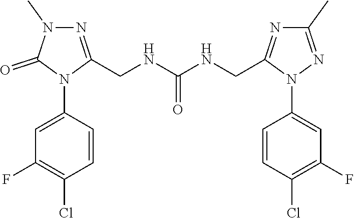 [SMILES: Cc1nc(CNC(=O)NCc2nn(C)c(=O)n2-c2ccc(Cl)c(F)c2)n(-c2ccc(Cl)c(F)c2)n1]

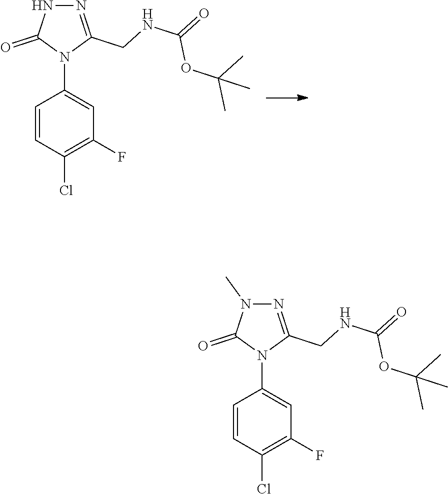 [SMILES: CC(C)(C)OC(=O)NCc1n[nH]c(=O)n1-c1ccc(Cl)c(F)c1 ; Cn1nc(CNC(=O)OC(C)(C)C)n(-c2ccc(Cl)c(F)c2)c1=O]

Into a 50-mL round-bottom flask purged and maintained with an inert atmosphere of nitrogen, was placed tert-butyl N-[[4-(4-chloro-3-fluorophenyl)-5-oxo-1H-1,2,4-triazol-3-yl]methyl]carbamate (300.00 mg, 0.875 mmol, 1.00 eq.; prepared as described in the synthesis of Intermediate I-7, Step 3), K₂CO₃ (362.89 mg, 2.626 mmol, 3.00 equiv), and DMF (5.00 mL). This was followed by the addition of CH₃I (248.46 mg, 1.750 mmol, 2.00 equiv) dropwise with stirring at 0° C. over 1 min. The resulting solution was stirred overnight at ambient temperature. The crude product was purified by Flash-Prep-HPLC providing 220 mg (70.45%) of tert-butyl N-[[4-(4-chloro-3-fluorophenyl)-1-methyl-5-oxo-1,2,4-triazol-3-yl]methyl]carbamate as an off-white solid. LCMS (ES) [M+1]⁺ m/z 357.

Step 2

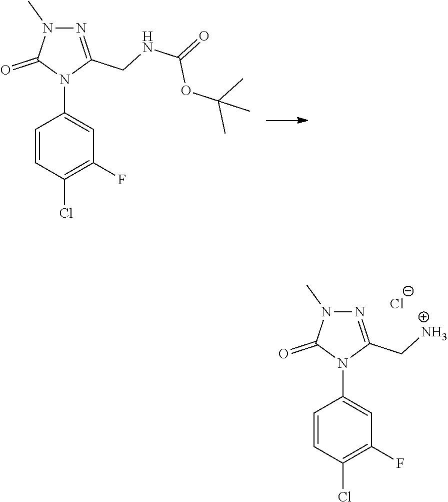 [SMILES: Cn1nc(CNC(=O)OC(C)(C)C)n(-c2ccc(Cl)c(F)c2)c1=O ; Cn1nc(C[NH3+])n(-c2ccc(Cl)c(F)c2)c1=O ; [Cl-]]

Into a 100-mL round-bottom flask purged and maintained with an inert atmosphere of nitrogen, was placed tert-butyl N-[[4-(3-fluorophenyl)-1-methyl-5-oxo-1,2,4-triazol-3-yl]methyl]carbamate (200.00 mg, 0.620 mmol, 1.00 equiv), 1,4-dioxane (10.00 mL) and HCl (gas) in 1,4-dioxane (10.00 mL). The resulting solution was stirred overnight at ambient temperature. The reaction mixture was then concentrated under vacuum providing 150 mg (82.48%) of [4-(4-chloro-3-fluorophenyl)-1-methyl-5-oxo-4,5-dihydro-1H-1,2,4-triazol-3-yl]methanaminium chloride as an off-white solid. LCMS (ES) [M+1-HCl]⁺ m/z 257.

Step 3

The title compound was synthesized according to General Procedure 7; Method A using 1-[1-(4-chloro-3-fluorophenyl)-3-methyl-1H-1,2,4-triazol-5-yl]methanamine (Intermediate I-6; 123.16 mg, 0.512 mmol, 1.00 eq.) and [4-(4-chloro-3-fluorophenyl)-1-methyl-5-oxo-4,5-dihydro-1H-1,2,4-triazol-3-yl]methanaminium chloride (from step 2 above; 150.00 mg, 0.512 mmol, 1.00 eq.) providing 3-{[4-(4-chloro-3-fluorophenyl)-1-methyl-5-oxo-4,5-dihydro-1H-1,2,4-triazol-3-yl]methyl}-1-{[1-(4-chloro-3-fluorophenyl)-3-methyl-1H-1,2,4-triazol-5-yl]methyl}urea (105 mg; 39.21%) as a white solid. ¹H NMR (300 MHz, DMSO-d6): δ 7.84-7.65 (m, 3H), 7.60 (dd, J=10.0, 2.4 Hz, 1H), 7.47 (ddd, J=8.7, 2.5, 1.2 Hz, 1H), 7.30 (ddd, J=8.6, 2.4, 1.2 Hz, 1H), 6.62 (t, J=5.7 Hz, 1H), 6.43 (t, J=5.7 Hz, 1H), 4.31 (d, J=5.6 Hz, 2H), 4.09 (d, J=5.6 Hz, 2H), 3.36 (s, 3H), 2.30 (s, 3H). LCMS (ES) [M+1]⁺ m/z: 523.2.

### Example 1.107

### Synthesis of 1-{[1-(4-chloro-3-fluorophenyl)-1H-1,2,4-triazol-5-yl]methyl}-3-{[3-cyclopropyl-1-(quinolin-7-yl)-1H-1,2,4-triazol-5-yl]methyl}urea (Compound 91)

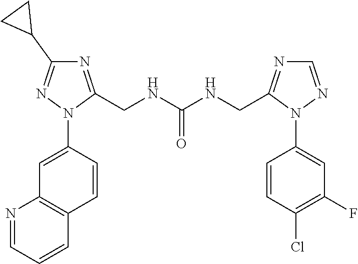 [SMILES: O=C(NCc1ncnn1-c1ccc(Cl)c(F)c1)NCc1nc(C2CC2)nn1-c1ccc2cccnc2c1]

The title compound was synthesized according to General Procedure 7; Method A using 1-[1-(4-chloro-3-fluorophenyl)-1H-1,2,4-triazol-5-yl]methanamine (Intermediate I-4; 85.42 mg, 0.377 mmol, 1.00 eq.) and 1-[3-cyclopropyl-1-(quinolin-7-yl)-1H-1,2,4-triazol-5-yl]methanamine (100.00 mg, 0.377 mmol, 1.00 eq.; synthesized according to General Procedure 5 using 7-hydrazinylquinoline hydrochloride and cyclopropanecarboximidamide hydrochloride) providing 1-{[1-(4-chloro-3-fluorophenyl)-1H-1,2,4-triazol-5-yl]methyl}-3-{[3-cyclopropyl-1-(quinolin-7-yl)-1H-1,2,4-triazol-5-yl]methyl}urea (78.2 mg, 40.06%) as a white solid. 1H NMR (300 MHz, DMSO-d6): δ 8.99 (dd, J=4.2, 1.8 Hz, 1H), 8.48 (d, J=8.1 Hz, 1H), 8.20 (d, J=2.1 Hz, 1H), 8.15 (d, J=8.8 Hz, 1H), 8.11 (s, 1H), 7.85-7.72 (m, 3H), 7.63 (dd, J=8.3, 4.2 Hz, 1H), 7.50 (ddd, J=8.7, 2.4, 1.0 Hz, 1H), 6.74 (t, J=5.6 Hz, 2H), 4.42 (d, J=5.5 Hz, 2H), 4.39 (d, J=5.6 Hz, 2H), 3.30 (s, 1H), 2.06 (ddd, J=13.1, 8.2, 4.8 Hz, 1H), 0.97 (dt, J=8.2, 2.8 Hz, 2H), 0.89 (dt, J=5.0, 2.7 Hz, 2H). LCMS (ES) [M+1]⁺ m/z: 518.

### Example 1.108

### Synthesis of 1-{[1-(4-chloro-3-fluorophenyl)-1H-1,2,4-triazol-5-yl]methyl}-3-{[3-ethyl-1-(quinolin-7-yl)-1H-1,2,4-triazol-5-yl]methyl}urea (Compound 92)

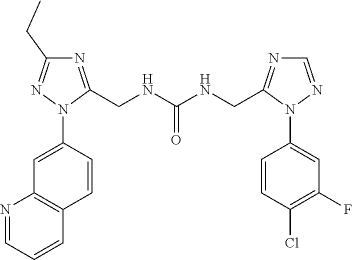 [SMILES: CCc1nc(CNC(=O)NCc2ncnn2-c2ccc(Cl)c(F)c2)n(-c2ccc3cccnc3c2)n1]

The title compound was synthesized according to General Procedure 7; Method A using 1-[2-(4-chloro-3-fluorophenyl)-1,2,4-triazol-3-yl]methanamine hydrochloride (Intermediate I-4; 171.38 mg, 0.651 mmol, 1.1 equiv) and 1-[5-ethyl-2-(quinolin-7-yl)-1,2,4-triazol-3-yl]methanamine (150.00 mg, 0.592 mmol, 1.00 eq.; synthesized according to General Procedure 5 using 7-hydrazinylquinoline hydrochloride and propionimidamide hydrochloride) providing 1-{[1-(4-chloro-3-fluorophenyl)-1H-1,2,4-triazol-5-yl]methyl}-3-{[3-ethyl-1-(quinolin-7-yl)-1H-1,2,4-triazol-5-yl]methyl}urea (97.1 mg; 32.41%) as a white solid. 1-H NMR (300 MHz, DMSO-d6): δ 8.99 (dd, J=4.2, 1.7 Hz, 1H), 8.48 (dd, J=8.3, 1.8 Hz, 1H), 8.24-8.12 (m, 2H), 8.10 (s, 1H), 7.86-7.72 (m, 3H), 7.63 (dd, J=8.3, 4.2 Hz, 1H), 7.54-7.44 (m, 1H), 6.77 (q, J=5.9 Hz, 2H), 4.46 (d, J=5.5 Hz, 2H), 4.40 (d, J=5.6 Hz, 2H), 2.71 (q, J=7.6 Hz, 2H), 1.28 (t, J=7.6 Hz, 3H). LCMS (ES) [M+1]⁺ m/z: 506.2.

### Example 1.109

### Synthesis of 1-{[1-(4-chloro-3-fluorophenyl)-3-(hydroxymethyl)-1H-1,2,4-triazol-5-yl]methyl}-3-{[3-methyl-1-(quinolin-7-yl)-1H-1,2,4-triazol-5-yl]methyl}urea (Compound 93)

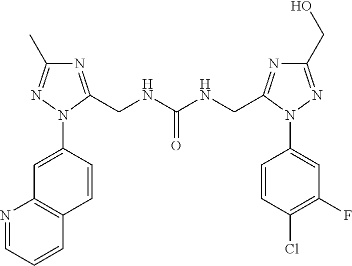 [SMILES: Cc1nc(CNC(=O)NCc2nc(CO)nn2-c2ccc(Cl)c(F)c2)n(-c2ccc3cccnc3c2)n1]

Step 1

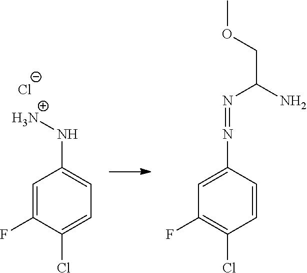

Into a 250-mL round-bottom flask, was placed (4-chloro-3-fluorophenyl)hydrazine hydrochloride (10.00 g, 50.754 mmol, 1.00 eq.), methanol (100 mL), TEA (15.41 g, 152.261 mmol, 3.00 eq.), 2-methoxyethanimidamide hydrochloride (6.32 g, 50.754 mmol, 1.00 eq.) and the resulting solution was stirred for 16 h at 60° C. The reaction mixture was then cooled to ambient temperature and concentrated. The residue was applied onto a silica gel column and eluted with ethyl acetate/petroleum ether (1:1). The collected fractions were combined and concentrated providing 5.2 g (44.23%) of (1-amino-2-methoxyethyl)(4-chloro-3-fluorophenyl)diazene as a yellow solid. LCMS (ES) [M+1]⁺ m/z 232.

Step 2

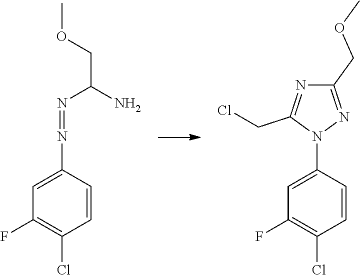 [SMILES: COCC(N)N=Nc1ccc(Cl)c(F)c1 ; COCc1nc(CCl)n(-c2ccc(Cl)c(F)c2)n1]

Into a 250-mL round-bottom flask, was placed (1-amino-2-methoxyethyl)(4-chloro-3-fluorophenyl)diazene (5.00 g, 21.583 mmol, 1.00 eq.), dioxane (50.00 mL, 590.204 mmol, 27.35 eq.), pyridine (5.12 g, 64.750 mmol, 3.00 eq.), and chloroacetyl chloride (3.66 g, 32.375 mmol, 1.50 eq.) and the resulting solution was stirred for 2 h at 100° C. The reaction mixture was then cooled to ambient temperature and concentrated. The residue was diluted with 200 mL of Ethyl acetate and washed with 2 ×100 mL of water. The organic layer was dried over anhydrous sodium sulfate and concentrated under vacuum providing 3.5 g (55.89%) of 1-(4-chloro-3-fluorophenyl)-5-(chloromethyl)-3-(methoxymethyl)-1,2,4-triazole as a yellow solid. LCMS (ES) [M+1]+m/z 290.

Step 3

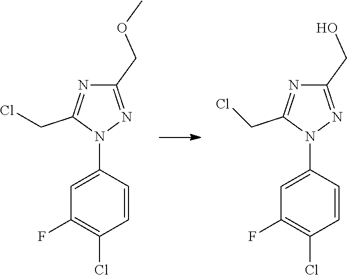 [SMILES: COCc1nc(CCl)n(-c2ccc(Cl)c(F)c2)n1 ; OCc1nc(CCl)n(-c2ccc(Cl)c(F)c2)n1]

Into a 100-mL round-bottom flask, was placed 1-(4-chloro-3-fluorophenyl)-5-(chloromethyl)-3-(methoxymethyl)-1,2,4-triazole (3.00 g, 10.341 mmol, 1.00 eq.), and DCM (30.00 mL). BBr₃ (103 mL, 103.405 mmol, 10.00 eq., 1M) was then added drop-wise at −78° C. and the resulting solution was stirred for 2 h at −40° C. The reaction mixture was then warmed to ambient temperature, diluted with water, and then adjusted to pH 7 with sodium bicarbonate. The organic layer was dried over anhydrous sodium sulfate and concentrated under vacuum. The residue was applied onto a silica gel column eluted with 100% petroleum ether to 35% ethyl acetate in petroleum ether. The collected fractions were combined and concentrated providing 1.6 g (56.04%) of [1-(4-chloro-3-fluorophenyl)-5-(chloromethyl)-1,2,4-triazol-3-yl]methanol as an off-white solid. LCMS (ES) [M+1]⁺ m/z 276.

Step 4

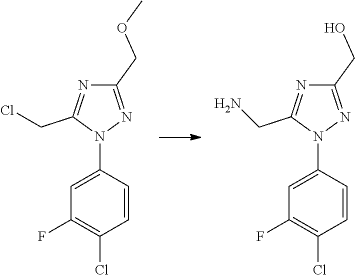 [SMILES: COCc1nc(CCl)n(-c2ccc(Cl)c(F)c2)n1 ; NCc1nc(CO)nn1-c1ccc(Cl)c(F)c1]

Into a 100-mL round-bottom flask, was placed (1-(4-chloro-3-fluorophenyl)-5-(chloromethyl)-1H-1,2,4-triazol-3-yl)methanol (500.00 mg, 1.81 mmol, 1.00 eq.), and NH₃·H₂O (20.00 mL) and the resulting solution was stirred for 3 h at 50° C. The reaction mixture was then cooled to ambient temperature and concentrated. The residue was purified by preparative HPLC (Prep-C18, 20-45 uM, 120 g, Tianjin Bonna-Agela Technologies; gradient elution of 20% MeCN in water to 30% MeCN in water over a 10 min period, water contains 0.1% NH₃H₂O) to provide 350 mg (75.3%) of (5-(aminomethyl)-1-(4-chloro-3-fluorophenyl)-1H-1,2,4-triazol-3-yl)methanol as a white solid. LCMS (ES) [M+1]⁺ m/z 257.

Step 5

The title compound was synthesized according to General Procedure 7; Method A using (5-(aminomethyl)-1-(4-chloro-3-fluorophenyl)-1H-1,2,4-triazol-3-yl)methanol (from step 4 above; 100.00 mg, 0.390 mmol, 1.00 eq.) and 1-[3-methyl-1-(quinolin-7-yl)-1H-1,2,4-triazol-5-yl]methanamine (Intermediate I-10; 93.23 mg, 0.390 mmol, 1.00 eq.) providing 1-{[1-(4-chloro-3-fluorophenyl)-3-(hydroxymethyl)-1H-1,2,4-triazol-5-yl]methyl}-3-{[3-methyl-1-(quinolin-7-yl)-1H-1,2,4-triazol-5-yl]methyl}urea (87.2 mg, 42.88%) as a white solid. 1H NMR (300 MHz, DMSO-d6): δ 9.00 (dd, J=4.3, 1.8 Hz, 1H), 8.48 (dd, J=8.0, 1.5 Hz, 1H), 8.24-8.12 (m, 2H), 7.86-7.72 (m, 3H), 7.63 (dd, J=8.3, 4.2 Hz, 1H), 7.49 (ddd, J=8.5, 2.3, 1.0 Hz, 1H), 6.76 (q, J=5.4 Hz, 2H), 5.37 (t, J=6.0 Hz, 1H), 4.46 (d, J=5.8 Hz, 4H), 4.39 (d, J=5.6 Hz, 2H), 2.35 (s, 3H). LCMS (ES) [M+1]⁺ m/z: 522.

### Example 1.110

### Synthesis of 1-{[1-(4-chloro-3-fluorophenyl)-3-(2-hydroxyethyl)-1H-1,2,4-triazol-5-yl]methyl}-3-{[3-methyl-1-(quinolin-7-yl)-1H-1,2,4-triazol-5-yl]methyl}urea (Compound 94)

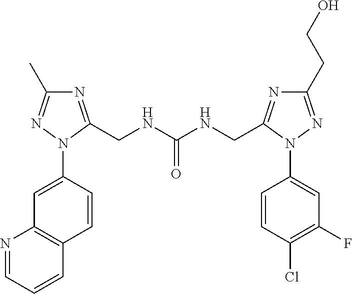 [SMILES: Cc1nc(CNC(=O)NCc2nc(CCO)nn2-c2ccc(Cl)c(F)c2)n(-c2ccc3cccnc3c2)n1]

Step 1

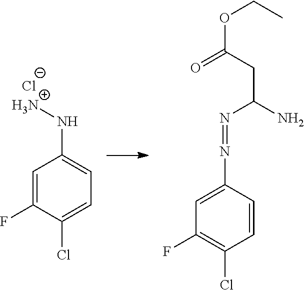 [SMILES: [NH3+]Nc1ccc(Cl)c(F)c1 ; [Cl-] ; CCOC(=O)CC(N)N=Nc1ccc(Cl)c(F)c1]

Into a 100-mL round-bottom flask, was placed (4-chloro-3-fluorophenyl)hydrazine hydrochloride (2.00 g, 10.15 mmol, 1.00 eq.), and pyridine (20.00 mL) followed by the portion-wise addition of ethyl 3-ethoxy-3-iminopropanoate hydrochloride (1.99 g, 10.17 mmol, 1.00 eq.) at ambient temperature. The resulting solution was stirred overnight at ambient temperature. The reaction mixture was then concentrated and the crude product was re-crystallized from EA/PE in the ratio of 50%. The solids were collected by filtration, and dried under infrared light providing 1.5 g (53.99%) of ethyl 2-[N-[(4-chloro-3-fluorophenyl)amino]carbamimidoyl]acetate as an off-white solid. LCMS (ES) [M+H]⁺ m/z: 274.

Step 2

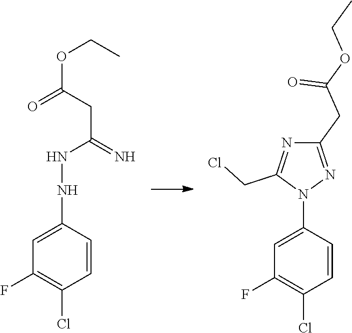 [SMILES: CCOC(=O)CC(=N)NNc1ccc(Cl)c(F)c1 ; CCOC(=O)Cc1nc(CCl)n(-c2ccc(Cl)c(F)c2)n1]

Into a 100-mL round-bottom flask, was placed ethyl 2-[N-[(4-chloro-3-fluorophenyl)amino]carbamimidoyl]acetate (1.50 g, 5.48 mmol, 1.00 eq.), dioxane (20.00 mL), and pyridine (1.30 g, 16.44 mmol, 3.00 equiv) followed by the addition of chloroacetyl chloride (0.93 g, 8.23 mmol, 1.50 equiv) drop-wise with stirring at ambient temperature. The resulting solution was stirred for 5 h at 100° C. The reaction mixture was then cooled to room temperature and extracted with 3×20 mL of ethyl acetate, The organic layers were combined and dried over anhydrous sodium sulfate and concentrated and the residue was applied onto a silica gel column eluted with 100% petroleum ether to 20% THF in petroleum ether providing 400 mg (21.97%) of ethyl 2-[1-(4-chloro-3-fluorophenyl)-5-(chloromethyl)-1,2,4-triazol-3-yl]acetate as yellow oil. LCMS (ES) [M+H]⁺ m/z: 332.

Step 3

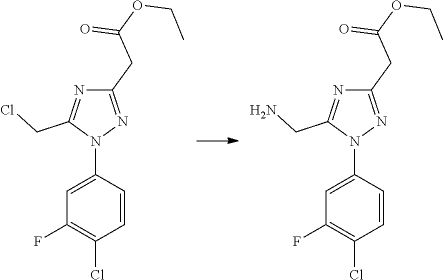 [SMILES: CCOC(=O)Cc1nc(CCl)n(-c2ccc(Cl)c(F)c2)n1 ; CCOC(=O)Cc1nc(CN)n(-c2ccc(Cl)c(F)c2)n1]

Into a 40-mL vial, was placed ethyl 2-[1-(4-chloro-3-fluorophenyl)-5-(chloromethyl)-1,2,4-triazol-3-yl]acetate (350.00 mg, 1.05 mmol, 1.00 equiv), dioxane (15.00 mL), and NH₃₁₁₂₀ (3.00 mL) and the resulting solution was stirred for 3 h at 50° C. The reaction mixture was then cooled to ambient temperature and concentrated. The crude product (300 mg) was purified by Prep-HPLC with the following conditions: Column,)(Bridge Prep C18 OBD Column, 19 cm, 150 mm, 5 um; mobile phase, Water (0.1% NH₄HCO₃) and CAN (30% Phase B up to 60% in 11 min); Detector, 254 providing 170 mg (51.59%) of ethyl 2-[5-(aminomethyl)-1-(4-chloro-3-fluorophenyl)-1,2,4-triazol-3-yl]acetate as yellow solid. LCMS (ES) [M+H]⁺ m/z: 313.

Step 4

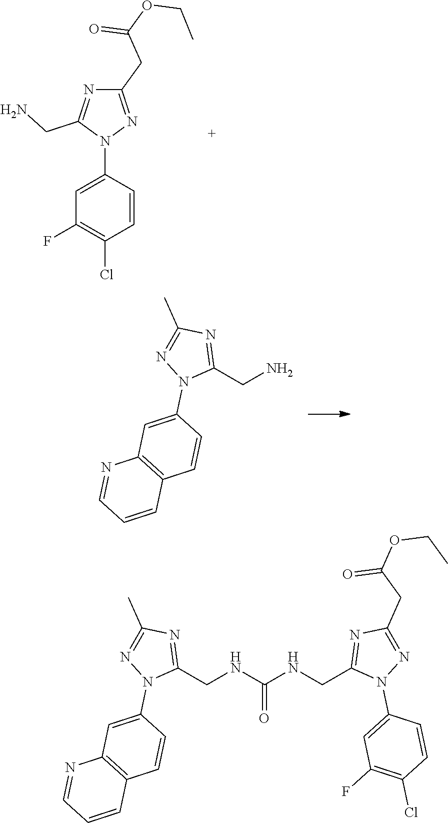 [SMILES: CCOC(=O)Cc1nc(CN)n(-c2ccc(Cl)c(F)c2)n1 ; Cc1nc(CN)n(-c2ccc3cccnc3c2)n1 ; CCOC(=O)Cc1nc(CNC(=O)NCc2nc(C)nn2-c2ccc3cccnc3c2)n(-c2ccc(Cl)c(F)c2)n1]

The intermediate indicated compound was synthesized according to General Procedure 7; Method A using 1-[3-methyl-1-(quinolin-7-yl)-1H-1,2,4-triazol-5-yl]methanamine (Intermediate I-10; 114.77 mg, 0.48 mmol, 1.00 eq.) and ethyl 2-[5-(aminomethyl)-1-(4-chloro-3-fluorophenyl)-1,2,4-triazol-3-yl]acetate (from step 3 above; 150.00 mg, 0.48 mmol, 1.00 eq.) providing ethyl 2-[1-(4-chloro-3-fluorophenyl)-5-{[({[3-methyl-1-(quinolin-7-yl)-1H-1,2,4-triazol-5-yl]methyl}carbamoyl)amino]methyl}-1H-1,2,4-triazol-3-yl]acetate (190 mg; 68.53%) as a white solid. LCMS: (ES, m/z): [M+H]⁺: 578

Step 5

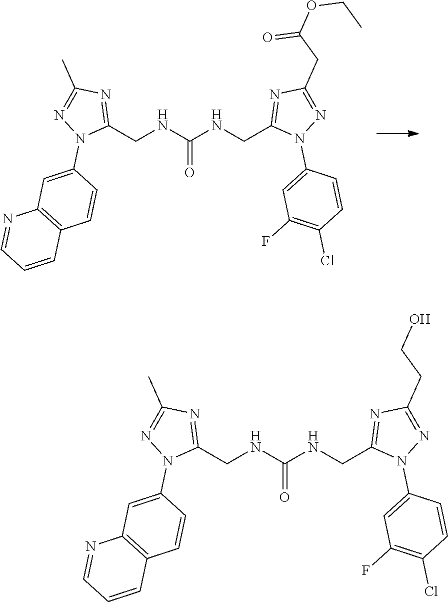 [SMILES: CCOC(=O)Cc1nc(CNC(=O)NCc2nc(C)nn2-c2ccc3cccnc3c2)n(-c2ccc(Cl)c(F)c2)n1 ; Cc1nc(CNC(=O)NCc2nc(CCO)nn2-c2ccc(Cl)c(F)c2)n(-c2ccc3cccnc3c2)n1]

Into a 40-mL vial, was placed ethyl 2-[1-(4-chloro-3-fluorophenyl)-5-{[({[3-methyl-1-(quinolin-7-yl)-1H-1,2,4-triazol-5-yl]methyl}carbamoyl)amino]methyl}-1H-1,2,4-triazol-3-yl]acetate (180.00 mg, 0.31 mmol, 1.00 eq.) in EtOH (5.00 mL) followed by the portion-wise addition of NaBH₄ (47.13 mg, 1.24 mmol, 4.00 eq.) at 0° C. The resulting solution was stirred for 2 h at ambient temperature. The crude product (150 mg) was purified by Prep-HPLC with the following conditions: Column,)(Bridge Prep C18 OBD Column, 19 cm, 150 mm, Sum; mobile phase, Water (0.1% NH₄HCO₃) and CAN (20% Phase B up to 50% in 11 min); Detector, 254 providing 1-{[1-(4-chloro-3-fluorophenyl)-3-(2-hydroxyethyl)-1H-1,2,4-triazol-5-yl]methyl}-3-{[3-methyl-1-(quinolin-7-yl)-1H-1,2,4-triazol-5-yl]methyl}urea (71.1 mg; 42.60%) as a white solid. 1H NMR (300 MHz, DMSO-d6): δ 8.99 (dd, J=4.2, 1.7 Hz, 1H), 8.49-8.45 (m, 1H), 8.24-8.11 (m, 2H), 7.81 (dd, J=8.7, 2.2 Hz, 1H), 7.81-7.69 (m, 2H), 7.63 (dd, J=8.3, 4.2 Hz, 1H), 7.49-7.45 (m, 1H), 6.78-6.72 (m, 2H), 4.69 (d, J=4.6 Hz, 1H), 4.46 (d, J=5.5 Hz, 2H), 4.37 (d, J=5.6 Hz, 2H), 3.78-3.72 (m, 2H), 2.79 (t, J=7.0 Hz, 2H), 2.34 (s, 3H). LCMS: (ES, m/z): [M+H]⁺: 536.

### Example 1.111

### Synthesis of methyl 2-chloro-5-(5-{[({[1-(4-chloro-3-fluorophenyl)-3-methyl-1H-1,2,4-triazol-5-yl]methyl}carbamoyl)amino]methyl}-3-methyl-1H-1,2,4-triazol-1-yl)benzoate (Compound 95)

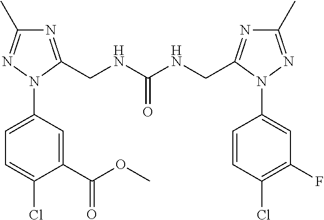 [SMILES: COC(=O)c1cc(-n2nc(C)nc2CNC(=O)NCc2nc(C)nn2-c2ccc(Cl)c(F)c2)ccc1Cl]

The title compound was synthesized according to General Procedure 7; Method A using 1-[1-(4-chloro-3-fluorophenyl)-3-methyl-1H-1,2,4-triazol-5-yl]methanamine (Intermediate I-6; 197.00 mg, 0.819 mmol, 1.00 eq.) and methyl 5-[5-(aminomethyl)-3-methyl-1H-1,2,4-triazol-1-yl]-2-chlorobenzoate (230.00 mg, 0.819 mmol, 1.00 eq.; synthesized according to General Procedure 1 and 5 starting from commercial methyl 5-amino-2-chlorobenzoate) providing methyl 2-chloro-5-(5-{[({[1-(4-chloro-3-fluorophenyl)-3-methyl-1H-1,2,4-triazol-5-yl]methyl}carbamoyl)amino]methyl}-3-methyl-1H-1,2,4-triazol-1-yl)benzoate (187 mg, 41.70%) as a white solid. 1H NMR (300 MHz, DMSO-d6): δ 7.98 (d, J=2.6 Hz, 1H), 7.86-7.70 (m, 4H), 7.47 (ddd, J=8.8, 2.4, 1.2 Hz, 1H), 6.97-6.50 (m, 2H), 4.35 (d, J=5.5 Hz, 4H), 3.87 (s, 3H), 2.30 (d, 6H). LCMS (ES) [M+1]⁺ m/z: 547.

### Example 1.112

### Synthesis of 1-({1-[4-chloro-3-(hydroxymethyl)phenyl]-3-methyl-1H-1,2,4-triazol-5-yl}methyl)-3-{[1-(4-chloro-3-fluorophenyl)-3-methyl-1H-1,2,4-triazol-5-yl]methyl}urea (Compound 96)

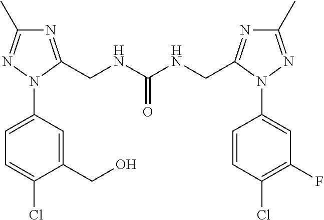 [SMILES: Cc1nc(CNC(=O)NCc2nc(C)nn2-c2ccc(Cl)c(CO)c2)n(-c2ccc(Cl)c(F)c2)n1]

To a stirred solution of methyl 2-chloro-5-(5-{[({[1-(4-chloro-3-fluorophenyl)-3-methyl-1H-1,2,4-triazol-5-yl]methyl carbamoyl)amino]methyl}-3-methyl-1H-1,2,4-triazol-1-yl)benzoate (Example 1.111 used to prepare Compound 95; 185 mg, 0.338 mmol, 1.00 eq.) in THF (2.00 mL) were added 1N LiAlH₄-THF (1 mL, 3 eq.) drop-wise at 0° C. under a nitrogen atmosphere. The resulting mixture was stirred for 1 h at 0° C. under a nitrogen atmosphere. The reaction was then quenched with Na₂SO₄·10H₂O at 0° C., allowed to warm to ambient temperature and filtered. The filter cake was washed with MeOH (1×20 mL) and the filtrate was concentrated under reduced pressure. The crude product was purified by Prep-HPLC to afford 1-({1-[4-chloro-3-(hydroxymethyl)phenyl]-3-methyl-1H-1,2,4-triazol-5-yl}methyl)-3-{[1-(4-chloro-3-fluorophenyl)-3-methyl-1H-1,2,4-triazol-5-yl]methyl}urea (80 mg, 45.58%) as a white solid. 1H NMR (300 MHz, DMSO-d6) δ 7.82-7.71 (m, 2H), 7.67 (d, J=2.5 Hz, 1H), 7.57 (d, J=8.4 Hz, 1H), 7.53-7.43 (m, 2H), 6.78-6.66 (m, 2H), 5.56 (t, J=5.7 Hz, 1H), 4.60 (d, J=5.7 Hz, 2H), 4.37 (d, J=5.7 Hz, 2H), 4.34 (d, J=5.6 Hz, 2H), 2.30 (d, J=1.0 Hz, 6H). LCMS (ES) [M+1]⁺ m/z: 519.

### Example 1.113

### Synthesis of 1-{[1-(4-chloro-3-fluorophenyl)-3-(2-methoxyethyl)-1H-1,2,4-triazol-5-yl]methyl}-3-{[1-(quinolin-7-yl)-1H-1,2,4-triazol-5-yl]methyl}urea (Compound 97)

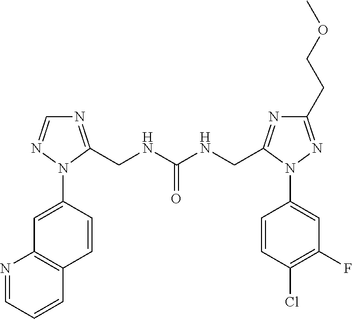 [SMILES: COCCc1nc(CNC(=O)NCc2ncnn2-c2ccc3cccnc3c2)n(-c2ccc(Cl)c(F)c2)n1]

The title compound was synthesized according to General Procedure 7; Method A using 1-[2-(4-chloro-3-fluorophenyl)-5-(2-methoxyethyl)-1,2,4-triazol-3-yl]methanamine (200.00 mg, 0.702 mmol, 1.00 eq.; synthesized according to the procedure described in Example 1.110 used to prepare Compound 94, steps 1-3 but substituting ethyl 3-ethoxy-3-iminopropanoate hydrochloride with 3-methoxypropanimidamide) and 1-[1-(quinolin-7-yl)-1H-1,2,4-triazol-5-yl]methanamine (Intermediate I-9; 158.23 mg, 0.702 mmol, 1 eq.) providing 1-{[1-(4-chloro-3-fluorophenyl)-3-(2-methoxyethyl)-1H-1,2,4-triazol-5-yl]methyl}-3-{[1-(quinolin-7-yl)-1H-1,2,4-triazol-5-yl]methyl}urea (120 mg, 31.87%) as a white solid. ¹H NMR (300 MHz, DMSO-d6) δ 9.01 (dd, J=4.2, 1.7 Hz, 1H), 8.50 (dd, J=8.8, 1.7 Hz, 1H), 8.25 (d, J=2.1 Hz, 1H), 8.19 (d, J=8.2 Hz, 2H), 7.83 (dd, J=8.7, 2.2 Hz, 1H), 7.80-7.69 (m, 2H), 7.65 (dd, J=8.3, 4.2 Hz, 1H), 7.47 (ddd, J=8.7, 2.5, 1.1 Hz, 1H), 6.77 (q, J=5.8 Hz, 2H), 4.50 (d, J=5.5 Hz, 2H), 4.37 (d, J=5.6 Hz, 2H), 3.69 (t, J=6.8 Hz, 2H), 2.87 (t, J=6.9 Hz, 2H). LCMS: (ES, m/z): [M+H]⁺: 536

### Example 1.114

### Synthesis of 3-{[4-(4-chloro-3-fluorophenyl)-5-oxo-4,5-dihydro-1H-1,2,4-triazol-3-yl]methyl}-1-{[1-(quinolin-7-yl)-1H-1,2,4-triazol-5-yl]methyl}urea (Compound 98)

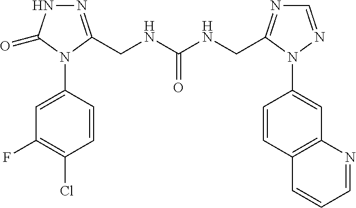 [SMILES: O=C(NCc1ncnn1-c1ccc2cccnc2c1)NCc1n[nH]c(=O)n1-c1ccc(Cl)c(F)c1]

The title compound was synthesized according to General Procedure 7; Method A 3-(aminomethyl)-4-(4-chloro-3-fluorophenyl)-4,5-dihydro-1H-1,2,4-triazol-5-one (Intermediate I-7; 107.72 mg, 0.444 mmol, 1.00 eq.) and 1-[1-(quinolin-7-yl)-1H-1,2,4-triazol-5-yl]methanamine (Intermediate I-9; 100.00 mg, 0.444 mmol, 1.00 eq.) providing 3-{[4-(4-chloro-3-fluorophenyl)-5-oxo-4,5-dihydro-1H-1,2,4-triazol-3-yl]methyl}-1-{[1-(quinolin-7-yl)-1H-1,2,4-triazol-5-yl]methyl}urea (36 mg, 16.42%) as a white solid. 1-H NMR (300 MHz, DMSO-d6) δ 11.84 (br, 1H), 9.02 (dd, J=4.2, 1.8 Hz, 1H), 8.51 (d, J=7.8 Hz, 1H), 8.27-8.15 (m, 3H), 7.83 (dd, J=8.7, 2.2 Hz, 1H), 7.74-7.61 (m, 2H), 7.57 (dd, J=10.1, 2.3 Hz, 1H), 7.29 (dt, J=8.6, 1.7 Hz, 1H), 6.66 (t, J=5.6 Hz, 1H), 6.44 (t, J=5.7 Hz, 1H), 4.45 (d, J=5.4 Hz, 2H), 4.06 (d, J=5.4 Hz, 2H). LCMS: (ES, m/z): [M+H]⁺: 494.1.

### Example 1.115

### Synthesis of 1-[(1-{4-chloro-3-[(methylamino)methyl]phenyl}-3-methyl-1H-1,2,4-triazol-5-yl)methyl]-3-{[1-(4-chloro-3-fluorophenyl)-3-methyl-1H-1,2,4-triazol-5-yl]methyl}urea (Compound 99)

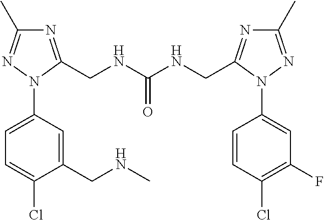 [SMILES: CNCc1cc(-n2nc(C)nc2CNC(=O)NCc2nc(C)nn2-c2ccc(Cl)c(F)c2)ccc1Cl]

Step 1

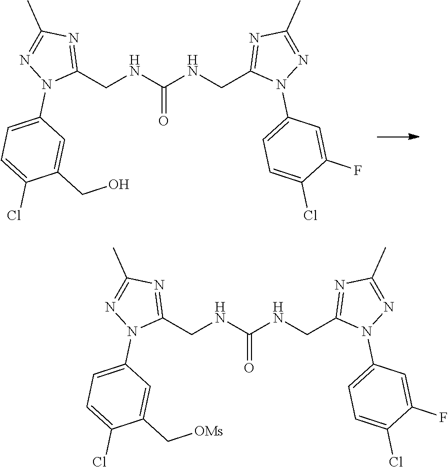 [SMILES: Cc1nc(CNC(=O)NCc2nc(C)nn2-c2ccc(Cl)c(CO)c2)n(-c2ccc(Cl)c(F)c2)n1 ; Cc1nc(CNC(=O)NCc2nc(C)nn2-c2ccc(Cl)c(COS(C)(=O)=O)c2)n(-c2ccc(Cl)c(F)c2)n1]

To a stirred mixture of 1-({1-[4-chloro-3-(hydroxymethyl)phenyl]-3-methyl-1H-1,2,4-triazol-5-yl}methyl)-3-{[1-(4-chloro-3-fluorophenyl)-3-methyl-1H-1,2,4-triazol-5-yl]methyl}urea (Example 1.112 used to prepare Compound 96; 70.00 mg, 0.135 mmol, 1.00 eq.) and Et₃N (40.92 mg, 0.404 mmol, 3 eq.) in DCM (2.00 mL) was added MsCl (30.88 mg, 0.270 mmol, 2 equiv) drop-wise at 0° C. under air atmosphere. The resulting mixture was stirred for 2 h at ambient temperature under air atmosphere. The reaction was then quenched with water at ambient temperature and the resulting mixture was extracted with DCM (1×20 mL). The combined organic layers were washed with brine (1×10 mL), dried over anhydrous Na₂SO₄, filtered and concentrated under reduced pressure. The crude product [2-chloro-5-(5-[[([[2-(4-chloro-3-fluorophenyl)-5-methyl-1,2,4-triazol-3-yl]methyl]carbamoyl)amino]methyl]-3-methyl-1,2,4-triazol-1-yl)phenyl]methyl methanesulfonate (60 mg, 74.51%) was used in the next step directly without further purification. LCMS (ES) [M+1]⁺ m/z: 597.

Step 2

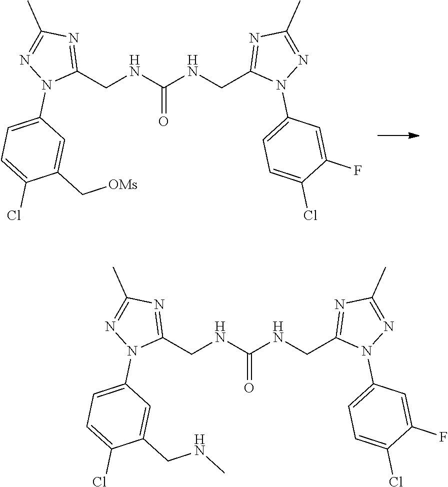 [SMILES: Cc1nc(CNC(=O)NCc2nc(C)nn2-c2ccc(Cl)c(COS(C)(=O)=O)c2)n(-c2ccc(Cl)c(F)c2)n1 ; CNCc1cc(-n2nc(C)nc2CNC(=O)NCc2nc(C)nn2-c2ccc(Cl)c(F)c2)ccc1Cl]

A solution of [2-chloro-5-(5-[[([[2-(4-chloro-3-fluorophenyl)-5-methyl-1,2,4-triazol-3-yl]methyl]carbamoyl)amino]methyl]-3-methyl-1,2,4-triazol-1-yl)phenyl]methyl methanesulfonate (70.00 mg, 0.117 mmol, 1.00 equiv) in methylamine-THF (1 mL) was stirred for 1 h at ambient temperature under air atmosphere. The reaction mixture was then concentrated under reduced pressure and the crude product was purified by Prep-HPLC to afford 1-[(1-{4-chloro-3-[(methylamino)methyl]phenyl}-3-methyl-1H-1,2,4-triazol-5-yl)methyl]-3-{[1-(4-chloro-3-fluorophenyl)-3-methyl-1H-1,2,4-triazol-5-yl]methyl}urea (28 mg, 44.89%) as a white solid. 1H NMR (300 MHz, DMSO-d6) δ 7.97-7.70 (m, 2H), 7.65 (d, J=2.6 Hz, 1H), 7.57 (d, J=8.5 Hz, 1H), 7.52-7.44 (m, 2H), 6.90-6.58 (m, 2H), 4.37 (d, J=5.5 Hz, 2H), 4.34 (d, J=5.6 Hz, 2H), 3.75 (s, 2H), 2.30 (s, 9H). LCMS (ES) [M+1]⁺ m/z: 532.

### Example 1.116

### Synthesis of 1-{[1-(4-chloro-3-fluorophenyl)-3-(2-methoxyethyl)-1H-1,2,4-triazol-5-yl]methyl}-3-{[1-(quinolin-7-yl)-1H-1,2,4-triazol-5-yl]methyl}urea (Compound 100)

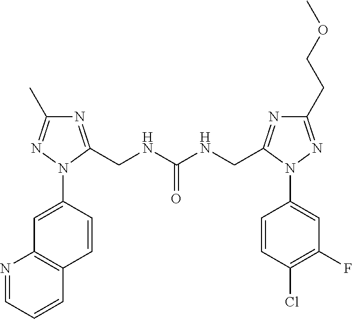 [SMILES: COCCc1nc(CNC(=O)NCc2nc(C)nn2-c2ccc3cccnc3c2)n(-c2ccc(Cl)c(F)c2)n1]

The title compound was synthesized according to General Procedure 7; Method A using 1-[2-(4-chloro-3-fluorophenyl)-5-(2-methoxyethyl)-1,2,4-triazol-3-yl]methanamine (118.80 mg, 0.417 mmol, 1.00 eq.; synthesized according to the procedure described in Example 1.110 used to prepare Compound 94, steps 1-3 but substituting ethyl 3-ethoxy-3-iminopropanoate hydrochloride with 3-methoxypropanimidamide) and 1-[3-methyl-1-(quinolin-7-yl)-1H-1,2,4-triazol-5-yl]methanamine (Intermediate I-10; 99.84 mg, 0.417 mmol, 1.00 eq.) providing 1-{[1-(4-chloro-3-fluorophenyl)-3-(2-methoxy ethyl)-1H-1,2,4-triazol-5-yl]methyl}-3-{[1-(quinolin-7-yl)-1H-1,2,4-triazol-5-yl]methyl}urea (85.9 mg, 37.43%) as a white solid. 1-H NMR (300 MHz, DMSO-d6) δ 9.00 (dd, J=4.2, 1.7 Hz, 1H), 8.48 (dd, J=8.1, 1.7 Hz, 1H), 8.24-8.12 (m, 2H), 7.86-7.70 (m, 3H), 7.63 (dd, J=8.3, 4.2 Hz, 1H), 7.47 (ddd, J=8.7, 2.4, 1.1 Hz, 1H), 6.76 (t, J=5.4 Hz, 1H), 6.74 (t, J=5.4 Hz, 1H), 4.46 (d, J=5.5 Hz, 2H), 4.38 (d, J=5.6 Hz, 2H), 3.69 (t, J=6.8 Hz, 2H), 3.24 (s, 3H), 2.88 (t, J=6.8 Hz, 2H), 2.35 (s, 3H). LCMS: (ES, m/z): [M+H]⁺: 550.1.

### Example 1.117

### Synthesis of 1-{[1-(4-chloro-3-fluorophenyl)-3-methyl-1H-1,2,4-triazol-5-yl]methyl}-3-{[1-(8-fluoroquinoxalin-6-yl)-1H-1,2,4-triazol-5-yl]methyl}urea (Compound 101)

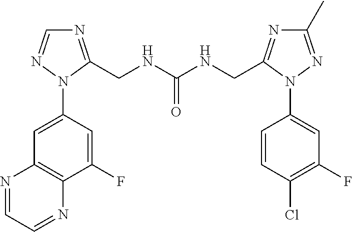 [SMILES: Cc1nc(CNC(=O)NCc2ncnn2-c2cc(F)c3nccnc3c2)n(-c2ccc(Cl)c(F)c2)n1]

Step 1

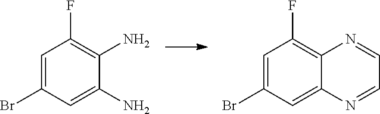 [SMILES: Nc1cc(Br)cc(F)c1N ; Fc1cc(Br)cc2nccnc12]

Into a 100-mL round-bottom flask, was placed 5-bromo-3-fluorobenzene-1,2-diamine (6.5 g, 31.712 mmol, 1.00 eq.), ethyl alcohol (20.00 mL), water (4.00 mL), NaHCO₃(2.66 g, 31.712 mmol, 1.00 eq.), and glyoxal (1.84 g, 31.712 mmol, 1.00 eq.) and the resulting solution was stirred for 16 h at 80° C. The reaction mixture was then concentrated, and the residue was washed with 1×50 ml of water. The solids were collected by filtration providing 4.5 g (62.50%) of 7-bromo-5-fluoroquinoxaline as a red solid. LCMS (ES) [M+1]⁺ m/z 227.

Step 2

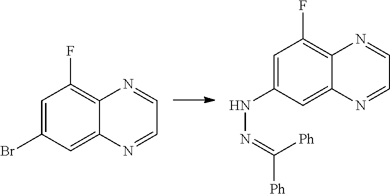 [SMILES: Fc1cc(Br)cc2nccnc12 ; Fc1cc(NN=C(c2ccccc2)c2ccccc2)cc2nccnc12]

Into a 100-mL round-bottom flask, was placed 7-bromo-5-fluoroquinoxaline (2.27 g, 9.998 mmol, 1.00 eq.), toluene (50.00 mL), (diphenylmethylidene)hydrazine (3.92 g, 19.997 mmol, 2.00 eq.), tert-butoxysodium (1.92 g, 19.997 mmol, 2.00 eq.), BINAP (1.25 g, 2.000 mmol, 0.20 eq.), and Pd(AcO)₂ (0.22 g, 1.000 mmol, 0.10 eq.). The resulting solution was stirred for 3 h at 100° C. The reaction mixture was then cooled to ambient temperature and concentrated. The residue was applied onto a silica gel column and eluted with 100% petroleum ether to 35% ethyl acetate in petroleum ether providing 2.7 g (78.87%) of 7-[2-(diphenylmethylidene)hydrazin-1-yl]-5-fluoroquinoxaline as a red solid. LCMS (ES) [M+1]⁺ m/z 343.

Step 3

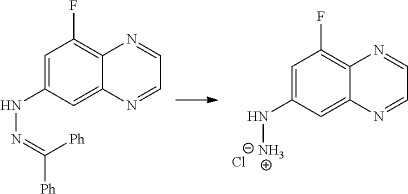 [SMILES: Fc1cc(NN=C(c2ccccc2)c2ccccc2)cc2nccnc12 ; [Cl-] ; [NH3+]Nc1cc(F)c2nccnc2c1]

Into a 100-mL round-bottom flask, was placed 7-[2-(diphenylmethylidene)hydrazin-1-yl]-5-fluoroquinoxaline (2.50 g, 7.302 mmol, 1 eq.), and HCl(gas)in 1,4-dioxane (50.00 mL, 4M) and the resulting solution was stirred for 16 h at 25° C. The solids thus generated were collected by filtration providing 1.2 g (76.57%) of 5-fluoro-7-hydrazinylquinoxaline hydrochloride as a red solid. LCMS (ES) [M+1-HCl]⁺ m/z 179.

Step 4

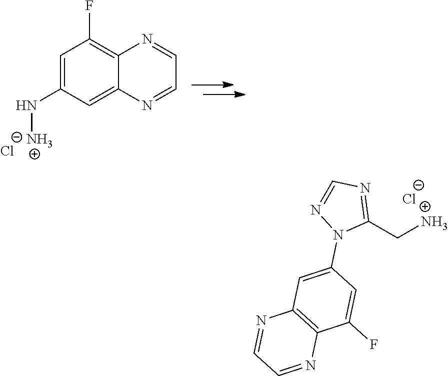 [SMILES: [NH3+]Nc1cc(F)c2nccnc2c1 ; [Cl-] ; [NH3+]Cc1ncnn1-c1cc(F)c2nccnc2c1 ; [Cl-]]

The indicated intermediate compound was synthesized according to General Procedures 2 and 3 using 5-fluoro-7-hydrazinylquinoxaline hydrochloride (1.00 g) providing [1-(8-fluoroquinoxalin-6-yl)-1H-1,2,4-triazol-5-yl]methanaminium chloride (520 mg). LCMS (ES) [M+1]⁺ m/z 245.

Step 5

The title compound was synthesized according to General Procedure 7; Method A using [1-(8-fluoroquinoxalin-6-yl)-1H-1,2,4-triazol-5-yl]methanaminium chloride (From step 4 above; 80.00 mg, 0.328 mmol, 1.00 eq.) and using 1-[1-(4-chloro-3-fluorophenyl)-3-methyl-1H-1,2,4-triazol-5-yl]methanamine (Intermediate I-6; 78.83 mg, 0.328 mmol, 1.00 eq.) providing 1-{[1-(4-chloro-3-fluorophenyl)-3-methyl-1H-1,2,4-triazol-5-yl]methyl}-3-{[1-(8-fluoroquinoxalin-6-yl)-1H-1,2,4-triazol-5-yl]methyl}urea (73.7 mg, 44.04%) as a white solid. 1H NMR (300 MHz, DMSO-d6) δ 9.13 (s, 1H), 9.12 (s, 1H), 8.29-8.18 (m, 2H), 8.05 (dd, J=10.7, 2.2 Hz, 1H), 7.81-7.69 (m, 2H), 7.46 (ddd, J=8.8, 2.5, 1.2 Hz, 1H), 6.73 (t, J=5.8 Hz, 1H), 6.72 (t, J=5.8 Hz, 1H) 4.54 (d, J=5.4 Hz, 2H), 4.32 (, J=5.7 Hz, 2H), 2.28 (s, 3H). LCMS: (ES, m/z): [M+H]⁺: 511.

### Example 1.118

### Synthesis of 1-{[1-(4-chloro-3-fluorophenyl)-3-(2-methoxyethyl)-1H-1,2,4-triazol-5-yl]methyl}-3-{[1-(quinolin-7-yl)-1H-1,2,4-triazol-5-yl]methyl}urea (Compound 102)

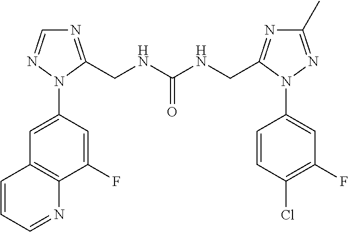 [SMILES: Cc1nc(CNC(=O)NCc2ncnn2-c2cc(F)c3ncccc3c2)n(-c2ccc(Cl)c(F)c2)n1]

Step 1

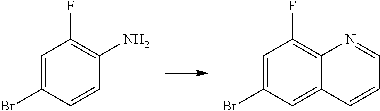 [SMILES: Nc1ccc(Br)cc1F ; Fc1cc(Br)cc2cccnc12]

To a stirred solution of 4-bromo-2-fluoroaniline (8.00 g, 42.102 mmol, 1.00 eq.) and 4-nitrobenzenesulfonic acid (14.28 g, 70.286 mmol, 1.67 eq.) in glycerol (8.53 g, 0.093 mmol, 2.2 eq.) was added 70% H₂SO₄ (40.00 mL, 750.423 mmol) drop-wise at ambient temperature under air atmosphere. The resulting mixture was stirred for 16 h at 135° C. under air atmosphere. The mixture was then allowed to cool to ambient temperature, it was basified to pH 10 with 50% NaOH at 0° C., filtered, and the filter cake was washed with EtOAc. The filtrate was extracted with EtOAc (2×100 mL) and the combined organic layers were washed with brine (1×100 mL), dried over anhydrous Na₂SO₄, filtered and concentrated under reduced pressure. The crude product 6-bromo-8-fluoroquinoline (7.5 g, 78.81%) was used in the next step directly without further purification. LCMS (ES) [M+1]⁺ m/z: 226.

Step 2

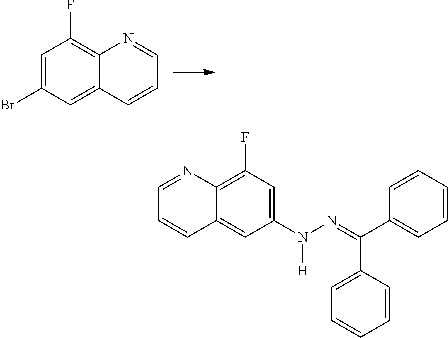 [SMILES: Fc1cc(Br)cc2cccnc12 ; Fc1cc(NN=C(c2ccccc2)c2ccccc2)cc2cccnc12]

To a solution of 6-bromo-8-fluoroquinoline (6.00 g, 26.543 mmol, 1.00 eq.) and (diphenylmethylidene)hydrazine (10.42 g, 53.095 mmol, 2.00 eq.) in toluene (120 mL) were added t-BuONa (5.10 g, 53.086 mmol, 2 eq.), X-Phos (2.53 g, 5.309 mmol, 0.20 eq.) and Pd(OAc)₂ (0.60 g, 2.654 mmol, 0.10 eq.). After stirring for 4 h at 100° C. under a nitrogen atmosphere, the mixture was cooled to ambient temperature and concentrated under reduced pressure. The residue was purified by silica gel column chromatography, eluted with 100% PE to 50% EtOAc in PE to afford 6-[2-(diphenylmethylidene)hydrazin-1-yl]-8-fluoroquinoline (6 g, 66.21%) as a yellow solid. LCMS (ES) [M+1]⁺ m/z: 342.

Step 3

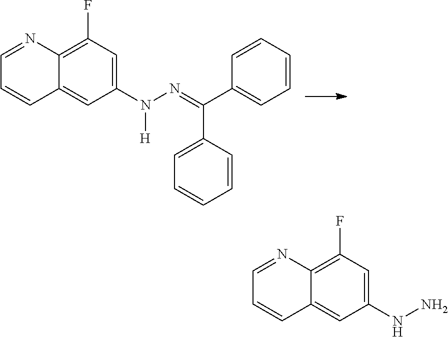 [SMILES: Fc1cc(NN=C(c2ccccc2)c2ccccc2)cc2cccnc12 ; NNc1cc(F)c2ncccc2c1]

A solution of 6-[2-(diphenylmethylidene)hydrazin-1-yl]-8-fluoroquinoline (6.00 g, 17.575 mmol, 1.00 eq.) in HCl (60.00 mL, 1974.713 mmol, 112.36 eq.) and EtOH (12.00 mL, 206.562 mmol, 11.75 eq.) was stirred for 4 h at 80° C. under nitrogen atmosphere. The mixture was then allowed to cool to ambient temperature, it was basified to pH 10 with saturated NaOH (aq.) at 0° C. and it was extracted with CH₂C₁₂/MeOH (10:1) (3×100 mL). The combined organic layers were washed with brine (1×100 mL), dried over anhydrous Na₂SO₄, filtered and concentrated under reduced pressure. The crude product was re-crystallized from EtOAc (30 mL) to afford 8-fluoro-6-hydrazinylquinoline (3.2 g, 102.76%) as a yellow solid. LCMS (ES) [M+1]⁺ m/z: 178.

Step 4

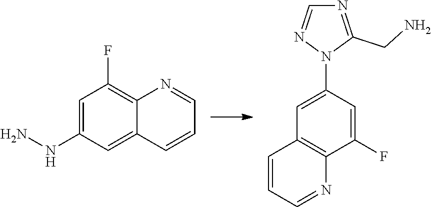 [SMILES: NNc1cc(F)c2ncccc2c1 ; NCc1ncnn1-c1cc(F)c2ncccc2c1]

The indicated intermediate compound was synthesized according to General Procedures 2 and 3 using 8-fluoro-6-hydrazinylquinoline (1.88 g) providing 1-[2-(8-fluoroquinolin-6-yl)-1,2,4-triazol-3-yl]methanamine (583 mg). LCMS (ES) [M+1]⁺ m/z 244.

Step 5

The title compound was synthesized according to General Procedure 7; Method A using 1-[2-(8-fluoroquinolin-6-yl)-1,2,4-triazol-3-yl]methanamine (from step 4 above; 220.00 mg, 0.904 mmol, 1.00 eq.) and 1-[1-(4-chloro-3-fluorophenyl)-3-methyl-1H-1,2,4-triazol-5-yl]methanamine (Intermediate I-6; 217.67 mg, 0.904 mmol, 1 eq.) providing 1-{[1-(4-chloro-3-fluorophenyl)-3-(2-methoxyethyl)-1H-1,2,4-triazol-5-yl]methyl}-3-{[1-(quinolin-7-yl)-1H-1,2,4-triazol-5-yl]methyl}urea (150 mg, 32.53%) as a white solid. ¹H NMR (300 MHz, DMSO-d6): δ 9.05 (dd, J=4.2, 1.6 Hz, 1H), 8.51 (dt, J=8.5, 1.6 Hz, 1H), 8.21-8.08 (m, 2H), 7.93 (dd, J=11.2, 2.2 Hz, 1H), 7.79-7.69 (m, 3H), 7.46 (ddd, J=9.0, 2.4, 1.3 Hz, 1H), 6.77-6.66 (m, 2H), 4.53 (d, J=5.6 Hz, 2H), 4.33 (d, J=5.6 Hz, 2H), 2.28 (s, 3H). LCMS (ES) [M+1]⁺ m/z: 510.

### Example 1.119

### Synthesis of 1-{[1-(4-chloro-3-fluorophenyl)-3-methyl-1H-1,2,4-triazol-5-yl]methyl}-3-{[1-(8-methylquinoxalin-6-yl)-1H-1,2,4-triazol-5-yl]methyl}urea (Compound 103)

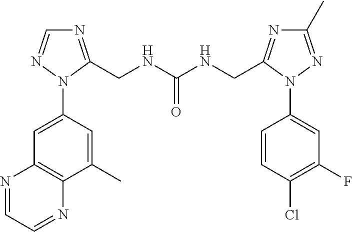 [SMILES: Cc1nc(CNC(=O)NCc2ncnn2-c2cc(C)c3nccnc3c2)n(-c2ccc(Cl)c(F)c2)n1]

Step 1

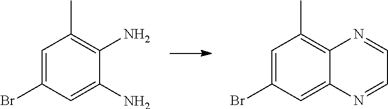

Into a 50-mL round-bottom flask purged and maintained with an inert atmosphere of nitrogen, was placed a solution of 5-bromo-3-methylbenzene-1,2-diamine (600.00 mg, 2.984 mmol, 1.00 eq.) in EtOH (12 mL), glyoxal (173.18 mg, 2.984 mmol, 1 eq.), and NaHCO₃ (501.36 mg, 5.968 mmol, 2.0 eq.) and the resulting solution was stirred for 12 h at 60° C. in an oil bath. The reaction mixture was then diluted with 20 mL of H₂O and extracted with 3×20 mL of ethyl acetate, dried over anhydrous sodium sulfate and concentrated. The residue was applied onto a silica gel column and eluted with 100% petroleum ether to 15% ethyl acetate in petroleum ether providing 390 mg (58.59%) of 7-bromo-5-methylquinoxaline as a light yellow solid. LCMS (ES) [M+1]+m/z: 223.

Step 2

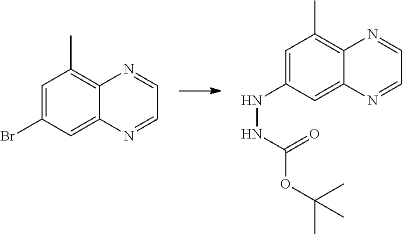 [SMILES: Cc1cc(Br)cc2nccnc12 ; Cc1cc(NNC(=O)OC(C)(C)C)cc2nccnc12]

Into a 25-mL round-bottom flask purged and maintained with an inert atmosphere of nitrogen, was placed a solution of 7-bromo-5-methylquinoxaline (300.00 mg, 1.345 mmol, 1.00 eq.) in dioxane (10 mL), tert-butoxycarbohydrazide (213.29 mg, 1.614 mmol, 1.2 eq.), BrettPhos Pd G3 (121.91 mg, 0.134 mmol, 0.1 eq.), and tBuONa (154.93 mg, 1.614 mmol, 1.2 eq.) and the resulting solution was stirred for 12 h at 100° C. in an oil bath. The reaction mixture was then cooled to ambient temperature, diluted with 20 mL of H₂O, extracted with 3×30 mL of dichloromethane, dried over anhydrous sodium sulfate and concentrated under vacuum. The residue was applied onto a silica gel column and eluted with 100% dichloromethane to 8% methanol in dichloromethane providing 220 mg (59.63%) of N′-(8-methylquinoxalin-6-yl)tert-butoxycarbohydrazide as a light yellow solid. LCMS (ES) [M+1]⁺ m/z: 275.

Step 3

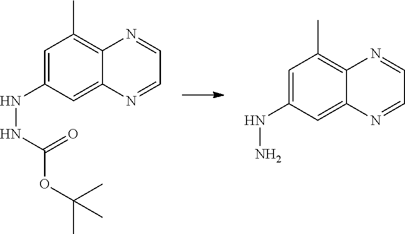 [SMILES: Cc1cc(NNC(=O)OC(C)(C)C)cc2nccnc12 ; Cc1cc(NN)cc2nccnc12]

Into a 25-mL round-bottom flask purged and maintained with an inert atmosphere of nitrogen, was placed a solution of N′-(8-methylquinoxalin-6-yl)tert-butoxycarbohydrazide (200.00 mg, 0.729 mmol, 1.00 eq.) in HCl-EA(2M) (8 mL). The resulting solution was stirred for 2 h at ambient temperature then it was concentrated under vacuum providing 110 mg (86.61%) of 7-hydrazinyl-5-methylquinoxaline as an off-white solid. LCMS (ES) [M+1]⁺ m/z:175.

Step 4

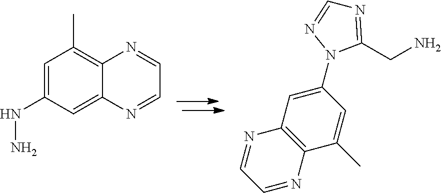 [SMILES: Cc1cc(NN)cc2nccnc12 ; Cc1cc(-n2ncnc2CN)cc2nccnc12]

The indicated intermediate compound was synthesized according to General Procedures 2 and 3 using 7-hydrazinyl-5-methylquinoxaline (400.00 mg) providing 1-[1-(8-methylquinoxalin-6-yl)-1H-1,2,4-triazol-5-yl]methanamine (190 mg). LCMS (ES) [M+1]⁺ m/z 245.

Step 5

Into a 20-mL round-bottom flask purged and maintained with an inert atmosphere of nitrogen, was placed a solution of 1-[1-(8-methylquinoxalin-6-yl)-1H-1,2,4-triazol-5-yl]methanamine (From step 4 above; 200.00 mg, 0.832 mmol, 1.00 eq.) in DCM (10 mL), TEA (252.69 mg, 2.497 mmol, 3 eq.), triphosgene (74.10 mg, 0.250 mmol, 0.3 eq.), 1-[1-(4-chloro-3-fluorophenyl)-3-methyl-1H-1,2,4-triazol-5-yl]methanamine (Intermediate I-6; 200.33 mg, 0.832 mmol, 1 eq.) and the resulting solution was stirred for 2 h at 0° C. in a water/ice bath. The reaction was then quenched by the addition of 20 mL of water/ice and the resulting solution was extracted with 3×20 mL of dichloromethane, dried over anhydrous sodium sulfate and concentrated. The crude product was purified by Flash-Prep-HPLC with the following conditions (IntelFlash-1): Column, C18 silica gel; mobile phase, ACN:H2O (NH3, 0.05%)=15% increasing to ACN:H2O(NH3, 0.05%)=30% within 15; Detector, 254 providing 1-{[1-(4-chloro-3-fluorophenyl)-3-methyl-1H-1,2,4-triazol-5-yl]methyl}-3-{[1-(8-methylquinoxalin-6-yl)-1H-1,2,4-triazol-5-yl]methyl}urea (130 mg, 30.81%) as an off-white solid. ¹H NMR (300 MHz, DMSO-d6) δ 9.05 (s, 2H), 8.20-8.19 (m, 2H), 7.96-7.93 (m, 1H), 7.79-7.68 (m, 2H), 7.46 (d, J=8.0 Hz, 1H), 6.73 (t, J=5.5 Hz, 1H), 6.71 (t, J=5.6 Hz, 1H), 4.53 (d, J=5.5 Hz, 2H), 4.34 (d, J=5.6 Hz, 2H), 2.79 (s, 3H), 2.28 (s, 3H). LCMS (ES) [M+1]⁺ m/z: 507.

### Example 1.120

### Synthesis of 1-{[1-(4-chloro-3-fluorophenyl)-3-(fluoromethyl)-1H-1,2,4-triazol-5-yl]methyl}-3-{[1-(quinolin-7-yl)-1H-1,2,4-triazol-5-yl]methyl}urea (Compound 104)

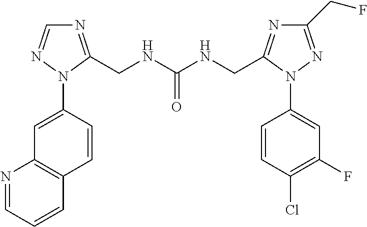 [SMILES: O=C(NCc1ncnn1-c1ccc2cccnc2c1)NCc1nc(CF)nn1-c1ccc(Cl)c(F)c1]

Step 1

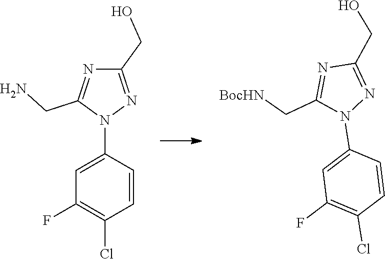 [SMILES: NCc1nc(CO)nn1-c1ccc(Cl)c(F)c1 ; CC(C)(C)OC(=O)NCc1nc(CO)nn1-c1ccc(Cl)c(F)c1]

Into a 250-mL round-bottom flask, was placed [5-(aminomethyl)-1-(4-chloro-3-fluorophenyl)-1,2,4-triazol-3-yl]methanol (2.20 g, 8.571 mmol, 1.00 eq.; synthesized as described in Example 1.109 used to prepare Compound 93, Steps 1-4), DCM (50.00 mL), DMAP (1256.57 mg, 10.286 mmol, 1.20 eq.), and Boc₂O (1870.66 mg, 8.571 mmol, 1.00 eq.) and the resulting solution was stirred for 16 h at 25° C. The reaction mixture was then washed with 2×50 mL of water, the organic layer was separated, dried over anhydrous sodium sulfate and concentrated under vacuum providing 2.8 g (91.56%) of tert-butyl N-[[2-(4-chloro-3-fluorophenyl)-5-(hydroxymethyl)-1,2,4-triazol-3-yl]methyl]carbamate as a light yellow solid, which was used in the following step without further purification. LCMS (ES) [M+1]⁺ m/z: 357.

Step 2

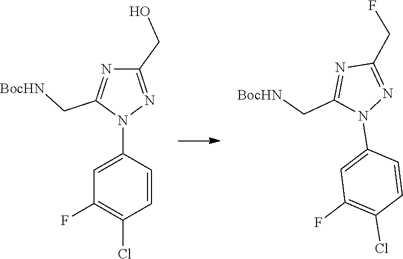 [SMILES: CC(C)(C)OC(=O)NCc1nc(CO)nn1-c1ccc(Cl)c(F)c1 ; CC(C)(C)OC(=O)NCc1nc(CF)nn1-c1ccc(Cl)c(F)c1]

Into a 100-mL round-bottom flask, was placed tert-butyl N-[[2-(4-chloro-3-fluorophenyl)-5-(hydroxymethyl)-1,2,4-triazol-3-yl]methyl]carbamate (1.80 g, 5.045 mmol, 1.00 eq.), DCM (36.00 mL, 423.851 mmol, 112.24 eq.) followed by the drop-wise addition of DAST (0.81 g, 5.045 mmol, 1.00 eq.) at −10° C. and the resulting solution was stirred for 1 h at 0° C. The reaction was then quenched by the addition of 20 mL of water and the organic layer was separated, dried over anhydrous sodium sulfate and concentrated under vacuum. The residue was applied onto a silica gel column and eluted with ethyl acetate/petroleum ether (1:1) providing 951.8 mg (52.58%) of tert-butyl ((1-(4-chloro-3-fluorophenyl)-3-(fluoromethyl)-1H-1,2,4-triazol-5-yl)methyl)carbamate as a white solid. LCMS (ES) [M+1]⁺ m/z: 359.

Step 3

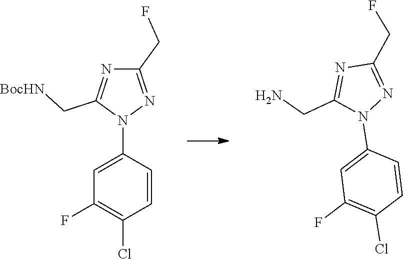 [SMILES: CC(C)(C)OC(=O)NCc1nc(CF)nn1-c1ccc(Cl)c(F)c1 ; NCc1nc(CF)nn1-c1ccc(Cl)c(F)c1]

Into a 50-mL round-bottom flask, was placed tert-butyl N-[[2-(4-chloro-3-fluorophenyl)-5-(fluoromethyl)-1,2,4-triazol-3-yl]methyl]carbamate (200.00 mg, 0.557 mmol, 1.00 eq.), HCl/dioxane (10.00 mL, 4M) and the resulting solution was stirred for 1 h at 50° C. The reaction mixture was then concentrated under vacuum and the residue was purified by preparative HPLC (Prep-C18, 20-45M, 120 g, Tianjin Bonna-Agela Technologies; gradient elution of 20% MeCN in water to 30% MeCN in water over a 10 min period, water contains 0.1% NH₃H₂O) to provide 1-[1-(4-chloro-3-fluorophenyl)-3-(fluoromethyl)-1H-1,2,4-triazol-5-yl]methanamine as a white solid (120 mg, 83.22%). LCMS (ES) [M+1]⁺ m/z 259.

Step 4

The title compound was synthesized according to General Procedure 7; Method A using 1-[1-(4-chloro-3-fluorophenyl)-3-(fluoromethyl)-1H-1,2,4-triazol-5-yl]methanamine (from step 3 above; 60.00 mg, 0.232 mmol, 1.00 eq.) and 1-[1-(quinolin-7-yl)-1H-1,2,4-triazol-5-yl]methanamine (Intermediate I-9; 89.94 mg, 0.696 mmol, 3.00 eq.) providing 1-{[1-(4-chloro-3-fluorophenyl)-3-(fluoromethyl)-1H-1,2,4-triazol-5-yl]methyl}-3-{[1-(quinolin-7-yl)-1H-1,2,4-triazol-5-yl]methyl}urea (44.1 mg, 37.28%) as a white solid. 1H NMR (300 MHz, DMSO-d6): δ 9.01 (dd, J=4.2, 1.7 Hz, 1H), 8.50 (d, J=8.3 Hz, 1H), 8.25 (d, J=2.1 Hz, 1H), 8.19 (d, J=8.8 Hz, 1H), 8.18 (s, 1H), 7.87-7.74 (m, 3H), 7.65 (dd, J=8.3, 4.2 Hz, 1H), 7.51 (ddd, J=8.7, 2.4, 1.3 Hz, 1H), 6.79 (t, J=5.5 Hz, 2H), 5.50 (s, 1H), 5.35 (s, 1H), 4.50 (d, J=5.5 Hz, 2H), 4.40 (d, J=5.6 Hz, 2H).LCMS (ES) [M+1]⁺ m/z: 510.

### Example 1.121

### Synthesis of 1-{[1-(4-chloro-3-fluorophenyl)-3-methyl-1H-1,2,4-triazol-5-yl]methyl}-3-{[1-(5-methylquinazolin-7-yl)-1H-1,2,4-triazol-5-yl]methyl}urea (Compound 105)

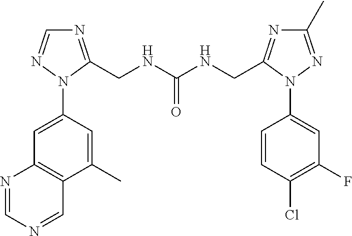 [SMILES: Cc1nc(CNC(=O)NCc2ncnn2-c2cc(C)c3cncnc3c2)n(-c2ccc(Cl)c(F)c2)n1]

The title compound was synthesized according to General Procedure 7; Method A using (1-(5-methylquinazolin-7-yl)-1H-1,2,4-triazol-5-yl)methanamine (140.00 mg, 0.583 mmol, 1.00 eq.; synthesized according to General Procedures 2 and 3 starting from 5-methyl-7-hydrazineylquinazoline synthesized as described in Example 1.124 used to prepare Compound 108, steps 1-6 starting with 3-bromo-5-methylaniline) and 1-[1-(4-chloro-3-fluorophenyl)-3-methyl-1H-1,2,4-triazol-5-yl]methanamine (Intermediate I-6; 154.26 mg, 0.641 mmol, 1.1 eq.) providing 1-{[1-(4-chloro-3-fluorophenyl)-3-methyl-1H-1,2,4-triazol-5-yl]methyl}-3-{[1-(5-methylquinazolin-7-yl)-1H-1,2,4-triazol-5-yl]methyl}urea (51.7 mg, 17.50%) as a white solid. ¹H NMR (300 MHz, DMSO-d6): δ 9.30 (s, 1H), 8.95 (d, J=0.9 Hz, 1H), 8.25 (s, 1H), 7.97 (s, 1H), 7.85-7.67 (m, 3H), 7.44 (ddd, J=8.7, 2.4, 1.1 Hz, 1H), 6.60 (t, J=5.6 Hz, 1H), 6.53 (t, J=5.6 Hz, 1H), 4.33 (d, J=5.6 Hz, 2H), 4.19 (d, J=5.5 Hz, 2H), 2.59 (s, 3H), 2.28 (s, 3H). LCMS (ES) [M+1]⁺ m/z: 507.

### Example 1.122

### Synthesis of 1-{[1-(4-chloro-3-fluorophenyl)-3-methyl-1H-1,2,4-triazol-5-yl]methyl}-3-({6-[(4-fluorophenyl)methyl]-7-oxo-4,6-diazaspiro[2.4]hept-4-en-5-yl}methyl)urea (Compound 106)

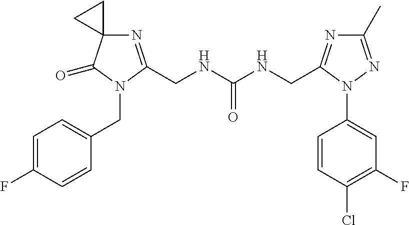 [SMILES: Cc1nc(CNC(=O)NCC2=NC3(CC3)C(=O)N2Cc2ccc(F)cc2)n(-c2ccc(Cl)c(F)c2)n1]

Step 1

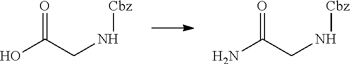 [SMILES: O=C(O)CNC(=O)OCc1ccccc1 ; NC(=O)CNC(=O)OCc1ccccc1]

Into a 3 L 4-necked round-bottom flask purged and maintained with an inert atmosphere of nitrogen, was placed ((benzyloxy)carbonyl)glycine (50.00 g, 239.005 mmol, 1.00 eq.) in THF (1.00 L) followed by the drop-wise addition of TEA (29.02 g, 286.806 mmol, 1.20 eq.) with stirring at 0° C. over 5 min. To this was added ethyl chloroformate (28.53 g, 262.905 mmol, 1.10 eq.) drop-wise with stirring at 0° C. over 15 min. followed by NH₃ (g) (enough) at 0° C. The resulting solution was stirred for 1 h at ambient temperature. The solids thus generated were collected by filtration and the filtrate was concentrated. This resulted in 25 g (50.24%) of benzyl (2-amino-2-oxoethyl)carbamate as a white solid. LCMS (ES) [M+1]⁺ m/z 209.

Step 2

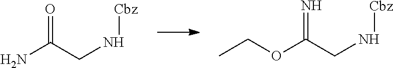 [SMILES: NC(=O)CNC(=O)OCc1ccccc1 ; CCOC(=N)CNC(=O)OCc1ccccc1]

Into a 500-mL round-bottom flask purged and maintained with an inert atmosphere of nitrogen, was placed benzyl (2-amino-2-oxoethyl)carbamate (10.00 g, 48.027 mmol, 1.00 eq.) in DCM (150.00 mL) followed by the addition of tetrafluoroboranuide; triethyloxidanium (27.59 g, 144.080 mmol, 3.00 eq.) in several batches at 0° C. and the resulting solution was stirred for 24 h at ambient temperature. The reaction mixture was then diluted with 200 mL of DCM, washed with 2 ×150 mL of NaHCO₃ and with 2 ×150 mL of brine then it was dried over anhydrous sodium sulfate and concentrated providing 6 g of crude ethyl 2-(((benzyloxy)carbonyl)amino)acetimidate as yellow oil which was used in the following step without further purification. LCMS (ES) [M+1]⁺ m/z 237.

Step 3

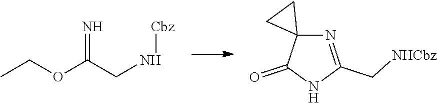 [SMILES: CCOC(=N)CNC(=O)OCc1ccccc1 ; O=C(NCC1=NC2(CC2)C(=O)N1)OCc1ccccc1]

Into a 250-mL round-bottom flask purged and maintained with an inert atmosphere of nitrogen, was placed ethyl 2-(((benzyloxy)carbonyl)amino)acetimidate (6.00 g, 25.395 mmol, 1.00 eq.), toluene (100.00 mL), ethyl 1-aminocyclopropane-1-carboxylate (1.80 g, 13.936 mmol, 0.55 eq.), and DIEA (1.71 g, 13.205 mmol, 0.52 equiv) and the resulting solution was stirred for 8 h at 100° C. in an oil bath. AcOH (0.6 ml) was then added and the resulting solution was stirred for 2 h at 100° C. in an oil bath. The reaction mixture was then cooled to ambient temperature and concentrated. The residue was diluted with 300 mL of DCM, washed with 1×200 mL of H2O 2O and with 1×200 mL of brine. The crude product was purified by Flash-Prep-HPLC providing 0.8 g (11.53%) of benzyl ((7-oxo-4,6-diazaspiro[2.4]hept-4-en-5-yl)methyl)carbamate as a brown solid. LCMS (ES) [M+1]⁺ m/z 274.

Step 4

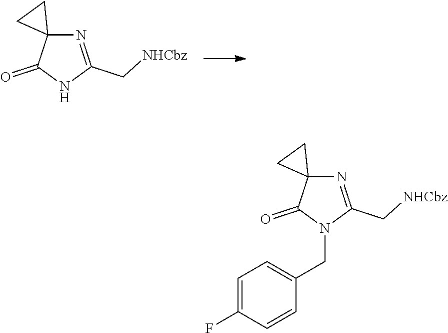 [SMILES: O=C(NCC1=NC2(CC2)C(=O)N1)OCc1ccccc1 ; O=C(NCC1=NC2(CC2)C(=O)N1Cc1ccc(F)cc1)OCc1ccccc1]

Into a 100-mL round-bottom flask purged and maintained with an inert atmosphere of nitrogen, was placed benzyl ((7-oxo-4,6-diazaspiro[2.4]hept-4-en-5-yl)methyl)carbamate (800.00 mg, 2.927 mmol, 1.00 eq.) in DMF (15.00 mL) followed by the addition of K₂CO₃ (809.13 mg, 5.855 mmol, 2.00 eq.) in several batches at ambient temperature. To this was added 1-(bromomethyl)-4-fluorobenzene (719.33 mg, 3.805 mmol, 1.30 eq.) drop-wise with stirring at 0° C. and the resulting solution was stirred overnight at ambient temperature. The reaction mixture was then diluted with 100 mL of H₂O, extracted with 3 ×150 mL of ethyl acetate, washed with 3×200 ml of brine, dried over anhydrous sodium sulfate and concentrated. The residue was applied onto a silica gel column and eluted with 100% PE to 50% THF in PE providing 500 mg (44.78%) of benzyl ((6-(4-fluorobenzyl)-7-oxo-4,6-diazaspiro[2.4]hept-4-en-5-yl)methyl)carbamate as yellow oil. LCMS (ES) [M+1]⁺ m/z 382.

Step 5

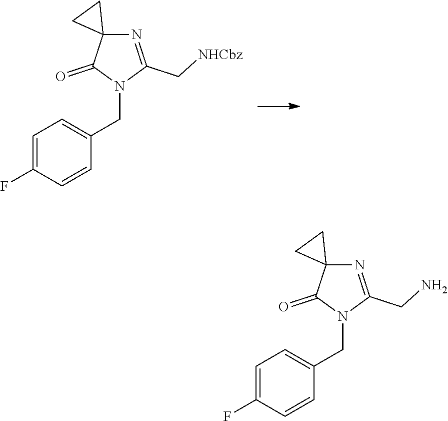 [SMILES: O=C(NCC1=NC2(CC2)C(=O)N1Cc1ccc(F)cc1)OCc1ccccc1 ; NCC1=NC2(CC2)C(=O)N1Cc1ccc(F)cc1]

Into a 100-mL round-bottom flask, was placed benzyl ((6-(4-fluorobenzyl)-7-oxo-4,6-diazaspiro[2.4]hept-4-en-5-yl)methyl)carbamate (0.50 g, 1.311 mmol, 1.00 eq.), and HBr in AcOH (6.00 mL) and the resulting solution was stirred for 40 min at ambient temperature. The pH of the solution was then adjusted to <7 with NaHCO₃(3 mol/L) at 0° C. and the resulting solution was extracted with 5×150 mL of DCM:MeOH=10:1, dried over anhydrous sodium sulfate and concentrated. The residue was applied onto a silica gel column and eluted with 100% dichloromethane to 10% methanol in dichloromethane providing 200 mg (61.70%) of 5-(aminomethyl)-6-(4-fluorobenzyl)-4,6-diazaspiro[2.4]hept-4-en-7-one as a yellow semi-solid. LCMS (ES) [M+1]⁺ m/z 248.

Step 6

The title compound was synthesized according to General Procedure 7; Method A using 1-[1-(4-chloro-3-fluorophenyl)-3-methyl-1H-1,2,4-triazol-5-yl]methanamine (Intermediate I-6; 74.94 mg, 0.311 mmol, 1.10 eq.) and 5-(aminomethyl)-6-(4-fluorobenzyl)-4,6-diazaspiro[2.4]hept-4-en-7-one (from step 5 above; 70.00 mg, 0.283 mmol, 1.00 eq.) providing 1-{[1-(4-chloro-3-fluorophenyl)-3-methyl-1H-1,2,4-triazol-5-yl]methyl}-3-({6-[(4-fluorophenyl)methyl]-7-oxo-4,6-diazaspiro[2.4]hept-4-en-5-yl}methyl)urea (47.6 mg, 32.72%) as a white solid. ¹H NMR (300 MHz, DMSO-d6): δ 7.83-7.72 (m, 2H), 7.48 (ddd, J=8.7, 2.4, 1.2 Hz, 1H), 7.34-7.24 (m, 2H), 7.22-7.11 (m, 2H), 6.76 (t, J=5.7 Hz, 1H), 6.52 (t, J=5.5 Hz, 1H), 4.74 (s, 2H), 4.37 (d, J=5.7 Hz, 2H), 4.06 (d, J=5.5 Hz, 2H), 2.29 (s, 3H), 1.66-1.61 (m, 2H), 1.51-1.45 (m, 2H). LCMS (ES) [M+1]⁺ m/z: 514.1.

### Example 1.123

### Synthesis of 1-{[1-(4-chloro-3-fluorophenyl)-3-(fluoromethyl)-1H-1,2,4-triazol-5-yl]methyl}-3-{[4-(4-chloro-3-fluorophenyl)-5-oxo-4,5-dihydro-1H-1,2,4-triazol-3-yl]methyl}urea (Compound 107)

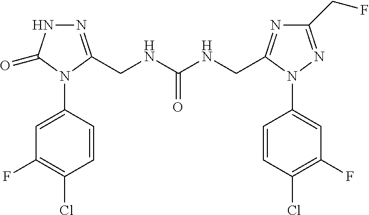 [SMILES: O=C(NCc1nc(CF)nn1-c1ccc(Cl)c(F)c1)NCc1n[nH]c(=O)n1-c1ccc(Cl)c(F)c1]

The title compound was synthesized according to General Procedure 7; Method A using (1-(4-chloro-3-fluorophenyl)-3-(fluoromethyl)-1H-1,2,4-triazol-5-yl)methanamine (from Example 1.120, Steps 1-3; 60.00 mg, 0.232 mmol, 1.00 eq.) and 3-(aminomethyl)-4-(4-chloro-3-fluorophenyl)-4,5-dihydro-1H-1,2,4-triazol-5-one (Intermediate I-7; 56.28 mg, 0.232 mmol, 1.00 eq.) providing 1-{[1-(4-chloro-3-fluorophenyl)-3-(fluoromethyl)-1H-1,2,4-triazol-5-yl]methyl}-3-{[4-(4-chloro-3-fluorophenyl)-5-oxo-4,5-dihydro-1H-1,2,4-triazol-3-yl]methyl}urea (67.2 mg, 54.94%) as a white solid. 1H NMR (300 MHz, DMSO-d6): δ 11.86 (br, 1H), 7.88-7.76 (m, 2H), 7.69 (t, J=8.4 Hz, 1H), 7.58 (dd, J=10.1, 2.3 Hz, 1H), 7.52 (ddd, J=8.7, 2.4, 1.2 Hz, 1H), 7.29 (ddd, J=8.6, 2.4, 1.2 Hz, 1H), 6.65 (t, J=5.6 Hz, 1H), 6.43 (t, J=5.6 Hz, 1H), 5.51 (s, 1H), 5.36 (s, 1H), 4.35 (d, J=5.6 Hz, 2H), 4.07 (d, J=5.5 Hz, 2H). LCMS (ES) [M+1]⁺ m/z: 527.

### Example 1.124

### Synthesis of 1-{[1-(4-chloro-3-fluorophenyl)-3-methyl-1H-1,2,4-triazol-5-yl]methyl}-3-{[1-(5-fluoroquinazolin-7-yl)-1H-1,2,4-triazol-5-yl]methyl}urea (Compound 108)

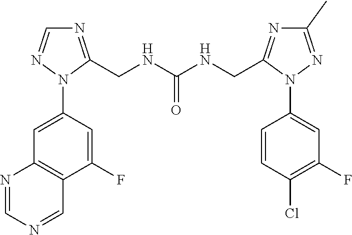 [SMILES: Cc1nc(CNC(=O)NCc2ncnn2-c2cc(F)c3cncnc3c2)n(-c2ccc(Cl)c(F)c2)n1]

Step 1

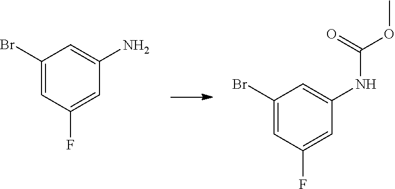

Into a 1-L round-bottom flask, was placed 3-bromo-5-fluoroaniline (21.00 g, 110.518 mmol, 1.00 eq.), dimethyl carbonate (10.95 g, 121.561 mmol, 1.10 eq.), MeONa (11.94 g, 221.035 mmol, 2 eq.), and MeOH (300.00 mL) and the resulting solution was stirred for 16 h at 60° C. The reaction mixture was then cooled to ambient temperature and extracted with 3×300 mL of ethyl acetate and the organic layers were combined and dried over anhydrous sodium sulfate and concentrated under vacuum. The residue was applied onto a silica gel column and eluted with 100% PE to 20% THF in PE providing 18 g (65.66%) of methyl (3-bromo-5-fluorophenyl)carbamate as an off-white solid. LCMS (ES) [M+1]⁺ m/z: 248.

Step 2

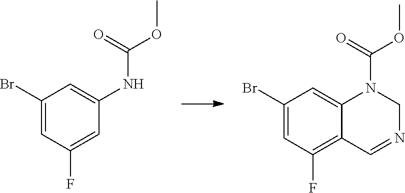 [SMILES: COC(=O)Nc1cc(F)cc(Br)c1 ; COC(=O)N1CN=Cc2c(F)cc(Br)cc21]

Into a 500-mL round-bottom flask, was placed methyl (3-bromo-5-fluorophenyl)carbamate (18.00 g, 72.566 mmol, 1.00 eq.), hexamethylenetetramine (67.05 g, 478.957 mmol, 6.60 eq.), and TFA (200.00 mL) and the resulting solution was stirred for 4 h at 80° C. After cooling, the mixture was then diluted with cold water, neutralized with NaHCO₃ and extracted with EtOAc (3×300 mL). The organic phase was evaporated under reduced pressure. The undissolved solid was separated by filtration and the solution was evaporated under reduced pressure to give 10 g (48.00%) of methyl 7-bromo-5-fluoroquinazoline-1(2H)-carboxylate as an off-white solid. LCMS (ES) [M+1]⁺ m/z: 287.

Step 3

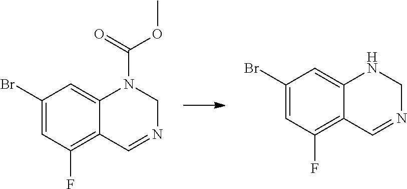 [SMILES: COC(=O)N1CN=Cc2c(F)cc(Br)cc21 ; Fc1cc(Br)cc2c1C=NCN2]

Into a 500-mL round-bottom flask, was placed methyl 7-bromo-5-fluoroquinazoline-1(2H)-carboxylate (10.00 g, 34.833 mmol, 1.00 eq.), 10% KOH (150 mL), EtOH (50.00 mL), and H₂O (50.00 mL) and the resulting solution was stirred for 4 h at 80° C. The reaction mixture was then cooled to ambient temperature and extracted with 3×300 mL of dichloromethane and the organic layers were combined, dried over anhydrous sodium sulfate and concentrated under vacuum. The residue was applied onto a silica gel column and eluted with 100% petroleum ether to 40% THF in petroleum ether providing 6 g (75.20%) of 7-bromo-5-fluoro-1,2-dihydroquinazoline as colorless oil. LCMS (ES) [M+1]⁺ m/z: 229.

Step 4

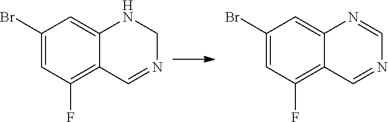 [SMILES: Fc1cc(Br)cc2c1C=NCN2 ; Fc1cc(Br)cc2ncncc12]

Into a 250-mL round-bottom flask, was placed 7-bromo-5-fluoro-1,2-dihydroquinazoline (6.00 g, 26.195 mmol, 1.00 eq.), DDQ (11.89 g, 52.378 mmol, 2.00 eq.), and DCE (40.00 mL) and the resulting solution was stirred for 8 h at 90° C. The reaction mixture was then cooled to ambient temperature and extracted with 3×60 mL of dichloromethane and the organic layers were combined, dried over anhydrous sodium sulfate and concentrated under vacuum. The residue was applied onto a silica gel column and eluted with 100% petroleum ether to 40% THF in petroleum ether providing 1.7 g (28.58%) of 7-bromo-5-fluoroquinazoline as colorless oil. LCMS (ES) [M+1]⁺ m/z: 227.

Step 5

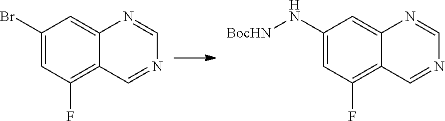 [SMILES: Fc1cc(Br)cc2ncncc12 ; CC(C)(C)OC(=O)NNc1cc(F)c2cncnc2c1]

Into a 100-mL round-bottom flask, was placed 7-bromo-5-fluoroquinazoline (1.70 g, 7.488 mmol, 1.00 eq.), tert-butoxycarbohydrazide (1.19 g, 9.004 mmol, 1.20 eq.), Pd₂(dba)₃ CHCl₃ (0.77 g, 0.749 mmol, 0.1 eq.), BINAP (0.23 g, 0.374 mmol, 0.05 eq.), Cs₂CO₃ (4.88 g, 14.976 mmol, 2 eq.), and DMF (30.00 mL) and the resulting solution was stirred for 16 h at 100° C. The reaction mixture was then cooled to ambient temperature and extracted with 3×30 mL of dichloromethane and the organic layers were combined, dried over anhydrous sodium sulfate and concentrated under vacuum. The residue was applied onto a silica gel column and eluted with 100% petroleum ether to 25% ethyl acetate in petroleum ether providing 1.1 g (52.79%) of tert-butyl 2-(5-fluoroquinazolin-7-yl)hydrazine-1-carboxylate as an off-white solid. LCMS (ES) [M+1]⁺ m/z: 279.

Step 6

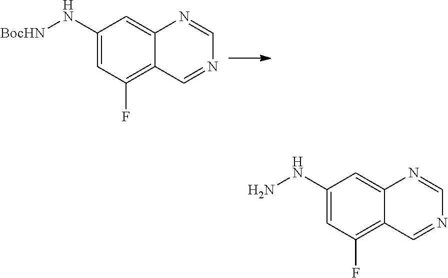 [SMILES: CC(C)(C)OC(=O)NNc1cc(F)c2cncnc2c1 ; NNc1cc(F)c2cncnc2c1]

Into a 100-mL round-bottom flask, was placed tert-butyl 2-(5-fluoroquinazolin-7-yl)hydrazine-1-carboxylate (1.00 g, 3.593 mmol, 1.00 eq.) followed by the addition of HCl(g)in EA (20.00 mL, 350.350 mmol, 97.50 eq.) and the resulting solution was stirred for 24 h at ambient temperature. The solids thus formed were collected by filtration providing 500 mg (78.10%) of 5-fluoro-7-hydrazineylquinazoline as an off-white solid. LCMS (ES) [M+1]⁺ m/z: 179.

Step 7

The title compound was synthesized according to General Procedure 7; Method A using (1-(5-fluoroquinazolin-7-yl)-1H-1,2,4-triazol-5-yl)methanamine (180.00 mg, 0.737 mmol, 1.00 eq.; synthesized according to General Procedures 2 and 3 starting from 5-fluoro-7-hydrazineylquinazoline from step 6 above) and 1-[1-(4-chloro-3-fluorophenyl)-3-methyl-1H-1,2,4-triazol-5-yl]methanamine (Intermediate I-6; 195.11 mg, 0.811 mmol, 1.10 eq.) providing 1-{[1-(4-chloro-3-fluorophenyl)-3-methyl-1H-1,2,4-triazol-5-yl]methyl}-3-{[1-(5-fluoroquinazolin-7-yl)-1H-1,2,4-triazol-5-yl]methyl}urea (38.3 mg, 10.17%) as a white solid. 1H NMR (300 MHz, DMSO-d6): δ 9.34 (s, 1H), 8.99 (s, 1H), 8.28 (s, 1H), 8.15 (dd, J=9.3, 2.5 Hz, 1H), 8.00 (dd, J=10.6, 1.8 Hz, 1H), 7.83-7.66 (m, 2H), 7.43 (d, J=8.9 Hz, 1H), 6.60 (t, J=5.3 Hz, 1H), 6.49 (t, J=5.9 Hz, 1H), 4.38 (d, J=5.3 Hz, 2H), 4.14 (d, J=5.4 Hz, 2H), 2.28 (s, 3H). LCMS (ES) [M+1]⁺ m/z: 511.

### Example 1.125

### Synthesis of 1-{[1-(4-chloro-3-fluorophenyl)-1H-1,2,4-triazol-5-yl]methyl}-3-({6-[(4-fluorophenyl)methyl]-7-oxo-4,6-diazaspiro[2.4]hept-4-en-5-yl}methyl)urea (Compound 109)

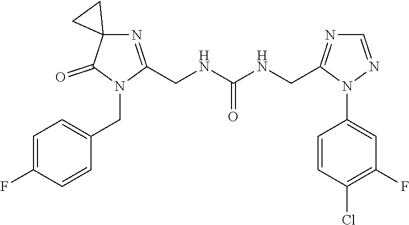 [SMILES: O=C(NCC1=NC2(CC2)C(=O)N1Cc1ccc(F)cc1)NCc1ncnn1-c1ccc(Cl)c(F)c1]

The title compound was synthesized according to General Procedure 7; Method A using 5-(aminomethyl)-6-[(4-fluorophenyl)methyl]-4,6-diazaspiro[2.4]hept-4-en-7-one (Example 1.122 to prepare Compound 106, step 5; 60.00 mg, 0.243 mmol, 1.00 eq.) and 1-[1-(4-chloro-3-fluorophenyl)-1H-1,2,4-triazol-5-yl]methanamine (Intermediate I-4; 54.99 mg, 0.243 mmol, 1.00 eq.) providing 1-{[1-(4-chloro-3-fluorophenyl)-1H-1,2,4-triazol-5-yl]methyl}-3-({6-[(4-fluorophenyl)methyl]-7-oxo-4,6-diazaspiro[2.4]hept-4-en-5-yl}methyl)urea (23.2 mg, 19.13%) as a white solid. ¹H NMR (300 MHz, DMSO-d6): δ 8.12 (s, 1H), 7.86-7.77 (m, 2H), 7.51 (d, J=8.9 Hz, 1H), 7.29-7.14 (m, 4H), 6.77 (t, J=5.6 Hz, 1H), 6.53 (t, J=5.1 Hz, 1H), 4.74 (s, 2H), 4.41 (d, J=5.5 Hz, 2H), 4.05 (d, J=5.6 Hz, 2H), 1.63 (t, J=3.7 Hz, 2H), 1.49 (t, J=3.8 Hz, 2H). LCMS (ES) [M+1]⁺ m/z: 500.

### Example 1.126

### Synthesis of 3-({6-[(4-fluorophenyl)methyl]-7-oxo-4,6-diazaspiro[2.4]hept-4-en-5-yl}methyl)-1-{[1-(quinolin-7-yl)-1H-1,2,4-triazol-5-yl]methyl}urea (Compound 110)

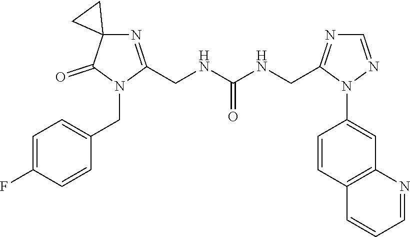 [SMILES: O=C(NCC1=NC2(CC2)C(=O)N1Cc1ccc(F)cc1)NCc1ncnn1-c1ccc2cccnc2c1]

The title compound was synthesized according to General Procedure 7; Method A using 5-(aminomethyl)-6-[(4-fluorophenyl)methyl]-4,6-diazaspiro[2.4]hept-4-en-7-one (Example 1.122 to prepare Compound 106, step 5; 100.00 mg, 0.404 mmol, 1.00 eq.) and 1-[1-(quinolin-7-yl)-1H-1,2,4-triazol-5-yl]methanamine (Intermediate I-9; 100.21 mg, 0.445 mmol, 1.00 eq.) providing 3-({6-[(4-fluorophenyl)methyl]-7-oxo-4,6-diazaspiro[2.4]hept-4-en-5-yl}methyl)-1-{[1-(quinolin-7-yl)-1H-1,2,4-triazol-5-yl]methyl}urea (36.4 mg, 18.05%) as a white solid. 1H NMR (300 MHz, DMSO-d6): δ 9.01 (dd, J=4.2, 1.7 Hz, 1H), 8.50 (dd, J=8.5, 1.8 Hz, 1H), 8.26 (d, J=2.2 Hz, 1H), 8.26-8.11 (m, 2H), 7.84 (dd, J=8.7, 2.2 Hz, 1H), 7.65 (dd, J=8.3, 4.2 Hz, 1H), 7.32-7.23 (m, 2H), 7.23-7.10 (m, 2H), 6.81 (t, J=5.7 Hz, 1H), 6.54 (t, J=5.5 Hz, 1H), 4.72 (s, 2H), 4.51 (d, J=5.5 Hz, 2H), 4.04 (d, J=5.4 Hz, 2H), 1.65-1.61 (m, 2H), 1.53-1.42 (m, 2H). LCMS (ES) [M+1]⁺ m/z: 499.2.

### Example 1.127

### Synthesis of 1-{[1-(4-chloro-3-fluorophenyl)-3-methyl-1H-1,2,4-triazol-5-yl]methyl}-3-{[7-oxo-6-(quinolin-7-yl)-4,6-diazaspiro[2.4]hept-4-en-5-yl]methyl}urea (Compound 111)

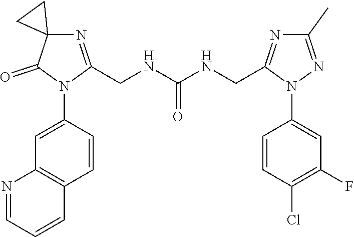 [SMILES: Cc1nc(CNC(=O)NCC2=NC3(CC3)C(=O)N2c2ccc3cccnc3c2)n(-c2ccc(Cl)c(F)c2)n1]

Step 1

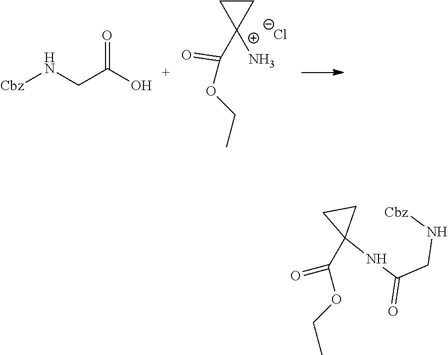

Into a 250-mL round-bottom flask, was placed ethyl 1-aminocyclopropane-1-carboxylate hydrochloride (4.99 g, 30.115 mmol, 1.00 eq.), dimethylformamide (50 mL), ((benzyloxy)carbonyl)glycine (6.30 g, 30.115 mmol, 1.00 eq.), HATU (17.18 g, 45.172 mmol, 1.50 eq.), and DIEA (15.57 g, 120.458 mmol, 4.00 eq.) and the resulting solution was stirred for 16 h at 25° C. The reaction mixture was then diluted with 500 mL of EA, washed with 4×200 mL of water and the organic layer was dried over anhydrous sodium sulfate and concentrated under vacuum providing 7.5 g (77.74%) of ethyl 1-(2-[[(benzyloxy)carbonyl]amino]acetamido)cyclopropane-1-carboxylate as a yellow solid. LCMS (ES) [M+1]⁺ m/z: 321.

Step 2

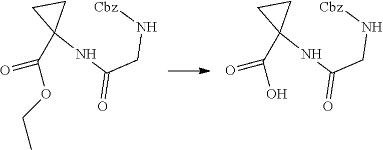 [SMILES: CCOC(=O)C1(NC(=O)CNC(=O)OCc2ccccc2)CC1 ; O=C(CNC(=O)OCc1ccccc1)NC1(C(=O)O)CC1]

Into a 250-mL round-bottom flask, was placed ethyl 1-(2-[[(benzyloxy)carbonyl]amino]acetamido)cyclopropane-1-carboxylate (7.00 g, 21.851 mmol, 1.00 eq.), methanol (25 mL), tetrahydrofuran (25 mL), water (25 mL), and sodium hydroxide (1.75 g, 43.703 mmol, 2.00 equiv) and the resulting solution was stirred for 16 h at 25° C. The mixture was then concentrated and the residue was diluted with 200 mL of water. The pH of the solution was adjusted to 5 with HCl (1 mol/L) and the solids thus formed were collected by filtration providing 5.2 g (81.42%) of 1-(2-[[(benzyloxy)carbonyl]amino]acetamido)cyclopropane-1-carboxylic acid as a yellow solid. LCMS (ES) [M+1]⁺ m/z:293.

Step 3

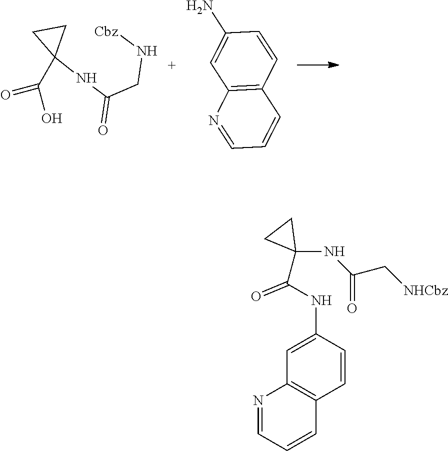 [SMILES: O=C(CNC(=O)OCc1ccccc1)NC1(C(=O)O)CC1 ; Nc1ccc2cccnc2c1 ; O=C(CNC(=O)OCc1ccccc1)NC1(C(=O)Nc2ccc3cccnc3c2)CC1]

Into a 100-mL round-bottom flask, was placed 1-(2-[[(benzyloxy)carbonyl]amino]acetamido)cyclopropane-1-carboxylic acid (3.00 g, 10.264 mmol, 1 eq.), dimethylformamide (50 mL), quinolin-7-amine (1.48 g, 10.264 mmol, 1.00 eq.), HATU (5.85 g, 15.396 mmol, 1.50 eq.), and DIEA (3.98 g, 30.791 mmol, 3.00 eq.) and the resulting solution was stirred for 16 h at 25° C. The crude reaction mixture was filtered and subjected to reverse phase preparative HPLC (Prep-C18, 20-45 uM, 120 g, Tianjin Bonna-Agela Technologies; gradient elution of 20% MeCN in water to 30% MeCN in water over a 10 min period, water contains 0.1% NH₃H₂O) to provide benzyl N-[({1-[(quinolin-7-yl)carbamoyl]cyclopropyl}carbamoyl)methyl]carbamate as a light yellow solid (2.2 g, 51.22%). LCMS (ES) [M+1]⁺ m/z: 419.

Step 4

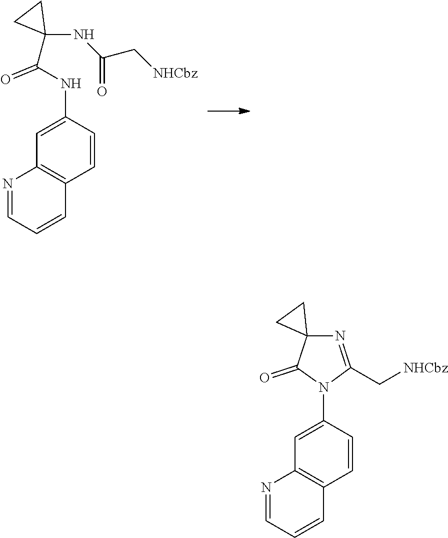 [SMILES: O=C(CNC(=O)OCc1ccccc1)NC1(C(=O)Nc2ccc3cccnc3c2)CC1 ; O=C(NCC1=NC2(CC2)C(=O)N1c1ccc2cccnc2c1)OCc1ccccc1]

Into a 100-mL round-bottom flask, was placed benzyl N-[([1-[(quinolin-7-yl)carbamoyl]cyclopropyl]carbamoyl)methyl]carbamate (2.2 g, 5.257 mmol, 1.00 eq.), pyridine (20.00 mL), (E)-O-(trimethylsilyl)-1-[(trimethylsilyl)amino]ethanone (2.15 g, 10.515 mmol, 2.00 eq.) and the resulting solution was stirred for 16 h at 100° C. The reaction mixture was then cooled to ambient temperature and concentrated. The residue was applied onto a silica gel column and eluted with ethyl acetate/petroleum ether (1:1) providing 510 mg (24.22%) of benzyl N-[[7-oxo-6-(quinolin-7-yl)-4,6-diazaspiro[2.4]hept-4-en-5-yl]methyl]carbamate as a yellow solid. LCMS (ES) [M+1]⁺ m/z: 401.

Step 5

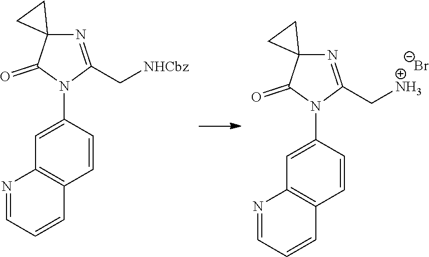 [SMILES: O=C(NCC1=NC2(CC2)C(=O)N1c1ccc2cccnc2c1)OCc1ccccc1 ; [NH3+]CC1=NC2(CC2)C(=O)N1c1ccc2cccnc2c1 ; [Br-]]

Into a 50-mL round-bottom flask, was placed benzyl N-[[7-oxo-6-(quinolin-7-yl)-4,6-diazaspiro[2.4]hept-4-en-5-yl]methyl]carbamate (500.00 mg, 1.249 mmol, 1.00 eq.), acetic acid (10 mL), HBr in AcOH(40%) (2.00 mL) and the resulting solution was stirred for 2 h at 25° C. The reaction solution was then diluted with 100 mL of EA and the solids thus obtained were collected by filtration providing 240 mg (55.36%) of 5-(aminomethyl)-6-(quinolin-7-yl)-4,6-diazaspiro[2.4]hept-4-en-7-one hydrobromide as a brown solid. LCMS (ES) [M+1-HBr]⁺ m/z: 267.

Step 6

The title compound was synthesized according to General Procedure 7; Method A using 1-[1-(4-chloro-3-fluorophenyl)-3-methyl-1H-1,2,4-triazol-5-yl]methanamine (Intermediate I-6; 152.49 mg, 0.634 mmol, 1.00 eq.) and 5-(aminomethyl)-6-(quinolin-7-yl)-4,6-diazaspiro[2.4]hept-4-en-7-one hydrobromide (from step 5 above; 220.00 mg, 0.634 mmol, 1.00 eq.) providing 1-{[1-(4-chloro-3-fluorophenyl)-3-methyl-1H-1,2,4-triazol-5-yl]methyl}-3-{[7-oxo-6-(quinolin-7-yl)-4,6-diazaspiro[2.4]hept-4-en-5-yl]methyl}urea (82.1 mg, 24.31%) as a white solid. ¹H NMR (300 MHz, DMSO-d6): δ 8.98 (dd, J=4.1, 1.8 Hz, 1H), 8.45 (dd, J=8.7, 1.8 Hz, 1H), 8.12 (d, J=8.7 Hz, 1H), 8.07 (d, J=2.1 Hz, 1H), 7.82-7.68 (m, 2H), 7.67-7.57 (m, 2H), 7.46 (d, J=8.9 Hz, 1H), 6.79 (t, J=5.6 Hz, 1H), 6.47 (t, J=5.3 Hz, 1H), 4.31 (d, J=5.5 Hz, 2H), 4.06 (d, J=5.1 Hz, 2H), 2.55 (s, 1H), 2.29 (s, 3H), 1.75 (q, J=4.3, 3.6 Hz, 2H), 1.59 (q, J=4.8, 4.3 Hz, 2H). LCMS (ES) [M+1]⁺ m/z: 533.

### Example 1.128

### Synthesis of 1-{[1-(4-chloro-3-fluorophenyl)-3-methyl-1H-1,2,4-triazol-5-yl]methyl}-3-{[8-oxo-7-(quinolin-7-yl)-5,7-diazaspiro[3.4]oct-5-en-6-yl]methyl}urea (Compound 112)

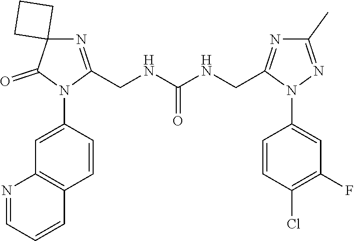 [SMILES: Cc1nc(CNC(=O)NCC2=NC3(CCC3)C(=O)N2c2ccc3cccnc3c2)n(-c2ccc(Cl)c(F)c2)n1]

Step 1

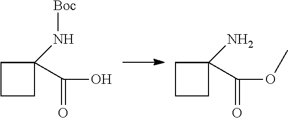

Into a 100-mL 3-necked round-bottom flask, was placed 1-[(tert-butoxycarbonyl)amino]cyclobutane-1-carboxylic acid (5.0 g, 23.2 mmol, 1.0 eq.), H₂SO₄ (2.28 g, 23.2 mmol, 1.0 eq.), and MeOH (50 mL) and the resulting solution was stirred overnight at 75° C. The reaction was then cooled to ambient temperature and concentrated. The residue was dissolved in EtOAc (100 mL) and washed with NaHCO₃(2 N). The organic layers were combined, dried over anhydrous sodium sulfate and concentrated. The residue was applied onto a silica gel column and eluted with 100% PE to 30% THF in PE providing methyl 1-aminocyclobutane-1-carboxylate (2.3 g, 76.7%) as yellow gum. LCMS (ES) [M+1]⁺ m/z: 130.

Step 2

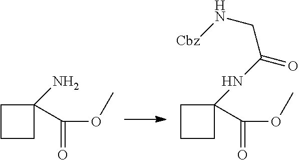

Into a 100-mL 3-necked round-bottom flask purged and maintained with an inert atmosphere of nitrogen, was placed methyl 1-aminocyclobutane-1-carboxylate (2.1 g, 16.259 mmol, 1.00 eq.), DMF (20 mL), ((benzyloxy)carbonyl)glycine (3.7 g, 17.9 mmol, 1.1 eq.), DIEA (3.2 g, 24.4 mmol, 1.5 eq.) and HATU (7.4 g, 19.5 mmol, 1.2 eq.) and the resulting solution was stirred for 16 h at ambient temperature. The reaction solution was then diluted with of water, extracted with 3×100 mL of ethyl acetate, washed with 3×50 mL of brine, dried over anhydrous sodium sulfate and concentrated. The residue was applied onto a silica gel column and eluted with 100% petroleum ether to 10% ethyl acetate in petroleum ether providing methyl 1-(2-[[(benzyloxy)carbonyl]amino]acetamido)cyclobutane-1-carboxylate (2.3 g, 44.16%) as colorless oil. LCMS (ES) [M+1]⁺ m/z: 321.

Step 3

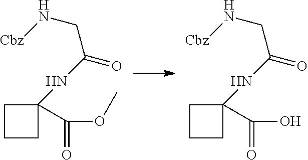 [SMILES: COC(=O)C1(NC(=O)CNC(=O)OCc2ccccc2)CCC1 ; O=C(CNC(=O)OCc1ccccc1)NC1(C(=O)O)CCC1]

Into a 100-mL 3-necked round-bottom flask, was placed methyl 1-(2-[[(benzyloxy)carbonyl]amino]acetamido)cyclobutane-1 carboxylate (2.0 g, 6.2 mmol, 1.0 eq.) and MeOH (20 mL, 123.5 mmol, 19.7 eq.). After the reaction was cooled to 0° C., a solution of NaOH (0.75 g, 18.7 mmol, 3.0 eq.) in H₂O (5 mL) was added drop-wise and the resulting solution was stirred for 2 h while allowing to reach ambient temperature. The pH of the solution was then adjusted to 6 with HCl (2 mol/L) and it was extracted with 3 ×15 mL of ethyl acetate, then concentrated to give 1-(2-[[(benzyloxy)carbonyl]amino]acetamido)cyclobutane-1-carboxylic acid (1.5 g, 78.43%)as a yellow solid. LCMS (ES) [M+1]⁺ m/z: 307.

Step 4

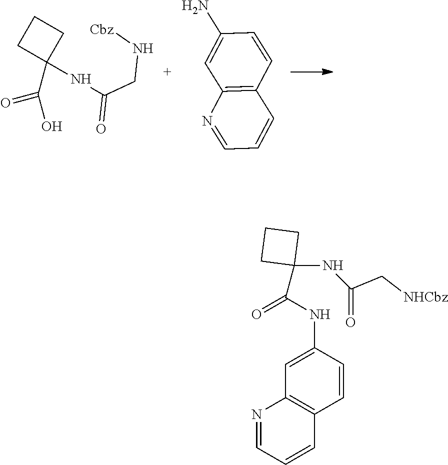 [SMILES: O=C(CNC(=O)OCc1ccccc1)NC1(C(=O)O)CCC1 ; Nc1ccc2cccnc2c1 ; O=C(CNC(=O)OCc1ccccc1)NC1(C(=O)Nc2ccc3cccnc3c2)CCC1]

Into a 100-mL 3-necked round-bottom flask purged and maintained with an inert atmosphere of nitrogen, was placed 1-(2-[[(benzyloxy)carbonyl]amino]acetamido)cyclobutane-1-carboxylic acid (1.5 g, 4.9 mmol, 1.0 eq.), DMF (10 mL), quinolin-7-amine (0.78 g, 5.4 mmol, 1.1 eq.), and DIEA (0.95 g, 7.3 mmol, 1.5 eq.) and the reaction was cooled to 0° C. followed by the addition of HATU (2.2 g, 5.9 mmol, 1.2 eq.) in several batches at 0° C. The resulting solution was stirred for 16 h at room temperature then it was diluted with water, extracted with 3×100 mL of ethyl acetate, washed with 3×50 mL of brine, dried over anhydrous sodium sulfate and concentrated. The residue was applied onto a silica gel column and eluted with 100% petroleum ether to 50% ethyl acetate in petroleum ether providing benzyl N-[([1-[(quinolin-7 yl) carbamoyl]cyclobutyl]carbamoyl) methyl]carbamate (1.3 g, 61.4%) as a yellow solid. LCMS (ES) [M+1]⁺ m/z: 433.

Step 5

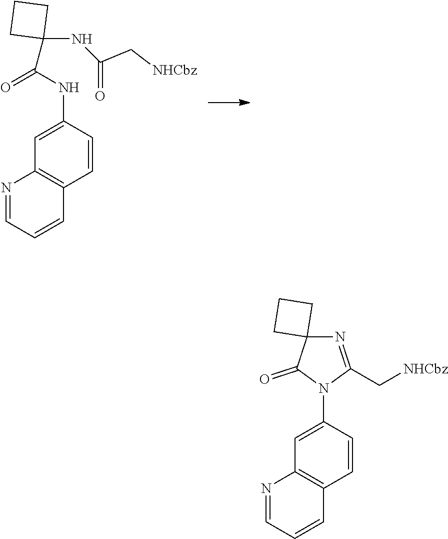 [SMILES: O=C(CNC(=O)OCc1ccccc1)NC1(C(=O)Nc2ccc3cccnc3c2)CCC1 ; O=C(NCC1=NC2(CCC2)C(=O)N1c1ccc2cccnc2c1)OCc1ccccc1]

Into a 100-mL 3-necked round-bottom flask, was placed benzyl N-[([1-[(quinolin-7-yl)carbamoyl]cyclobutyl]carbamoyl)methyl]carbamate (600 mg, 1.39 mmol, 1.0 eq.) and pyridine (130 mg, 1.7 mmol, 1.2 eq.) in DCM (5 mL). After the reaction was cooled to 0° C., (E)-O-(trimethylsilyl)-1-[(trimethylsilyl)amino]ethenone (310 mg, 1.39 mmol, 1.0 eq.) was added in portions and the resulting solution was stirred for 16 h at 100° C. The reaction was then quenched by the addition of 30 mL of water, cooled and extracted with 3×30 mL of ethyl acetate, dried over anhydrous sodium sulfate and concentrated under vacuum providing benzyl N-[[8-oxo-7-(quinolin-7-yl)-5,7-diazaspiro[3.4]oct-5-en-6-yl]methyl]carbamate (180 mg, 31.3%) as an off white solid. LCMS (ES) [M+1]⁺ m/z: 415.

Step 6

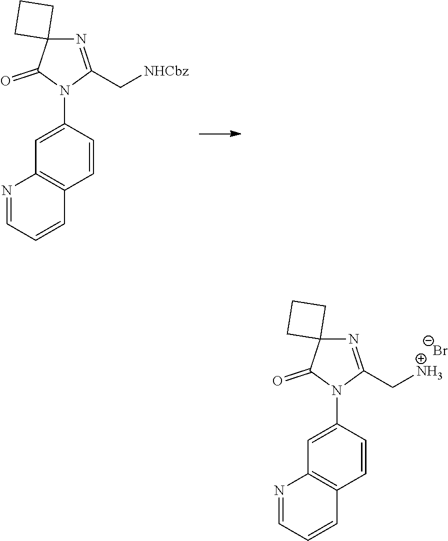 [SMILES: O=C(NCC1=NC2(CCC2)C(=O)N1c1ccc2cccnc2c1)OCc1ccccc1 ; [NH3+]CC1=NC2(CCC2)C(=O)N1c1ccc2cccnc2c1 ; [Br-]]

Into a 50-mL 3-necked round-bottom flask purged and maintained with an inert atmosphere of nitrogen, was placed benzyl N-[[8-oxo-7-(quinolin-7-yl)-5,7-diazaspiro[3.4]oct-5-en-6-yl]methyl]carbamate) and AcOH (2 mL). HBr in AcOH(40%) (0.3 mL) was then added at 0° C. and the resulting solution was stirred for 1 h at ambient temperature. To the reaction solution was then added EtOAc, the solids were removed by filtration and the filtrate was concentrated under vacuum to give 6-(aminomethyl)-7-(quinolin-7-yl)-5,7-diazaspiro[3.4]oct-5-en-8-one hydrobromide (100 mg) as an off white solid. LCMS (ES) [M+1-HBr]⁺ m/z: 281.

Step 7

The title compound was synthesized according to General Procedure 7; Method A using 1-[1-(4-chloro-3-fluorophenyl)-3-methyl-1H-1,2,4-triazol-5-yl]methanamine (Intermediate I-6; 120.00 mg, 0.499 mmol, 1.00 eq.) and 6-(aminomethyl)-7-(quinolin-7-yl)-5,7-diazaspiro[3.4]oct-5-en-8-one hydrobromide (from step 6 above; 146.76 mg, 0.524 mmol, 1.05 eq.) providing 1-{[1-(4-chloro-3-fluorophenyl)-3-methyl-1H-1,2,4-triazol-5-yl]methyl}-3-{[8-oxo-7-(quinolin-7-yl)-5,7-diazaspiro[3.4]oct-5-en-6-yl]methyl}urea (82.7 mg, 45.3%) as a white solid. ¹H NMR (300 MHz, DMSO-d6): δ 8.97 (dd, J=4.2, 1.8 Hz, 1H), 8.45 (dd, J=8.7, 2.1 Hz, 1H), 8.10 (d, J=8.7 Hz, 1H), 8.02 (d, J=2.1 Hz, 1H), 7.79-7.76 (m, 2H), 7.73-7.60 (m, 2H), 7.47 (ddd, J=8.5, 2.5, 1.2 Hz, 1H), 6.89 (t, J=5.7 Hz, 1H), 6.50 (t, J=5.1 Hz, 1H), 4.33 (d, J=5.4 Hz, 2H), 3.99 (d, J=4.8 Hz, 2H), 2.52-2.31 (m, 4H), 2.29 (s, 3H), 2.19-2.01 (m, 2H). LCMS (ES) [M+1]⁺ m/z: 547.

### Example 1.129

### Synthesis of 1-{[1-(4-chloro-3-fluorophenyl)-3-methyl-1H-1,2,4-triazol-5-yl]methyl}-3-{[1-(5-methylquinolin-7-yl)-1H-1,2,4-triazol-5-yl]methyl}urea (Compound 113)

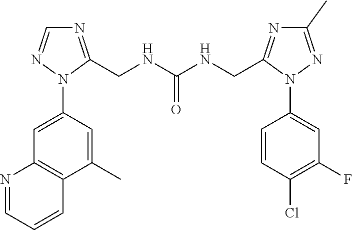 [SMILES: Cc1nc(CNC(=O)NCc2ncnn2-c2cc(C)c3cccnc3c2)n(-c2ccc(Cl)c(F)c2)n1]

Step 1

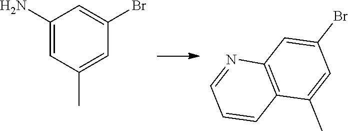

Into a 1000-mL round-bottom flask, was placed 3-bromo-5-methylaniline (20.00 g, 107.497 mmol, 1.00 eq.), 4-ethoxy-3-(ethoxymethyl)but-1-ene (20.41 g, 128.980 mmol, 1.20 eq.), and HCl (300.00 mL) and the resulting solution was stirred for 6 h at 80° C. The reaction mixture was then cooled to ambient temperature and concentrated. The residue was extracted with 3×300 mL of dichloromethane and the organic layers were combined, dried over anhydrous sodium sulfate and concentrated under vacuum. The residue was applied onto a silica gel column and eluted with THF/PE (1:4). The crude product was purified by Prep-HPLC with the following conditions: (2 #SHIMADZU (HPLC-01)): Column, Welch XB-C18, 21.2*250 mm, Sum; mobile phase, Water (0.05% FA) and ACN (10% PhaseB up to 50% in 25 min) providing 1.7 g (7.12%) of 7-bromo-5-methylquinoline as colorless oil. LCMS (ES) [M+1]⁺ m/z: 222.

Step 2

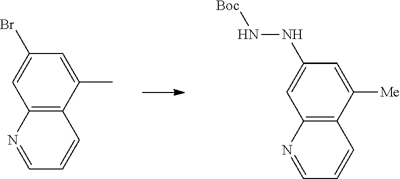 [SMILES: Cc1cc(Br)cc2ncccc12 ; Cc1cc(NNC(=O)OC(C)(C)C)cc2ncccc12]

Into a 100-mL round-bottom flask purged and maintained with an inert atmosphere of nitrogen, was placed 7-bromo-5-methylquinoline (1.60 g, 7.204 mmol, 1.00 eq.), tert-butoxycarbohydrazide (1.14 g, 8.645 mmol, 1.2 eq.), Pd₂(dba)₃CHCl₃ (0.745 g, 0.720 mmol, 0.1 eq.), BINAP (0.22 g, 0.360 mmol, 0.05 eq.), Cs₂CO₃ (4.69 g, 14.409 mmol, 2 eq.), and DMF (30.00 mL) and the resulting solution was stirred for 16 h at 100° C. The reaction mixture was then cooled to ambient temperature and extracted with 3×50 mL of chloromethane and the organic layers were combined, dried over anhydrous sodium sulfate and concentrated. The residue was applied onto a silica gel column and eluted with 100% petroleum ether to 30% ethyl acetate in petroleum ether providing 1 g (50.78%) of tert-butyl 2-(5-methylquinolin-7-yl)hydrazine-1-carboxylate as an off-white solid. LCMS (ES) [M+1]⁺ m/z: 274.

Step 3

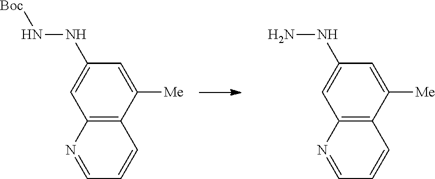 [SMILES: Cc1cc(NNC(=O)OC(C)(C)C)cc2ncccc12 ; Cc1cc(NN)cc2ncccc12]

Into a 100-mL round-bottom flask, was placed tert-butyl 2-(5-methylquinolin-7-yl)hydrazine-1-carboxylate (1.00 g, 3.797 mmol, 1.00 eq.) followed by the addition of HCl(g)in EA (15.00 mL) and the resulting solution was stirred for 4 h at ambient temperature. The solids thus formed were collected by filtration providing 0.6 g (91.22%) of 7-hydrazineyl-5-methylquinoline as an off-white solid. LCMS (ES) [M+1]⁺ m/z:174.

Step 4

The title compound was synthesized according to General Procedure 7; Method A using (1-(5-methylquinolin-7-yl)-1H-1,2,4-triazol-5-yl)methanamine (300.00 mg, 1.254 mmol, 1.00 eq.; synthesized according to General Procedures 2 and 3 starting from 7-hydrazineyl-5-methylquinoline from step 3 above) and 1-[1-(4-chloro-3-fluorophenyl)-3-methyl-1H-1,2,4-triazol-5-yl]methanamine (Intermediate I-6; 331.91 mg, 1.379 mmol, 1.1 eq.) providing 1-{[1-(4-chloro-3-fluorophenyl)-3-methyl-1H-1,2,4-triazol-5-yl]methyl}-3-{[1-(5-methylquinolin-7-yl)-1H-1,2,4-triazol-5-yl]methyl}urea (109.8 mg, 17.31%) as a white solid. ¹H NMR (300 MHz, DMSO-d6): δ 9.00 (dd, J=4.2, 1.6 Hz, 1H), 8.56 (dt, J=8.3, 1.4 Hz, 1H), 8.17 (s, 1H), 8.09 (d, J=2.2 Hz, 1H), 7.85-7.70 (m, 2H), 7.70-7.58 (m, 2H), 7.47 (ddd, J=8.7, 2.5, 1.2 Hz, 1H), 6.77 (t, J=5.6 Hz, 1H), 6.74 (t, J=5.6 Hz, 1H), 4.51 (d, J=5.6 Hz, 2H), 4.35 (d, J=5.7 Hz, 2H), 2.74 (s, 3H), 2.28 (s, 3H). LCMS (ES) [M+1]⁺ m/z: 506.

### Example 1.130

### Synthesis of 1,3-bis({[1-(2-fluoro-4-methylphenyl)-1H-1,2,3,4-tetrazol-5-yl]methyl})urea (Compound 114)

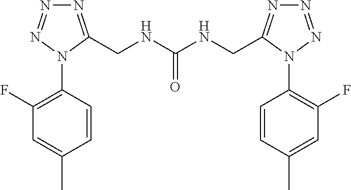 [SMILES: Cc1ccc(-n2nnnc2CNC(=O)NCc2nnnn2-c2ccc(C)cc2F)c(F)c1]

The title compound was synthesized according to General Procedure 6, Method A using 1-[1-(2-fluoro-4-methylphenyl)-1H-1,2,3,4-tetrazol-5-yl]methanamine (50 mg; synthesized according to General Procedure 4 starting from commercial 2-fluoro-4-methylaniline) to provide 1,3-bis({-[1-(2-fluoro-4-methylphenyl)-1H-1,2,3,4-tetrazol-5-yl]methyl})urea as a white solid (5.4 mg; 10% yield). 1-H NMR (400 MHz, DMSO-d6) δ 7.55 (t, J=8.0 Hz, 2H), 7.38 (d, J=11.2 Hz, 2H), 7.23 (d, J=8.2 Hz, 2H), 6.79 (t, J=5.8 Hz, 2H), 4.33 (d, J=5.7 Hz, 4H), 2.40 (s, 6H). LCMS (ES) [M+1]⁺ m/z 441.0.

### Example 1.131

### Synthesis of 1,3-bis({1-[4-(trifluoromethyl)phenyl]-1H-1,2,3,4-tetrazol-5-yl}methyl)urea (Compound 115)

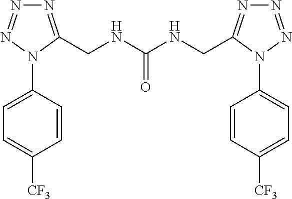 [SMILES: O=C(NCc1nnnn1-c1ccc(C(F)(F)F)cc1)NCc1nnnn1-c1ccc(C(F)(F)F)cc1]

The title compound was synthesized according to General Procedure 6, Method A using 1-{1-[4-(trifluoromethyl)phenyl]-1H-1,2,3,4-tetrazol-5-yl}methanamine (62 mg; synthesized according to General Procedure 4 starting from commercial 4-trifluoromethylaniline) to provide 1,3-bis({1-[4-(trifluoromethyl)phenyl]-1H-1,2,3,4-tetrazol-5-yl}methyl)urea as a white solid (15.8 mg; 24% yield). 1H NMR (400 MHz, DMSO-d6) δ 8.00 (d, J=8.5 Hz, 4H), 7.89 (d, J=8.3 Hz, 4H), 6.91 (t, J=5.7 Hz, 2H), 4.48 (d, J=5.6 Hz, 4H). LCMS (ES) [M+1]⁺ m/z 513.0.

### Example 1.132

### Synthesis of 1,3-bis({[1-(6-chloropyridin-3-yl)-1H-1,2,4-triazol-5-yl]methyl})urea (Compound 116)

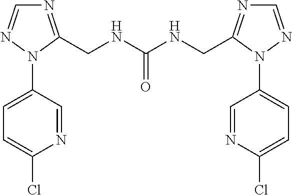 [SMILES: O=C(NCc1ncnn1-c1ccc(Cl)nc1)NCc1ncnn1-c1ccc(Cl)nc1]

The title compound was synthesized according to General Procedure 6, Method A using [1-(6-chloropyridin-3-yl)-1H-1,2,4-triazol-5-yl]methanaminium chloride (75 mg; synthesized according to General Procedures 2 and 3 starting from commercial 2-chloro-5-hydrazinylpyridine) to provide 1,3-bis({[1-(6-chloropyridin-3-yl)-1H-1,2,4-triazol-5-yl]methyl})urea as a white solid (28.5 mg; 36% yield). 1-H NMR (400 MHz, DMSO-d6) δ 8.65 (d, J=2.7 Hz, 2H), 8.14 (d, J=1.3 Hz, 2H), 8.11 (dd, J=8.5, 2.7 Hz, 2H), 7.71 (d, J=8.5 Hz, 2H), 6.71 (t, J=5.7 Hz, 2H), 4.35 (d, J=5.6 Hz, 4H). LCMS (ES) [M+1]⁺ m/z 446.0.

### Example 1.133

### Synthesis of 1,3-bis({[1-(6-methoxypyridin-3-yl)-1H-1,2,4-triazol-5-yl]methyl})urea (Compound 117)

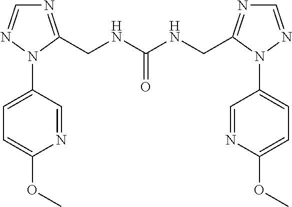 [SMILES: COc1ccc(-n2ncnc2CNC(=O)NCc2ncnn2-c2ccc(OC)nc2)cn1]

The title compound was synthesized according to General Procedure 6, Method A using [1-(6-methoxypyridin-3-yl)-1H-1,2,4-triazol-5-yl]methanaminium chloride (89.5 mg; synthesized according to General Procedures 2 and 3 starting from commercial 2-(6-methoxypyridin-3-yl)hydrazinium chloride) to provide 1,3-bis({[1-(6-methoxypyridin-3-yl)-1H-1,2,4-triazol-5-yl]methyl})urea as a white solid (35 mg; 37% yield). 1H NMR (400 MHz, DMSO-d6) δ 8.36 (t, J=2.2 Hz, 2H), 8.08 (d, J=1.8 Hz, 2H), 7.89 (dt, J=8.8, 2.3 Hz, 2H), 6.97 (dd, J=8.7, 1.8 Hz, 2H), 6.71-6.67 (m, 2H), 4.28 (d, J=5.5 Hz, 4H), 3.90 (d, J=1.8 Hz, 6H). LCMS (ES) [M+1]⁺ m/z 437.0.

### Example 1.134

### Synthesis of 3-{[1-(4-chloro-3-fluorophenyl)-1H-1,2,4-triazol-5-yl]methyl}-1-{[1-(6-methoxypyridin-3-yl)-1H-1,2,4-triazol-5-yl]methyl}urea (Compound 118)

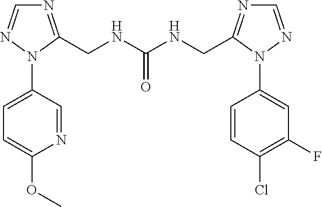 [SMILES: COc1ccc(-n2ncnc2CNC(=O)NCc2ncnn2-c2ccc(Cl)c(F)c2)cn1]

The title compound was synthesized according to General Procedure 7, Method A using N-{[1-(4-chloro-3-fluorophenyl)-1H-1,2,4-triazol-5-yl]methyl}-1H-imidazole-1-carboxamide (Intermediate I-4, 79 mg; 0.25 mmol; 1.00 eq.) and [1-(6-methoxypyridin-3-yl)-1H-1,2,4-triazol-5-yl]methanaminium chloride (59.53 mg; 0.25 mmol; 1.00 eq. synthesized according to General Procedures 2 and 3 starting from commercial 2-(6-methoxypyridin-3-yl)hydrazinium chloride) to provide 3-{[1-(4-chloro-3-fluorophenyl)-1H-1,2,4-triazol-5-yl]methyl}-1-{[1-(6-methoxypyridin-3-yl)-1H-1,2,4-triazol-5-yl]methyl}urea as a colorless solid (45 mg; 40% yield). 1H NMR (400 MHz, DMSO-d6) δ 8.36 (dd, J=2.7, 0.6 Hz, 1H), 8.10 (s, 1H), 8.08 (s, 1H), 7.89 (dd, J=8.8, 2.7 Hz, 1H), 7.82-7.74 (m, 2H), 7.48 (ddd, J=8.6, 2.5, 1.2 Hz, 1H), 6.97 (dd, J=8.8, 0.7 Hz, 1H), 6.71 (t, J=5.6 Hz, 2H), 4.38 (d, J=5.6 Hz, 2H), 4.29 (d, J=5.6 Hz, 2H), 3.89 (s, 3H). LCMS (ES) [M+1]⁺ m/z 458.0.

### Example 1.135

### Synthesis of 3-{[1-(4-chloro-3-fluorophenyl)-1H-1,2,4-triazol-5-yl]methyl}-1-{[1-(6-hydroxypyridin-3-yl)-1H-1,2,4-triazol-5-yl]methyl}urea (Compound 119)

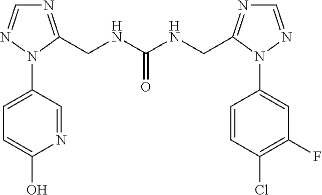 [SMILES: O=C(NCc1ncnn1-c1ccc(O)nc1)NCc1ncnn1-c1ccc(Cl)c(F)c1]

3-{[1-(4-chloro-3-fluorophenyl)-1H-1,2,4-triazol-5-yl]methyl}-1-{[1-(6-methoxypyridin-3-yl)-1H-1,2,4-triazol-5-yl]methyl}urea (Example 1.134 used to prepare Compound 118; 21 mg) was taken up in dioxane (1 mL), treated with 0.5 mL of HCl (4 N in dioxane) and heated to 70° C. After 1.5 h the mixture was cooled to ambient temperature, diluted with water and directly purified by reverse phase preparative HPLC (Waters eluting with a gradient of 5-95% MeCN (containing 0.1% formic acid) in water, 13 minutes 5 injections) to provide 3-{[1-(4-chloro-3-fluorophenyl)-1H-1,2,4-triazol-5-yl]methyl}-1-{[1-(6-hydroxypyridin-3-yl)-1H-1,2,4-triazol-5-yl]methyl}urea (12.3 mg; 60% yield) as a white solid. ¹H NMR (400 MHz, DMSO-d6) δ 11.93 (s, 1H), 8.10 (s, 1H), 8.01 (s, 1H), 7.78 (ddd, J=13.1, 5.3, 3.3 Hz, 3H), 7.54-7.46 (m, 2H), 6.70 (dt, J=8.3, 5.6 Hz, 2H), 6.41 (d, J=9.6 Hz, 1H), 4.39 (d, J=5.6 Hz, 2H), 4.25 (d, J=5.6 Hz, 2H). LCMS (ES) [M+1]⁺ m/z 444.0.

### Example 1.136

### Synthesis of 3-{[1-(4-chloro-3-fluorophenyl)-1H-1,2,4-triazol-5-yl]methyl}-3-ethyl-1-{[1-(6-methoxypyridin-3-yl)-1H-1,2,4-triazol-5-yl]methyl}urea (Compound 120)

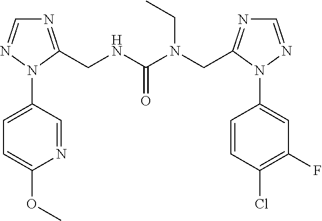 [SMILES: CCN(Cc1ncnn1-c1ccc(Cl)c(F)c1)C(=O)NCc1ncnn1-c1ccc(OC)nc1]

The title compound was synthesized according to General Procedure 7, Method A using 2-(6-methoxypyridin-3-yl)hydrazinium chloride (71.00 mg; 0.29 mmol; 1.00 eq. synthesized according to General Procedures 2 and 3 starting from commercial 2-methoxy-5-hydrazinylpyridine) and {[1-(4-chloro-3-fluorophenyl)-1H-1,2,4-triazol-5-yl]methyl}(ethyl)amine (Example 1.46 used to prepare Compound 30, step 2; 68.08 mg; 0.27 mmol; 1.00 eq.) to provide 3-{[1-(4-chloro-3-fluorophenyl)-1H-1,2,4-triazol-5-yl]methyl}-3-ethyl-1-{[1-(6-methoxypyridin-3-yl)-1H-1,2,4-triazol-5-yl]methyl}urea as a colorless solid (55 mg; 42% yield). 1H NMR (400 MHz, DMSO-d6) δ 8.36 (d, J=2.8 Hz, 1H), 8.07 (s, 1H), 8.04 (s, 1H), 7.88 (dd, J=8.8, 2.7 Hz, 1H), 7.78-7.71 (m, 2H), 7.44 (ddd, J=8.7, 2.4, 1.2 Hz, 1H), 7.03 (t, J=5.4 Hz, 1H), 6.95 (d, J=8.8 Hz, 1H), 4.55 (s, 2H), 4.26 (d, J=5.3 Hz, 2H), 3.89 (s, 3H), 3.18 (q, J=7.0 Hz, 2H), 0.93 (t, J=7.0 Hz, 3H). LCMS (ES) [M+1]⁺ m/z 486.0.

### Example 1.137

### Synthesis of 3-{[1-(4-chloro-3-fluorophenyl)-1H-1,2,4-triazol-5-yl]methyl}-3-ethyl-1-{[1-(6-hydroxypyridin-3-yl)-1H-1,2,4-triazol-5-yl]methyl}urea (Compound 121)

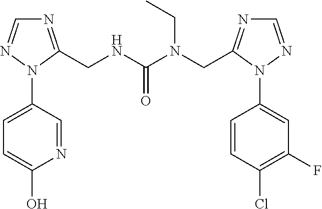 [SMILES: CCN(Cc1ncnn1-c1ccc(Cl)c(F)c1)C(=O)NCc1ncnn1-c1ccc(O)nc1]

3-{[1-(4-chloro-3-fluorophenyl)-1H-1,2,4-triazol-5-yl]methyl}-3-ethyl-1-{[1-(6-methoxypyridin-3-yl)-1H-1,2,4-triazol-5-yl]methyl}urea (Example 1.136 used to prepare Example 120; 30 mg) was taken up in dioxane (1.5 mL), treated with 0.6 mL of HCl (4 N in dioxane) and heated to 70° C. After 4 h the mixture was cooled to ambient temperature, diluted with water directly purified by reverse phase preparative HPLC (Waters eluting with a gradient of 5-95% MeCN (containing 0.1% formic acid) in water, 13 minutes 4 injections with collection by Mass) to provide 3-{[1-(4-chloro-3-fluorophenyl)-1H-1,2,4-triazol-5-yl]methyl}-3-ethyl-1-{[1-(6-hydroxypyridin-3-yl)-1H-1,2,4-triazol-5-yl]methyl}urea as a white solid (11.5 mg; 39% yield). 1-H NMR (400 MHz, DMSO-d6) δ 11.91 (s, 1H), 8.07 (s, 1H), 7.98 (s, 1H), 7.84-7.67 (m, 3H), 7.50 (dd, J=9.7, 3.0 Hz, 1H), 7.45 (ddd, J=8.7, 2.6, 1.2 Hz, 1H), 7.03 (t, J=5.4 Hz, 1H), 6.40 (d, J=9.7 Hz, 1H), 4.57 (s, 2H), 4.22 (d, J=5.3 Hz, 2H), 3.19 (t, J=7.1 Hz, 2H), 0.96 (t, J=7.0 Hz, 3H). LCMS (ES) [M+1]⁺ m/z 472.0.

### Example 1.138

### Synthesis of 3-{[1-(4-chloro-3-fluorophenyl)-1H-1,2,4-triazol-5-yl]methyl}-1-ethyl-{[[1-(6-methoxypyridin-3-yl)-1H-1,2,4-triazol-5-yl]methyl}urea (Compound 122)

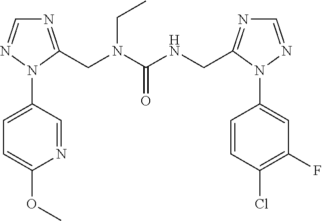 [SMILES: CCN(Cc1ncnn1-c1ccc(OC)nc1)C(=O)NCc1ncnn1-c1ccc(Cl)c(F)c1]

Step 1

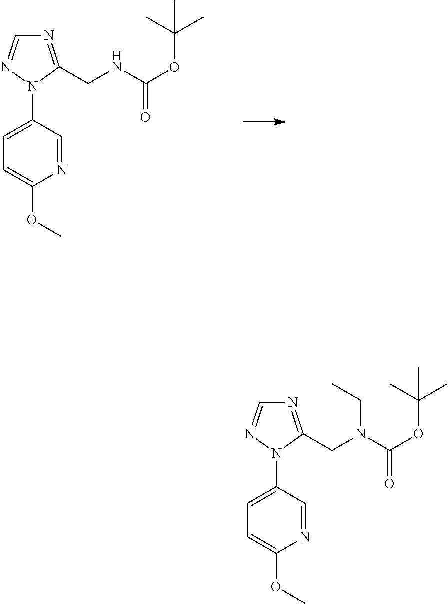 [SMILES: COc1ccc(-n2ncnc2CNC(=O)OC(C)(C)C)cn1 ; CCN(Cc1ncnn1-c1ccc(OC)nc1)C(=O)OC(C)(C)C]

To a stirred solution of tert-butyl N-{[1-(6-methoxypyridin-3-yl)-1H-1,2,4-triazol-5-yl]methyl}carbamate (200.00 mg; 0.66 mmol; 1.00 eq. synthesized according to General Procedures 2 starting from commercial 2-(6-methoxypyridin-3-yl)hydrazinium chloride) in N,N-dimethylformamide (3.00 mL) cooled to 0° C. was added sodium hydride (65.50 mg; 1.64 mmol; 2.50 eq.) in one portion and the mixture was stirred at 0° C. for 30 min. Iodoethane (0.26 mL; 3.28 mmol; 5.00 eq.) was then added drop-wise and the mixture was allowed to warm to ambient temperature. After 2 h the mixture was diluted with 1:1 PhMe/EtOAc and washed with an equal amount of water. The organic phase dried over MgSO4, filtered and concentrated. The residue was purified on a silica gel column eluting with 0-50% EtOAc in heptanes. to provide tert-butyl N-ethyl-N-{[1-(6-methoxypyridin-3-yl)-1H-1,2,4-triazol-5-yl]methyl}carbamate as an orange oil. (189 mg; 87% yield).

Step 2

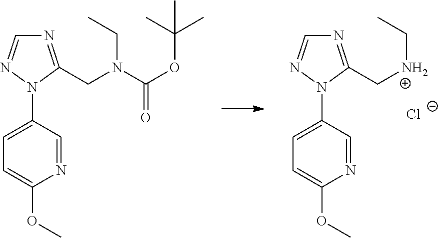 [SMILES: CCN(Cc1ncnn1-c1ccc(OC)nc1)C(=O)OC(C)(C)C ; CC[NH2+]Cc1ncnn1-c1ccc(OC)nc1 ; [Cl-]]

To a mixture of tert-butyl N-ethyl-N-{[1-(6-methoxypyridin-3-yl)-1H-1,2,4-triazol-5-yl]methyl}carbamate (180.00 mg; 0.54 mmol; 1.00 eq.) in 1,2-dichloroethane (2.70 mL) cooled to 0° C. was added hydrochloric acid (0.67 mL; 4.00 mol/L; 2.70 mmol; 5.00 eq.) slowly. The mixture was stirred while allowing to slowly warm to ambient temperature. After 5 h the mixture was filtered and air-dried to provide ethyl({[1-(6-methoxypyridin-3-yl)-1H-1,2,4-triazol-5-yl]methyl})azanium chloride as a yellow powder. The crude material was taken on to the next step without further purification assuming 100% yield (˜140 mg).

Step 3

The title compound was synthesized according to General Procedure 7, Method A using 1-[1-(4-chloro-3-fluorophenyl)-1H-1,2,4-triazol-5-yl]methanamine (Intermediate I-4; 24.7 mg; 0.11 mmol; 1.00 eq.) and ethyl({[1-(6-methoxypyridin-3-yl)-1H-1,2,4-triazol-5-yl]methyl})azanium chloride (29.44 mg; 0.11 mmol; 1.00 eq. from step 2 above) to provide 3-{[1-(4-chloro-3-fluorophenyl)-1H-1,2,4-triazol-5-yl]methyl}-1-ethyl-1-{[1-(6-methoxypyridin-3-yl)-1H-1,2,4-triazol-5-yl]methyl}urea as a colorless solid (15.7 mg; 30% yield). 1-H NMR (400 MHz, DMSO-d6) δ 8.32 (dd, J=2.8, 0.7 Hz, 1H), 8.07 (s, 1H), 8.04 (s, 1H), 7.84 (dd, J=8.8, 2.7 Hz, 1H), 7.79-7.71 (m, 2H), 7.47 (ddd, J=8.6, 2.4, 1.2 Hz, 1H), 7.04 (t, J=5.4 Hz, 1H), 6.93 (dd, J=8.8, 0.7 Hz, 1H), 4.46 (s, 2H), 4.34 (d, J=5.3 Hz, 2H), 3.89 (s, 3H), 3.17 (q, J=6.9 Hz, 2H), 0.91 (t, J=7.0 Hz, 3H). LCMS (ES) [M+1]⁺ m/z 486.0.

### Example 1.139

### Synthesis of 3-{[1-(4-chloro-3-fluorophenyl)-3-methyl-1H-1,2,4-triazol-5-yl]methyl}-1-{[1-(6-methoxypyridin-3-yl)-1H-1,2,4-triazol-5-yl]methyl}urea (Compound 123)

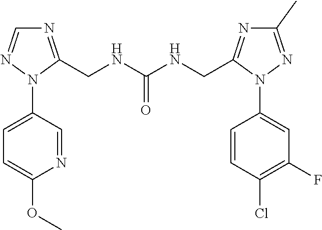 [SMILES: COc1ccc(-n2ncnc2CNC(=O)NCc2nc(C)nn2-c2ccc(Cl)c(F)c2)cn1]

The title compound was synthesized according to General Procedure 7, Method A using 1-[1-(4-chloro-3-fluorophenyl)-3-methyl-1H-1,2,4-triazol-5-yl]methanamine (Intermediate I-6; 37.3 mg; 0.16 mmol; 1.00 eq.) and [1-(6-methoxypyridin-3-yl)-1H-1,2,4-triazol-5-yl]methanaminium chloride (37.5 mg; 0.16 mmol; 1.00 eq. synthesized according to General Procedures 2 and 3 starting from commercial 2-(6-methoxypyridin-3-yl)hydrazinium chloride) to provide 3-{[1-(4-chloro-3-fluorophenyl)-3-methyl-1H-1,2,4-triazol-5-yl]methyl}-1-{[1-(6-methoxypyridin-3-yl)-1H-1,2,4-triazol-5-yl]methyl}urea as a white solid (10.3 mg, 14.05%). 1-H NMR (400 MHz, DMSO-d6) δ 8.36 (d, J=2.7 Hz, 1H), 8.07 (s, 1H), 7.89 (dd, J=8.8, 2.7 Hz, 1H), 7.78-7.70 (m, 2H), 7.45 (ddd, J=8.7, 2.5, 1.2 Hz, 1H), 6.96 (d, J=8.8 Hz, 1H), 6.69 (td, J=5.7, 3.4 Hz, 2H), 4.33 (d, J=5.6 Hz, 2H), 4.29 (d, J=5.6 Hz, 2H), 3.90 (s, 3H), 2.27 (s, 3H). LCMS (ES) [M+1]⁺ m/z 472.

### Example 1.140

### Synthesis of 3-{[1-(4-chloro-3-fluorophenyl)-3-methyl-1H-1,2,4-triazol-5-yl]methyl}-1-ethyl-1-{[1-(6-methoxypyridin-3-yl)-1H-1,2,4-triazol-5-yl]methyl}urea (Compound 124)

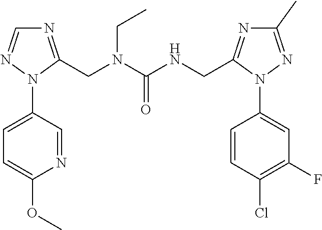 [SMILES: CCN(Cc1ncnn1-c1ccc(OC)nc1)C(=O)NCc1nc(C)nn1-c1ccc(Cl)c(F)c1]

The title compound was synthesized according to General Procedure 7, Method A using 1-[1-(4-chloro-3-fluorophenyl)-3-methyl-1H-1,2,4-triazol-5-yl]methanamine (Intermediate I-6; 37.3 mg; 0.16 mmol; 1.00 eq.) and ethyl(1-[1-(6-methoxypyridin-3-yl)-1H-1,2,4-triazol-5-yl]methyl})azanium chloride (29.44 mg; 0.11 mmol; 1.00 eq. Example 1.138 used to prepare Compound 122, Step 2) to provide 3-{[1-(4-chloro-3-fluorophenyl)-3-methyl-1H-1,2,4-triazol-5-yl]methyl}-1-ethyl-1-{[1-(6-methoxypyridin-3-yl)-1H-1,2,4-triazol-5-yl]methyl}urea as a white solid (6.8 mg, 11.3%). 1-H NMR (400 MHz, DMSO-d6) δ 8.32 (d, J=2.7 Hz, 1H), 8.04 (s, 1H), 7.84 (dd, J=8.8, 2.7 Hz, 1H), 7.75-7.68 (m, 2H), 7.44 (ddd, J=8.6, 2.4, 1.1 Hz, 1H), 7.00 (t, J=5.4 Hz, 1H), 6.93 (d, J=8.8 Hz, 1H), 4.46 (s, 2H), 4.29 (d, J=5.3 Hz, 2H), 3.89 (s, 3H), 3.17 (q, J=7.0 Hz, 2H), 2.26 (s, 3H), 0.90 (t, J=7.0 Hz, 3H). LCMS (ES) [M+1]⁺ m/z 500.

### Example 1.141

### Synthesis of 1-{[1-(4-chloro-3-fluorophenyl)-3-methyl-1H-1,2,4-triazol-5-yl]methyl}-3-{[1-(isoquinolin-3-yl)-1H-1,2,4-triazol-5-yl]methyl}urea (Compound 125)

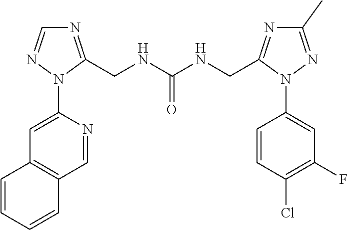 [SMILES: Cc1nc(CNC(=O)NCc2ncnn2-c2cc3ccccc3cn2)n(-c2ccc(Cl)c(F)c2)n1]

The title compound was synthesized according to General Procedure 7, Method A using [1-(isoquinolin-3-yl)-1H-1,2,4-triazol-5-yl]methanaminium chloride (28 mg; 0.11 mmol 1 eq. synthesized according to General Procedures 2 and 3 starting from commercial 3-hydrazinylisoquinoline) and 1-[1-(4-chloro-3-fluorophenyl)-3-methyl-1H-1,2,4-triazol-5-yl]methanamine (Intermediate I-6; 25.6 mg, 0.11 mmol, 1 eq.) to provide 1-{[1-(4-chloro-3-fluorophenyl)-3-methyl-1H-1,2,4-triazol-5-yl]methyl}-3-{[1-(isoquinolin-3-yl)-1H-1,2,4-triazol-5-yl]methyl}urea as a colorless solid (6.1 mg; 11.6% yield). 1H NMR (400 MHz, DMSO-d6) δ 9.35 (d, J=0.9 Hz, 1H), 8.30 (d, J=1.1 Hz, 1H), 8.26-8.21 (m, 1H), 8.19-8.09 (m, 2H), 7.86 (ddd, J=8.3, 6.9, 1.3 Hz, 1H), 7.79-7.66 (m, 3H), 7.46 (ddd, J=8.6, 2.4, 1.1 Hz, 1H), 6.89 (t, J=5.7 Hz, 1H), 6.67 (t, J=5.8 Hz, 1H), 4.80 (d, J=5.8 Hz, 2H), 4.35 (d, J=5.6 Hz, 2H), 2.27 (s, 3H). LCMS (ES) [M+1]⁺ m/z 492.

### Example 1.142

### Synthesis of 1-{[1-(4-chloro-3-fluorophenyl)-3-methyl-1H-1,2,4-triazol-5-yl]methyl}-3-{[1-(quinolin-3-yl)-1H-1,2,4-triazol-5-yl]methyl}urea (Compound 126)

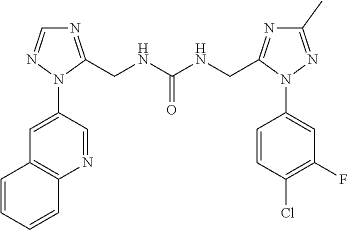 [SMILES: Cc1nc(CNC(=O)NCc2ncnn2-c2cnc3ccccc3c2)n(-c2ccc(Cl)c(F)c2)n1]

The title compound was synthesized according to General Procedure 7, Method A using [1-(quinolin-3-yl)-1H-1,2,4-triazol-5-yl]methanaminium chloride (66 mg; 0.25 mmol 1 eq. synthesized according to General Procedures 1, 2 and 3 starting from commercial quinolin-3-amine) and 1-[1-(4-chloro-3-fluorophenyl)-3-methyl-1H-1,2,4-triazol-5-yl]methanamine (Intermediate I-6; 60.3 mg, 0.25 mmol, 1 eq.) to provide 1-{[1-(4-chloro-3-fluorophenyl)-3-methyl-1H-1,2,4-triazol-5-yl]methyl}-3-{[1-(quinolin-3-yl)-1H-1,2,4-triazol-5-yl]methyl}urea as a colorless solid (37.5 mg; 30.4% yield). 1H NMR (400 MHz, DMSO-d6) δ 9.06 (d, J=2.5 Hz, 1H), 8.64 (dd, J=2.5, 0.8 Hz, 1H), 8.19 (s, 1H), 8.10 (dd, J=8.5, 1.1 Hz, 1H), 8.04 (dd, J=8.3, 1.5 Hz, 1H), 7.86 (ddd, J=8.5, 6.9, 1.5 Hz, 1H), 7.78-7.64 (m, 3H), 7.43 (ddd, J=8.7, 2.4, 1.1 Hz, 1H), 6.74 (t, J=5.7 Hz, 1H), 6.67 (t, J=5.6 Hz, 1H), 4.48 (d, J=5.7 Hz, 2H), 4.27 (d, J=5.6 Hz, 2H), 2.25 (s, 3H). LCMS (ES) [M+1]⁺ m/z 492.

### Example 1.143

### Synthesis of 1-{[1-(4-chloro-3-fluorophenyl)-3-methyl-1H-1,2,4-triazol-5-yl]methyl}-3-{[1-(quinazolin-6-yl)-1H-1,2,4-triazol-5-yl]methyl}urea (Compound 127)

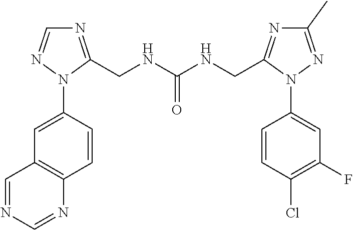 [SMILES: Cc1nc(CNC(=O)NCc2ncnn2-c2ccc3ncncc3c2)n(-c2ccc(Cl)c(F)c2)n1]

The title compound was synthesized according to General Procedure 7, Method A using [1-(quinazolin-6-yl)-1H-1,2,4-triazol-5-yl]methanaminium chloride (84 mg; 0.32 mmol 1.1 eq. synthesized according to General Procedures 2 and 3 starting from commercial 2-(quinazolin-6-yl)hydrazinium chloride) and 1-[1-(4-chloro-3-fluorophenyl)-3-methyl-1H-1,2,4-triazol-5-yl]methanamine (Intermediate I-6; 67.6 mg, 0.28 mmol, 1 eq.) to provide 1-{[1-(4-chloro-3-fluorophenyl)-3-methyl-1H-1,2,4-triazol-5-yl]methyl}-3-{[1-(quinazolin-6-yl)-1H-1,2,4-triazol-5-yl]methyl}urea as a colorless solid (25.6 mg; 18.5% yield). 1H NMR (400 MHz, DMSO-d6) δ 9.65 (s, 1H), 9.37 (s, 1H), 8.40 (d, J=2.3 Hz, 1H), 8.22 (dd, J=9.0, 2.3 Hz, 1H), 8.16 (d, J=9.2 Hz, 2H), 7.78-7.65 (m, 2H), 7.44 (ddd, J=8.6, 2.5, 1.2 Hz, 1H), 6.73 (t, J=5.7 Hz, 1H), 6.68 (t, J=5.7 Hz, 1H), 4.51 (d, J=5.7 Hz, 2H), 4.29 (d, J=5.6 Hz, 2H), 2.25 (s, 3H). LCMS (ES) [M+1]⁺ m/z 493.

### Example 1.144

### Synthesis of 1-{[1-(4-chloro-3-fluorophenyl)-3-methyl-1H-1,2,4-triazol-5-yl]methyl}-3-[(1-{imidazo[1,2-a]pyridin-7-yl}-1H-1,2,4-triazol-5-yl)methyl]urea (Compound 128)

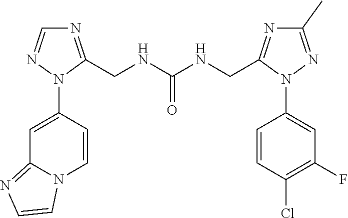 [SMILES: Cc1nc(CNC(=O)NCc2ncnn2-c2ccn3ccnc3c2)n(-c2ccc(Cl)c(F)c2)n1]

The title compound was synthesized according to General Procedure 7, Method A using (1-{imidazo[1,2-a]pyridin-7-yl}-1H-1,2,4-triazol-5-yl)methanaminium chloride (61 mg; 0.24 mmol 1 eq. synthesized according to General Procedures 1, 2 and 3 starting from commercial imidazo[1,2-a]pyridin-7-amine) and 1-[1-(4-chloro-3-fluorophenyl)-3-methyl-1H-1,2,4-triazol-5-yl]methanamine (Intermediate I-6; 58.6 mg, 0.24 mmol, 1 eq.) to provide 1-{[1-(4-chloro-3-fluorophenyl)-3-methyl-1H-1,2,4-triazol-5-yl]methyl}-3-[(1-{imidazo[1,2-a]pyridin-7-yl}-1H-1,2,4-triazol-5-yl)methyl]urea as a colorless solid (35.9 mg; 30.7% yield). 1H NMR (400 MHz, DMSO-d6) δ 8.69 (d, J=7.3 Hz, 1H), 8.12 (s, 1H), 8.06 (s, 1H), 7.88 (d, J=2.1 Hz, 1H), 7.77-7.65 (m, 3H), 7.48-7.41 (m, 1H), 7.12 (dd, J=7.3, 2.2 Hz, 1H), 6.73 (dt, J=7.9, 5.6 Hz, 2H), 4.46 (d, J=5.6 Hz, 2H), 4.34 (d, J=5.6 Hz, 2H), 2.27 (s, 3H). LCMS (ES) [M+1]⁺ m/z 481.

### Example 1.145

### Synthesis of 1-{[1-(4-chloro-3-fluorophenyl)-3-methyl-1H-1,2,4-triazol-5-yl]methyl}-3-{[1-(pyridin-3-yl)-1H-1,2,4-triazol-5-yl]methyl}urea (Compound 129)

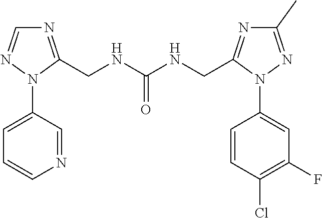 [SMILES: Cc1nc(CNC(=O)NCc2ncnn2-c2cccnc2)n(-c2ccc(Cl)c(F)c2)n1]

The title compound was synthesized according to General Procedure 7, Method A using [1-(pyridin-3-yl)-1H-1,2,4-triazol-5-yl]methanaminium chloride (49 mg; 0.23 mmol 1 eq. synthesized according to General Procedures 2 and 3 starting from commercial 2-(pyridin-3-yl)hydrazinium chloride) and 1-[1-(4-chloro-3-fluorophenyl)-3-methyl-1H-1,2,4-triazol-5-yl]methanamine (Intermediate I-6; 55.4 mg, 0.23 mmol, 1 eq.) to provide 1-{[1-(4-chloro-3-fluorophenyl)-3-methyl-1H-1,2,4-triazol-5-yl]methyl}-3-{[1-(pyridin-3-yl)-1H-1,2,4-triazol-5-yl]methyl}urea as a colorless solid (25.4 mg; 25% yield). 1H NMR (400 MHz, DMSO-d6) δ 8.79 (dd, J=2.5, 0.8 Hz, 1H), 8.68 (dd, J=4.8, 1.5 Hz, 1H), 8.13 (s, 1H), 8.04 (ddd, J=8.2, 2.6, 1.5 Hz, 1H), 7.79-7.68 (m, 2H), 7.58 (ddd, J=8.2, 4.8, 0.8 Hz, 1H), 7.45 (ddd, J=8.7, 2.4, 1.1 Hz, 1H), 6.71 (dt, J=11.6, 5.6 Hz, 2H), 4.36 (d, J=5.6 Hz, 2H), 4.33 (d, J=5.6 Hz, 2H), 2.27 (s, 3H). LCMS (ES) [M+1]⁺ m/z 442.

### Example 1.146

### Synthesis of 1-{[1-(4-chloro-3-fluorophenyl)-3-methyl-1H-1,2,4-triazol-5-yl]methyl}-3-{[1-(5-methoxypyridin-2-yl)-1H-1,2,4-triazol-5-yl]methyl}urea (Compound 130)

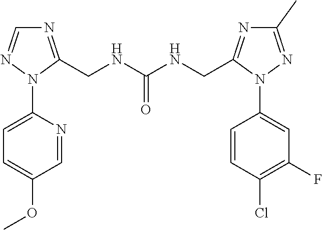 [SMILES: COc1ccc(-n2ncnc2CNC(=O)NCc2nc(C)nn2-c2ccc(Cl)c(F)c2)nc1]

Step 1

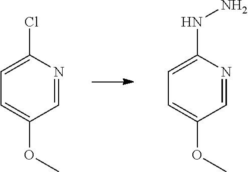

A solution of 6-chloro-3-pyridinyl methyl ether (1 000.00 mg; 6.97 mmol; 1.00 eq.) in hydrazine hydrate (7.50 mL; 7.50 V) was placed into a sealed vial and stirred at 120° C. After overnight the mixture was concentrated under reduced pressure, the residue was taken up in chloroform and washed with 1 N NaOH and the organic phase was dried over MgSO4, filtered and concentrated to provide crude 2-hydrazinyl-5-methoxypyridine as a yellow oil. This material was taken on to the next step without further purification.

Step 2

The title compound was synthesized according to General Procedure 7, Method A using [1-(5-methoxypyridin-2-yl)-1H-1,2,4-triazol-5-yl]methanaminium chloride (43 mg; 0.18 mmol 1 eq. synthesized according to General Procedures 2 and 3 starting from 2-hydrazinyl-5-methoxypyridine from step 1 above) and 1-[1-(4-chloro-3-fluorophenyl)-3-methyl-1H-1,2,4-triazol-5-yl]methanamine (Intermediate I-6; 42.8 mg, 0.18 mmol, 1 eq.) to provide 1-{[1-(4-chloro-3-fluorophenyl)-3-methyl-1H-1,2,4-triazol-5-yl]methyl}-3-{[1-(5-methoxypyridin-2-yl)-1H-1,2,4-triazol-5-yl]methyl}urea as a colorless solid (18.3 mg; 21.8% yield). 1H NMR (400 MHz, DMSO-d6) δ 8.24 (d, J=3.0 Hz, 1H), 8.06 (s, 1H), 7.75 (td, J=8.5, 8.1, 4.0 Hz, 3H), 7.66 (dd, J=9.0, 3.0 Hz, 1H), 7.49-7.44 (m, 1H), 6.88 (t, J=5.7 Hz, 1H), 6.62 (t, J=5.8 Hz, 1H), 4.65 (d, J=5.8 Hz, 2H), 4.36 (d, J=5.6 Hz, 2H), 3.89 (s, 3H), 2.28 (s, 3H). LCMS (ES) [M+1]+m/z 472.

### Example 1.147

### Synthesis of 1-{[1-(4-chloro-3-fluorophenyl)-3-methyl-1H-1,2,4-triazol-5-yl]methyl}-3-{[1-(6-cyclopropylpyridin-3-yl)-1H-1,2,4-triazol-5-yl]methyl}urea (Compound 131)

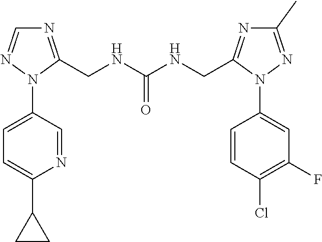 [SMILES: Cc1nc(CNC(=O)NCc2ncnn2-c2ccc(C3CC3)nc2)n(-c2ccc(Cl)c(F)c2)n1]

Step 1

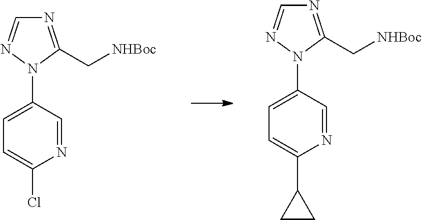 [SMILES: CC(C)(C)OC(=O)NCc1ncnn1-c1ccc(Cl)nc1 ; CC(C)(C)OC(=O)NCc1ncnn1-c1ccc(C2CC2)nc1]

A mixture of tert-butyl N-{[1-(6-chloropyridin-3-yl)-1H-1,2,4-triazol-5-yl]methyl}carbamate (53.00 mg; 0.17 mmol; 1.00 eq.; synthesized according to General Procedure 2 starting from commercial 2-chloro-5-hydrazinylpyridine), cyclopropylboronic acid (51.44 mg; 0.60 mmol; 3.50 eq.), tricyclohexylphosphane (4.80 mg; 0.02 mmol; 0.10 eq.) and potassium phosphate, tribasic (72.64 mg; 0.34 mmol; 2.00 eq.) in toluene (0.86 mL) and water (0.04 mL) was sparged with nitrogen for 10 minutes. Palladium acetate (3.84 mg; 0.02 mmol; 0.10 eq.) was then added and the mixture was heated to 100° C. After 2.5 h the mixture was diluted with EtOAc and washed with water and brine. The organic layer was dried over MgSO4, filtered and concentrated. The residue was purified by column chromatography eluting with 0-70% EtOAc in heptane to obtain tert-butyl N-{[1-(6-cyclopropylpyridin-3-yl)-1H-1,2,4-triazol-5-yl]methyl}carbamate as a yellow/orange semisolid (33.5 mg; 63%). LCMS (ES) [M+1]+m/z 316.

Step 2

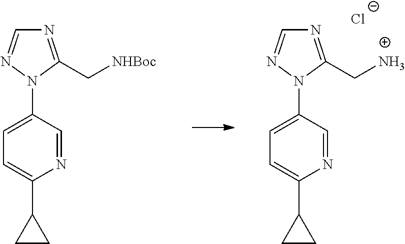 [SMILES: [Cl-] ; CC(C)(C)OC(=O)NCc1ncnn1-c1ccc(C2CC2)nc1 ; [NH3+]Cc1ncnn1-c1ccc(C2CC2)nc1]

To a mixture of tert-butyl N-{[1-(6-cyclopropylpyridin-3-yl)-1H-1,2,4-triazol-5-yl]methyl}carbamate (33.00 mg; 0.10 mmol; 1.00 eq.) in 1,2-dichloroethane (0.52 mL) at ambient temperature was added hydrochloric acid (0.13 mL; 4.00 mol/L in dioxane; 0.52 mmol; 5.00 eq.) slowly and the resulting mixture was stirred at ambient temperature. After 2 h the mixture was concentrated in vacuo to provide [1-(6-cyclopropylpyridin-3-yl)-1H-1,2,4-triazol-5-yl]methanaminium chloride as an off-white solid. This material was taken on to the next step without further purification assuming 100% yield (26 mg).

Step 3

The title compound was synthesized according to General Procedure 7, Method A using [1-(6-cyclopropylpyridin-3-yl)-1H-1,2,4-triazol-5-yl]methanaminium chloride (26.00 mg; 0.10 mmol; 1.00 eq. from step 2 above) and 1-[1-(4-chloro-3-fluorophenyl)-3-methyl-1H-1,2,4-triazol-5-yl]methanamine (Intermediate I-6; 24.86 mg; 0.10 mmol; 1.00 eq.) to provide 1-{[1-(4-chloro-3-fluorophenyl)-3-methyl-1H-1,2,4-triazol-5-yl]methyl}-3-{[1-(6-cyclopropylpyridin-3-yl)-1H-1,2,4-triazol-5-yl]methyl}urea as a colorless solid (4.5 mg; 9.04% yield). 1-H NMR (400 MHz, DMSO-d6) δ 8.56 (d, J=2.6 Hz, 1H), 8.09 (s, 1H), 7.85 (dd, J=8.4, 2.6 Hz, 1H), 7.74 (t, J=8.5 Hz, 2H), 7.45 (t, J=7.5 Hz, 2H), 6.70 (q, J=5.7 Hz, 2H), 4.32 (dd, J=8.3, 5.6 Hz, 4H), 2.27 (s, 3H), 2.19 (dt, J=7.9, 3.4 Hz, 1H), 1.05-0.90 (m, 4H). LCMS (ES) [M+1]⁺ m/z 482.

### Example 1.148

### Synthesis of 1-{[1-(4-chloro-3-fluorophenyl)-3-methyl-1H-1,2,4-triazol-5-yl]methyl}-3-{[1-(6-cyclobutylpyridin-3-yl)-1H-1,2,4-triazol-5-yl]methyl}urea (Compound 132)

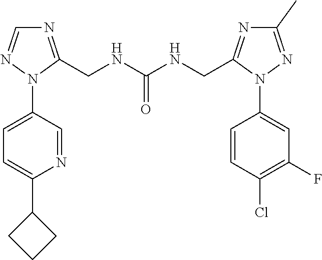 [SMILES: Cc1nc(CNC(=O)NCc2ncnn2-c2ccc(C3CCC3)nc2)n(-c2ccc(Cl)c(F)c2)n1]

Step 1

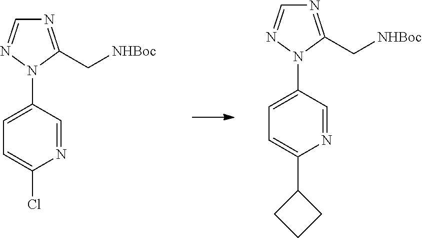 [SMILES: CC(C)(C)OC(=O)NCc1ncnn1-c1ccc(Cl)nc1 ; CC(C)(C)OC(=O)NCc1ncnn1-c1ccc(C2CCC2)nc1]

To a mixture of tert-butyl N-{[1-(6-chloropyridin-3-yl)-1H-1,2,4-triazol-5-yl]methyl}carbamate (71.00 mg; 0.23 mmol; 1.00 eq. synthesized according to General Procedure 2 starting from commercial 2-chloro-5-hydrazinylpyridine) and 1,1′-bis(diphenylphosphino)ferrocene]dichloropalladium(II) (16.77 mg; 0.02 mmol; 0.10 eq.) in 1,4-dioxane (2.29 mL) at ambient temperature under an atmosphere of Argon was added bromo(cyclobutyl)zinc (0.92 mL; 0.50 mol/L; 0.46 mmol; 2.00 eq.) drop-wise. After complete addition the mixture was heated to 80° C. under an atmosphere of Argon for 1.5 h. The mixture was then cooled to ambient temperature, water was added, and the suspension was stirred vigorously for 10 minutes. The mixture was then extracted with EtOAc. The organic phase was washed once with brine and the combined aqueous phase was back-extracted with EtOAc. The combined organic phase was dried over MgSO4, filtered and concentrated. The residue was purified by column chromatography eluting with 0-60% EtOAc in heptane to obtain tert-butyl N-{[1-(6-cyclobutylpyridin-3-yl)-1H-1,2,4-triazol-5-yl]methyl}carbamate (12 mg; 16% yield).

Step 2

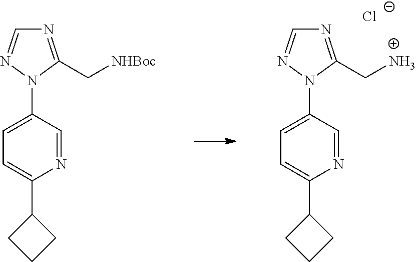 [SMILES: [Cl-] ; CC(C)(C)OC(=O)NCc1ncnn1-c1ccc(C2CCC2)nc1 ; [NH3+]Cc1ncnn1-c1ccc(C2CCC2)nc1]

To a mixture of tert-butyl N-{[1-(6-cyclobutylpyridin-3-yl)-1H-1,2,4-triazol-5-yl]methyl}carbamate (12.00 mg; 0.04 mmol; 1.00 eq.) in 1,2-dichloroethane (0.18 mL) at ambient temperature was added hydrochloric acid (0.05 mL; 4.00 mol/L in dioxane; 0.18 mmol; 5.00 eq.) slowly and the mixture was stirred at ambient temperature. After 2 h the mixture was concentrated in vacuo to provide [1-(6-cyclobutylpyridin-3-yl)-1H-1,2,4-triazol-5-yl]methanaminium chloride as an off-white solid. This material was taken on to the next step without further purification assuming 100% yield (9.6 mg).

Step 3

The title compound was synthesized according to General Procedure 7, Method A using [1-(6-cyclobutylpyridin-3-yl)-1H-1,2,4-triazol-5-yl]methanaminium chloride (9.60 mg; 0.04 mmol; 1.00 eq. from step 2 above) and 1-[1-(4-chloro-3-fluorophenyl)-3-methyl-1H-1,2,4-triazol-5-yl]methanamine (Intermediate I-6; 8.69 mg; 0.04 mmol; 1.00 eq.) to provide 1-{[1-(4-chloro-3-fluorophenyl)-3-methyl-1H-1,2,4-triazol-5-yl]methyl}-3-{[1-(6-cyclobutylpyridin-3-yl)-1H-1,2,4-triazol-5-yl]methyl}urea as a colorless solid (6.0 mg; 33.5% yield). 1H NMR (400 MHz, DMSO-d6) δ 8.70 (d, J=2.6 Hz, 1H), 8.11 (d, J=1.2 Hz, 1H), 7.92 (dd, J=8.3, 2.6 Hz, 1H), 7.78-7.70 (m, 2H), 7.47-7.43 (m, 1H), 7.41 (d, J=8.3 Hz, 1H), 6.71 (dt, J=10.7, 5.6 Hz, 2H), 4.33 (dd, J=5.4, 3.7 Hz, 4H), 3.74-3.69 (m, 1H), 2.31-2.24 (m, 5H), 2.07-1.95 (m, 2H), 1.84 (dd, J=10.6, 6.4 Hz, 2H). LCMS (ES) [M+1]⁺ m/z 496.

### Example 1.149

### Synthesis of 1-{[1-(4-chloro-3-fluorophenyl)-3-methyl-1H-1,2,4-triazol-5-yl]methyl}-3-{[1-(5-cyclopropylpyridin-3-yl)-1H-1,2,4-triazol-5-yl]methyl}urea (Compound 133)

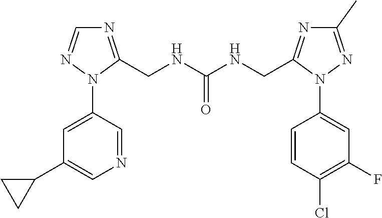 [SMILES: Cc1nc(CNC(=O)NCc2ncnn2-c2cncc(C3CC3)c2)n(-c2ccc(Cl)c(F)c2)n1]

Step 1

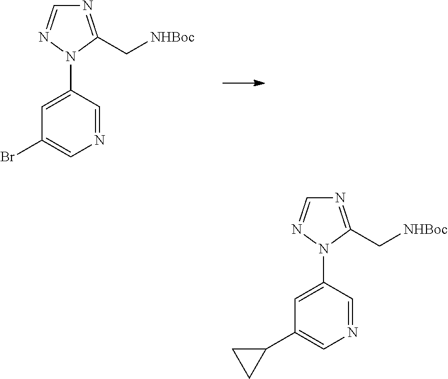 [SMILES: CC(C)(C)OC(=O)NCc1ncnn1-c1cncc(Br)c1 ; CC(C)(C)OC(=O)NCc1ncnn1-c1cncc(C2CC2)c1]

A mixture of tert-butyl N-{[1-(5-bromopyridin-3-yl)-1H-1,2,4-triazol-5-yl]methyl}carbamate (56.00 mg; 0.16 mmol; 1.00 eq. synthesized according to General Procedures 1 and 2 starting from commercial 5-bromo-3-pyridinamine), cyclopropylboronic acid (47.53 mg; 0.55 mmol; 3.50 eq.), tricyclohexylphosphane (4.43 mg; 0.02 mmol; 0.10 eq.) and potassium phosphate, tribasic (67.12 mg; 0.32 mmol; 2.00 eq.) in toluene (0.79 mL) and water (0.04 mL) was sparged with nitrogen for 5 minutes. Palladium acetate (3.55 mg; 0.02 mmol; 0.10 eq.) was then added and the mixture was heated to 100° C. for 2 h. The mixture was then diluted with EtOAc and washed with water and brine. The organic layer was dried over MgSO4, filtered and concentrated. The residue was purified by column chromatography (4 G ISCO Gold) eluting with 0-50% EtOAc in heptane to obtain tert-butyl N-{[1-(5-cyclopropylpyridin-3-yl)-1H-1,2,4-triazol-5-yl]methyl}carbamate as a tan solid (35.7 mg; 72%). LCMS (ES) [M+1]⁺ m/z 316.

Step 2

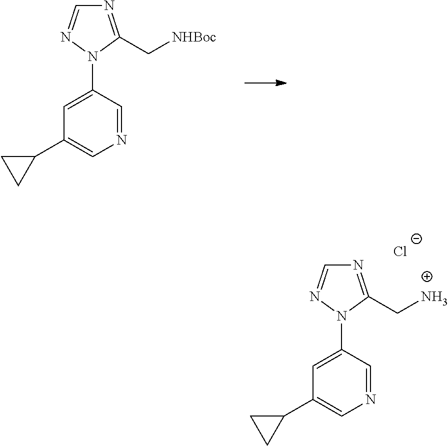 [SMILES: CC(C)(C)OC(=O)NCc1ncnn1-c1cncc(C2CC2)c1 ; [Cl-] ; [NH3+]Cc1ncnn1-c1cncc(C2CC2)c1]

To a mixture of tert-butyl N-{[1-(5-cyclopropylpyridin-3-yl)-1H-1,2,4-triazol-5-yl]methyl}carbamate (35.00 mg; 0.11 mmol; 1.00 eq.) in 1,2-dichloroethane (0.55 mL) at ambient temperature was added hydrochloric acid (0.14 mL; 4.00 mol/L in dioxane; 0.55 mmol; 5.00 eq.) slowly and the mixture was stirred at ambient temperature for 2 h. The mixture was then concentrated in vacuo to provide [1-(5-cyclopropylpyridin-3-yl)-1H-1,2,4-triazol-5-yl]methanaminium chloride as a white solid. This material was taken on to the next step without further purification assuming 100% yield (27 mg).

Step 3

The title compound was synthesized according to General Procedure 7, Method A using [1-(5-cyclopropylpyridin-3-yl)-1H-1,2,4-triazol-5-yl]methanaminium chloride (27.00 mg; 0.11 mmol; 1.00 eq. from step 2 above) and 1-[1-(4-chloro-3-fluorophenyl)-3-methyl-1H-1,2,4-triazol-5-yl]methanamine (Intermediate I-6; 25.81 mg; 0.11 mmol; 1.00 eq.) to provide 1-{[1-(4-chloro-3-fluorophenyl)-3-methyl-1H-1,2,4-triazol-5-yl]methyl}-3-{[1-(5-cyclopropylpyridin-3-yl)-1H-1,2,4-triazol-5-yl]methyl}urea as a colorless solid (16 mg; 31% yield). 1H NMR (400 MHz, DMSO-d6) δ 8.53 (d, J=2.3 Hz, 1H), 8.51 (d, J=2.0 Hz, 1H), 8.11 (s, 1H), 7.78-7.69 (m, 2H), 7.63 (t, J=2.2 Hz, 1H), 7.48-7.43 (m, 1H), 6.70 (q, J=5.9 Hz, 2H), 4.33 (dd, J=5.7, 2.7 Hz, 4H), 2.27 (s, 3H), 2.03 (tt, J=8.4, 5.1 Hz, 1H), 1.05-0.96 (m, 2H), 0.84-0.76 (m, 2H). LCMS (ES) [M+1]⁺ m/z 482.

### Example 1.150

### Synthesis of 1-{[1-(4-chloro-3-fluorophenyl)-3-methyl-1H-1,2,4-triazol-5-yl]methyl}-3-{[1-(5-methoxypyridin-3-yl)-1H-1,2,4-triazol-5-yl]methyl}urea (Compound 134)

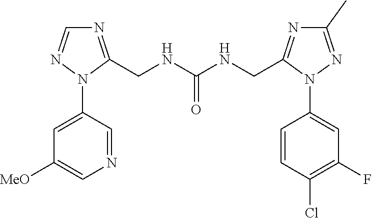 [SMILES: COc1cncc(-n2ncnc2CNC(=O)NCc2nc(C)nn2-c2ccc(Cl)c(F)c2)c1]

Step 1

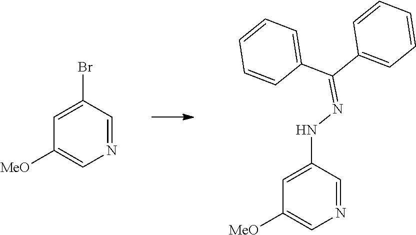 [SMILES: COc1cncc(Br)c1 ; COc1cncc(NN=C(c2ccccc2)c2ccccc2)c1]

A mixture of 3-bromo-5-methoxypyridine (500.00 mg; 2.66 mmol; 1.00 eq.), diphenylmethanone hydrazone (730.62 mg; 3.72 mmol; 1.40 eq.) and 1,1′-bis(diphenylphosphino)ferrocene (88.45 mg; 0.16 mmol; 0.06 eq.) in toluene (5.32 mL) was purged with Argon. Sodium tert-butoxide (383.35 mg; 3.99 mmol; 1.50 eq.) and palladium acetate (17.91 mg; 0.08 mmol; 0.03 eq.) were then added and the mixture was subjected to 2 cycles of evacuation/back filling with Argon and heated to 100° C. for 3 h. The reaction mixture was then cooled to ambient temperature, diluted with EtOAc and washed with water. The organic layer was dried over MgSO4, filtered and concentrated. The residue was purified by column chromatography eluting with 0-60% EtOAc in heptane to provide 3-[2-(diphenylmethylidene)hydrazin-1-yl]-5-methoxypyridine as an orange foam (692 mg; 85% yield). LCMS (ES) [M+1]⁺ m/z 304.

Step 2

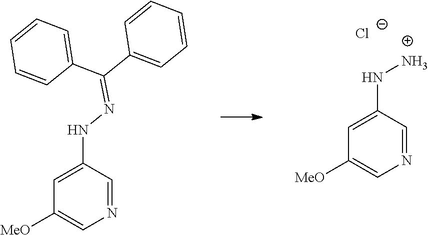 [SMILES: COc1cncc(NN=C(c2ccccc2)c2ccccc2)c1 ; COc1cncc(N[NH3+])c1 ; [Cl-]]

A suspension of 3-[2-(diphenylmethylidene)hydrazin-1-yl]-5-methoxypyridine (190.00 mg; 0.63 mmol; 1.00 eq.), in water (0.19 mL) at ambient temperature was treated with hydrochloric acid (0.78 mL; 4.00 mol/L in dioxane; 3.13 mmol; 5.00 eq.) and the resulting mixture was stirred at 85° C. for 8 h. The mixture was then cooled to ambient temperature and concentrated in vacuo. The residue was taken up in Et₂O, filtered, washed with DCM and air dried to provide 2-(5-methoxypyridin-3-yl)hydrazinium chloride as a brown solid. This material (84 mg; 77%) was taken on to the next step without further purification.

Step 3

The title compound was synthesized according to General Procedure 7, Method A using [1-(5-methoxypyridin-3-yl)-1H-1,2,4-triazol-5-yl]methanaminium chloride (22.00 mg; 0.09 mmol; 1.00 eq. synthesized according to General Procedures 2 and 3 starting from 2-(5-methoxypyridin-3-yl)hydrazinium chloride from step 2 above) and 1-[1-(4-chloro-3-fluorophenyl)-3-methyl-1H-1,2,4-triazol-5-yl]methanamine (Intermediate I-6; 21.91 mg; 0.09 mmol; 1.00 eq.) to provide 1-{[1-(4-chloro-3-fluorophenyl)-3-methyl-1H-1,2,4-triazol-5-yl]methyl}-3-{[1-(5-methoxypyridin-3-yl)-1H-1,2,4-triazol-5-yl]methyl}urea as a colorless solid (11.9 mg; 27.7% yield). 1H NMR (400 MHz, DMSO-d6) δ 8.40 (d, J=2.6 Hz, 1H), 8.37 (d, J=2.0 Hz, 1H), 8.12 (s, 1H), 7.78-7.70 (m, 2H), 7.67 (t, J=2.4 Hz, 1H), 7.48-7.42 (m, 1H), 6.71 (dt, J=11.5, 5.7 Hz, 2H), 4.39 (d, J=5.7 Hz, 2H), 4.32 (d, J=5.6 Hz, 2H), 3.85 (s, 3H), 2.27 (s, 3H). LCMS (ES) [M+1]⁺ m/z 472.

### Example 1.151

### Synthesis of 1-{[1-(4-chloro-3-fluorophenyl)-3-methyl-1H-1,2,4-triazol-5-yl]methyl}-3-{[1-(7-fluoroquinolin-3-yl)-1H-1,2,4-triazol-5-yl]methyl}urea (Compound 135)

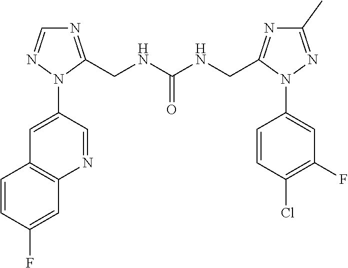 [SMILES: Cc1nc(CNC(=O)NCc2ncnn2-c2cnc3cc(F)ccc3c2)n(-c2ccc(Cl)c(F)c2)n1]

The title compound was synthesized according to General Procedure 7, Method A using [1-(7-fluoroquinolin-3-yl)-1H-1,2,4-triazol-5-yl]methanaminium chloride (55.00 mg; 0.20 mmol; 1.00 eq. synthesized according to General Procedures 1, 2 and 3 starting from commercial 7-fluoroquinolin-3-amine dihydrochloride) and 1-[1-(4-chloro-3-fluorophenyl)-3-methyl-1H-1,2,4-triazol-5-yl]methanamine (Intermediate I-6; 47.32 mg; 0.20 mmol; 1.00 eq.) to provide 1-{[1-(4-chloro-3-fluorophenyl)-3-methyl-1H-1,2,4-triazol-5-yl]methyl}-3-{[1-(7-fluoroquinolin-3-yl)-1H-1,2,4-triazol-5-yl]methyl}urea as a colorless solid (21.2 mg; 21.1% yield). 1H NMR (400 MHz, DMSO-d6) δ 9.08 (d, J=2.4 Hz, 1H), 8.69 (d, J=2.5 Hz, 1H), 8.18 (s, 1H), 8.14 (dd, J=9.1, 6.2 Hz, 1H), 7.86 (dd, J=10.2, 2.5 Hz, 1H), 7.77-7.68 (m, 2H), 7.65 (td, J=8.8, 2.6 Hz, 1H), 7.46-7.39 (m, 1H), 6.72 (t, J=5.6 Hz, 1H), 6.65 (t, J=5.7 Hz, 1H), 4.47 (d, J=5.6 Hz, 2H), 4.26 (d, J=5.6 Hz, 2H), 2.25 (s, 3H). LCMS (ES) [M+1]⁺ m/z 510.

### Example 1.152

### Synthesis of 3-{[1-(4-chloro-3-fluorophenyl)-3-methyl-1H-1,2,4-triazol-5-yl]methyl}-1-{[1-(8-fluoroquinolin-3-yl)-1H-1,2,4-triazol-5-yl]methyl}urea (Compound 136)

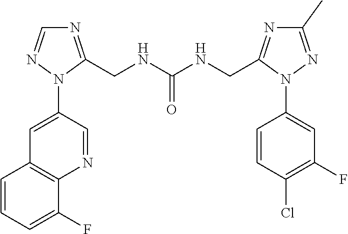 [SMILES: Cc1nc(CNC(=O)NCc2ncnn2-c2cnc3c(F)cccc3c2)n(-c2ccc(Cl)c(F)c2)n1]

The synthesis of the title compound is described in General Procedure 7, Method A.

### Example 1.153

### Synthesis of 1-{[1-(4-chloro-3-fluorophenyl)-3-methyl-1H-1,2,4-triazol-5-yl]methyl}-3-{[1-(5-methylpyridin-3-yl)-1H-1,2,4-triazol-5-yl]methyl}urea (Compound 137)

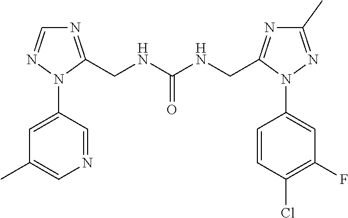 [SMILES: Cc1cncc(-n2ncnc2CNC(=O)NCc2nc(C)nn2-c2ccc(Cl)c(F)c2)c1]

The title compound was synthesized according to General Procedure 7, Method A using [1-(5-methylpyridin-3-yl)-1H-1,2,4-triazol-5-yl]methanaminium chloride (26.00 mg; 0.12 mmol; 1.00 eq. synthesized as described in Example 1.149 used to prepare Compound 133, steps 1 and 2 using methylboronic acid instead of cyclopropylboronic acid) and 1-[1-(4-chloro-3-fluorophenyl)-3-methyl-1H-1,2,4-triazol-5-yl]methanamine (Intermediate I-6; 27.73 mg; 0.12 mmol; 1.00 eq.) to provide 1-{[1-(4-chloro-3-fluorophenyl)-3-methyl-1H-1,2,4-triazol-5-yl]methyl -3-{[1-(5-methylpyridin-3-yl)-1H-1,2,4-triazol-5-yl]methyl}urea as a colorless solid (21.6 mg; 41.1% yield). 1H NMR (400 MHz, DMSO-d6) δ 8.59 (d, J=2.4 Hz, 1H), 8.53-8.49 (m, 1H), 8.11 (s, 1H), 7.87-7.83 (m, 1H), 7.77-7.71 (m, 2H), 7.45 (ddd, J=8.6, 2.4, 1.1 Hz, 1H), 6.70 (dt, J=7.6, 5.7 Hz, 2H), 4.37 (d, J=5.6 Hz, 2H), 4.33 (d, J=5.6 Hz, 2H), 2.35 (s, 3H), 2.27 (s, 3H). LCMS (ES) [M+1]⁺ m/z 456.

### Example 1.154

### Synthesis of 1-{[1-(4-chloro-3-fluorophenyl)-3-methyl-1H-1,2,4-triazol-5-yl]methyl}-3-{[3-methyl-1-(quinolin-3-yl)-1H-1,2,4-triazol-5-yl]methyl}urea (Compound 138)

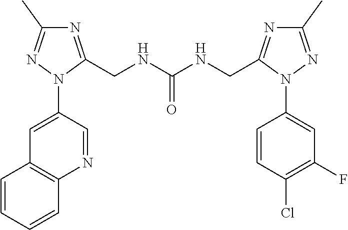 [SMILES: Cc1nc(CNC(=O)NCc2nc(C)nn2-c2cnc3ccccc3c2)n(-c2ccc(Cl)c(F)c2)n1]

The title compound was synthesized according to General Procedure 7, Method A using [3-methyl-1-(quinolin-3-yl)-1H-1,2,4-triazol-5-yl]methanamine (43.00 mg; 0.18 mmol; 1.00 eq. synthesized according to General Procedure 5 starting from commercial 2-(quinolin-3-yl)hydrazinium chloride) and 1-[1-(4-chloro-3-fluorophenyl)-3-methyl-1H-1,2,4-triazol-5-yl]methanamine (Intermediate I-6; 43.25 mg; 0.18 mmol; 1.00 eq.) to provide 1-{[1-(4-chloro-3-fluorophenyl)-3-methyl-1H-1,2,4-triazol-5-yl]methyl}-3-{[3-methyl-1-(quinolin-3-yl)-1H-1,2,4-triazol-5-yl]methyl}urea as a colorless solid (29.5 mg; 32.4% yield). 1H NMR (400 MHz, DMSO-d6) δ 9.04 (d, J=2.5 Hz, 1H), 8.60 (d, J=2.5 Hz, 1H), 8.09 (d, J=8.4 Hz, 1H), 8.03 (dd, J=8.3, 1.5 Hz, 1H), 7.84 (ddd, J=8.4, 6.9, 1.5 Hz, 1H), 7.78-7.64 (m, 3H), 7.43 (ddd, J=8.5, 2.5, 1.2 Hz, 1H), 6.73 (t, J=5.7 Hz, 1H), 6.67 (t, J=5.7 Hz, 1H), 4.43 (d, J=5.6 Hz, 2H), 4.29 (d, J=5.6 Hz, 2H), 2.33 (s, 3H), 2.25 (s, 3H). LCMS (ES) [M+1]⁺ m/z 506.

### Example 1.155

### Synthesis of 1-{[1-(4-chloro-3-fluorophenyl)-3-methyl-1H-1,2,4-triazol-5-yl]methyl}-3-{[1-(6-fluoroquinolin-3-yl)-1H-1,2,4-triazol-5-yl]methyl}urea (Compound 139)

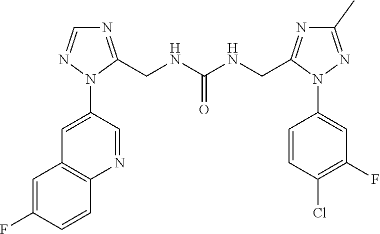 [SMILES: Cc1nc(CNC(=O)NCc2ncnn2-c2cnc3ccc(F)cc3c2)n(-c2ccc(Cl)c(F)c2)n1]

The title compound was synthesized according to General Procedure 7, Method A using [1-(6-fluoroquinolin-3-yl)-1H-1,2,4-triazol-5-yl]methanaminium chloride (72.00 mg; 0.26 mmol; 1.00 eq. synthesized according to General Procedures 1, 2 and 3 starting from commercial 6-fluoroquinolin-3-amine) and 1-[1-(4-chloro-3-fluorophenyl)-3-methyl-1H-1,2,4-triazol-5-yl]methanamine (Intermediate I-6; 61.95 mg; 0.26 mmol; 1.00 eq.) to provide 1-{[1-(4-chloro-3-fluorophenyl)-3-methyl-1H-1,2,4-triazol-5-yl]methyl}-3-{[1-(6-fluoroquinolin-3-yl)-1H-1,2,4-triazol-5-yl]methyl}urea as a colorless solid (26.3 mg; 20.0% yield). 1H NMR (400 MHz, DMSO-d6) δ 9.05 (d, J=2.4 Hz, 1H), 8.62 (d, J=2.5 Hz, 1H), 8.25-8.08 (m, 2H), 7.83 (dd, J=9.3, 2.9 Hz, 1H), 7.80-7.64 (m, 3H), 7.43 (ddd, J=8.7, 2.5, 1.2 Hz, 1H), 6.73 (t, J=5.7 Hz, 1H), 6.66 (t, J=5.7 Hz, 1H), 4.49 (d, J=5.6 Hz, 2H), 4.26 (d, J=5.6 Hz, 2H), 2.25 (s, 3H). LCMS (ES) [M+1]⁺ m/z 510.

### Example 1.156

### Synthesis of 1-{[1-(4-chloro-3-fluorophenyl)-3-methyl-1H-1,2,4-triazol-5-yl]methyl}-3-{[1-(5-fluoroquinolin-3-yl)-1H-1,2,4-triazol-5-yl]methyl}urea (Compound 140)

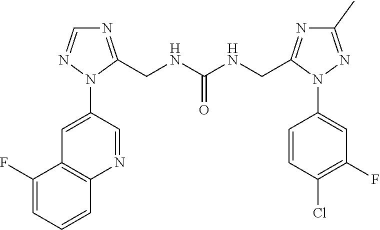 [SMILES: Cc1nc(CNC(=O)NCc2ncnn2-c2cnc3cccc(F)c3c2)n(-c2ccc(Cl)c(F)c2)n1]

The title compound was synthesized according to General Procedure 7, Method A using [1-(5-fluoroquinolin-3-yl)-1H-1,2,4-triazol-5-yl]methanaminium chloride (31.00 mg; 0.11 mmol; 1.00 eq. synthesized according to General Procedures 1, 2 and 3 starting from commercial 5-fluoroquinolin-3-amine) and 1-[1-(4-chloro-3-fluorophenyl)-3-methyl-1H-1,2,4-triazol-5-yl]methanamine (Intermediate I-6; 26.67 mg; 0.11 mmol; 1.00 eq.) to provide 1-{[1-(4-chloro-3-fluorophenyl)-3-methyl-1H-1,2,4-triazol-5-yl]methyl}-3-{[1-(5-fluoroquinolin-3-yl)-1H-1,2,4-triazol-5-yl]methyl}urea as a colorless solid (5.3 mg; 9.4% yield). 1H NMR (400 MHz, DMSO-d6) δ 9.11 (d, J=2.4 Hz, 1H), 8.74 (d, J=2.4 Hz, 1H), 8.20 (s, 1H), 7.97 (d, J=8.5 Hz, 1H), 7.86 (td, J=8.2, 6.0 Hz, 1H), 7.77-7.66 (m, 2H), 7.55 (dd, J=10.0, 7.8 Hz, 1H), 7.42 (ddd, J=8.7, 2.5, 1.2 Hz, 1H), 6.74 (t, J=5.7 Hz, 1H), 6.66 (t, J=5.6 Hz, 1H), 4.47 (d, J=5.6 Hz, 2H), 4.25 (d, J=5.6 Hz, 2H), 2.25 (s, 3H). LCMS (ES) [M+1]⁺ m/z 510.

### Example 1.157

### Synthesis of 1-{[1-(4-chloro-3-fluorophenyl)-3-methyl-1H-1,2,4-triazol-5-yl]methyl}-3-{[1-(6-ethylpyridin-3-yl)-1H-1,2,4-triazol-5-yl]methyl}urea (Compound 141)

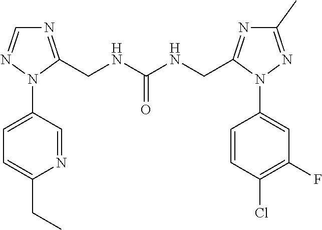 [SMILES: CCc1ccc(-n2ncnc2CNC(=O)NCc2nc(C)nn2-c2ccc(Cl)c(F)c2)cn1]

Step 1

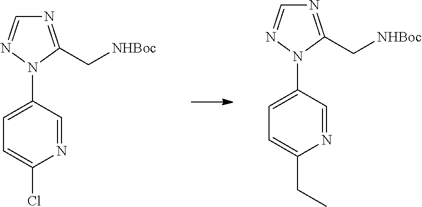 [SMILES: CC(C)(C)OC(=O)NCc1ncnn1-c1ccc(Cl)nc1 ; CCc1ccc(-n2ncnc2CNC(=O)OC(C)(C)C)cn1]

To a mixture of dichloropalladium (0.86 mg; 0.00 mmol; 0.01 eq.) and 1,1′-bis(diphenylphosphino)ferrocene (2.68 mg; 0.00 mmol; 0.01 eq.) in dioxane (0.1 mL) at ambient temperature under nitrogen was added a solution of tert-butyl N-{[1-(6-chloropyridin-3-yl)-1H-1,2,4-triazol-5-yl]methyl}carbamate (100.00 mg; 0.32 mmol; 1.00 eq. synthesized according to General Procedure 2 starting from commercial 2-chloro-5-hydrazinylpyridine) in 1,4-dioxane (1.29 mL). Diethylzinc (0.29 mL; 15.00% w/w; 0.32 mmol; 1.00 eq.) was then added drop-wise and the resulting mixture was heated to 50° C. under a nitrogen atmosphere. After 1 h 0.5 eq. of zinc reagent were added, and the temperature was increased to 60° C. After an additional 1.5 h the mixture was cooled to ambient temperature, diluted with EtOAc, washed with water, dried over MgSO4, filtered and concentrated. The residue was purified by column chromatography eluting with 0-80% EtOAc in heptane to provide tert-butyl N-{[1-(6-ethylpyridin-3-yl)-1H-1,2,4-triazol-5-yl]methyl}carbamate as a clear oil (47 mg; 48% yield) which turned into a white film upon drying over the weekend.

Step 2

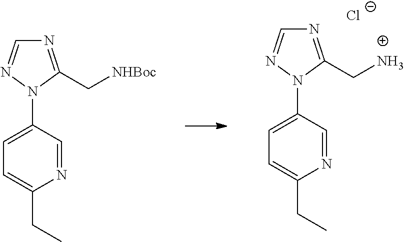 [SMILES: [Cl-] ; CCc1ccc(-n2ncnc2CNC(=O)OC(C)(C)C)cn1 ; CCc1ccc(-n2ncnc2C[NH3+])cn1]

To a mixture of tert-butyl N-{[1-(6-ethylpyridin-3-yl)-1H-1,2,4-triazol-5-yl]methyl}carbamate (47.00 mg; 0.15 mmol; 1.00 eq.) in 1,2-dichloroethane (0.77 mL) at ambient temperature was added hydrochloric acid (0.19 mL; 4.00 mol/L in dioxane; 0.77 mmol; 5.00 eq.) slowly and the resulting mixture was stirred at ambient temperature for 1.5 h. The mixture was then concentrated in vacuo to provide [1-(6-ethylpyridin-3-yl)-1H-1,2,4-triazol-5-yl]methanaminium chloride as a tan solid which was carried on to the next step without further purification assuming 100% yield (37 mg).

Step 3

The title compound was synthesized according to General Procedure 7, Method A using [1-(6-ethylpyridin-3-yl)-1H-1,2,4-triazol-5-yl]methanaminium chloride (37.00 mg; 0.15 mmol; 1.00 eq. from step 2 above) and 1-[1-(4-chloro-3-fluorophenyl)-3-methyl-1H-1,2,4-triazol-5-yl]methanamine (Intermediate I-6; 37.15 mg; 0.15 mmol; 1.00 eq.) to provide 1-{[1-(4-chloro-3-fluorophenyl)-3-methyl-1H-1,2,4-triazol-5-yl]methyl}-3-{[1-(6-ethylpyridin-3-yl)-1H-1,2,4-triazol-5-yl]methyl}urea as a colorless solid (22.7 mg; 31.3% yield). 1-H NMR (400 MHz, DMSO-d6) δ 8.66 (t, J=1.6 Hz, 1H), 8.10 (s, 1H), 7.92 (dd, J=8.3, 2.6 Hz, 1H), 7.78-7.70 (m, 2H), 7.47-7.42 (m, 2H), 6.70 (dt, J=8.9, 5.6 Hz, 2H), 4.33 (dd, J=5.7, 1.6 Hz, 4H), 2.82 (q, J=7.6 Hz, 2H), 2.27 (s, 3H), 1.24 (t, J=7.6 Hz, 3H). LCMS (ES) [M+1]⁺ m/z 470.

### Example 1.158

### Synthesis of 1-{[1-(4-chloro-3-fluorophenyl)-3-methyl-1H-1,2,4-triazol-5-yl]methyl}-3-ethyl-3-{[1-(quinolin-3-yl)-1H-1,2,4-triazol-5-yl]methyl}urea (Compound 142)

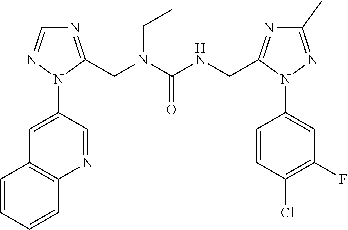 [SMILES: CCN(Cc1ncnn1-c1cnc2ccccc2c1)C(=O)NCc1nc(C)nn1-c1ccc(Cl)c(F)c1]

The title compound was synthesized according to General Procedure 7, Method A using 1-[1-(4-chloro-3-fluorophenyl)-3-methyl-1H-1,2,4-triazol-5-yl]methanamine (Intermediate I-6; 54.00 mg; 0.22 mmol; 1.00 eq.) and [ethyl({[1-(quinolin-3-yl)-1H-1,2,4-triazol-5-yl]methyl})azanium chloride (65.02 mg; 0.22 mmol; 1.00 eq. synthesized as described in Example 1.138 for the preparation of Compound 122, steps 1 and 2 using tert-butyl N-{[1-(quinolin-3-yl)-1H-1,2,4-triazol-5-yl]methyl}carbamate synthesized according to General Procedures 1 and 2 starting from commercial quinolin-3-amine) to provide 1-{[1-(4-chloro-3-fluorophenyl)-3-methyl-1H-1,2,4-triazol-5-yl]methyl}-3-ethyl-3-{[1-(quinolin-3-yl)-1H-1,2,4-triazol-5-yl]methyl}urea as a colorless solid (51.4 mg; 44.0% yield). 1-H NMR (400 MHz, DMSO-d6) δ 9.03 (d, J=2.5 Hz, 1H), 8.59 (d, J=2.5 Hz, 1H), 8.16 (s, 1H), 8.10 (d, J=8.5 Hz, 1H), 8.01 (dd, J=8.3, 1.5 Hz, 1H), 7.85 (ddd, J=8.4, 6.9, 1.5 Hz, 1H), 7.69 (ddd, J=10.6, 7.0, 3.3 Hz, 3H), 7.41 (ddd, J=8.6, 2.5, 1.2 Hz, 1H), 7.00 (t, J=5.4 Hz, 1H), 4.65 (s, 2H), 4.20 (d, J=5.3 Hz, 2H), 3.19 (q, J=7.0 Hz, 2H), 2.24 (s, 3H), 0.92 (t, J=7.0 Hz, 3H). LCMS (ES) [M+1]⁺ m/z 520.

### Example 1.159

### Synthesis of 1-{[1-(4-chloro-3-fluorophenyl)-1H-1,2,4-triazol-5-yl]methyl}-1-ethyl-3-{[3-methyl-1-(quinolin-3-yl)-1H-1,2,4-triazol-5-yl]methyl}urea (Compound 143)

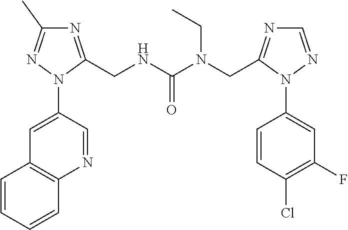 [SMILES: CCN(Cc1ncnn1-c1ccc(Cl)c(F)c1)C(=O)NCc1nc(C)nn1-c1cnc2ccccc2c1]

The title compound was synthesized according to General Procedure 7, Method A using [3-methyl-1-(quinolin-3-yl)-1H-1,2,4-triazol-5-yl]methanamine (11.00 mg; 0.05 mmol; 1.00 eq. synthesized according to General Procedure 5 starting from commercial 2-(quinolin-3-yl)hydrazinium chloride) and {[1-(4-chloro-3-fluorophenyl)-1H-1,2,4-triazol-5-yl]methyl}(ethyl)amine (Example 1.46 for the preparation of Compound 30, step 2; 11.71 mg; 0.05 mmol; 1.00 eq.) to provide 1-{[1-(4-chloro-3-fluorophenyl)-1H-1,2,4-triazol-5-yl]methyl}-1-ethyl-3-{[3-methyl-1-(quinolin-3-yl)-1H-1,2,4-triazol-5-yl]methyl}urea as a colorless solid (7.8 mg; 32.6% yield). 1H NMR (400 MHz, DMSO-d6) δ 9.02 (d, J=2.4 Hz, 1H), 8.57 (d, J=2.4 Hz, 1H), 8.08 (d, J=8.4 Hz, 1H), 8.02 (d, J=9.9 Hz, 2H), 7.84 (ddd, J=8.4, 6.9, 1.5 Hz, 1H), 7.80-7.61 (m, 3H), 7.42 (ddd, J=8.7, 2.5, 1.2 Hz, 1H), 7.03 (t, J=5.4 Hz, 1H), 4.42 (d, J=8.6 Hz, 2H), 4.39 (s, 2H), 3.10 (q, J=7.0 Hz, 2H), 2.32 (s, 3H), 0.79 (t, J=7.0 Hz, 3H). LCMS (ES) [M+1]⁺ m/z 520.

### Example 1.160

### Synthesis of 1-{[1-(4-chloro-3-fluorophenyl)-1H-1,2,4-triazol-5-yl]methyl}-3-{[3-methyl-1-(quinolin-3-yl)-1H-1,2,4-triazol-5-yl]methyl}urea (Compound 144)

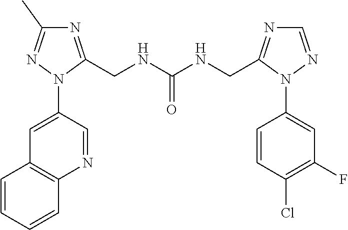 [SMILES: Cc1nc(CNC(=O)NCc2ncnn2-c2ccc(Cl)c(F)c2)n(-c2cnc3ccccc3c2)n1]

The title compound was synthesized according to General Procedure 7, Method A using [3-methyl-1-(quinolin-3-yl)-1H-1,2,4-triazol-5-yl]methanamine (20.00 mg; 0.08 mmol; 1.00 eq. synthesized according to General Procedure 5 starting from commercial 2-(quinolin-3-yl)hydrazinium chloride) and [1-(4-chloro-3-fluorophenyl)-1H-1,2,4-triazol-5-yl]methanamine (Intermediate I-4; 18.94 mg; 0.08 mmol; 1.00 eq.) to provide 1-{[1-(4-chloro-3-fluorophenyl)-1H-1,2,4-triazol-5-yl]methyl}-3-{[3-methyl-1-(quinolin-3-yl)-1H-1,2,4-triazol-5-yl]methyl}urea as a colorless solid (12.2 mg; 29.7% yield). 1H NMR (400 MHz, DMSO-d6) δ 9.04 (d, J=2.5 Hz, 1H), 8.60 (d, J=2.5 Hz, 1H), 8.14-8.05 (m, 2H), 8.03 (dd, J=8.4, 1.5 Hz, 1H), 7.84 (ddd, J=8.4, 6.9, 1.5 Hz, 1H), 7.80-7.72 (m, 2H), 7.69 (ddd, J=8.2, 6.9, 1.2 Hz, 1H), 7.46 (ddd, J=8.6, 2.4, 1.2 Hz, 1H), 6.73 (t, J=5.6 Hz, 1H), 6.68 (t, J=5.7 Hz, 1H), 4.42 (d, J=5.6 Hz, 2H), 4.34 (d, J=5.6 Hz, 2H), 2.33 (s, 3H). LCMS (ES) [M+1]⁺ m/z 492.

### Example 1.161

### Synthesis of 1-{[1-(4-chloro-3-fluorophenyl)-1H-1,2,4-triazol-5-yl]methyl}-3-{[1-(7-fluoroquinolin-3-yl)-1H-1,2,4-triazol-5-yl]methyl}urea (Compound 145)

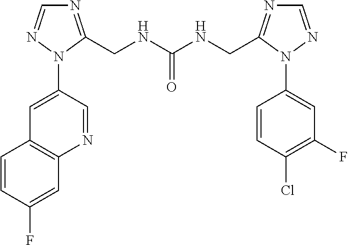 [SMILES: O=C(NCc1ncnn1-c1ccc(Cl)c(F)c1)NCc1ncnn1-c1cnc2cc(F)ccc2c1]

The title compound was synthesized according to General Procedure 7, Method A using [1-(7-fluoroquinolin-3-yl)-1H-1,2,4-triazol-5-yl]methanaminium chloride (36.00 mg; 0.15 mmol; 1.00 eq. synthesized according to General Procedures 1, 2 and 3 starting from commercial 7-fluoroquinolin-3-amine dihydrochloride) and [1-(4-chloro-3-fluorophenyl)-1H-1,2,4-triazol-5-yl]methanamine (Intermediate I-4; 33.54 mg; 0.15 mmol; 1.00 eq.) to provide 1-{[1-(4-chloro-3-fluorophenyl)-1H-1,2,4-triazol-5-yl]methyl}-3-{[1-(7-fluoroquinolin-3-yl)-1H-1,2,4-triazol-5-yl]methyl}urea as a colorless solid (21.6 mg; 29.4% yield). ¹H NMR (400 MHz, DMSO-d6) δ 9.08 (d, J=2.4 Hz, 1H), 8.69 (d, J=2.5 Hz, 1H), 8.18 (s, 1H), 8.15 (dd, J=9.1, 6.3 Hz, 1H), 8.07 (s, 1H), 7.87 (dd, J=10.3, 2.6 Hz, 1H), 7.80-7.71 (m, 2H), 7.66 (td, J=8.8, 2.6 Hz, 1H), 7.45 (dd, J=8.9, 2.5 Hz, 1H), 6.75 (t, J=5.7 Hz, 1H), 6.70 (t, J=5.7 Hz, 1H), 4.46 (d, J=5.6 Hz, 2H), 4.31 (d, J=5.6 Hz, 2H). LCMS (ES) [M+1]⁺ m/z 496.

### Example 1.162

### Synthesis of 1,3-bis({[1-(3,4-dimethylphenyl)-1H-1,2,3,4-tetrazol-5-yl]methyl})urea (Compound 146)

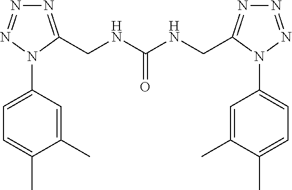 [SMILES: Cc1ccc(-n2nnnc2CNC(=O)NCc2nnnn2-c2ccc(C)c(C)c2)cc1C]

The synthesis of the title compound is described in General Procedure 6; Method A.

### II. BIOLOGICAL EXAMPLES

### Example 2.1

### Pyruvate Kinase Activation Methods

Protein Production and Purification

Human pyruvate kinase R (PKR), residues 14-574, and human pyruvate kinase M2, residues 1-531, were cloned into expression plasmids and obtained from ATUM Bio (Newark, Calif.). Proteins were translated as fusions with 6×-His, 8×-Arg, and SUMO at the N-terminus. A third construct of human PKR was truncated, residues 50-574, and similarly cloned for use in crystallography experiments. All proteins were cloned, expressed in E. coli, and purified using similar protocols. Cells were grown at 30° C. in Luria-Bertani broth supplemented with 0.4% glucose to OD600=0.8 and induced with 0.4 mM IPTG at 16° C. for 18 hours. Cells were harvested by centrifugation, resuspended in 50 mM potassium phosphate pH 8.0, 500 mM NaCl, 25 mM imidazole pH 8.0, and 3 mM (3-mercaptoethanol. Cells were lysed using a LM20 microfluidizer from Microfluidics (Westwood, Mass.). The crude lysate was immediately supplemented with 0.2 mM phenylmethylsulfonyl fluoride (PMSF) and centrifuged at 20,000 g for 20 minutes. The soluble fraction was subsequently incubated with 2 mL Ni-NTA (GE Healthcare) per 1,000 ODs for 1 hour at 4° C. Following incubation with the Ni-NTA resin, lysate was removed by pelleting resin at 2,500 g for 3 minutes and washed 3 times with 9 bed volumes of 50 mM potassium phosphate pH 8.0, 500 mM NaCl, 25 mM imidazole, and 3 mM 3-mercaptoethanol. Following the batch wash Ni-NTA resin was loaded onto a gravity column and His-tagged protein was eluted with 6 bed volumes of 10 mM Tris/HCl pH 8.0, 200 mM NaCl, 500 mM imidazole, and 3 mM (3-mercaptoethanol. Eluted protein was dialyzed overnight against 10 mM Tris/HCl pH 8.0, 200 mM NaCl, and 1 mM DTT and the 6×-His-8×-Arg-SUMO-tag was cleaved using a 20:1 molar ratio of protein:3C protease. The protein was purified by anion exchange chromatography on a HiTrapQ or MonoQ 10/100 GL column (GE Healthcare) via a linear NaCl gradient and twice by size exclusion chromatography using a Superdex S200 26/60 column (GE Healthcare) run in 10 mM Tris/HCl pH 8.0, 200 mM NaCl, 10 mM MgCl₂, 1 mM DTT. Proteins were concentrated to −12 mg/mL for crystallization and flash frozen for storage.

### Example 2.2

Biochemical Assay for Activation of Pyruvate Kinase R

Activation of pyruvate kinase R (PKR) and pyruvate kinase M2 (PKM2) were measured using a luminescence based measurement of ATP generation and the Kinase-Glo luminescent kinase reagent kit. 0.1 nM of PKR or PKM2 was incubated for 1 hour with 0.3 mM ADP, 2 mM FBP, 1% DMSO, compound and Assay Buffer mix (500 mM Tris/HCl pH 7.5, 500 mM NaCl, 50 mM MgCl₂, BSA, and 2 mM DTT, in a total reaction volume of 30 uL. After one hour, PEP was added to the reaction at a final concentration of 0.1 mM and incubated for another hour at room temperature. 30 uL of KinaseGlo reagent was added and the reaction was incubated for 15 minutes. Endpoint luminescence data was measured using an EnVision plate reader (PerkinElmer). The assay results are provided in Table 2.

TABLE 2

| Cmpd No. from Table 1 | PKR^{a} AC₅₀(nM)/% activation^{c} |
|---|---|
| 1 | 189/96 |
| 2 | 530/69 |
| 3 | 6000/53 |
| 4 | 1200/80 |
| 5 | 420/47 |
| 6 | 5.4/33 |
| 7 | 44/46 |
| 8 | 320/59 |
| 9 | 120/64 |
| 10 | 80/74 |
| 11 | 88/49 |
| 12 | 180/89 |
| 13 | 1000/55 |
| 14 | 1400/84 |
| 15 | 310/57 |
| 16 | 55/93 |
| 17 | 13/82 |
| 18 | 37/85 |
| 19 | 46/75 |
| 20 | 51/97 |
| 21 | 67/63 |
| 22 | 33/83 |
| 23 | 230/72 |
| 24 | 24/75 |
| 25 | 2500/80 |
| 26 | 73/93 |
| 27 | 5.4/33 |
| 28 | 1200/29 |
| 29 | 110/78 |
| 30 | 18/48 |
| 31 | 14/76 |
| 32 | 210/74 |
| 33 | 9/67 |
| 34 | 830/89 |
| 35 | 3200/28 |
| 36 | 130/95 |
| 37 | 33/80 |
| 38 | 55/53 |
| 39 | 51/63 |
| 40 | 150/73 |
| 41 | 40/81 |
| 42 | 540/74 |
| 43 | 100/84 |
| 44 | 81/94 |
| 45 | 110/80 |
| 46 | 38/74 |
| 47 | 42/85 |
| 48 | 83/74 |
| 49 | 150/79 |
| 50 | 32/64 |
| 51 | 66/75 |
| 52 | 24/37 |
| 53 | 120/73 |
| 54 | 85/71 |
| 55 | 79/90 |
| 56 | 1100/106 |
| 57 | 64/87 |
| 58 | 220/88 |
| 59 | 1200/86 |
| 60 | 180/82 |
| 61 | 11/36 |
| 62 | 210/66 |
| 63 | 63/99 |
| 64 | 120/46 |
| 65 | 1800/62 |
| 66 | 13/41 |
| 67 | 44/58 |
| 68 | 3100/65 |
| 69 | 540/92 |
| 70 | 110/64 |
| 71 | 930/48 |
| 72 | 1800/68 |
| 73 | 190/107 |
| 74 | 2800/36 |
| 75 | 33/47 |
| 76 | 460/74 |
| 77 | 51/93 |
| 78 | 110/88 |
| 79 | 160/53 |
| 80 | 200/106 |
| 81 | 280/64 |
| 82 | 270/85 |
| 83 | 72/83 |
| 84 | 520/94 |
| 85 | 36/67 |
| 86 | 66/90 |
| 87 | 83/87 |
| 88 | 70/68 |
| 89 | 31/91 |
| 90 | 40/97 |
| 91 | 49/61 |
| 92 | 52/72 |
| 93 | 150/100 |
| 94 | 730/87 |
| 95 | 68/87 |
| 96 | 34/100 |
| 97 | 510/92 |
| 98 | 71/60 |
| 99 | 69/69 |
| 100 | 510/106 |
| 101 | 93/77 |
| 102 | 35/71 |
| 103 | 110/73 |
| 104 | 36/80 |
| 105 | 880/59 |
| 106 | 71/125 |
| 107 | 30/107 |
| 108 | 900/74 |
| 109 | 110/96 |
| 110 | 61/83 |
| 111 | 140/105 |
| 112 | 802/126 |
| 113 | 40/116 |
| 114 | 670/61 |
| 115 | 1700/148 |
| 116 | 89/39 |
| 117 | 2600/53 |
| 118 | 47/36 |
| 119 | 1100/31 |
| 120 | 220/51 |
| 121 | 7800/47 |
| 122 | 190/42 |
| 123 | 140/70 |
| 124 | 270/79 |
| 125 | 3800/81 |
| 126 | 67/84 |
| 127 | 47/45 |
| 128 | 24/59 |
| 129 | 430/72 |
| 130 | 300/84 |
| 131 | 78/94 |
| 132 | 490/64 |
| 133 | 160/86 |
| 134 | 170/110 |
| 135 | 86/117 |
| 136 | 47/94 |
| 137 | 180/99 |
| 138 | 44/129 |
| 139 | 61/96 |
| 140 | 25/72 |
| 141 | 140/71 |
| 142 | 250/127 |
| 143 | 140/112 |
| 144 | 27/94 |
| 145 | 51/76 |
| 146 | 290/100 |

^{a}Activity data was measured in PKR biochemical assay.
^{c}The maximal activation level achieved with each compound relative to the activation level achieved by the literature compound AG-348 @ 10 μM.

### Example 2.3

Cell Based Assay for Activation of Pyruvate Kinase M2 in Lung Carcinoma Cell Line

H1299 cells were seeded in 96-well plates at 2,000 cells/well (100 uL).

Treated plates were incubated overnight at 37° C. with 5% CO₂. Compounds were diluted in complete media and added to cells in the presence of 1% DMSO. Cells were incubated with compound for 90 minutes at 37° C. and 5% CO₂ before washing three times with PBS to remove residual compound and then lysed in lysis buffer (Cell Signaling). Cell lysate was analyzed using the NADH coupled assay described below with a reaction mixture of 180 uM NADH, 2 mM ADP, 0.5 units of LDH and 0.5 mM of PEP. PKM2 activity was measured at steady state using a coupled enzyme activation system based on NADH consumption. PKM2 produces pyruvate and the coupled system uses lactate dehydrogenase (LDH) to reduce pyruvate to lactate with the concomitant oxidation of NADH to NAD⁺. Conversion of NADH to NAD⁺ was monitored using a SPECTROstar Nano plate reader (BMG Labtech) at a wavelength of 340 nm and subtraction of background absorbance measured at 750 nm. The change in NADH absorbance after PEP addition was monitored and slope obtained by subtracting baseline at 750 nm followed by least squares fitting to a simple linear regression model. A 10-point curve was generated to calculate the AC₅₀ values by fitting the rates of NADH consumption against increasing concentration of compound.

Efforts have been made to ensure accuracy with respect to numbers used (e.g., amounts, temperature, etc.) but some experimental errors and deviations should be accounted for.

One skilled in the art will recognize many methods and materials similar or equivalent to those described herein, which could be used in the practicing the subject matter described herein. The present disclosure is in no way limited to just the methods and materials described.

Unless defined otherwise, technical and scientific terms used herein have the same meaning as commonly understood by one of ordinary skill in the art to which this subject matter belongs, and are consistent with: Singleton et al (1994) Dictionary of Microbiology and Molecular Biology, 2nd Ed., J. Wiley & Sons, New York, N.Y.; and Janeway, C., Travers, P., Walport, M., Shlomchik (2001) Immunobiology, 5th Ed., Garland Publishing, New York.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

Where a range of values is provided, it is understood that each intervening value, to the tenth of the unit of the lower limit, unless the context clearly dictates otherwise, between the upper and lower limit of the range and any other stated or intervening value in that stated range, is encompassed. The upper and lower limits of these small ranges which may independently be included in the smaller rangers is also encompassed, subject to any specifically excluded limit in the stated range. Where the stated range includes one or both of the limits, ranges excluding either or both of those included limits are also included.

Many modifications and other embodiments set forth herein will come to mind to one skilled in the art to which this subject matter pertains having the benefit of the teachings presented in the foregoing descriptions and the associated drawings. Therefore, it is to be understood that the subject matter is not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed is:

1. A compound of Formula I 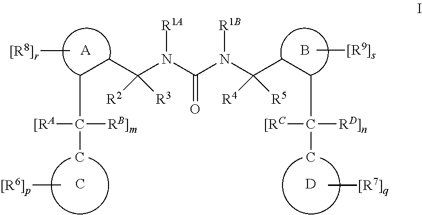
or a pharmaceutically acceptable salt thereof, wherein,
m and n are each independently 0, 1, 2, or 3;
r and s are each independently 0, 1, 2, or 3;
p and q are each independently 0, 1, 2, 3, 4, or 5;
R^{A}, R^{B}, R^{C}, and R^{D}, in each instance, are independently selected from the group consisting of hydrogen, halogen, C₁-C₆ alkyl, hydroxy, hydroxy-C₁-C₆ alkyl, and C₁-C₆ alkoxy;
R⁶ and R⁷, in each instance, are independently selected from the group consisting of halogen, C₁-C₆ alkoxy, C₃-C₇ cycloalkyl, C₁-C₆ alkyl, —C(O)OR¹⁰, hydroxy, hydroxy-C₁-C₆ alkyl, halo-C₁-C₆ alkoxy, halo-C₁-C₆ alkyl, oxo, NR^{E1}R^{G1}, and —C₁-C₆ alkyl-NR^{x}R^{y};
wherein, R¹⁰, R^{E1}, R^{G1}, R^{x}, and R^{y} are each independently hydrogen or C₁-C₆ alkyl;
Ring C and Ring D are each independently selected from the group consisting of C₄-C₇ cycloalkyl, 4- to 10-membered monocyclic or bicyclic fused heterocyclyl, 6- to 10-membered aryl, and 5- to 10-membered heteroaryl, each optionally substituted with R⁶ or R⁷;
wherein said 4- to 10-membered monocyclic or bicyclic fused heterocyclyl or 5- to 10-membered heteroaryl each independently contains 1, 2, 3, or 4 ring heteroatoms selected from N, O, and S;
R^{1A} and R^{1B} are each independently selected from the group consisting of hydrogen, C₁-C₆ alkyl, halogen, halo-C₁-C₆ alkyl, hydroxy-C₁-C₆ alkyl, hydroxy, C₁-C₆ alkoxy, and C₁-C₆ alkoxy-C₁-C₆ alkyl;
or, R^{1A} and R^{1B}, together with the nitrogen atom to which each is attached form a 5- to 7-membered heterocyclyl;
R², R³, R⁴, and R⁵ are each independently hydrogen, halogen, or C₁-C₆ alkyl;
Ring A and Ring B are each independently a 7- to 10-membered spirocyclic heterocyclyl, 5- or 6-membered partially unsaturated monocyclic heterocyclyl, or a 5- or 6-membered heteroaryl, each optionally substituted with R⁸ or R⁹;
wherein, Ring A and Ring B each independently contains 1, 2, 3, or 4 ring heteroatoms selected from N, O, and S;
and,
R⁸ and R⁹, in each instance, are independently selected from the group consisting of halogen, halo-C₁-C₆ alkyl, an oxo group formed by an oxygen double bonded to a ring carbon, C₁-C₆ alkyl, C₃-C₅ cycloalkyl, hydroxy, hydroxy-C₁-C₆ alkyl, C₁-C₃ alkoxy-C₁-C₆ alkyl, NR^{E2}R^{G2}, —C(O)NR⁴⁰R⁵⁰, and —CH₂C(O)OR⁶⁰;
wherein, R^{E2}, R^{G2}, R⁴⁰, R⁵⁰ and R⁶⁰ are each independently hydrogen or C₁-C₆ alkyl.

2. The compound of claim 1, wherein R^{1A} and R^{1B} are each independently hydrogen, —CH₃, —CH₂CH₃, —CH₂CH₂CH₃, or —CH(CH₃)₂.

3. The compound of claim 1 or 2, wherein R^{1A} and R^{1B} are each hydrogen.

4. The compound of any one of claims 1-3, wherein R², R³, R⁴, and R⁵ are each hydrogen.

5. The compound of any one of claims 1-4, wherein Ring A and Ring B each contain at least one N.

6. The compound of any one of claims 1-5, wherein Ring A and Ring B are each independently a 5-membered heteroaryl or 5-membered partially unsaturated monocyclic heterocyclyl.

7. The compound of any one of claims 1-6, wherein Ring A and Ring B are each independently triazolyl or tetrazolyl.

8. The compound of any one of claims 1-7, wherein R⁸ and R⁹, if present, in each instance, are selected from the group consisting of —CH₃, —CH₂CH₃, —CH₂CH₂CH₃, —CH(CH₃)₂, cyclopropyl, —CH₂OH, —CH₂CH₂OH, —CH₂CH₂OCH₃, —CH₂F, and an oxo group formed by an oxygen double bonded to a ring carbon.

9. The compound of any one of claims 1-8, wherein R⁸ and R⁹, if present, in each instance, are —CH₃.

10. The compound of any one of claims 1-9, wherein r is 1.

11. The compound of any one of claims 1-9, wherein r is 0.

12. The compound of any one of claims 1-11, wherein s is 1.

13. The compound of any one of claims 1-11, wherein s is 0.

14. The compound of any one of claims 1-13, wherein Ring C 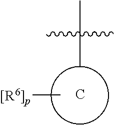
and Ring D 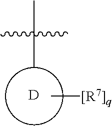
are each independently selected from the group consisting of 5- to 10-membered monocyclic or bicyclic fused heterocyclyl, 6- to 10-membered aryl, and 5- to 10-membered heteroaryl, each optionally substituted with R⁶ or R⁷.

15. The compound of any one of claims 1-14, wherein Ring C 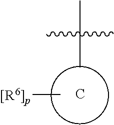
and Ring D 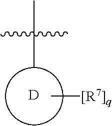
are each independently selected from the group consisting of phenyl, cyclohexyl, tetrahydrofuranyl, quinolinyl, pyrimidinyl, pyridinyl, benzothiazolyl, dihydrobenzofuranyl, quinoxalinyl, benzoimidazolyl, benzodioxolyl, naphthyridinyl, and imidazopyridinyl, each optionally substituted with R⁶ or R⁷.

16. The compound of any one of claims 1-15, wherein Ring C 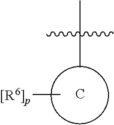
and Ring D 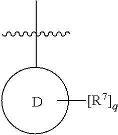
are each optionally substituted phenyl.

17. The compound of any one of claims 1-15, wherein Ring C 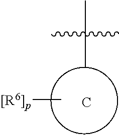
is optionally substituted phenyl and Ring 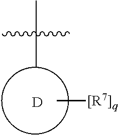
is optionally substituted tetrahydrofuranyl.

18. The compound of any one of claims 1-15, wherein Ring C 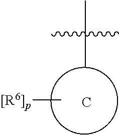
is optionally substituted tetrahydrofuranyl and ring D 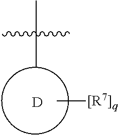
is optionally substituted quinolinyl.

19. The compound of any one of claims 1-15, wherein Ring C 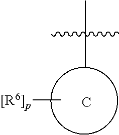
is optionally substituted phenyl and Ring D 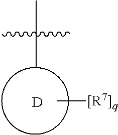
is optionally substituted benzothiazolyl.

20. The compound of any one of claims 1-15, wherein Ring C 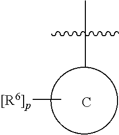
is optionally substituted phenyl and Ring D 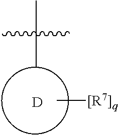
is optionally substituted quinoxalinyl.

21. The compound of any one of claims 1-15, wherein Ring C 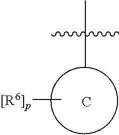
and Ring D 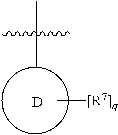
are each optionally substituted quinolinyl.

22. The compound of any one of claims 1-15, wherein Ring C 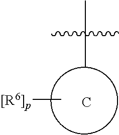
is optionally substituted pyridinyl and Ring D 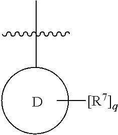
is optionally substituted phenyl.

23. The compound of any one of claims 1-22, wherein R⁶ and R⁷, if present, in each instance are independently selected from the group consisting of chloro, fluoro, —CH₃, hydroxy, —CH₂OH, —CF₃, —OCH₃, —CH₂CH₃, cyclobutyl, and cyclopropyl.

24. The compound of claim 23, wherein m and n are each independently selected from the group consisting of 0, 1, and 2.

25. The compound of claim 23 or 24, wherein at least one of said Ring C 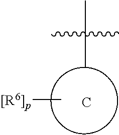
and Ring D 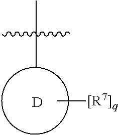
is optionally substituted phenyl.

26. The compound of claim 23 or 24, wherein at least one of said Ring C 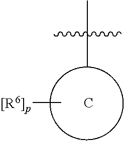
and Ring D 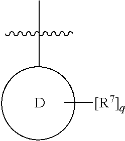
is optionally substituted tetrahydrofuranyl.

27. The compound of claim 23 or 24, wherein at least one of said Ring C 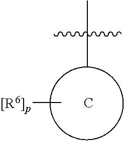
and Ring D 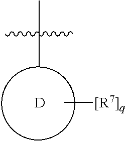
is optionally substituted pyridinyl.

28. The compound of any one of claim 15, 16, 17, 19, 20, 22, or 25, wherein said optionally substituted phenyl is selected from the group consisting of 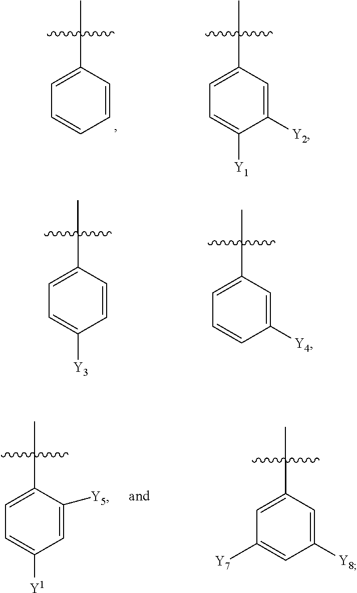
wherein Y₁, Y₂, Y₃, Y₄, Y₅, Y₆, Y₇, and Y₈ are each independently selected from the group consisting of fluoro, chloro, —OCH₃, hydroxy, —CH₂OH, and —CH₃.

29. The compound of claim 28, wherein said optionally substituted phenyl is

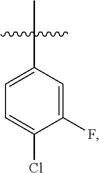 [SMILES: *c1ccc(Cl)c(F)c1]

30. The compound of any one of claim 15, 17, 18, or 26, wherein said optionally substituted tetrahydrofuranyl is selected from the group consisting of

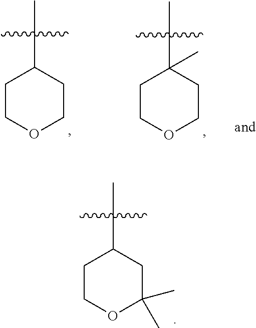

31. The compound of any one of claim 15, 22 or 27, wherein said optionally substituted pyridinyl is selected from the group consisting of 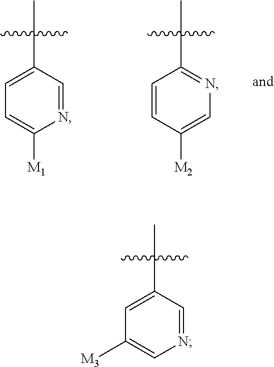
wherein M₁, M₂, and M₃ are each independently selected from the group consisting of hydroxy, cyclopropyl, cyclobutyl, —OCH₃, and —CH₃.

32. The compound of any one of claims 1-31, wherein R^{A}, R^{B}, R^{C}, and R^{D}, if present, are each hydrogen.

33. The compound of any one of claims 1-32, wherein m is 1.

34. The compound of any one of claims 1-32, wherein m is 0.

35. The compound of any one of claims 1-34, wherein n is 0.

36. The compound of claim 5, wherein Ring A is a 7- to 10-membered spirocyclic heterocyclyl, optionally substituted with R⁸, and ring B is 5-membered heteroaryl, optionally substituted with R⁹.

37. The compound of claim 36, wherein R⁸ is oxo.

38. The compound of claim 36 or 37, wherein r is 1.

39. The compound of any one of claims 36-38, wherein ring B is triazolyl, optionally substituted with R⁹.

40. The compound of claim 39, wherein R⁹ is —CH₃.

41. The compound of claim 40, wherein s is 1.

42. The compound of claim 39, wherein s is 0.

43. The compound of any one of claims 36-42, wherein Ring C 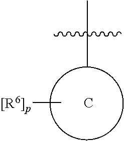
and Ring D 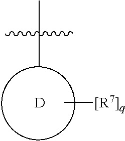
are each independently optionally substituted phenyl or quinolinyl.

44. The compound of claim 43, wherein Ring C 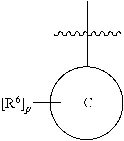
and Ring D 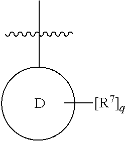
are each optionally substituted phenyl.

45. The compound of claim 43, wherein one of Ring C 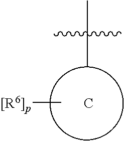
and Ring D 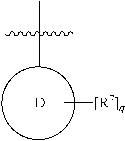
is optionally substituted phenyl and the other is optionally substituted quinolinyl.

46. The compound of any one of claims 43-45, wherein R⁶ and R⁷, if present, in each instance are independently selected from the group consisting of fluoro and chloro.

47. The compound of claim 46, wherein p and q are each independently selected from the group consisting of 0, 1, and 2.

48. The compound of any one of claims 43-47, wherein said optionally substituted phenyl has a structure selected from

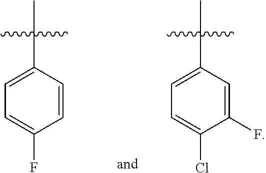 [SMILES: *c1ccc(F)cc1 ; *c1ccc(Cl)c(F)c1]

49. The compound of any one of claim 43 or 45-47, wherein said optionally substituted quinolinyl has the structure

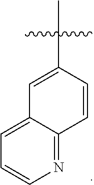 [SMILES: *c1ccc2ncccc2c1]

50. The compound of any one of claims 36-49, wherein R^{A}, R^{B}, R^{C}, and R^{D}, if present, are each hydrogen.

51. The compound of any one of claims 36-50, wherein m is 1.

52. The compound of any one of claims 36-50, wherein m is 0.

53. The compound of any one of claims 36-52, wherein n is 0.

54. The compound of claim 1, selected from a structure in Table 1, or a pharmaceutically acceptable salt thereof.

55. A pharmaceutical composition comprising a compound of any one of claims 1-54 or a pharmaceutically acceptable salt thereof, and a pharmaceutically acceptable excipient.

56. A method of treating a disease or disorder associated with modulation of a pyruvate kinase in a subject, comprising administering to the subject an effective amount of a compound of any one of claims 1-54 or the pharmaceutical composition of claim 55.

57. A method of activating PKR and/or PKM2 in a subject, comprising administering to the subject an effective amount of a compound of any one of claims 1-54 or the pharmaceutical composition of claim 55.

58. A method of treating a subject afflicted with a disease associated with decreased activity of PKR and/or PKM2, comprising administering to the subject an effective amount of a compound of any one of claims 1-54 or the pharmaceutical composition of claim 55.

59. The method of claim 58, wherein the disease is selected from the group consisting of sickle cell disease, thalassemia, hereditary non-spherocytic hemolytic anemia, hemolytic anemia, hereditary spherocytosis, hereditary elliptocytosis, abetalipoproteinemia, paroxysmal nocturnal hemoglobinuria, acquired hemolytic, and anemia of chronic diseases.

60. The method of claim 59, wherein said sickle cell disease is sickle cell anemia.

61. A method for regulating 2,3-diphosphoglycerate levels in blood comprising contacting the blood with an effective amount of a compound of any one of claims 1-54 or the pharmaceutical composition of claim 55.

62. A method for activating mutant pyruvate kinase R (PKR) in red blood cells in a subject in need thereof comprising administering to the subject an effective amount of a compound of any one of claims 1-54 or the pharmaceutical composition of claim 55.

63. A method for activating wild-type pyruvate kinase R (PKR) in red blood cells in a subject in need thereof comprising administering to the subject an effective amount of a compound of any one of claims 1-54 or the pharmaceutical composition of claim 55.
